(19) United States
(12) Patent Application Publication
(10) Pub. No.: US 2024/0117354 A1
(43) Pub. Date: Apr. 11, 2024

(54) COMPOSITIONS AND METHODS FOR MODULATING EXPRESSION OF GENES

(71) Applicant: Versameb AG, Basel (CH)

(72) Inventors: Justin ANTONY SELVARAJ, Weil am Rhein (DE); Klaas Pieter ZUIDEVELD, Riehen (CH); Petra HILLMANN-WÜLLNER, Zurich (CH); Friedrich METZGER, Freiburg (DE)

(21) Appl. No.: 18/542,970

(22) Filed: Dec. 18, 2023

Related U.S. Application Data
(63) Continuation of application No. PCT/IB2022/000344, filed on Jun. 22, 2022.
(60) Provisional application No. 63/213,830, filed on Jun. 23, 2021.

(51) Int. Cl. C12N 15/113; C07K 14/54; C07K 14/55; C07K 14/65

(52) U.S. Cl. CPC C12N 15/1135; C07K 14/5406; C07K 14/5434; C07K 14/55; C07K 14/65; C12N 15/1136; C12N 15/1137; C12Y 207/1103; C12N 2310/14

(57) ABSTRACT

The present invention relates to compositions of recombinant polynucleic acid or RNA constructs comprising a first RNA sequence, a second RNA sequence, and a linker RNA sequence, wherein the linker RNA links the first RNA sequence and the second RNA sequence. In addition, linkers that enhance the cleavage of recombinant RNA constructs are described. Also disclosed herein is the use of the compositions in treating diseases and in modulating expression of two or more genes.

### CROSS-REFERENCE TO RELATED APPLICATIONS

This application is a continuation of International Application No. PCT/IB2022/000344, filed Jun. 22, 2022, which claims the benefit of U.S. Provisional Application No. 63/213,830, filed Jun. 23, 2021, each of which is incorporated by reference herein in its entirety.

### SEQUENCE LISTING

The instant application contains a Sequence Listing which has been submitted electronically in XML format and is hereby incorporated by reference in its entirety. Said XML copy, created on Dec. 5, 2023, is named 57623-708_301_SL.xml and is 300,225 bytes in size.

### BACKGROUND

Numerous human diseases and disorders are caused by combinations of higher and/or lower expression levels of certain proteins compared to the expression levels of these proteins in humans without the disease or disorder. Combinatorial therapies to increase the expression and/or secretion of a target protein and to decrease the expression of one or more other, different target proteins, may have a therapeutic effect. For example, therapies for skin diseases muscular disease, or cancers that effectively and specifically decrease production of one or more target gene products and increase production of others, in parallel, are needed.

### BRIEF SUMMARY

Provided herein are compositions and methods for simultaneously modulating expression of two or more proteins or nucleic acid sequences using one recombinant polynucleic acid or RNA construct.

In some aspects, provided herein, is a composition comprising a recombinant RNA construct comprising a first RNA sequence, a second RNA sequence, and a linker RNA sequence, wherein: (i) the first RNA sequence is a first small interfering RNA (siRNA) sequence; (ii) the second RNA sequence is a second siRNA sequence or a first messenger RNA (mRNA) sequence encoding a gene of interest (GOI); and (iii) the linker RNA sequence links the first RNA sequence and the second RNA sequence, wherein the linker RNA sequence has a structure selected from the group consisting of: Formula (I): XₘCAACAAXₙ, wherein X is any nucleotide, m is an integer from 1 to 12, and n is an integer from 0 to 4 (SEQ ID NO: 151); and Formula (II): XₚTCCCXᵣ, wherein X is any nucleotide, p is an integer from 0 to 17, and r is an integer from 0 to 13 (SEQ ID NO: 152).

In some aspects, provided herein, is a composition comprising a recombinant RNA construct comprising a first RNA sequence, a second RNA sequence, and a linker RNA sequence, wherein: (i) the first RNA sequence is a first small interfering RNA (siRNA) sequence; (ii) the second RNA sequence is a second siRNA sequence or a first messenger RNA (mRNA) sequence encoding a gene of interest (GOI); and (iii) the linker RNA sequence links the first RNA sequence and the second RNA sequence, wherein the linker RNA sequence comprises or consists of ACAACAA (SEQ ID NO: 23).

In some aspects, provided herein, is a composition comprising a recombinant RNA construct comprising a first RNA sequence, a second RNA sequence, and a linker RNA sequence, wherein: (i) the first RNA sequence is a first small interfering (siRNA) sequence; (ii) the second RNA sequence is a second siRNA sequence or a first messenger (mRNA) sequence encoding a gene of interest (GOI); and (iii) the linker RNA sequence links the first RNA sequence and the second RNA sequence, wherein (a) the linker RNA sequence is not TTTATCTTAGAGGCATATCCCTACGTACCAACAA (SEQ ID NO: 22) or ATAGTGAGTCGTATTAACGTACCAACAA (SEQ ID NO: 21); or (b) the linker RNA sequence does not form a secondary structure according to RNAfold WebServer.

In some aspects, provided herein, are compositions described herein for use in modulating the expression of two or more genes in a cell. In some aspects, provided herein, is a pharmaceutical composition comprising a therapeutically effective amount of any one of the compositions described herein and a pharmaceutically acceptable excipient. In some aspects, provided herein, is a cell comprising any one of the compositions described herein. In some aspects, provided herein, is a vector comprising a recombinant polynucleic acid construct encoding any one of the compositions described herein.

In some aspects, provided herein, is a method of producing an siRNA and an mRNA from a single RNA transcript in a cell, comprising introducing into the cell any one of the compositions described herein or the vectors described herein. In some aspects, provided herein, is a method of modulating protein expression comprising introducing any one of the compositions described herein or any one of the vectors described herein into a cell, wherein the expression of a protein encoded by the target RNA is decreased. In some aspects, provided herein, is a method of modulating protein expression comprising introducing any one of the compositions described herein or any one of the vectors described herein into a cell, wherein the expression of a protein encoded by a gene of interest (GOI) is increased. In some aspects, provided herein, is a method of modulating protein expression comprising introducing any one of the compositions described herein or any one of the vectors described herein into a cell, wherein the expression of a protein encoded by the target RNA is decreased, and wherein the expression of a protein encoded by a gene of interest (GOI) is increased.

In some aspects, provided herein, is a method of treating a disease or condition comprising administering to a subject in need thereof any one of the compositions described herein or any one of the pharmaceutical compositions described herein.

In some aspects, provided herein, is a composition comprising a recombinant RNA construct comprising a first RNA sequence, a first linker RNA sequence, a second RNA sequence, and a second linker RNA sequence, wherein: (i) the first RNA sequence is a messenger RNA (mRNA) encoding Interleukin 4 (IL-4); (ii) the second RNA sequence comprises two or more small interfering RNAs (siRNAs) capable of binding to a Tumor Necrosis Factor alpha (TNF-α) mRNA; (iii) the first linker RNA sequence is present between the first RNA sequence and the second RNA sequence, and (iv) the second linker RNA sequence links each of the two or more siRNAs and comprises a sequence according to SEQ ID NO: 23.

In some aspects, provided herein, is a composition comprising a recombinant RNA construct comprising a first RNA sequence, a first linker RNA sequence, a second RNA sequence, and a second linker RNA sequence, wherein: (i) the first RNA sequence is a messenger RNA (mRNA) encoding Insulin-like Growth Factor 1 (IGF1); (ii) the second RNA sequence comprises two or more small interfering RNAs (siRNAs) capable of binding to a Activin Receptor-like Kinase 2 (ALK2) mRNA; (iii) the first linker RNA sequence is present between the first RNA sequence and the second RNA sequence, and (iii) the second linker RNA sequence links each of the two or more siRNAs and comprises a sequence according to SEQ ID NO: 23.

In some aspects, provided herein, is a composition comprising a recombinant RNA construct comprising a first RNA sequence, a first linker RNA sequence, a second RNA sequence, and a second linker RNA sequence, wherein: (i) the first RNA sequence is a messenger RNA (mRNA) encoding Interleukin 2 (IL-2); (ii) the second RNA sequence comprises two or more small interfering RNAs (siRNAs) capable of binding to a Vascular Endothelial Growth Factor A (VEGFA) mRNA; (iii) the first linker RNA sequence is present between the first RNA sequence and the second RNA sequence; and (iv) the second linker RNA sequence links each of the two or more siRNAs and comprises a sequence selected from the group consisting of SEQ ID NOs: 23 and 67-70.

In some aspects, provided herein, is a composition comprising a recombinant RNA construct comprising a first RNA sequence, a first linker RNA sequence, a second RNA sequence, and a second linker RNA sequence, wherein: (i) the first RNA sequence is a messenger RNA (mRNA) encoding Interleukin 12 (IL-12); (ii) the second RNA sequence comprises two or more small interfering RNAs (siRNAs) capable of binding to an mRNA of Cellular Myelocytomatosis (c-Myc), Kirsten Rat Sarcoma (KRAS), Protein kinase B-1 (Akt1), Akt2, and/or Akt3; (iii) the first linker RNA sequence is present between the first RNA sequence and the second RNA sequence; and (iv) the second linker RNA sequence links each of the two or more siRNAs and comprises a sequence according to SEQ ID NO: 23.

In some aspects, provided herein, is a composition comprising a recombinant polynucleic acid construct comprising a nucleic acid sequence selected from the group consisting of SEQ ID NOs: 1-18 and 76-108.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic representation of construct design. A polynucleic acid (e.g., DNA) construct may comprise a T7 promoter sequence upstream of the gene of interest sequence for T7 RNA polymerase binding and successful in vitro transcription of both the gene of interest (e.g., IGF-1 or IL-4) and siRNA in a single RNA transcript. Signal peptide of the gene of interest is highlighted in a grey box. Linkers to connect mRNA to siRNA or siRNA to siRNA are indicated with boxes with horizontal stripes or boxes with checkered stripes, respectively. T7: T7 promoter, siRNA: small interfering RNA.

FIGS. 2A-2B show plots for interference of TNF-α expression and overexpression of IL-4 protein level measured by ELISA in effect to A1-linker or A2 linker in THP-1 cells. FIG. 2A shows RNA interference of Compound 1 (Cpd.1) and compound 2 (Cpd.2) which comprises 3×TNF-α-targeting siRNA at 3′ with A1-linker and A2-linker respectively in an endogenous TNF-α expression in stimulated THP-1 cells. THP-1 cells were stimulated with E. coli-derived lipopolysaccharide (10 μg/mL) and R848 (1 μg/mL) and followed by the transfection (600 ng/well) of Cpd.1, Cpd.2 and scrambled siRNA (sc-siRNA). Untransfected samples were used as control and set to 100% and percent of TNF-α knock down was calculated. Data represent means±standard error of the mean of four replicates. Significance (***, p<0.001) for both Cpd.1 and Cpd.2 was assessed by one way ANOVA followed by Dunnet's multiple comparing tests related to control. FIG. 2B shows IL-4 expression of Cpd.1 and Cpd.2 in the same cell (THP-1) culture supernatant as in FIG. 1A. Data represent means±standard error of the mean of four replicates. Significance (**, <0.01) was assessed by Student's t-test of Cpd.1 and Cpd. 2 for IL-4 expression.

FIGS. 3A-3B show plots for interference of TNF-α expression and overexpression of IL-4 protein level measured by ELISA in effect to A1-linker or A2 linker in HEK293 cells. FIG. 3A shows RNA interference of Cpd.1 and Cpd.2 which comprises 3×TNF-α-targeting siRNA at 3′ with A1-linker and A2-linker respectively in TNF-α over expression model in HEK293 cells. HEK293 cells were co-transfected with TNF-α mRNA (600 ng/well) and either Cpd.1 or Cpd.2 (900 ng/well). TNF-α levels are calculated by ELISA. Untransfected samples were used as control and set to 100% and percent of TNF-α knock down was calculated. Data represent means±standard error of the mean of four replicates. Significance (**, p<0.01) for both Cpd.1 and Cpd.2 was assessed by one way ANOVA followed by Dunnet's multiple comparing tests related to control. FIG. 3B shows IL-4 expression of Cpd.1 and Cpd.2 in the same cell (HEK293) culture supernatant as in FIG. 2A. Data represent means±standard error of the mean of 4 replicates. Significance (*, <0.05) was assessed by Student's t-test of Cpd.1 and Cpd.2 for IL-4 expression.

FIGS. 4A-4D show plots for IGF-1 protein level measured by ELISA and interference of ALK2 expression assessed by qPCR in effect to A1-linker or A2 linker in A549 cells and HEK293 cells. FIG. 4A shows IGF-1 expression of Cpd.3 and Cpd.4 which comprises 3×ALK2-targeting siRNA at 3′ with A1-linker and A2-linker respectively in lung epithelial cells (A549 cells) which endogenously express ALK-2. A549 cells were transfected with either Cpd.3 or Cpd.4 RNA in increasing nM concentrations (0.65, 1.33, 2.7, 5.4 and 10.8). Data represent means±standard error of the mean of 4 replicates. Significance (***, p<0.001) between Cpd.3 and Cpd.4 was assessed by two-way ANOVA followed by Tukey's multiple comparing tests. FIG. 4B shows RNA interference of Cpd.3 and Cpd.4 in ALK2 expression in the same cell (A549) culture lysate as in FIG. 3A. ALK2 mRNA levels were calculated by qPCR through relative quantification (normalized by 18s gene). Untransfected samples were used as control and set to 100% and percent of ALK2 knock down was calculated. No significant differences were noted between Cpd.3 and Cpd.4 in ALK2 RNA interference. FIG. 4C and FIG. 4D show IGF-1 expression of Cpd.3 and Cpd.4 in HEK293 cells and A549 cells, respectively (1.33 nM/well). Data represent means±standard error of the mean of 12 replicates. Significance (*, <0.05 in HEK293; ***, <0.001 in A549 cells) was assessed by Student's t-test of Cpd.3 and Cpd.4 for IGF-1 expression.

FIGS. 5A-5C show plots for IGF-1 protein level measured by ELISA and interference of exogenously expressed Turbo GFP assessed by microscopy in effect to A1-linker or A2 linker in human tongue cell carcinoma cells (SCC-4). FIG. 5A shows IGF-1 expression of Cpd.5 to Cpd.9 which comprises either 1× or 2× Turbo GFP-targeting siRNA at 3′ with A1-linker and A2-linker. SCC4 cells were co-transfected with 0.3 μg of Turbo GFP encoding and 30 nM one of the Cpd.5 to Cpd.9. 24 hours post transfection IGF-1 levels were measured in cell culture supernatant by a specific ELISA. Data represent means±standard error of the mean of 4 replicates. Significance (***, p<0.001) for Cpd.5 was assessed by one way ANOVA followed by Dunnet's multiple comparing test compared to Cpd.6. Significance (***, p<0.001) for both Cpd.7 and Cpd.8 was assessed by one way ANOVA followed by Dunnet's multiple comparing test compared to Cpd.9. FIG. 5B shows interference of exogenously expressed Turbo GFP (300 ng/well) by microscopy in effect to Cpd.5 to Cpd.9 co-transfection. 30 nM of scrambled siRNA (sc-siRNA) used as a negative control. FIG. 5C shows the representative microscopical images of control, sc.siRNA, Cpd.5 and Cpd.6 with Turbo GFP expression. The Turbo GFP positive cells converted into greyscale using ImageJ analysis tool (representative figure from one well).

FIG. 6A is a plot showing dose-dependent secretion levels of IL-2 induced by compounds comprising different linkers (Cpd.10-Cpd.15; Linkers A1, A2, B-E) in A549 cells. IL-2 levels in the cell culture supernatant were measured by ELISA, 24 hours after transfection. The X-axis indicates concentrations of each compound (1, 3, 10 and 30 nM/well) used for transfection into A549 cells. The Y-axis shows measurement for IL-2 protein level (pg/mL) in the cell culture supernatant. Data represent means±standard error of the mean of 3 replicates.

FIG. 6B is a plot showing dose-dependent downregulation of endogenously expressed VEGFA induced by compounds comprising different linkers (Cpd.10-Cpd.15; Linkers A1, A2, B-E) in A549 cells. The X-axis indicates concentrations of each compound (1, 3, 10 and 30 nM/well) used for transfection into A549 cells. VEGFA protein levels in the supernatant of untransfected cells were set to 100%. The Y-axis indicates downregulation of VEGFA level normalized to untransfected samples (basal level). Data represent means±standard error of the mean of 3 replicates.

FIG. 6C is a table for comparison of IL-2 levels and VEGFA levels in A549 cells transfected with Cpd.10-Cpd.15 (Linkers A1, A2, B-E) with concentration of 10 nM. A549 cells transfected with Cpd.11 (A2 linker) and Cpd.14 (E linker) showed 1.5 to 2.5-fold higher IL-2 levels compared to A549 cells transfected with other compounds. A549 cells transfected with different compounds exhibited equivalent VEGFA downregulation.

FIG. 7A is a plot showing dose-dependent secretion levels of IL-2 induced by compounds comprising different linkers (Cpd.10-Cpd.15; Linkers A1, A2, B-E) in SCC-4 cells. IL-2 levels in the cell culture supernatant were measured by ELISA, 24 hours after transfection. The X-axis indicates concentrations of each compound (1, 3, 10 and 30 nM/well) used for transfection into SCC-4 cells. The Y-axis shows measurement for IL-2 protein level (pg/mL) in the cell culture supernatant. Data represent means±standard error of the mean of 3 replicates.

FIG. 7B is a plot showing dose-dependent downregulation of endogenously expressed VEGFA induced by compounds comprising different linkers (Cpd.10-Cpd.15; Linkers A1, A2, B-E) in SCC-4 cells. The X-axis indicates concentrations of each compound (1, 3, 10 and 30 nM/well) used for transfection into SCC-4 cells. VEGFA levels from untransfected cells were set to 100%. The Y-axis indicates downregulation of VEGFA level normalized to untransfected samples (basal level). Data represent means±standard error of the mean of 3 replicates.

FIG. 7C is a table for comparison of IL-2 levels and VEGFA levels in SCC-4 cells transfected with Cpd.10-Cpd.15 (Linkers A1, A2, B-E) with concentration of 10 nM. SCC-4 cells transfected with Cpd.11 (A2 linker) and Cpd.14 (E linker) showed 1.2 to 2.5-fold higher IL-2 levels compared to SCC-4 cells transfected with other compounds. SCC-4 cells transfected with Cpd.12 exhibited improved VEGFA downregulation compared to SCC-4 cells transfected with other compounds.

FIGS. 8A-8B show plots for IGF-1 protein level measured by ELISA in effect to A1-linker or A2 linker in human primary muscle cells (HSMM). FIG. 8A shows IGF-1 expression from Cpd.3 and Cpd.4, which comprise 3×ALK2-targeting siRNA at 3′ with A1-linker and A2-linker, respectively, in HSMM cells which are cultivated in growth medium. FIG. 8B shows IGF-1 expression from Cpd.3 and Cpd.4 in HSMM cells which are cultivated in differentiation medium. HSMM cells were transfected with either Cpd.3 or Cpd.4 RNA in increasing nM concentrations (0.1, 0.3, 1, 3, 10 and 30 nM). Data represent means±standard error of the mean of 4 replicates. Significance (***, p<0.001) between Cpd.3 and Cpd.4 was assessed by two-way ANOVA followed by Tukey's multiple comparing tests.

FIG. 9A shows the time-course secretion of IL-12 induced by Cpd.16 and Cpd.17 in A-172 cells up to 24 hours. IL-12 levels in the cell culture supernatant were measured by ELISA, from 0 to 24 hours after transfection (10 nM/well). The X-axis indicates time (hours) after transfection and the Y-axis shows measurement for IL-12 protein level (pg/mL) in cell culture supernatant. Data represent means±standard error of the mean of 3 replicates.

FIG. 9B is a plot showing dose-dependent activation of the STAT4 pathway in HEK-Blue™ IL-12 reporter cells induced by rhIL-12 or IL-12 (0.001 ng to 300 ng) derived from supernatant of human embryonic kidney (HEK293) cells that had been transfected with Cpd.16 or Cpd.17 (0.3 μg/well) and quantified by ELISA. The X-axis indicates different concentrations of Cpd.16- or Cpd.17-derived IL-12 or rhIL-12. The Y-axis indicates IL-12 signaling activation normalized to rhIL-12 (lowest SEAP values of rhIL-12 set to 0 and highest SEAP values of rhIL-12 set to 100%). Data represent means±standard error of the mean of 3 replicates per dose.

FIGS. 9C-9D are plots showing time-dependent downregulation of KRAS and pan-Akt (Akt1, Akt2, and Akt3) levels in A172 cells transfected with Cpd.16 (FIG. 9C) or Cpd.17 (FIG. 9D). RNA levels of KRAS and pan-Akt were measured from cell lysate by qPCR in technical triplicates, up to 24 hours after transfection. The X-axis indicates time (hours) after transfection. The Y-axis indicates downregulation of KRAS and pan-Akt levels normalized to untransfected samples (basal level). Data represent means±standard error of the mean of 3 replicates.

FIGS. 9E-9F are plots showing time-dependent downregulation of c-Myc levels in A172 cells transfected with Cpd.16 (FIG. 9E) or Cpd.17 (FIG. 9F). RNA levels of c-Myc were measured from cell lysate by qPCR in technical triplicates, 12-24 hours after transfection. The X-axis indicates time (hours) after transfection. The Y-axis indicates downregulation of c-Myc levels normalized to untransfected samples (basal level). Data represent means±standard error of the mean of 3 replicates.

### DETAILED DESCRIPTION

Provided herein are compositions and methods for modulating expression of two or more genes, comprising recombinant polynucleic acid or RNA constructs comprising at least one nucleic acid sequence encoding a gene of interest and/or at least one nucleic acid sequence encoding or comprising a genetic element that modulates expression of a target RNA. Recombinant polynucleic acid or RNA construct compositions provided herein may further comprise one or more linkers. In one instance, recombinant polynucleic acid or RNA constructs may comprise nucleic acid sequences encoding or comprising one or more genetic elements that modulate expression of one or more target RNAs and one or more linkers, wherein a linker may be present between each of one or more genetic elements that modulate expression of one or more target RNAs. In another instance, recombinant polynucleic acid or RNA constructs may comprise nucleic acid sequences encoding one or more genes of interest and one or more linkers, wherein a linker may be present between each of one or more genes of interest. In some instances, recombinant polynucleic acid or RNA constructs may comprise nucleic acid sequences encoding one or more genes of interest, nucleic acid sequences encoding or comprising one or more genetic elements that modulate expression of one or more target RNAs, and one or more linkers, wherein a linker may be present between nucleic acid sequences encoding one or more genes of interest and nucleic acid sequences encoding or comprising one or more genetic elements that modulate expression of one or more target RNAs, between each of one or more genetic elements that modulate expression of one or more target RNAs, and/or between each of one or more genes of interest.

Also provided herein are vectors comprising recombinant polynucleic acid constructs described herein or encoding recombinant RNA constructs described herein. Provided herein are cells comprising recombinant polynucleic acid or RNA construct composition or vectors described herein. Recombinant polynucleic acid or RNA construct compositions described herein can be formulated into pharmaceutical compositions. Further provided herein are compositions and methods to modulate expression of two or more genes in parallel.

Provided herein are compositions and methods for treating a disease or condition comprising administering to a subject in need thereof compositions or pharmaceutical compositions described herein. Recombinant polynucleic acid or RNA construct compositions provided herein may comprise a first RNA sequence, a second RNA sequence, and a linker RNA sequence, wherein a linker of the linker RNA sequence links the first RNA sequence and the second RNA sequence. In one example, the first RNA sequence or the second RNA sequence may comprise one or more messenger RNAs (mRNAs), and can increase the level of proteins encoded by mRNAs. In another example, the first RNA sequence or the second RNA sequence may be a genetic element that modulates expression of a target RNA. For example, the first RNA sequence or the second RNA sequence may comprise small interfering RNAs (siRNAs) capable of binding to one or more target RNAs, and can downregulate the levels of protein encoded by target RNAs. For example, mRNAs and target RNAs may be of genes associated diseases and conditions described herein.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### Definitions

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “embodiments,” “certain embodiments,” “preferred embodiments,” “specific embodiments,” “some embodiments,” “an embodiment,” “one embodiment” or “other embodiments” mean that a particular feature, structure, or characteristic described in connection with the embodiments is included in at least some embodiments, but not necessarily all embodiments, of the present disclosures. To facilitate an understanding of the present disclosure, a number of terms and phrases are defined below.

The term “RNA” as used herein includes RNA which encodes an amino acid sequence (e.g., mRNA, etc.) as well as RNA which does not encode an amino acid sequence (e.g., siRNA, shRNA, miRNA etc.). The RNA as used herein may be a coding RNA, i.e., an RNA which encodes an amino acid sequence. Such RNA molecules are also referred to as mRNA (messenger RNA) and are single-stranded RNA molecules. The RNA as used herein may be a non-coding RNA, i.e., an RNA which does not encode an amino acid sequence or is not translated into a protein. A non-coding RNA can include, but is not limited to, a small interfering RNA (siRNA), a short or small harpin RNA (shRNA), a microRNA (miRNA), a piwi-interacting RNA (piRNA), and a long non-coding RNA (lncRNA). siRNAs as used herein may comprise a double-stranded RNA (dsRNA) region, a hairpin structure, a loop structure, or any combinations thereof. In some embodiments, siRNAs may comprise at least one shRNA, at least one dsRNA region, or at least one loop structure. In some embodiments, siRNAs may be processed from a dsRNA or an shRNA. In some embodiments, siRNAs may be processed or cleaved by an endogenous protein, such as DICER, from an shRNA. In some embodiments, a hairpin structure or a loop structure may be cleaved or removed from an siRNA. For example, a hairpin structure or a loop structure of an shRNA may be cleaved or removed. In some embodiments, RNAs described herein may be made by synthetic, chemical, or enzymatic methodology known to one of ordinary skill in the art, made by recombinant technology known to one of ordinary skill in the art, or isolated from natural sources, or made by any combinations thereof. The RNA may comprise modified or unmodified nucleotides or mixtures thereof, e.g., the RNA may optionally comprise chemical and naturally occurring nucleoside modifications known in the art (e.g., N¹-Methylpseudouridine also referred herein as methylpseudouridine).

The terms “nucleic acid sequence,” “polynucleic acid sequence,” and “nucleotide sequence” are used herein interchangeably and have the identical meaning herein and refer to DNA or RNA. In some embodiments, a nucleic acid sequence is a polymer comprising or consisting of nucleotide monomers, which are covalently linked to each other by phosphodiester-bonds of a sugar/phosphate-backbone. The terms “nucleic acid sequence,” “polynucleic acid sequence,” and “nucleotide sequence” may encompass unmodified nucleic acid sequences, i.e., comprise unmodified nucleotides, or natural nucleotides. The terms “nucleic acid sequence,” “polynucleic acid sequence,” and “nucleotide sequence” may also encompass modified nucleic acid sequences, such as base-modified, sugar-modified or backbone-modified etc., DNA or RNA.

The terms “natural nucleotide” and “canonical nucleotide” are used herein interchangeably and have the identical meaning herein and refer to the naturally occurring nucleotide bases adenine (A), guanine (G), cytosine (C), uracil (U), thymine (T).

The term “unmodified nucleotide” is used herein to refer to natural nucleotides which are not naturally modified e.g., which are not epigenetically or post-transcriptionally modified in vivo. Preferably the term “unmodified nucleotides” is used herein to refer to natural nucleotides which are not naturally modified e.g., which are not epigenetically or post-transcriptionally modified in vivo and which are not chemically modified e.g., which are not chemically modified in vitro.

The term “modified nucleotide” is used herein to refer to naturally modified nucleotides such as epigenetically or post-transcriptionally modified nucleotides and to chemically modified nucleotides e.g., nucleotides which are chemically modified in vitro.

### Recombinant RNA Constructs

Provided herein are compositions comprising recombinant RNA constructs comprising at least one nucleic acid sequence encoding a gene of interest and/or at least one nucleic acid sequence comprising a genetic element that modulates expression of a target RNA. Recombinant RNA construct compositions provided herein may further comprise one or more linkers. In one instance, recombinant RNA constructs may comprise nucleic acid sequences comprising one or more genetic elements that modulate expression of one or more target RNAs and one or more linkers, wherein a linker may be present between each of one or more genetic elements that modulate expression of one or more target RNAs. In another instance, recombinant RNA constructs may comprise nucleic acid sequences encoding one or more genes of interest and one or more linkers, wherein a linker may be present between each of one or more genes of interest. In some instances, recombinant RNA constructs may comprise nucleic acid sequences encoding one or more genes of interest, nucleic acid sequences comprising one or more genetic elements that modulate expression of one or more target RNAs, and one or more linkers, wherein a linker may be present between nucleic acid sequences encoding one or more genes of interest and nucleic acid sequences comprising one or more genetic elements that modulate expression of one or more target RNAs; between each of one or more genetic elements that modulate expression of one or more target RNAs; and/or between each of one or more genes of interest.

Provided herein are compositions for modulating expression of two or more genes comprising recombinant RNA constructs comprising at least one nucleic acid sequence encoding a gene of interest and/or at least one nucleic acid sequence comprising a genetic element that modulates expression of a target RNA. Further provided herein are compositions for treating a disease or condition comprising recombinant RNA constructs comprising at least one nucleic acid sequence encoding a gene of interest and/or at least one nucleic acid sequence comprising a genetic element that modulates expression of a target RNA. Recombinant RNA construct compositions provided herein may comprise a first RNA sequence, a second RNA sequence, and a linker sequence, wherein the linker sequence links the first RNA sequence and the second RNA sequence. In one example, the first RNA sequence or the second RNA sequence may comprise one or more messenger RNAs (mRNAs), and can increase the level of proteins encoded by mRNAs. In another example, the first RNA sequence or the second RNA sequence may be a genetic element that modulates expression of a target RNA. In some embodiments, the genetic element that modulates expression of a target RNA may be a small interfering RNA (siRNA) capable of binding to one or more target RNAs. For example, the first RNA sequence or the second RNA sequence may comprise siRNAs capable of binding to one or more target RNAs, and can downregulate the levels of protein encoded by target RNAs. In some instances, the genetic element that modulates expression of a target RNA does not inhibit the expression of the gene of interest. In some instances, mRNAs and target RNAs may be of genes associated diseases and conditions described herein. Also provided herein are compositions and methods to modulate expression of two or more genes in parallel using a single RNA transcript.

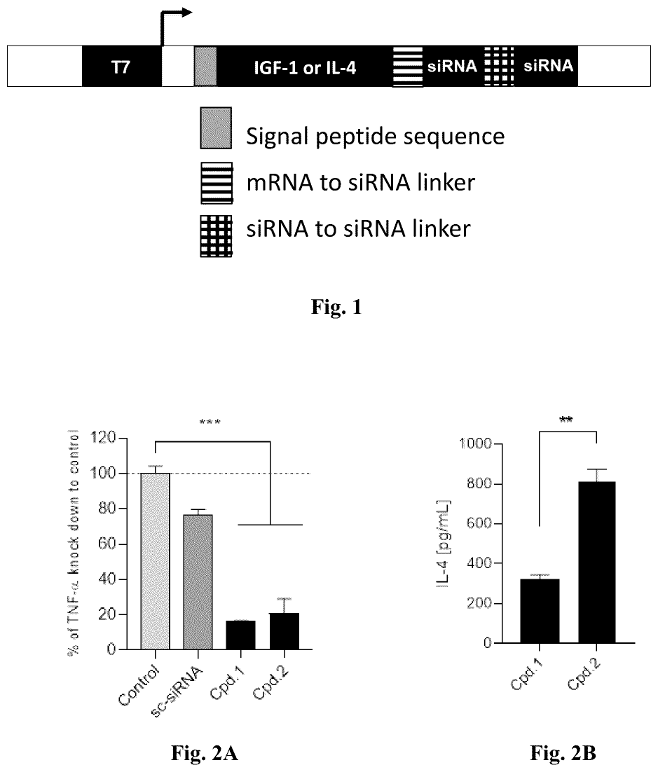

Further provided herein are recombinant polynucleic acid or RNA constructs comprising a gene of interest and a genetic element that reduces expression of another gene such as siRNA, wherein the gene of interest and the genetic element that reduces expression of another gene such as siRNA may be present in a sequential manner from the 5′ to 3′ direction, as illustrated in FIG. 1, or from 3′ to 5′ direction. In one example, the gene of interest (e.g., IGF-1, IL-2, IL-12, or IL-4) can be present 5′ to or upstream of the genetic element that reduces expression of another gene such as siRNA, and the gene of interest can be linked to siRNA by a linker (mRNA to siRNA/shRNA linker, can be also referred as a “spacer”), as illustrated in FIG. 1. In another example, the gene of interest may be present 3′ to or downstream of the genetic element that reduces expression of another gene such as siRNA, and siRNA can be linked to the gene of interest by a linker (siRNA/shRNA to mRNA linker, can be also referred as a “spacer”). Recombinant polynucleic acid or RNA constructs provided herein may comprise more than one species of siRNAs and each of more than one species of siRNAs can be linked by a linker (siRNA to siRNA linker or shRNA to shRNA linker). In some embodiments, the sequence of mRNA to siRNA/shRNA (or siRNA/shRNA to mRNA) linker and the sequence of siRNA to siRNA (or shRNA to shRNA) linker may be different. In some embodiments, the sequence of mRNA to siRNA/shRNA (or siRNA/shRNA to mRNA) linker and the sequence of siRNA to siRNA (or shRNA to shRNA) linker may be the same. Recombinant polynucleic acid or RNA constructs provided herein may comprise more than one gene of interest and each of more than one gene of interest can be linked by a linker (mRNA to mRNA linker). In some instances, a gene of interest may comprise a signal peptide sequence at the N-terminus as shown in FIG. 1. In some instances, a gene of interest may comprise unmodified (WT) signal peptide sequence or modified signal peptide sequence. Recombinant polynucleic acid constructs (e.g., DNA constructs) provided herein may also comprise a promoter sequence for RNA polymerase binding. For example, DNA constructs may comprise a promoter sequence for DNA-dependent RNA polymerase binding to express RNA constructs described herein. As an example, T7 promoter for T7 RNA polymerase binding is shown in FIG. 1. In some embodiments, RNA constructs described herein may not comprise a promoter sequence.

A recombinant polynucleic acid or a recombinant RNA can refer to a polynucleic acid or RNA that is not naturally occurring and is synthesized or manipulated in vitro. A recombinant polynucleic acid or RNA can be synthesized in a laboratory and can be prepared by using recombinant DNA or RNA technology by using enzymatic modification of DNA or RNA, such as enzymatic restriction digestion, ligation, cloning, and/or in vitro transcription. A recombinant polynucleic acid can be transcribed in vitro to produce a messenger RNA (mRNA) and recombinant mRNAs can be isolated, purified, and used for transfection into a cell. A recombinant polynucleic acid or RNA used herein can encode a protein, polypeptide, a target motif, a signal peptide, and/or a non-coding RNA such as small interfering RNA (siRNA). In some embodiments, under suitable conditions, a recombinant polynucleic acid or RNA can be incorporated into a cell and expressed within the cell.

Provided herein are recombinant RNA constructs comprising one or more nucleic acid sequence comprising an siRNA capable of binding to a target RNA and one or more nucleic acid sequence encoding a gene of interest, wherein the siRNA capable of binding to a target RNA is not a part of an intron sequence encoded by the gene of interest. In some instances, the gene of interest is expressed without RNA splicing. In some instances, the siRNA capable of binding to a target RNA is not encoded by or comprised of an intron sequence of the gene of interest. In some instances, the siRNA capable of binding to a target RNA binds to an exon of a target RNA. In some instances, the siRNA capable of binding to a target RNA specifically binds to one target RNA.

Recombinant RNA constructs provided herein may comprise multiple copies of a gene of interest, wherein each of the multiple copies of a gene of interest encodes the same protein. Also provided herein are compositions comprising recombinant RNA constructs comprising multiple genes of interest, wherein each of the multiple genes of interest encodes a different protein. Recombinant RNA constructs provided herein may comprise multiple species of siRNAs, wherein each of the multiple species of siRNAs is capable of binding to the same target RNA. In some embodiments, each of the multiple species of siRNAs may bind to the same region of the same target RNA. In some embodiments, each of the multiple species of siRNAs may bind to a different region of the same target RNA. In some embodiments, some of the multiple species of siRNAs may bind to the same target RNA and some of the multiple species of siRNAs may bind to a different region of the same target RNA. Also provided herein are recombinant RNA constructs comprising multiple species of siRNAs, wherein each of the multiple species of siRNAs is capable of binding to a different target RNA. In some embodiments, the target RNA is a noncoding RNA. In some embodiments, the target RNA is a messenger (mRNA).

Provided herein are compositions comprising recombinant RNA constructs comprising a first RNA sequence, a second RNA sequence, and a linker sequence, wherein the first RNA sequence and/or the second RNA sequence may encode a gene of interest or a genetic element that modulates expression of a target RNA. In one example, the first RNA sequence or the second RNA sequence may be an mRNA encoding a gene of interest. In another example, the first RNA sequence or the second RNA sequence may be a genetic element that reduces expression of a target RNA, such as a small interfering RNA (siRNA) capable of binding to a target RNA.

Recombinant RNA constructs provided herein may comprise more than one nucleic acid sequences encoding a gene of interest. For example, recombinant RNA constructs may comprise 2, 3, 4, 5, 6, 7, 8, 9, 10, 11, 12, 13, 14, 15, or more nucleic acid sequences encoding a gene of interest. In some instances, each of the two or more nucleic acid sequences may encode the same gene of interest, wherein the mRNA encoded by the same gene of interest is different from the siRNA target mRNA. In some instances, each of the two or more nucleic acid sequences may encode a different gene of interest, wherein the mRNA encoded by the different gene of interest is not a target of siRNA comprised in the same RNA construct. In some instances, recombinant RNA constructs may comprise three or more nucleic acid sequences encoding a gene of interest, wherein each of the three or more nucleic acid sequences may encode the same gene of interest or a different gene of interest, and wherein mRNAs encoded by the same or the different gene of interest are not a target of siRNA comprised in the same RNA construct. For example, recombinant RNA constructs may comprise four nucleic acid sequences encoding a gene of interest, wherein three of the four nucleic acid sequences encode the same gene of interest and one of the four nucleic acid sequences encodes a different gene of interest, and wherein mRNAs encoded by the same or different gene of interest are not a target of siRNA comprised in the same RNA construct.

Recombinant RNA constructs provided herein may comprise more than one species of siRNA targeting an RNA of a gene associated with a disease or a condition described herein. For example, recombinant RNA constructs provided herein may comprise 1-10 species of siRNA targeting the same RNA or different RNAs. In some instances, each of the 1-10 species of siRNA targeting the same RNA may comprise the same sequence, i.e., each of the 1-10 species of siRNA binds to the same region of the target RNA. In some instances, each of the 1-10 species of siRNA targeting the same RNA may comprise different sequences, i.e., each of the 1-10 species of siRNA binds to different regions of the target RNA. For instance, recombinant RNA constructs provided herein, may comprise 3 species of siRNA targeting one RNA and each of the 3 species of siRNA comprise the same nucleic acid sequence to target the same region of the RNA. In this example, each of the 3 species of siRNA may comprise the same nucleic acid sequence to target exon 1. In another example, each of the 3 species of siRNA may comprise different nucleic acid sequence to target different regions of the RNA. In this example, one of the 3 species of siRNA may comprise a nucleic acid sequence targeting exon 1 and another one of the 3 species of siRNA may comprise a nucleic acid sequence targeting exon 2, etc. In yet another example, each of the 3 species of siRNA may comprise different nucleic acid sequence to target different RNAs. In all aspects, siRNAs in recombinant RNA constructs provided herein may not affect the expression of the gene of interest, expressed by the mRNA in the same RNA construct compositions. In some embodiments, the target RNA is an mRNA.

Provided herein are compositions comprising recombinant RNA constructs, comprising a first RNA sequence, a second RNA sequence, and a linker RNA sequence, wherein the linker RNA sequence links the first RNA sequence and the second RNA sequence. In some instances, a linker described herein may have a structure of Formula (I) XₘCAACAAXₙ, wherein X is any nucleotide, m is an integer from 1 to 12, and n is an integer from 0 to 4 (SEQ ID NO: 151). In some instances, a linker described herein may have a structure of Formula (II): XₚTCCCXᵣ, wherein X is any nucleotide, p is an integer from 0 to 17, and r is an integer from 0 to 13 (SEQ ID NO: 152). In one embodiment, the first RNA sequence or the second RNA sequence may comprise one or more genetic elements that modulate the expression of one or more target RNAs and the linker RNA sequence may connect each of one or more genetic elements that modulate the expression of one or more target RNAs (e.g., siRNA to siRNA linker or shRNA to shRNA linker). In another embodiment, the first RNA sequence may encode a gene of interest and the second RNA sequence may comprise one or more genetic elements that modulate the expression of one or more target RNA, and the linker RNA sequence may connect the gene of interest and the one or more genetic elements that modulate the expression of one or more target RNAs (e.g., mRNA to siRNA linker, siRNA to mRNA, mRNA to shRNA linker, or shRNA to mRNA linker). In some embodiments, the sequence of mRNA to siRNA/shRNA (or siRNA/shRNA to mRNA) linker and the sequence of siRNA to siRNA (or shRNA to shRNA) linker may be different. In some embodiments, the sequence of mRNA to siRNA/shRNA (or siRNA/shRNA to mRNA) linker and the sequence of siRNA to siRNA (or shRNA to shRNA) linker may be the same. In some embodiments, the first RNA sequence may encode a gene of interest and the second RNA sequence may comprise one or more genetic elements that modulate the expression of one or more target RNA, and the same RNA linker sequence may connect the gene of interest and the one or more genetic elements that modulate the expression of one or more target RNAs (e.g., mRNA to siRNA/shRNA linker or siRNA/shRNA to mRNA linker) and between each of the one or more genetic elements that modulate the expression of one or more target RNAs (e.g., siRNA/shRNA to siRNA/shRNA linker).

In some embodiments, the length of a linker is from about 4 to about 50, from about 4 to about 45, or from about 4 to about 40, from about 4 to about 35, or from about 4 to about nucleotides. In some embodiments, the length of a linker is from about 4 to about 27 nucleotides. In some embodiments, the length of a linker is from about 4 to about 18 nucleotides. For example, the length of a linker is about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 nucleotides. In some embodiments, the length of a linker can be at most about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or at most about 50 nucleotides. In some embodiments, the length of a linker is 4 nucleotides. In some embodiments, the length of a linker is 7 nucleotides. In some embodiments, the length of a linker is 11 nucleotides. In some embodiments, the length of a linker is 12 nucleotides. In some embodiments, the length of a linker is 18 nucleotides. In some embodiments, the length of a linker is 16 nucleotides. In some embodiments, the length of a linker is 20 nucleotides. In some embodiments, the length of a linker is 23 nucleotides. In some embodiments, the length of a linker is 27 nucleotides.

In some instances, a linker described herein may have a structure of Formula (I) XₘCAACAAXₙ, wherein X is any nucleotide; m is an integer from 1 to 12, and n is an integer from 0 to 4; and m is 1 and n is 0 (SEQ ID NO: 151). In some instances, a linker described herein may comprise a sequence comprising CAACAA (SEQ ID NO: 71), TCCC (SEQ ID NO: 69), or ACAACAA (SEQ ID NO: 23). In some embodiments, a linker may comprise a sequence selected from the group consisting of ATCCCTACGTACCAACAA (SEQ ID NO: 67), ACGTACCAACAA (SEQ ID NO: 68), TCCC (SEQ ID NO: 69), ACAACAATCCC (SEQ ID NO: 70), and ACAACAA (SEQ ID NO: 23). In some embodiments, a linker may comprise a sequence comprising ACAACAA (SEQ ID NO: 23), ATAGTGAGTCGTATTATCCC (SEQ ID NO: 72), ATAGTGAGTCGTATTAACAACAATCCC (SEQ ID NO: 73), ATAGTGAGTCGTATTAACAACAA (SEQ ID NO: 74), ATAGTGAGTCGTATTAATCCCTACGTACCAACAA (SEQ ID NO: 75), or ATAGTGAGTCGTATTAACGTACCAACAA (SEQ ID NO: 21). In some embodiments, a linker may comprise a sequence comprising ACAACAA (SEQ ID NO: 23). In some embodiments, a linker described herein may comprise a sequence selected from the group consisting of SEQ ID NOs: 23, 67-75. In some embodiments, a linker described herein may not comprise a sequence comprising TTTATCTTAGAGGCATATCCCTACGTACCAACAA (SEQ ID NO: 22). In some embodiments, a linker described herein may not comprise a sequence comprising ATAGTGAGTCGTATTAACGTACCAACAA (SEQ ID NO: 21). In some embodiments, a linker described herein does not comprise

(SEQ ID NO: 22)
TTTATCTTAGAGGCATATCCCTACGTACCAACAA
or
(SEQ ID NO: 21)
ATAGTGAGTCGTATTAACGTACCAACAA

In some instances, a tRNA linker can be used. The tRNA system is evolutionarily conserved cross living organism and utilizes endogenous RNases P and Z to process multicistronic constructs (Dong et al., 2016). In some instances, tRNA linkers described herein may comprise a nucleic acid sequence comprising AACAAAGCACCAGTGGTCTAGTGGTAGAATAGTACCCTGCCACGGTACAGACCC GGGTTCGATTCCCGGCTGGTGCA (SEQ ID NO: 39). In some instances, a linker comprising a nucleic acid sequence comprising ATAGTGAGTCGTATTAACGTACCAACAA (SEQ ID NO: 21) may be used to link the first RNA sequence and the second RNA sequence. In some embodiments, a linker comprising a nucleic acid sequence comprising TTTATCTTAGAGGCATATCCCTACGTACCAACAA (SEQ ID NO: 22) may be used to connect each of the 1-20 or more siRNA species.

In some instances, linkers described herein may not form a secondary structure. For example, linkers described herein may not bind to or base-pairs with a nucleic acid sequence of recombinant RNA constructs provided herein. In some instances, linkers described herein may not form a secondary structure within linker sequences. For example, linkers described herein may not have base-pairing within linker sequences. In some embodiments, a inker RNA sequence described herein does not form a secondary structure according to RNAfold WebServer. In some embodiments, an siRNA sequence described herein may form a secondary structure according to RNAfold WebServer.

Further provided herein are recombinant RNA construct compositions comprising 1-20 or more siRNA species, wherein each of the 1-20 or more siRNA species are connected by a linker having a structure selected from the group consisting of Formula (I): XₘCAACAAXₙ, wherein X is any nucleotide, m is an integer from 1 to 12, and n is an integer from 0 to 4 (SEQ ID NO: 151); and Formula (II): XₚTCCCXᵣ, wherein X is any nucleotide, p is an integer from 0 to 17, and r is an integer from 0 to 13 (SEQ ID NO: 152).

In some instances, recombinant RNA constructs provided herein may be cleaved. For example, recombinant RNA constructs provided herein may be cleaved endogenously after cellular uptake. In some embodiments, recombinant RNA constructs may be cleaved by an intracellular protein or an endogenous protein. In some embodiments, recombinant RNA constructs may be cleaved by DICER, e.g., an endogenous DICER. In some embodiments, recombinant RNA constructs comprising a first RNA sequence, a second RNA sequence, and a linker RNA sequence, wherein the linker RNA sequence links the first RNA sequence and the second RNA sequence, may be cleaved between the first RNA sequence and the second RNA sequence. In some embodiments, recombinant RNA constructs provided herein comprise a first RNA sequence, a second RNA sequence, and a linker. In this embodiment, the first RNA sequence or the second RNA sequence may comprise one or more genetic elements that modulate the expression of one or more target RNAs and recombinant RNA constructs may be cleaved between each of one or more genetic elements that modulate the expression of one or more target RNAs. In another embodiment, the first RNA sequence may encode a gene of interest and the second RNA sequence may comprise one or more genetic elements that modulate the expression of one or more target RNA, and recombinant RNA constructs may be cleaved between the gene of interest and the one or more genetic elements that modulate the expression of one or more target RNAs. In some embodiments, the first RNA sequence may encode a gene of interest and the second RNA sequence may comprise one or more genetic elements that modulate the expression of one or more target RNA, and recombinant RNA constructs may be cleaved between the gene of interest and the one or more genetic elements that modulate the expression of one or more target RNAs and/or between each of the one or more genetic elements that modulate the expression of one or more target RNAs.

In some instances, the cleavage of recombinant RNA constructs is enhanced compared to the cleavage of a corresponding RNA construct that does not comprise a linker described herein. For example, the cleavage of recombinant RNA constructs comprising a first RNA sequence, a second RNA sequence, and one or more of linkers described herein is enhanced compared to the cleavage of an RNA construct that does not comprise a linker described herein. For example, the cleavage of recombinant RNA constructs comprising a first RNA sequence, a second RNA sequence, and a linker RNA sequence described herein is enhanced compared to the cleavage of an RNA construct that comprises a linker that does not have a structure selected from the group consisting of Formula (I): XₘCAACAAXₙ, wherein X is any nucleotide, m is an integer from 1 to 12, and n is an integer from 0 to 4 (SEQ ID NO: 151); and Formula (II): XₚTCCCXᵣ, wherein X is any nucleotide, p is an integer from 0 to 17, and r is an integer from 0 to 13 (SEQ ID NO: 152). For example, the cleavage of recombinant RNA constructs comprising a first RNA sequence, a second RNA sequence, and a linker RNA sequence described herein is enhanced compared to the cleavage of an RNA construct that comprises a linker that does not comprise a sequence comprising ACAACAA (SEQ ID NO: 23). For example, the cleavage of recombinant RNA constructs comprising a first RNA sequence, a second RNA sequence, and a linker RNA sequence described herein is enhanced compared to the cleavage of an RNA construct comprising a linker that forms a secondary structure.

In some instances, the expression of a gene of interest from recombinant RNA constructs provided herein is enhanced compared to the expression of a gene of interest from a corresponding recombinant RNA construct that does not comprise a linker described herein. For example, the expression of a gene of interest from recombinant RNA constructs comprising a first RNA sequence, a second RNA sequence, and a linker RNA sequence described herein is enhanced compared to the expression of a gene of interest from an RNA construct that does not comprise a linker described herein. For example, the expression of a gene of interest from recombinant RNA constructs comprising a first RNA sequence, a second RNA sequence, and a linker RNA sequence described herein is enhanced compared to the expression of a gene of interest from an RNA construct that comprises a linker that does not have a structure selected from the group consisting of Formula (I): XₘCAACAAXₙ, wherein X is any nucleotide, m is an integer from 1 to 12, and n is an integer from 0 to 4 (SEQ ID NO: 151); and Formula (II): XₚTCCCXᵣ, wherein X is any nucleotide, p is an integer from 0 to 17, and r is an integer from 0 to 13 (SEQ ID NO: 152). For example, the expression of a gene of interest from recombinant RNA constructs comprising a first RNA sequence, a second RNA sequence, and a linker RNA sequence described herein is enhanced compared to the expression of a gene of interest from an RNA construct that comprises a linker that does not comprise a sequence comprising ACAACAA (SEQ ID NO: 23). For example, the expression of a gene of interest from recombinant RNA constructs comprising a first RNA sequence, a second RNA sequence, and a linker RNA sequence described herein is enhanced compared to the expression of a gene of interest from an RNA construct that comprises a linker that does not comprise a sequence comprising ATCCCTACGTACCAACAA (SEQ ID NO: 67). For example, the expression of a gene of interest from recombinant RNA constructs comprising a first RNA sequence, a second RNA sequence, and a linker RNA sequence described herein is enhanced compared to the expression of a gene of interest from an RNA construct that comprises a linker that does not comprise a sequence comprising ACGTACCAACAA (SEQ ID NO: 68). For example, the expression of a gene of interest from recombinant RNA constructs comprising a first RNA sequence, a second RNA sequence, and a linker RNA sequence described herein is enhanced compared to the expression of a gene of interest from an RNA construct that comprises a linker that does not comprise a sequence comprising TCCC (SEQ ID NO: 69). For example, the expression of a gene of interest from recombinant RNA constructs comprising a first RNA sequence, a second RNA sequence, and a linker RNA sequence described herein is enhanced compared to the expression of a gene of interest from an RNA construct that comprises a linker that does not comprise a sequence comprising ACAACAATCCC (SEQ ID NO: 70). For example, the expression of a gene of interest from recombinant RNA constructs comprising a first RNA sequence, a second RNA sequence, and a linker RNA sequence described herein is enhanced compared to the expression of a gene of interest from an RNA construct comprising a linker that forms a secondary structure.

In some instances, the expression of a gene of interest from recombinant RNA constructs comprising a linker described herein may be enhanced compared to the expression of a gene of interest from a corresponding recombinant RNA construct with another linker described herein. For example, the expression of a gene of interest from recombinant RNA constructs comprising a first RNA sequence, a second RNA sequence, and a A2-linker RNA sequence described herein may be enhanced compared to the expression of a gene of interest from an RNA construct that comprises another linker described herein (e.g., B-linker, C-linker, D-linker, or E-linker). For example, the expression of a gene of interest from recombinant RNA constructs comprising a first RNA sequence, a second RNA sequence, and a B-linker RNA sequence described herein may be enhanced compared to the expression of a gene of interest from an RNA construct that comprises another linker described herein (e.g., A2-linker, C-linker, D-linker, or E-linker). For example, the expression of a gene of interest from recombinant RNA constructs comprising a first RNA sequence, a second RNA sequence, and a C-linker RNA sequence described herein may be enhanced compared to the expression of a gene of interest from an RNA construct that comprises another linker described herein (e.g., A2-linker, B-linker, D-linker, or E-linker). For example, the expression of a gene of interest from recombinant RNA constructs comprising a first RNA sequence, a second RNA sequence, and a D-linker RNA sequence described herein may be enhanced compared to the expression of a gene of interest from an RNA construct that comprises another linker described herein (e.g., A2-linker, B-linker, C-linker, or E-linker). For example, the expression of a gene of interest from recombinant RNA constructs comprising a first RNA sequence, a second RNA sequence, and a E-linker RNA sequence described herein may be enhanced compared to the expression of a gene of interest from an RNA construct that comprises another linker described herein (e.g., A2-linker, B-linker, C-linker, or D-linker). In some embodiments, a A2-linker may comprise a sequence comprising ACAACAA (SEQ ID NO: 23). In some embodiments, a B linker may comprise a sequence comprising ATCCCTACGTACCAACAA (SEQ ID NO: 67). In some embodiments, a C-linker may comprise a sequence comprising ACGTACCAACAA (SEQ ID NO: 68). In some embodiments, a D-linker may comprise a sequence comprising TCCC (SEQ ID NO: 69). In some embodiments, a E-linker may comprise a sequence comprising ACAACAATCCC (SEQ ID NO: 70).

In some embodiments, the relative increase or enhancement in the expression of a gene of interest or in the cleavage of recombinant RNA constructs is at least about 1.3 fold, about 1.4 fold, about 1.5 fold, about 1.6 fold, about 1.7 fold, about 1.8 fold, about 1.9 fold, about 2 fold, about 3 fold, about 4 fold, about 5 fold, about 6 fold, about 7 fold, about 8 fold, about 9 fold, 10 fold, about 15 fold, about 17 fold, about 18 fold, about 19 fold, about 20 fold, about 21 fold, about 22 fold, or at least about 25 fold. In some embodiments, the relative increase in the expression of the gene of interest or in the cleavage of recombinant RNA constructs is from about 1.3 fold to about 3 fold, from about 1.5 fold to about 4 fold, from about 2 fold to about 5 fold, from about 2 fold to about 10 fold, from about 2 fold to about 15 fold, from about 2 fold to about 17 fold, from about 2 fold to about 18 fold, from about 2 fold to about 19 fold, from about 2 fold to about 20 fold, from about 2 fold to about 21 fold, from about 2 fold to about 22 fold, from about 2 fold to about 25 fold, from about 2 fold to about 30 fold, from about 5 fold to about 10 fold, from about 5 fold to about 15 fold, from about 5 fold to about 17 fold, from about 5 fold to about 18 fold, from about 5 fold to about 19 fold, from about 5 fold to about 20 fold, from about 5 fold to about 21 fold, from about 5 fold to about 22 fold, from about 5 fold to about 25 fold, from about 5 fold to about 30 fold, from about 10 fold to about 15 fold, from about 10 fold to about 17 fold, from about 10 fold to about 18 fold, from about 10 fold to about 19 fold, from about 10 fold to about 20 fold, from about 10 fold to about 21 fold, from about 10 fold to about 22 fold, from about 10 fold to about 25 fold, from about 10 fold to about 30 fold, from about 15 fold to about 17 fold, from about 15 fold to about 18 fold, from about 15 fold to about 19 fold, from about 15 fold to about 20 fold, from about 15 fold to about 21 fold, from about 15 fold to about 22 fold, from about 15 fold to about 25 fold, from about 15 fold to about 30 fold, from about 17 fold to about 18 fold, from about 17 fold to about 19 fold, from about 17 fold to about 20 fold, from about 17 fold to about 21 fold, from about 17 fold to about 22 fold, from about 17 fold to about 25 fold, from about 17 fold to about 30 fold, from about 18 fold to about 19 fold, from about 18 fold to about 20 fold, from about 18 fold to about 21 fold, from about 18 fold to about 22 fold, from about 18 fold to about 25 fold, from about 18 fold to about 30 fold, from about 19 fold to about 20 fold, from about 19 fold to about 21 fold, from about 19 fold to about 22 fold, from about 19 fold to about 25 fold, from about 19 fold to about 30 fold, from about 20 fold to about 21 fold, from about 20 fold to about 22 fold, from about 20 fold to about 25 fold, from about 20 fold to about 30 fold, from about 21 fold to about 22 fold, from about 21 fold to about 25 fold, from about 21 fold to about 30 fold, from about 22 fold to about 25 fold, from about 22 fold to about 30 fold, or from about 25 fold to about 30 fold. In some embodiments, the relative increase in the expression of the gene of interest or in the cleavage of recombinant RNA constructs is about 1.3 fold, about 1.4 fold, about 1.5 fold, about 1.6 fold, about 1.7 fold, about 1.8 fold, about 1.9 fold, about 2 fold, about 3 fold, about 4 fold, about 5 fold, about 6 fold, about 7 fold, about 8 fold, about 9 fold, about 10 fold, about 15 fold, about 17 fold, about 18 fold, about 19 fold, about 20 fold, about 21 fold, about 22 fold, about 25 fold, or about 30 fold. In some embodiments, the relative increase in the expression of the gene of interest or in the cleavage of recombinant RNA constructs is at most about 2 fold, about 3 fold, about 5 fold, about 10 fold, about 15 fold, about 17 fold, about 18 fold, about 19 fold, about 20 fold, about 21 fold, about 22 fold, about 25 fold, or at most about 30 fold.

In some embodiments, recombinant RNA constructs provided herein may be naked RNA. In some embodiments, recombinant RNA constructs provided herein may further comprise a 5′ cap, a Kozak sequence, and/or internal ribosome entry site (IRES), and/or a poly(A) tail in a particular in order to improve translation. In some instances, recombinant RNA constructs may further comprise one or more regions promoting translation known to any skilled artisan. Non-limiting examples of the 5′ cap can include an anti-reverse CAP analog, Clean Cap, Cap 0, Cap 1, Cap 2, or Locked Nucleic Acid cap (LNA-cap). In some instances, 5′ cap may comprise m₂^{7,3′-O} G(5′)ppp(5′)G, m7G, m7G(5′)G, m7GpppG, or m7GpppGm. In some instances, recombinant RNA constructs provided herein may comprise an IRES upstream or 5′ of the RNA sequence encoding for a gene of interest. In some instances, recombinant RNA constructs provided herein may comprise an IRES immediately upstream or 5′ of the RNA sequence encoding for a gene of interest. In some instances, recombinant RNA constructs provided herein may comprise an IRES downstream or 3′ of the RNA sequence encoding at least one genetic element that modulates expression of a target RNA, wherein the RNA sequence encoding at least one genetic element that modulates expression of a target RNA is present upstream of the RNA sequence encoding for a gene of interest.

Recombinant RNA constructs provided herein may further comprise a poly(A) tail. In some instances, the poly(A) tail comprises 1 to 220 base pairs of poly(A) (SEQ ID NO: 153). For example, the poly(A) tail comprises 1, 3, 5, 8, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, or 220 base pairs of poly(A) (SEQ ID NO: 153). In some embodiments, the poly(A) tail comprises 1 to 20, 1 to 40, 1 to 60, 1 to 80, 1 to 100, 1 to 120, 1 to 140, 1 to 160, 1 to 180, 1 to 200, 1 to 220, 20 to 40, 20 to 60, 20 to 80, 20 to 100, 20 to 120, 20 to 140, 20 to 160, 20 to 180, 20 to 200, 20 to 220, 40 to 60, 40 to 80, 40 to 100, 40 to 120, 40 to 140, 40 to 160, 40 to 180, 40 to 200, 40 to 220, 60 to 80, 60 to 100, 60 to 120, 60 to 140, 60 to 160, 60 to 180, 60 to 200, 60 to 220, 80 to 100, 80 to 120, 80 to 140, 80 to 160, 80 to 180, 80 to 200, 80 to 220, 100 to 120, 100 to 140, 100 to 160, 100 to 180, 100 to 200, 100 to 220, 120 to 140, 120 to 160, 120 to 180, 120 to 200, 120 to 220, 140 to 160, 140 to 180, 140 to 200, 140 to 220, 160 to 180, 160 to 200, 160 to 220, 180 to 200, 180 to 220, or 200 to 220 base pairs of poly(A) (SEQ ID NO: 153). In some embodiments, the poly(A) tail comprises 1, 20, 40, 60, 80, 100, 120, 140, 160, 180, 200, or 220 base pairs of poly(A) (SEQ ID NO: 153). In some embodiments, the poly(A) tail comprises at least 1, 20, 40, 60, 80, 100, 120, 140, 160, 180, or at least 200 base pairs of poly(A) (SEQ ID NO: 154). In some embodiments, the poly(A) tail comprises at most 20, 40, 60, 80, 100, 120, 140, 160, 180, 200, or at most 220 base pairs of poly(A) (SEQ ID NO: 153). In some embodiments, the poly(A) tail comprises 120 base pairs of poly(A) (SEQ ID NO: 155).

Recombinant RNA constructs provided herein may further comprise a Kozak sequence. A Kozak sequence may refer to a nucleic acid sequence motif that functions as a protein translation initiation site. Kozak sequences are described at length in the literature, e.g., by Kozak, M., Gene 299(1-2):1-34, incorporated herein by reference herein in its entirety. In some embodiments, the Kozak sequence described herein may comprise a sequence comprising GCCACC (SEQ ID NO: 19). In some embodiments, recombinant RNA constructs provided herein may further comprise a nuclear localization signal (NLS).

In one aspect, recombinant RNA constructs described herein may not comprise a nucleotide variant. In some instances, recombinant RNA constructs described herein may comprise one or more uridines. In some instances, recombinant RNA constructs described herein may not comprise a modified uridine. In some instances, recombinant RNA constructs described herein may not comprise one or more N1-methylpseudouridines. In some embodiments, between 99% and 1%, between 98% and 2%, between 97% and 3%, between 96% and 4%, between 95% and 2%, between 94% and 6%, between 93% and 7%, between 92% and 8%, between 91% and 9%, between 90% and 10%, between 97% and 3%, of the one or more uridines comprised in the recombinant RNA constructs are unmodified. In some embodiments, at least 70%, 75%, 80%, 85%, 90%, 91%, 92%, 93%, 94%, 95%, 96%, 97%, 98%, 99%, 99.5%, 99.6%, 99.7%, 99.8%, or at least 99.9% of one or more uridines comprised in the recombinant RNA constructs are unmodified. In some embodiments, at most 1%, 5%, 10%, 20%, 30%, 40%, 50%, 60%, 70%, 75%, 80%, 85%, 90%, 91%, 92%, 93%, 94%, 95%, 96%, 97%, 98%, 99%, 99.5%, 99.6%, 99.7%, 99.8%, or at least 99.9% of one or more uridines comprised in the recombinant RNA constructs are modified. In one embodiment, recombinant RNA constructs described herein comprise solely unmodified nucleotides. For example, recombinant RNA constructs described herein comprise only natural nucleotides. For example, recombinant RNA constructs described herein comprise only canonical nucleotides. In a preferred embodiment, recombinant RNA constructs described herein comprise one or more uridines, wherein all of one or more uridines are unmodified.

In another aspect, recombinant RNA constructs described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenosine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine, N1-methylpseudouridine, and the like. In some cases, nucleotides may include modifications in their phosphate moieties, including modifications to a triphosphate moiety. Non-limiting examples of such modifications include phosphate chains of greater length and modifications with thiol moieties. In some embodiments, phosphate chains can comprise 4, 5, 6, 7, 8, 9, 10 or more phosphate moieties. In some embodiments, thiol moieties can include but are not limited to alpha-thiotriphosphate and beta-thiotriphosphates. In some embodiments, a recombinant RNA construct described herein does not comprise 5-methylcytosine and/or N6-methyladenosine.

In some instances, recombinant RNA constructs described herein may be modified at the base moiety, sugar moiety, or phosphate backbone. For example, modifications can be at one or more atoms that typically are available to form a hydrogen bond with a complementary nucleotide and/or at one or more atoms that are not typically capable of forming a hydrogen bond with a complementary nucleotide. In some embodiments, backbone modifications include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) allows prevents nuclease recognition and degradation. In some embodiments, backbone modifications include having peptide bonds instead of phosphorous in the backbone structure, or linking groups including carbamate, amides, and linear and cyclic hydrocarbon groups. For example, N-(2-aminoethyl)-glycine units may be linked by peptide bonds in a peptide nucleic acid. Oligonucleotides with modified backbones are reviewed in Micklefield, Backbone modification of nucleic acids: synthesis, structure and therapeutic applications, Curr. Med. Chem., 8 (10): 1157-79, 2001 and Lyer et al., Modified oligonucleotides-synthesis, properties and applications, Curr. Opin. Mol. Ther., 1 (3): 344-358, 1999.

Recombinant RNA constructs provided herein may comprise a combination of modified and unmodified nucleotides. In some instances, the adenosine-, guanosine-, and cytidine-containing nucleotides are unmodified or partially modified. In some instances, for modified RNA constructs, 1%, 5%, 10%, 20%, 30%, 40%, 50%, 60%, 70%, 80%, 90%, or 100% of uridine nucleotides may be modified. In some embodiments, 5% to 25% of uridine nucleotides are modified in recombinant RNA constructs. Non-limiting examples of the modified uridine nucleotides may comprise pseudouridines, N¹-methylpseudouridines, or N1-methylpseudo-UTP and any modified uridine nucleotides known in the art may be utilized. In some embodiments, recombinant RNA constructs may contain a combination of modified and unmodified nucleotides, wherein 1%, 5%, 10%, 20%, 30%, 40%, 50%, 60%, 70%, 80%, 90%, or 100% of uridine nucleotides may comprise pseudouridines, N¹-methylpseudouridines, N¹-methylpseudo-UTP, or any other modified uridine nucleotide known in the art. In some embodiments, recombinant RNA constructs may contain a combination of modified and unmodified nucleotides, wherein 1%, 5%, 10%, 20%, 30%, 40%, 50%, 60%, 70%, 80%, 90%, or 100% of the uridine nucleotides may comprise N¹-methylpseudouridines.

Recombinant RNA constructs provided herein may be codon-optimized. In general, codon optimization refers to a process of modifying a nucleic acid sequence for expression in a host cell of interest by replacing at least one codon (e.g., more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database,” and these tables can be adapted in a number of ways. Computer algorithms for codon optimizing a particular sequence for expression in a particular host cell are also available, such as Gene Forge® (Aptagen, PA) and GeneOptimizer® (ThermoFischer, MA) which is preferred. In some embodiments, recombinant RNA constructs may not be codon-optimized.

In some instances, recombinant RNA constructs may comprise a nucleic acid sequence comprising a sequence selected from the group consisting of SEQ ID NOs: 1-9 and 76-84.

RNA Interference and Small Interfering RNA (siRNA)

RNA interference (RNAi) or RNA silencing is a process in which RNA molecules inhibit gene expression or translation, by neutralizing target mRNA molecules. RNAi process is described in Mello & Conte (2004) Nature 431, 338-342, Meister & Tuschl (2004) Nature 431, 343-349, Hannon & Rossi (2004) Nature 431, 371-378, and Fire (2007) Angew. Chem. Int. Ed. 46, 6966-6984. Briefly, in a natural process, the reaction initiates with a cleavage of long double-stranded RNA (dsRNA) into small dsRNA fragments or siRNAs with a hairpin structure (i.e., shRNAs) by a dsRNA-specific endonuclease Dicer. These small dsRNA fragments or siRNAs are then integrated into RNA-induced silencing complex (RISC) and guide the RISC to the target mRNA sequence. During interference, the siRNA duplex unwinds, and the anti-sense strand remains in complex with RISC to lead RISC to the target mRNA sequence to induce degradation and subsequent suppression of protein translation. Unlike commercially available synthetic siRNAs, siRNAs in the present invention can utilize endogenous Dicer and RISC pathway in the cytoplasm of a cell to get cleaved from recombinant RNA constructs (e.g., recombinant RNA constructs comprising an mRNA and two or more siRNAs) after cellular uptake and follow the natural process detailed above, as siRNAs in the recombinant RNA constructs of the present invention comprise a hairpin loop structure. In addition, as the rest of the recombinant RNA constructs (i.e., mRNA) is left intact after cleavage of siRNAs by Dicer, the desired protein expression from the gene of interest in the recombinant RNA constructs of the present invention is attained.

Provided herein are compositions comprising recombinant RNA constructs comprising at least one nucleic acid sequence comprising a siRNA capable of binding to a target RNA. In some instances, the target RNA is a noncoding RNA. In some instances, the target RNA is an mRNA. In some embodiments, the siRNA is capable of binding to a target mRNA in the 5′ untranslated region. In some embodiments, the siRNA is capable of binding to a target mRNA in the 3′ untranslated region. In some embodiments, the siRNA is capable of binding to a target mRNA in an exon. In some embodiments, recombinant RNA constructs may comprise a nucleic acid sequence comprising a sense siRNA strand. In some embodiments, recombinant RNA constructs may comprise a nucleic acid sequence comprising an anti-sense siRNA strand. In some embodiments, recombinant RNA constructs may comprise a nucleic acid sequence comprising a sense siRNA strand and a nucleic acid sequence comprising an anti-sense siRNA strand. Details of siRNA comprised in the present invention are described in Cheng, et al. (2018) J. Mater. Chem. B., 6, 4638-4644, which is incorporated by reference herein.

For example, in some instances, recombinant RNA constructs may comprise at least 1 species or copy of siRNA, i.e., a nucleic acid sequence comprising a sense strand of siRNA and a nucleic acid sequence comprising an anti-strand of siRNA. 1 species or 1 copy of siRNA, as described herein, can refer to 1 species or 1 copy of sense strand siRNA and 1 species or 1 copy of anti-sense strand siRNA. In some instances, recombinant RNA constructs may comprise more than 1 species or 1 copy of siRNA, e.g., 2, 3, 4, 5, 6, 7, 8, 9, 10, or more species or copies of siRNA comprising a sense strand of siRNA and an anti-strand of siRNA. In some embodiments, recombinant RNA constructs may comprise 1 to 20 species or copies of siRNA. In some embodiments, recombinant RNA constructs may comprise at least 1, 2, 3, 4, 5, 6, 7, 8, 9, or at least 10 species or copies of siRNA. In some embodiments, recombinant RNA constructs may comprise at most 3, 4, 5, 6, 7, 8, 9, 10, 11, 12, 13, 14, 15, 16, 17, 18, 19, or at most 20 species or copies of siRNA. In some embodiments, the recombinant polynucleic acid or RNA construct has 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10 species or copies of siRNA. In some embodiments, the recombinant polynucleic acid or RNA construct has 1, 2, 3, 4, 5, 6, 7, 8, 9, or 10 species or copies of siRNA. In some embodiments, the recombinant polynucleic acid or RNA construct has at least 1, 2, 3, 4, 5, 6, 7, 8, or 9 species or copies of siRNA. In some embodiments, the recombinant polynucleic acid or RNA construct has at most 2, 3, 4, 5, 6, 7, 8, 9, or 10 species or copies of siRNA. In some embodiments, recombinant RNA constructs may comprise between 2 siRNAs and 10 siRNAs, between 3 siRNAs and 10 siRNAs, between 4 siRNAs and 10 siRNAs, between 5 siRNAs and 10 siRNAs, between 6 siRNAs and 10 siRNAs, between 7 siRNAs and 10 siRNAs, between 9 siRNAs and 10 siRNAs, preferably between 2 siRNAs and 6 siRNAs, between 3 siRNAs and 6 siRNAs, or between 4 siRNAs and 6 siRNAs.

Provided herein are compositions of recombinant RNA constructs comprising 1-20 or more siRNA species or copies, wherein each of the 1-20 or more siRNA species or copies is capable of binding to a target RNA. In some embodiments, a target RNA is an mRNA or a non-coding RNA. In some instances, each of the siRNA species or copies binds to the same target RNA. In one instance, each of the siRNA species or copies may comprise the same sequence and bind to the same region or sequence of the same target RNA. For example, recombinant RNA constructs may comprise 1, 2, 3, 4, 5, or more siRNA species or copies and each of the 1, 2, 3, 4, 5, or more siRNA species or copies comprise the same sequence targeting the same region of a target RNA, i.e., recombinant RNA constructs may comprise 1, 2, 3, 4, 5, or more redundant species or copies of siRNA. In another instance, each of the siRNA species or copies may comprise a different sequence and bind to a different region or sequence of the same target RNA. For example, recombinant RNA constructs may comprise 1, 2, 3, 4, 5, or more siRNA species or copies and each of the 1, 2, 3, 4, 5, or more siRNA species or copies may comprise a different sequence targeting a different region of the same target RNA. In this example, one siRNA of the 1, 2, 3, 4, 5, or more siRNA species or copies may target exon 1 and another siRNA of the 1, 2, 3, 4, 5, or more siRNA species or copies may target exon 2 of the same mRNA, etc. In some instances, recombinant RNA constructs may comprise 1, 2, 3, 4, 5, or more siRNA species or copies capable of binding to the same and different regions of the same target RNA. For example, recombinant RNA constructs may comprise 1, 2, 3, 4, 5, or more siRNA species or copies and 2 of the 1, 2, 3, 4, 5, or more siRNA species or copies may comprise the same sequence and bind to the same regions of the target RNA and 3 or more of the 1, 2, 3, 4, 5, or more siRNA species or copies may comprise a different sequence and bind to different regions of the same target RNA. In some instances, each of the siRNA species or copies binds to a different target RNA. In some instances, recombinant RNA constructs may comprise 1, 2, 3, 4, 5, or more siRNA species or copies capable of binding to the same and different target RNAs. For example, recombinant RNA constructs may comprise 1, 2, 3, 4, 5, or more siRNA species or copies and 2 of the 1, 2, 3, 4, 5, or more siRNA species or copies may comprise a sequence capable of binding to the same or different regions of the same target RNA and 3 or more of the 1, 2, 3, 4, 5, or more siRNA species or copies may comprise a sequence capable of binding to a different target RNA. In some embodiments, a target RNA may be an mRNA and/or a non-coding RNA. In some instances, each of the siRNA species or copies may comprise the same sequence that can bind to different target RNAs. For example, each of the siRNA species or copies may bind to a sequence common to, or shared by, two or more target RNAs. Examples include, but are not limited to, an siRNA sequence that can bind to a sequence common to, or shared by, Protein kinase B-1 (Akt1), Akt2, and Akt3 (pan-Akt3).

Provided herein are compositions of recombinant RNA constructs comprising 1-20 or more siRNA species, wherein each of the 1-20 or more siRNA species are connected by a linker described herein. In some instances, the linker may be a non-cleavable linker. In some instances, the linker may be a cleavable linker such as a self-cleavable linker. In some instances, the linker may be cleaved by a protein, e.g., an intracellular or an endogenous protein. In some instances, the linker has a structure selected from the group consisting of Formula (I): XₘCAACAAXₙ, wherein X is any nucleotide, m is an integer from 1 to 12, and n is an integer from 0 to 4 (SEQ ID NO: 151); and Formula (II): XₚTCCCXᵣ, wherein X is any nucleotide, p is an integer from 0 to 17, and r is an integer from 0 to 13 (SEQ ID NO: 152). In some instances, the linker may comprise a sequence comprising ACAACAA (SEQ ID NO: 23), ATCCCTACGTACCAACAA (SEQ ID NO: 67), ACGTACCAACAA (SEQ ID NO: 68), TCCC (SEQ ID NO: 69), or ACAACAATCCC (SEQ ID NO: 70). In some embodiments, the linker may comprise a sequence comprising ACAACAA (SEQ ID NO: 23), ATAGTGAGTCGTATTATCCC (SEQ ID NO: 72), ATAGTGAGTCGTATTAACAACAATCCC (SEQ ID NO: 73), ATAGTGAGTCGTATTAACAACAA (SEQ ID NO: 74), ATAGTGAGTCGTATTAATCCCTACGTACCAACAA (SEQ ID NO: 75), or ATAGTGAGTCGTATTAACGTACCAACAA (SEQ ID NO: 21). In some embodiments, the linker comprises a sequence comprising ACAACAA (SEQ ID NO: 23). In some embodiments, the linker does not comprise a sequence comprising

(SEQ ID NO: 22)
TTTATCTTAGAGGCATATCCCTACGTACCAACAA
or
(SEQ ID NO: 21)
ATAGTGAGTCGTATTAACGTACCAACAA

In some instances, the length of a linker is from about 4 to about 50, from about 4 to about 45, or from about 4 to about 40, from about 4 to about 35, or from about 4 to about 30 nucleotides. In some embodiments, the length of a linker is from about 4 to about 27 nucleotides. In some embodiments, the length of a linker is from about 4 to about 18 nucleotides. For example, the length of a linker is about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 nucleotides. In some embodiments, the length of a linker can be at most about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or at most about 50 nucleotides. In some embodiments, the length of a linker is 4 nucleotides. In some embodiments, the length of a linker is 7 nucleotides. In some embodiments, the length of a linker is 11 nucleotides. In some embodiments, the length of a linker is 12 nucleotides. In some embodiments, the length of a linker is 18 nucleotides.

In some instances, the linker may have a structure of Formula (I) XₘCAACAAXₙ, wherein X is any nucleotide; m is an integer from 1 to 12, and n is an integer from 0 to 4 (SEQ ID NO: 151); and m is 1 and n is 0. In some instances, the linker may comprise a sequence comprising CAACAA (SEQ ID NO: 71), TCCC (SEQ ID NO: 69), or ACAACAA (SEQ ID NO: 23). In some embodiments, the linker may comprise a sequence selected from the group consisting of ATCCCTACGTACCAACAA (SEQ ID NO: 67), ACGTACCAACAA (SEQ ID NO: 68), TCCC (SEQ ID NO: 69), ACAACAATCCC (SEQ ID NO: 70), and ACAACAA (SEQ ID NO: 23). In some embodiments, the linker may comprise a sequence comprising ACAACAA (SEQ ID NO: 23). In some embodiments, the linker may comprise a sequence comprising ACAACAA (SEQ ID NO: 23), ATAGTGAGTCGTATTATCCC (SEQ ID NO: 72), ATAGTGAGTCGTATTAACAACAATCCC (SEQ ID NO: 73), ATAGTGAGTCGTATTAACAACAA (SEQ ID NO: 74), ATAGTGAGTCGTATTAATCCCTACGTACCAACAA (SEQ ID NO: 75), or ATAGTGAGTCGTATTAACGTACCAACAA (SEQ ID NO: 21). In some embodiments, the linker may comprise a sequence selected from the group consisting of SEQ ID NOs: 23, 67-75.

In some instances, the linker may be a tRNA linker. The tRNA system is evolutionarily conserved across living organism and utilizes endogenous RNases P and Z to process multicistronic constructs (Dong et al., 2016). In some embodiments, the tRNA linker may comprise a nucleic acid sequence comprising AACAAAGCACCAGTGGTCTAGTGGTAGAATAGTACCCTGCCACGGTACAGACCC GGGTTCGATTCCCGGCTGGTGCA (SEQ ID NO: 39). In some embodiments, a linker comprising a nucleic acid sequence comprising TTTATCTTAGAGGCATATCCCTACGTACCAACAA (SEQ ID NO: 22) may be used to connect each of the 1-20 or more siRNA species.

In some instances, specific binding of an siRNA to its target mRNA results in interference with the normal function of the target mRNA, leading to modulation, e.g., downregulation, of expression level, function, and/or activity of a protein encoded by the target mRNA, and there is a sufficient degree of complementarity to avoid non-specific binding of the siRNA to non-target nucleic acid sequences under conditions in which specific binding is desired, i.e., under physiological conditions in the case of in vivo assays or therapeutic treatment, and under conditions in which assays are performed in the case of in vitro assays.

In some instances, the downregulation of expression level, function, and/or activity of a protein encoded by a target mRNA by siRNAs from recombinant RNA constructs provided herein may be enhanced compared to the downregulation of expression level, function, and/or activity of a protein encoded by a target mRNA by siRNAs from a corresponding recombinant RNA construct that does not comprise a linker described herein. For example, the downregulation of expression level, function, and/or activity of a protein encoded by a target mRNA by siRNAs from recombinant RNA constructs comprising a first RNA sequence, a second RNA sequence, and a linker RNA sequence described herein may be enhanced compared to the downregulation of expression level, function, and/or activity of a protein encoded by a target mRNA by siRNAs from an RNA construct that does not comprise a linker described herein. For example, the downregulation of expression level, function, and/or activity of a protein encoded by a target mRNA by siRNAs from recombinant RNA constructs comprising a first RNA sequence, a second RNA sequence, and a linker RNA sequence described herein may be enhanced compared to the downregulation of expression level, function, and/or activity of a protein encoded by a target mRNA by siRNAs from an RNA construct that comprises a linker that does not have a structure selected from the group consisting of Formula (I): XₘCAACAAXₙ, wherein X is any nucleotide, m is an integer from 1 to 12, and n is an integer from 0 to 4 (SEQ ID NO: 151); and Formula (II): XₚTCCCXᵣ, wherein X is any nucleotide, p is an integer from 0 to 17, and r is an integer from 0 to 13 (SEQ ID NO: 152). For example, the downregulation of expression level, function, and/or activity of a protein encoded by a target mRNA by siRNAs from recombinant RNA constructs comprising a first RNA sequence, a second RNA sequence, and a linker RNA sequence described herein may be enhanced compared to the downregulation of expression level, function, and/or activity of a protein encoded by a target mRNA by siRNAs from an RNA construct that comprises a linker that does not comprise a sequence comprising ACAACAA (SEQ ID NO: 23). For example, the downregulation of expression level, function, and/or activity of a protein encoded by a target mRNA by siRNAs from recombinant RNA constructs comprising a first RNA sequence, a second RNA sequence, and a linker RNA sequence described herein may be enhanced compared to the downregulation of expression level, function, and/or activity of a protein encoded by a target mRNA by siRNAs from an RNA construct that comprises a linker that does not comprise a sequence comprising ATCCCTACGTACCAACAA (SEQ ID NO: 67). For example, the downregulation of expression level, function, and/or activity of a protein encoded by a target mRNA by siRNAs from recombinant RNA constructs comprising a first RNA sequence, a second RNA sequence, and a linker RNA sequence described herein may be enhanced compared to the downregulation of expression level, function, and/or activity of a protein encoded by a target mRNA by siRNAs from an RNA construct that comprises a linker that does not comprise a sequence comprising ACGTACCAACAA (SEQ ID NO: 68). For example, the downregulation of expression level, function, and/or activity of a protein encoded by a target mRNA by siRNAs from recombinant RNA constructs comprising a first RNA sequence, a second RNA sequence, and a linker RNA sequence described herein may be enhanced compared to the downregulation of expression level, function, and/or activity of a protein encoded by a target mRNA by siRNAs from an RNA construct that comprises a linker that does not comprise a sequence comprising TCCC (SEQ ID NO: 69). For example, the downregulation of expression level, function, and/or activity of a protein encoded by a target mRNA by siRNAs from recombinant RNA constructs comprising a first RNA sequence, a second RNA sequence, and a linker RNA sequence described herein may be enhanced compared to the downregulation of expression level, function, and/or activity of a protein encoded by a target mRNA by siRNAs from an RNA construct that comprises a linker that does not comprise a sequence comprising ACAACAATCCC (SEQ ID NO: 70).

In some instances, the downregulation of expression level, function, and/or activity of a protein encoded by a target mRNA by siRNAs from recombinant RNA constructs comprising a linker described herein may be enhanced compared to the downregulation of expression level, function, and/or activity of a protein encoded by a target mRNA by siRNAs from a corresponding recombinant RNA construct with another linker described herein. For example, the downregulation of expression level, function, and/or activity of a protein encoded by a target mRNA by siRNAs from recombinant RNA constructs comprising a first RNA sequence, a second RNA sequence, and a A2-linker RNA sequence described herein may be enhanced compared to the downregulation of expression level, function, and/or activity of a protein encoded by a target mRNA by siRNAs from an RNA construct that comprises another linker described herein (e.g., B-linker, C-linker, D-linker, or E-linker). For example, the downregulation of expression level, function, and/or activity of a protein encoded by a target mRNA by siRNAs from recombinant RNA constructs comprising a first RNA sequence, a second RNA sequence, and a B-linker RNA sequence described herein may be enhanced compared to the downregulation of expression level, function, and/or activity of a protein encoded by a target mRNA by siRNAs from an RNA construct that comprises another linker described herein (e.g., A2-linker, C-linker, D-linker, or E-linker). For example, the downregulation of expression level, function, and/or activity of a protein encoded by a target mRNA by siRNAs from recombinant RNA constructs comprising a first RNA sequence, a second RNA sequence, and a C-linker RNA sequence described herein may be enhanced compared to the downregulation of expression level, function, and/or activity of a protein encoded by a target mRNA by siRNAs from an RNA construct that comprises another linker described herein (e.g., A2-linker, B-linker, D-linker, or E-linker). For example, the downregulation of expression level, function, and/or activity of a protein encoded by a target mRNA by siRNAs from recombinant RNA constructs comprising a first RNA sequence, a second RNA sequence, and a D-linker RNA sequence described herein may be enhanced compared to the downregulation of expression level, function, and/or activity of a protein encoded by a target mRNA by siRNAs from an RNA construct that comprises another linker described herein (e.g., A2-linker, B-linker, C-linker, or E-linker). For example, the downregulation of expression level, function, and/or activity of a protein encoded by a target mRNA by siRNAs from recombinant RNA constructs comprising a first RNA sequence, a second RNA sequence, and a E-linker RNA sequence described herein may be enhanced compared to the downregulation of expression level, function, and/or activity of a protein encoded by a target mRNA by siRNAs from an RNA construct that comprises another linker described herein (e.g., A2-linker, B-linker, C-linker, or D-linker). In some embodiments, a A2-linker may comprise a sequence comprising ACAACAA (SEQ ID NO: 23). In some embodiments, a B linker may comprise a sequence comprising ATCCCTACGTACCAACAA (SEQ ID NO: 67). In some embodiments, a C-linker may comprise a sequence comprising ACGTACCAACAA (SEQ ID NO: 68). In some embodiments, a D-linker may comprise a sequence comprising TCCC (SEQ ID NO: 69). In some embodiments, a E-linker may comprise a sequence comprising ACAACAATCCC (SEQ ID NO: 70).

A protein as used herein can refer to molecules typically comprising one or more peptides or polypeptides. A peptide or polypeptide is typically a chain of amino acid residues, linked by peptide bonds. A peptide usually comprises between 2 and 50 amino acid residues. A polypeptide usually comprises more than 50 amino acid residues. A protein is typically folded into 3-dimensional form, which may be required for the protein to exert its biological function. A protein as used herein can include a fragment of a protein, a variant of a protein, and a fusion protein. A functional variant as used herein may refer to a full-length molecule, a fragment thereof, or a variant thereof. For example, a variant molecule may comprise a sequence modified by insertion, deletion, and/or substitution of one or more amino acids, in the case of protein sequence, or one or more nucleotides, in the case of nucleic acid sequence. For example, a variant molecule may comprise or encode a mutant protein, including, but not limited to, a gain-of-function or a loss-of-function mutant. A fragment may be a shorter portion of a full-length sequence of a nucleic acid molecule like DNA or RNA, or a protein. Accordingly, a fragment, typically, comprises a sequence that is identical to the corresponding stretch within the full-length sequence. In some embodiments, a fragment of a sequence may comprise at least 5% to at least 80% of a full-length nucleotide or amino acid sequence from which the fragment is derived. In some embodiments, a protein can be a mammalian protein. In some embodiments, a protein can be a human protein. In some embodiments, a protein may be a protein secreted from a cell. In some embodiments, a protein may be a protein on cell membranes. In some embodiments, a protein as referred to herein can be a protein that is secreted and acts either locally or systemically as a modulator of target cell signaling via receptors on cell surfaces, often involved in immunologic reactions or other host proteins involved in viral infection. Nucleotide and amino acid sequences of proteins useful in the context of the present invention, including proteins that are encoded by a gene of interest, are known in the art and available in the literature. For example, nucleotide and amino acid sequences of proteins useful in the context of the present invention, including proteins that are encoded by a gene of interest are available in the UniProt database.

Provided herein are compositions of recombinant RNA constructs comprising an siRNA capable of binding to a target mRNA to modulate expression of the target mRNA. In some instances, expression of the target mRNA (e.g., the level of protein encoded by the target mRNA) is downregulated by the siRNA capable of binding to the target mRNA. In some embodiments, expression of the target mRNA is inhibited by the siRNA capable of binding to the target mRNA. Inhibition or downregulation of expression of the target mRNA, as described herein, can refer to, but is not limited to, interference with the target mRNA to interfere with translation of the protein from the target mRNA; thus, inhibition or downregulation of expression of the target mRNA can refer to, but is not limited to, a decreased level of proteins expressed from the target mRNA compared to a level of proteins expressed from the target mRNA in the absence of recombinant RNA constructs comprising siRNA capable of binding to the target mRNA. Levels of protein expression can be measured by using any methods well known in the art and these include, but are not limited to Western-blotting, flow cytometry, ELISAs, radioimmunoassays (RIAs), and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Provided herein are compositions comprising recombinant RNA constructs comprising at least one nucleic acid sequence comprising siRNA capable of binding to a target mRNA and at least one nucleic acid sequence encoding a gene of interest wherein the target mRNA is different from an mRNA encoded by the gene of interest. Provided herein are compositions comprising recombinant RNA constructs comprising at least one nucleic acid sequence comprising siRNA capable of binding to a target mRNA and at least one nucleic acid sequence encoding a gene of interest wherein the siRNA does not affect expression of the gene of interest. In some instances, the siRNA is not capable of binding to an mRNA encoded by the gene of interest. In some instances, the siRNA does not inhibit the expression of the gene of interest. In some instances, the siRNA does not downregulate the expression of the gene of interest. Inhibiting or downregulating the expression of the gene of interest, as described herein, can refer to, but is not limited to, interfering with translation of proteins from recombinant RNA constructs; thus, inhibiting or downregulating the expression of the gene of interest can refer to, but is not limited to, a decreased level of protein compared to a level of protein expressed in the absence of recombinant RNA constructs comprising siRNA capable of binding to the target mRNA. Levels of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Provided herein are compositions comprising recombinant RNA constructs comprising at least one nucleic acid sequence comprising an siRNA capable of binding to a target mRNA. A list of non-limiting examples of target mRNAs that the siRNA is capable of binding to includes an mRNA of a gene comprising Tumor Necrosis Factor alpha (TNF-alpha or TNF-α), Activin Receptor-like Kinase 2 (ALK2), Turbo Green Fluorescence Protein (Turbo GFP), Vascular Endothelial Growth Factor A (VEGFA), Cellular Myelocytomatosis (c-Myc), Kirsten Rat Sarcoma (KRAS), Protein kinase B-1 (Akt1), Protein kinase B-2 (Akt2), Protein kinase B-3 (Akt3), or a functional variant thereof. In some embodiments, Turbo GFP sequence can be derived from marine copepod Pontellina plumate. A functional variant as used herein may refer to a full-length molecule, a fragment thereof, or a variant thereof. For example, a variant molecule may comprise a sequence modified by insertion, deletion, and/or substitution of one or more amino acids, in the case of protein sequence, or one or more nucleotides, in the case of nucleic acid sequence.

In some embodiments, recombinant RNA constructs described herein may encode or comprise one or more siRNAs, wherein each of the one or more siRNAs is capable of binding to a different mRNA. For example, recombinant RNA constructs may encode or comprise at least 3 siRNAs, wherein each of the 3 siRNAs is capable of binding to a different mRNA. In some embodiments, recombinant RNA constructs may encode or comprise at least 3 siRNAs, wherein one of the at least 3 siRNAs binds to c-Myc, one of the at least 3 siRNA binds to KRAS and one of the at least 3 siRNA binds to Akt1, Akt2, and/or Akt3. In some embodiments, recombinant RNA constructs may encode or comprise at least 3 siRNAs, wherein one of the at least 3 siRNAs binds to c-Myc, one of the at least 3 siRNA binds to KRAS and one of the at least 3 siRNA binds to pan-Akt (Akt1, Akt2, and Akt3).

In some embodiments, TNF-alpha comprises a sequence listed in SEQ ID NO: 32. In some embodiments, ALK2 comprises a sequence listed in SEQ ID NO: 33. In some embodiments, Turbo GFP comprises a sequence listed in SEQ ID NO: 34. In some embodiments, VEGFA comprises a sequence listed in SEQ ID NO: 115. In some embodiments, c-Myc comprises a sequence listed in SEQ ID NO: 122. In some embodiments, KRAS comprises a sequence listed in SEQ ID NO: 123. In some embodiments, Akt1 comprises a sequence listed in SEQ ID NO: 124. In some embodiments, Akt2 comprises a sequence listed in SEQ ID NO: 125. In some embodiments, Akt3 comprises a sequence listed in SEQ ID NO: 126.

In some aspects, the siRNA comprises a sense strand encoded by a sequence selected from the group consisting of SEQ ID NOs: 50-57 and 127-132. In some aspects, the siRNA comprises an anti-sense strand encoded by a sequence selected from the group consisting of SEQ ID NOs: 58-65 and 133-138. In some aspects, the siRNA comprises a sense strand encoded by a sequence selected from the group consisting of SEQ ID NOs: 50-57 and 127-132, and the corresponding anti-sense strand encoded by a sequence selected from the group consisting of SEQ ID NOs: 58-65 and 133-138.

### Gene of Interest

Provided herein are recombinant RNA constructs comprising one or more copies of nucleic acid sequence encoding a gene of interest. For example, recombinant RNA constructs may comprise 1, 2, 3, 4, 5, 6, 7, 8, 9, 10, or more copies of nucleic acid sequence encoding a gene of interest. In some instances, each of the 1, 2, 3, 4, 5, 6, 7, 8, 9, 10, or more copies of nucleic acid sequence encoding a gene of interest encodes the same gene of interest. In some instances, recombinant RNA constructs may comprise 1, 2, 3, 4, 5, 6, 7, 8, 9, 10, or more copies of nucleic acid sequence encoding a cytokine.

Also provided herein are recombinant RNA constructs comprising two or more copies of nucleic acid sequence encoding a gene of interest, wherein each of the two or more nucleic acid sequence may encode a different gene of interest. In some cases, each of the two or more nucleic acid sequences encoding different gene of interest may comprise a nucleic acid sequence encoding a secretory protein. In some cases, each of the two or more nucleic acid sequences encoding different gene of interest may comprise a nucleic acid sequence encoding a cytokine, e.g., Interleukin 4 (IL-4), Interleukin 2 (IL-2), or Interleukin 12 (IL-12). In some embodiments, each of the two or more nucleic acid sequences encoding different gene of interest may encode a different secretory protein. In some cases, each of the two or more nucleic acid encoding different gene of interest may comprise a nucleic acid sequence encoding Insulin-like Growth Factor 1 (IGF-1). Further provided herein are recombinant RNA constructs comprising a linker described herein. In some embodiments, the linker may connect each of the two or more nucleic acid sequences encoding a gene of interest. In some cases, the linker may be a non-cleavable linker. In some cases, the linker may be a cleavable linker. In some cases, the linker may be a self-cleavable linker. In some cases, the linker may be cleaved by a protein, e.g., an intracellular protein or an endogenous protein. In some instances, the linker is selected from the group consisting of Formula (I): XₘCAACAAXₙ, wherein X is any nucleotide, m is an integer from 1 to 12, and n is an integer from 0 to 4 (SEQ ID NO: 151); and Formula (II): XₚTCCCXᵣ, wherein X is any nucleotide, p is an integer from 0 to 17, and r is an integer from 0 to 13 (SEQ ID NO: 152). In some instances, the linker comprises a sequence comprising ACAACAA (SEQ ID NO: 23). In some embodiments, the linker is selected from the group consisting of SEQ ID NOs: 23, 67-75.

Other examples of the linker include, but are not limited to, a flexible linker, a 2A peptide linker (or 2A self-cleaving peptides) such as T2A, P2A, E2A, or F2A, and a tRNA linker, etc. The tRNA system is evolutionarily conserved across living organism and utilizes endogenous RNases P and Z to process multicistronic constructs (Dong et al., 2016). In some embodiments, the tRNA linker may comprise a nucleic acid sequence comprising AACAAAGCACCAGTGGTCTAGTGGTAGAATAGTACCCTGCCACGGTACAGACCC GGGTTCGATTCCCGGCTGGTGCA (SEQ ID NO: 39).

Provided herein are recombinant RNA constructs comprising an RNA encoding for a gene of interest for modulating the expression of the gene of interest. For example, expression of a protein encoded by the mRNA of the gene of interest can be modulated. For example, the expression of the gene of interest is upregulated by expressing a protein encoded by mRNA of the gene of interest in recombinant RNA constructs. For example, the expression of the gene of interest is upregulated by increasing the level of protein encoded by mRNA of the gene of interest in recombinant RNA constructs. The level of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Provided herein are recombinant RNA constructs comprising an RNA encoding for a gene of interest wherein the gene of the interest encodes a protein of interest. In some instances, the protein of interest is a therapeutic protein. In some instances, the protein of interest is of human origin i.e., is a human protein. In some instances, the gene of interest encodes a secretory protein. In some embodiments, the gene of interest encodes Insulin-like Growth Factor 1 (IGF-1). In some embodiments, the protein of interest is IGF-1. In some instances, the gene of interest encodes a cytokine. In some embodiments, the cytokine comprises an interleukin. In some embodiments, the protein of interest is Interleukin 4 (IL-4) or a functional variant thereof. In some embodiments, the protein of interest is Interleukin 2 (IL-2) or a functional variant thereof. In some embodiments, the protein of interest is Interleukin 12 (IL-12) or a functional variant thereof.

In some instances, recombinant RNA constructs comprising a nucleic acid sequence encoding a gene of interest may comprise a nucleic acid sequence encoding human insulin-like growth factor 1 (IGF-1). In some instances, IGF-1 as used herein may refer to the natural sequence of human IGF-1 (Uniprot database: P05019 and in the Genbank database: NM_001111285.3), a fragment, or a functional variant thereof. In one embodiment, recombinant RNA constructs can be naked RNA comprising a nucleic acid sequence encoding IGF-1. In this embodiment, recombinant RNA constructs may comprise a nucleic acid sequence encoding the mature human IGF-1. The natural DNA sequence encoding human IGF-1 may be codon-optimized. The natural sequence of human IGF-1 comprises a signal peptide having 21 amino acids (nucleotides 1-63), a pro-peptide having 27 amino acids (nucleotides 64-144), a mature human IGF-1 having 70 amino acids (nucleotides 145-354), and E-peptide having 77 amino acids (nucleotides 355-585). In some embodiments, recombinant RNA constructs may comprise a nucleic acid sequence encoding a pro-peptide (also called pro-domain) of IGF-1, a nucleic acid sequence encoding a mature protein of IGF-1, or an E-peptide (also called E-domain) of IGF-1 (i.e., IGF-1 with a carboxyl-terminal extension). In some embodiments, recombinant RNA constructs do not comprise a nucleic acid sequence encoding an E-peptide of IGF-1. In some embodiments, recombinant RNA constructs may comprise a nucleic acid sequence encoding a pro-peptide of IGF-1, a nucleic acid sequence encoding a mature protein of IGF-1, and a nucleic acid sequence encoding the signal peptide of brain-derived neurotrophic factor (BDNF). In some embodiments, IGF-1 is a human IGF-1.

In some embodiments, recombinant RNA constructs may comprise a nucleic acid sequence encoding a pro-peptide of IGF-1, preferably of human IGF-1 having 27 amino acids, and a nucleic sequence encoding a mature IGF-1, preferably a mature human IGF-1 having 70 amino acids, and preferably do not comprise a nucleotide sequence encoding an E-peptide of IGF-1, and preferably do not comprise a nucleic acid sequence encoding a human E-peptide of IGF-1. In some embodiments, recombinant RNA constructs may comprise a nucleic acid sequence encoding a pro-peptide of IGF-1, preferably of human IGF-1 having 27 amino acids, a nucleic sequence encoding a mature IGF-1, preferably a mature human IGF-1 having 70 amino acids, and a nucleic acid sequence encoding the signal peptide of brain-derived neurotrophic factor (BDNF). In some embodiments, recombinant RNA constructs do not comprise a nucleic sequence encoding an E-peptide of IGF-1, more preferably does not comprise a nucleic acid sequence encoding a human E-peptide of IGF-1.

In some embodiments, recombinant RNA constructs provided herein may comprise a nucleic acid sequence encoding a pro-peptide of human IGF-1 having 27 amino acids and a nucleic acid sequence encoding a mature human IGF-1 having 70 amino acids, and preferably do not comprise a nucleic acid sequence encoding an E-peptide of human IGF-1, wherein the nucleic acid sequence encoding the pro-peptide of human IGF-1 having 27 amino acids and the nucleic acid sequence encoding the mature human IGF-1 having 70 amino acids, and the nucleic acid sequence encoding the E-peptide are as referred to in the Uniprot database as UniProtKB-P05019. In some embodiments, IGF-1 described herein may have an amino acid sequence comprising SEQ ID NO: 29 or SEQ ID NO: 31.

In some instances, recombinant RNA constructs provided herein may comprise an mRNA encoding IGF-1. In some embodiments, the mRNA encoding IGF-1 may refer to an mRNA comprising a nucleotide sequence encoding the pro-peptide of human IGF-1 having 27 amino acids and/or a nucleotide sequence encoding the mature human IGF-1 having 70 amino acids. The nucleotide sequence encoding the pro-peptide of human IGF-1 and the nucleotide sequence encoding the mature human IGF-1 may be codon-optimized. In some instances, recombinant RNA constructs provided herein may comprise 1 copy of IGF-1 mRNA. In some instances, recombinant RNA constructs provided herein may comprise 2 or more copies of IGF-1 mRNA.

In some instances, Interleukin 4 (IL-4) or IL-4 as used herein may refer to the natural sequence of human IL-4 (Uniprot database: P05112 and in the Genbank database: NM_000589.4), a fragment, or a functional variant thereof. The natural DNA sequence encoding human IL-4 may be codon-optimized. The natural sequence of human IL-4 comprises a signal peptide having 24 amino acids (nucleotides 1-72) and a mature human IL-4 having 153 amino acids (nucleotides 73-459). In some embodiments, the signal peptide is unmodified IL-4 signal peptide. In some embodiments, the signal peptide is IL-4 signal peptide modified by insertion, deletion, and/or substitution of at least one amino acid. In some embodiments, Interleukin 4 (IL-4) or IL-4 as used herein may refer to the mature human IL-4. In some embodiments, a mature protein can refer to a protein synthesized in the endoplasmic reticulum and secreted via the Golgi apparatus in a cell expressing and secreting the protein. In some embodiments, a mature IL-4 may refer to an IL-4 protein synthesized in the endoplasmic reticulum and secreted via the Golgi apparatus in a cell expressing and secreting IL-4. In some embodiments, a mature human IL-4 may refer to an IL-4 protein synthesized in the endoplasmic reticulum and secreted via the Golgi apparatus in a human cell expressing and secreting human IL-4 and normally contains the amino acids encoded by nucleotide as shown in SEQ ID NO: 26. In some embodiments, IL-4 described herein may have an amino acid sequence comprising SEQ ID NO: 27.

The mRNA encoding IL-4 may refer to an mRNA comprising a nucleotide sequence encoding the pro-peptide of human IL-4 having 153 amino acids or a nucleotide sequence encoding the mature human IL-4 having 129 amino acids. The nucleotide sequence encoding the pro-peptide of human IL-4 and the nucleotide sequence encoding the mature human IL-4 may be codon-optimized. In some instances, recombinant RNA constructs provided herein may comprise 1 copy of IL-4 mRNA. In some instances, recombinant RNA constructs provided herein may comprise 2 or more copies of IL-4 mRNA.

In some instances, Interleukin 2 (IL-2) or IL-2 as used herein may refer to the natural sequence of human IL-2 (Uniprot database: P60568 or Q0GK43 and in the Genbank database: NM_000586.3), a fragment, or a functional variant thereof. The natural DNA sequence encoding human IL-2 may be codon-optimized. The natural sequence of human IL-2 may consist of a signal peptide having 20 amino acids (nucleotides 1-60) and the mature human IL-2 having 133 amino acids (nucleotides 61-459). In some embodiments, the signal peptide is unmodified IL-2 signal peptide. In some embodiments, the signal peptide is IL-2 signal peptide modified by insertion, deletion, and/or substitution of at least one amino acid. In some embodiments, the signal peptide of IL-2 may comprise a sequence comprising SEQ ID NO: 112. In some embodiments, Interleukin 2 (IL-2) or IL-2 as used herein may refer to the mature human IL-2. In some embodiments, a mature protein can refer to a protein synthesized in the endoplasmic reticulum and secreted via the Golgi apparatus in a cell expressing and secreting the protein. In some embodiments, a mature IL-2 may refer to an IL-2 protein synthesized in the endoplasmic reticulum and secreted via the Golgi apparatus in a cell expressing and secreting IL-2. In some embodiments, a mature human IL-2 may refer to an IL-2 protein synthesized in the endoplasmic reticulum and secreted via the Golgi apparatus in a human cell expressing and secreting human IL-2 and normally contains the amino acids encoded by nucleotide as shown in SEQ ID NO: 111. In some embodiments, the IL-2 fragment described herein may be at least partially functional, i.e., can perform an IL-2 activity at a similar or lower level compared to a wildtype or a full length IL-2. In some embodiments, the IL-2 fragment described herein may be fully functional, i.e., can perform an IL-2 activity at the same level compared to a wildtype or a full length IL-2. In some embodiments, the IL-2 variant, an IL-2 mutein, or the IL-2 mutant may comprise an IL-2 amino acid sequence modified by insertion, deletion, and/or substitution of at least one amino acid. In some embodiments, the IL-2 variant, an IL-2 mutein, or the IL-2 mutant may be at least partially functional, i.e., can perform an IL-2 activity at a similar or lower level compared to a wildtype IL-2. In some embodiments, the IL-2 variant, an IL-2 mutein, or the IL-2 mutant may be fully functional, i.e., can perform an IL-2 activity at the same level compared to a wildtype IL-2. In some embodiments, the IL-2 variant, an IL-2 mutein, or the IL-2 mutant may perform an IL-2 activity at a higher level compared to a wildtype IL-2. In some embodiments, IL-2 described herein may have an amino acid sequence comprising SEQ ID NO: 109 or 110. In some embodiments, IL-2 may comprise an IL-2 fragment, an IL-2 variant, an IL-2 mutein, or an IL-2 mutant.

The mRNA encoding IL-2 may refer to an mRNA comprising a nucleotide sequence encoding the pro-peptide of human IL-2 having 153 amino acids or a nucleotide sequence encoding the mature human IL-2 having 133 amino acids. The nucleotide sequence encoding the pro-peptide of human IL-2 and the nucleotide sequence encoding the mature human IL-2 may be codon-optimized. In some instances, recombinant RNA constructs provided herein may comprise 1 copy of IL-2 mRNA. In some instances, recombinant RNA constructs provided herein may comprise 2 or more copies of IL-2 mRNA.

In some instances, interleukin 12 (IL-12) or IL-12 as used herein may refer to the natural sequence of human IL-12 alpha (Uniprot database: P29459 and in the Genbank database: NM_000882.3), the natural sequence of human IL-12 beta (Uniprot database: P29460 and in the Genbank database: NM_002187.2), a fragment thereof, or a functional variant thereof. The natural DNA sequence encoding human IL-12 may be codon-optimized. The natural sequence of human IL-12 alpha may consist of a signal peptide having 22 amino acids and the mature human IL-12 having 197 amino acids as shown in SEQ ID NO: 116. In some embodiments, the signal peptide is unmodified IL-12 alpha signal peptide. In some embodiments, the signal peptide is IL-12 alpha signal peptide modified by insertion, deletion, and/or substitution of at least one amino acid. The natural sequence of human IL-12 beta may consist of a signal peptide having 22 amino acids and the mature human IL-12 having 306 amino acids as shown in SEQ ID NO: 119. In some embodiments, the signal peptide is unmodified IL-12 beta signal peptide. In some embodiments, the signal peptide is IL-12 beta signal peptide modified by insertion, deletion, and/or substitution of at least one amino acid.

In some embodiments, interleukin 12 (IL-12) or IL-12 as used herein may refer to the mature human IL-12 alpha. In some embodiments, interleukin 12 (IL-12) or IL-12 as used herein may refer to the mature human IL-12 beta. In some embodiments, a mature protein can refer to a protein synthesized in the endoplasmic reticulum and secreted via the Golgi apparatus in a cell expressing and secreting the protein. In some embodiments, a mature IL-12 may refer to an IL-12 alpha protein synthesized in the endoplasmic reticulum and secreted via the Golgi apparatus in a cell expressing and secreting IL-12. In some embodiments, a mature IL-12 may refer to an IL-12 beta protein synthesized in the endoplasmic reticulum and secreted via the Golgi apparatus in a cell expressing and secreting IL-12. In some embodiments, a mature human IL-12 may refer to an IL-12 alpha protein synthesized in the endoplasmic reticulum and secreted via the Golgi apparatus in a human cell expressing and secreting human IL-12 and normally contains the amino acids encoded by nucleotide as shown in SEQ ID NO: 118. In some embodiments, a mature human IL-12 may refer to an IL-12 beta protein synthesized in the endoplasmic reticulum and secreted via the Golgi apparatus in a human cell expressing and secreting human IL-12 and normally contains the amino acids encoded by nucleotide as shown in SEQ ID NO: 121.

In some embodiments, IL-12 alpha may comprise an IL-12 alpha fragment, an IL-12 alpha variant, an IL-12 alpha mutein, or an IL-12 alpha mutant. In some embodiments, the IL-12 alpha fragment described herein may be at least partially functional, i.e., can perform an IL-12 alpha activity at a similar or lower level compared to a wildtype or a full-length IL-12 alpha. In some embodiments, the IL-12 alpha fragment described herein may be fully functional, i.e., can perform an IL-12 alpha activity at the same level compared to a wildtype or a full-length IL-12 alpha. In some embodiments, the IL-12 alpha variant, an IL-12 alpha mutein, or the IL-12 alpha mutant may comprise an IL-12 alpha amino acid sequence modified by insertion, deletion, and/or substitution of at least one amino acid. In some embodiments, the IL-12 alpha variant, an IL-12 alpha mutein, or the IL-12 alpha mutant may be at least partially functional, i.e., can perform an IL-12 alpha activity at a similar or lower level compared to a wildtype IL-12 alpha. In some embodiments, the IL-12 alpha variant, an IL-12 alpha mutein, or the IL-12 alpha mutant may be fully functional, i.e., can perform an IL-12 alpha activity at the same level compared to a wildtype IL-12 alpha. In some embodiments, the IL-12 alpha variant, an IL-12 alpha mutein, or the IL-12 alpha mutant may perform an IL-12 alpha activity at a higher level compared to a wildtype IL-12 alpha.

In some embodiments, IL-12 beta may comprise an IL-12 beta fragment, an IL-12 beta variant, an IL-12 beta mutein, or an IL-12 beta mutant. In some embodiments, the IL-12 beta fragment described herein may be at least partially functional, i.e., can perform an IL-12 beta activity at a similar or lower level compared to a wildtype or a full-length IL-12 beta. In some embodiments, the IL-12 beta fragment described herein may be fully functional, i.e., can perform an IL-12 beta activity at the same level compared to a wildtype or a full-length IL-12 beta. In some embodiments, the IL-12 beta variant, an IL-12 beta mutein, or the IL-12 beta mutant may comprise an IL-12 beta amino acid sequence modified by insertion, deletion, and/or substitution of at least one amino acid. In some embodiments, the IL-12 beta variant, an IL-12 beta mutein, or the IL-12 beta mutant may be at least partially functional, i.e., can perform an IL-12 beta activity at a similar or lower level compared to a wildtype IL-12 beta. In some embodiments, the IL-12 beta variant, an IL-12 beta mutein, or the IL-12 beta mutant may be fully functional, i.e., can perform an IL-12 beta activity at the same level compared to a wildtype IL-12 beta. In some embodiments, the IL-12 beta variant, an IL-12 beta mutein, or the IL-12 beta mutant may perform an IL-12 beta activity at a higher level compared to a wildtype IL-12 beta.

The mRNA encoding IL-12 may refer to an mRNA comprising a nucleotide sequence encoding the propeptide of human IL-12 alpha having 219 amino acids or a nucleotide sequence encoding the mature human IL-12 alpha having 197 amino acids. The nucleotide sequence encoding the propeptide of human IL-12 alpha and the nucleotide sequence encoding the mature human IL-12 may be codon-optimized. The mRNA encoding IL-12 may refer to an mRNA comprising a nucleotide sequence encoding the propeptide of human IL-12 beta having 328 amino acids or a nucleotide sequence encoding the mature human IL-12 beta having 306 amino acids. The nucleotide sequence encoding the propeptide of human IL-12 beta and the nucleotide sequence encoding the mature human IL-12 may be codon-optimized. In some instances, recombinant RNA constructs, provided herein, may comprise 1 copy of IL-12 mRNA. In some instances, recombinant RNA constructs, provided herein, may comprise 2 or more copies of IL-12 mRNA.

### Target Motif

Provided herein are compositions comprising recombinant RNA constructs comprising a target motif. A target motif or a targeting motif as used herein can refer to any short peptide present in the newly synthesized polypeptides or proteins that are destined to any parts of cell membranes, extracellular compartments, or intracellular compartments, except cytoplasm or cytosol. In some embodiments, a peptide may refer to a series of amino acid residues connected one to the other, typically by peptide bonds between the α-amino and carboxyl groups of adjacent amino acid residues. Intracellular compartments include, but are not limited to, intracellular organelles such as nucleus, nucleolus, endosome, proteasome, ribosome, chromatin, nuclear envelope, nuclear pore, exosome, melanosome, Golgi apparatus, peroxisome, endoplasmic reticulum (ER), lysosome, centrosome, microtubule, mitochondria, chloroplast, microfilament, intermediate filament, or plasma membrane. In some embodiments, a signal peptide can be referred to as a signal sequence, a targeting signal, a localization signal, a localization sequence, a transit peptide, a leader sequence, or a leader peptide. In some embodiments, a target motif is operably linked to a nucleic acid sequence encoding a gene of interest. In some embodiments, the term “operably linked” can refer to a functional relationship between two or more nucleic acid sequences, e.g., a functional relationship of a transcriptional regulatory or signal sequence to a transcribed sequence. For example, a target motif or a nucleic acid encoding a target motif is operably linked to a coding sequence if it is expressed as a preprotein that participates in targeting the polypeptide encoded by the coding sequence to a cell membrane, intracellular, or an extracellular compartment. For example, a signal peptide or a nucleic acid encoding a signal peptide is operably linked to a coding sequence if it is expressed as a preprotein that participates in the secretion of the polypeptide encoded by the coding sequence. For example, a promoter is operably linked if it stimulates or modulates the transcription of the coding sequence. Non-limiting examples of a target motif comprise a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, a centrosomal localization signal (CLS) or any other signal that targets a protein to a certain part of cell membrane, extracellular compartments, or intracellular compartments.

A signal peptide is a short peptide present at the N-terminus of newly synthesized proteins that are destined towards the secretory pathway. The signal peptide of the present invention can be 10-40 amino acids long. A signal peptide can be situated at the N-terminal end of the protein of interest or at the N-terminal end of a pro-protein form of the protein of interest. A signal peptide may be of eukaryotic origin. In some embodiments, a signal peptide may be a mammalian protein. In some embodiments, a signal peptide may be a human protein. In some instances, a signal peptide may be a homologous signal peptide (i.e., from the same protein) or a heterologous signal peptide (i.e., from a different protein or a synthetic signal peptide). In some instances, a signal peptide may be a naturally occurring signal peptide of a protein or a modified signal peptide.

Provided herein are compositions comprising recombinant RNA constructs comprising a target motif, wherein the target motif may be selected from the group consisting of (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest; (d) a target motif homologous to a protein encoded by the gene of interest, wherein the target motif homologous to the protein encoded by the gene of interest is modified by insertion, deletion, and/or substitution of at least one amino acid; and (e) a naturally occurring amino acid sequence which does not have the function of a target motif in nature, wherein the naturally occurring amino acid sequence is optionally modified by insertion, deletion, and/or substitution of at least one amino acid.

Provided herein are compositions comprising recombinant RNA constructs comprising a target motif, wherein the target motif is a signal peptide. In some embodiments, the signal peptide is selected from the group consisting of: (a) a signal peptide heterologous to a protein encoded by the gene of interest; (b) a signal peptide heterologous to a protein encoded by the gene of interest, wherein the signal peptide heterologous to the protein encoded by the gene of interest is modified by insertion, deletion, and/or substitution of at least one amino acid, with proviso that the protein is not an oxidoreductase; (c) a signal peptide homologous to a protein encoded by the gene of interest; (d) a signal peptide homologous to a protein encoded by the gene of interest, wherein the signal peptide homologous to the protein encoded by the gene of interest is modified by insertion, deletion, and/or substitution of at least one amino acid; and (e) a naturally occurring amino acid sequence which does not have the function of a signal peptide in nature, wherein the naturally occurring amino acid sequence is optionally modified by insertion, deletion, and/or substitution of at least one amino acid. In some instances, the amino acids 1-9 of the N-terminal end of the signal peptide have an average hydrophobic score of above 2.

In some instances, a target motif heterologous to a protein encoded by the gene of interest or a signal peptide heterologous to a protein encoded by the gene of interest as used herein can refer to a naturally occurring target motif or signal peptide which is different from the naturally occurring target motif or signal peptide of a protein. For example, the target motif or the signal peptide is not derived from the gene of interest. Usually a target motif or a signal peptide heterologous to a given protein is a target motif or a signal peptide from another protein, which is not related to the given protein. For example, a target motif or a signal peptide heterologous to a given protein has an amino acid sequence that is different from the amino acid sequence of the target motif or the signal peptide of the given protein by more than 50%, 60%, 70%, 80%, 90%, or by more than 95%. Although heterologous sequences may be derived from the same organism, they naturally (in nature) do not occur in the same nucleic acid molecule, such as in the same mRNA. The target motif or the signal peptide heterologous to a protein and the protein to which the target motif or the signal peptide is heterologous can be of the same or different origin. In some embodiments, they are of eukaryotic origin. In some embodiments, they are of the same eukaryotic organism. In some embodiments, they are of mammalian origin. In some embodiments, they are of the same mammalian organism. In some embodiments, they are human origin. For example, an RNA construct may comprise a nucleic acid sequence encoding the human IL-4 gene and a signal peptide of another human protein. In some embodiments, an RNA construct may comprise a signal peptide heterologous to a protein wherein the signal peptide and the protein are of the same origin, namely of human origin.

In some instance, a target motif homologous to a protein encoded by the gene of interest or a signal peptide homologous to a protein encoded by the gene of interest as used herein can refer to a naturally occurring target motif or signal peptide of a protein. A target motif or a signal peptide homologous to a protein is the target motif or the signal peptide encoded by the gene of the protein as it occurs in nature. A target motif or a signal peptide homologous to a protein is usually of eukaryotic origin. In some embodiments, a target motif or a signal peptide homologous to a protein is of mammalian origin. In some embodiments, a target motif or a signal peptide homologous to a protein is of human origin.

In some instances, a naturally occurring amino acid sequence which does not have the function of a target motif in nature or a naturally occurring amino acid sequence which does not have the function of a signal peptide in nature as used herein can refer to an amino acid sequence which occurs in nature and is not identical to the amino acid sequence of any target motif or signal peptide occurring in nature. A naturally occurring amino acid sequence which does not have the function of a target motif or a signal peptide in nature can be between 10-50 amino acids long. In some embodiments, a naturally occurring amino acid sequence which does not have the function of a target motif or a signal peptide in nature is of eukaryotic origin and not identical to any target motif or signal peptide of eukaryotic origin. In some embodiments, a naturally occurring amino acid sequence which does not have the function of a target motif or a signal peptide in nature is of mammalian origin and not identical to any target motif or signal peptide of mammalian origin. In some embodiments, a naturally occurring amino acid sequence which does not have the function of a target motif or a signal peptide in nature is of human origin and not identical to any target motif or signal peptide of human origin occurring in nature. A naturally occurring amino acid sequence which does not have the function of a target motif or a signal peptide in nature is usually an amino acid sequence of the coding sequence of a protein. The terms “naturally occurring,” “natural,” and “in nature” as used herein have the equivalent meaning.

In some instances, amino acids 1-9 of the N-terminal end of the signal peptide as used herein can refer to the first nine amino acids of the N-terminal end of the amino acid sequence of a signal peptide. Analogously, amino acids 1-7 of the N-terminal end of the signal peptide as used herein can refer to the first seven amino acids of the N-terminal end of the amino acid sequence of a signal peptide and amino acids 1-5 of the N-terminal end of the signal peptide can refer to the first five amino acids of the N-terminal end of the amino acid sequence of a signal peptide.

In some instances, amino acid sequence modified by insertion, deletion, and/or substitution of at least one amino acid can refer to an amino acid sequence which includes an amino acid substitution, insertion, and/or deletion of at least one amino acid within the amino acid sequence. For example, target motif heterologous to a protein encoded by the gene of interest is modified by insertion, deletion, and/or substitution of at least one amino acid or signal peptide heterologous to a protein encoded by the gene of interest is modified by insertion, deletion, and/or substitution of at least one amino acid as used herein can refer to an amino acid sequence of a naturally occurring target motif or signal peptide heterologous to a protein which includes an amino acid substitution, insertion, and/or deletion of at least one amino acid within its naturally occurring amino acid sequence. For example, target motif homologous to a protein encoded by the gene of interest is modified by insertion, deletion, and/or substitution of at least one amino acid or signal peptide homologous to a protein encoded by the gene of interest is modified by insertion, deletion, and/or substitution of at least one amino acid as used herein can refer to a naturally occurring target motif or signal peptide homologous to a protein which includes an amino acid substitution, insertion, and/or deletion of at least one amino acid within its naturally occurring amino acid sequence. In some embodiments, naturally occurring amino acid sequence may be modified by insertion, deletion, and/or substitution of at least one amino acid and a naturally occurring amino acid sequence can include an amino acid substitution, insertion, and/or deletion of at least one amino acid within its naturally occurring amino acid sequence. An amino acid substitution or a substitution may refer to replacement of an amino acid at a particular position in an amino acid or polypeptide sequence with another amino acid. For example, the substitution R34K refers to a polypeptide, in which the arginine (Arg or R) at position 34 is replaced with a lysine (Lys or K). For the preceding example, 34K indicates the substitution of an amino acid at position 34 with a lysine (Lys or K). In some embodiments, multiple substitutions are typically separated by a slash. For example, R34K/L38V refers to a variant comprising the substitutions R34K and L38V. An amino acid insertion or an insertion may refer to addition of an amino acid at a particular position in an amino acid or polypeptide sequence. For example, insert −34 designates an insertion at position 34. An amino acid deletion or a deletion may refer to removal of an amino acid at a particular position in an amino acid or polypeptide sequence. For example, R34- designates the deletion of arginine (Arg or R) at position 34.

In some instances, deleted amino acid is an amino acid with a hydrophobic score of below −0.8, −0.7, −0.6, −0.5, −0.4, −0.3, −0.2, −0.1, 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, or below 1.9. In some instances, the substitute amino acid is an amino acid with a hydrophobic score which is higher than the hydrophobic score of the substituted amino acid. For example, the substitute amino acid is an amino acid with a hydrophobic score of 2.8 and higher, or 3.8 and higher. In some instances, the inserted amino acid is an amino acid with a hydrophobic score of 2.8 and higher or 3.8 and higher.

In some instances, an amino acid sequence described herein may comprise 1 to 15 amino acid insertions, deletions, and/or substitutions. In some embodiments, an amino acid sequence described herein may comprise 1 to 7 amino acid insertions, deletions, and/or substitutions. In some instances, an amino acid sequence described herein may not comprise amino acid insertions, deletions, and/or substitutions. In some instances, an amino acid sequence described herein may comprise 1 to 15 amino acid insertions, deletions, and/or substitutions within the amino acids 1-30 of the N-terminal end of the amino acid sequence of the target motif or the signal peptide. In some embodiments, an amino acid sequence described herein may comprise 1 to 9 amino acid insertions, deletions, and/or substitutions within the amino acids 1-30 of the N-terminal end of the amino acid sequence of the target motif or the signal peptide. In some instances, an amino acid sequence described herein may comprise 1 to amino acid insertions, deletions, and/or substitutions within the amino acids 1-20 of the N-terminal end of the amino acid sequence of the target motif or the signal peptide. In some embodiments, an amino acid sequence described herein may comprise 1 to 9 amino acid insertions, deletions, and/or substitutions within the amino acids 1-20 of the N-terminal end of the amino acid sequence of the target motif or the signal peptide. In some instances, at least one amino acid of an amino acid sequence described herein may be optionally modified by deletion, and/or substitution.

In some instances, the average hydrophobic score of the first nine amino acids of the N-terminal end of the amino acid sequence of the modified signal peptide is increased 1.0 unit or above compared to the signal peptide without modification. In some instances, hydrophobic score or hydrophobicity score can be used synonymously to hydropathy score herein and can refer to the degree of hydrophobicity of an amino acid as calculated according to the Kyte-Doolittle scale (Kyte J., Doolittle R. F.; J. Mol. Biol. 157:105-132(1982)). The amino acid hydrophobic scores according to the Kyte-Doolittle scale are as follows:

TABLE A
Amino Acid Hydrophobic Scores

| Amino Acid | One Letter Code | Hydrophobic Score |
|---|---|---|
| Isoleucine | I | 4.5 |
| Valine | V | 4.2 |
| Leucine | L | 3.8 |
| Phenylalanine | F | 2.8 |
| Cysteine | C | 2.5 |
| Methionine | M | 1.9 |
| Alanine | A | 1.8 |
| Glycine | G | −0.4 |
| Threonine | T | −0.7 |
| Serine | S | −0.8 |
| Tryptophan | W | −0.9 |
| Tyrosine | Y | −1.3 |
| Proline | P | −1.6 |
| Histidine | H | −3.2 |
| Glutamic acid | E | −3.5 |
| Glutamine | Q | −3.5 |
| Aspartic acid | D | −3.5 |
| Asparagine | N | −3.5 |
| Lysine | K | −3.9 |
| Arginine | R | −4.5 |

In some instances, average hydrophobic score of an amino acid sequence can be calculated by adding the hydrophobic score according to the Kyte-Doolittle scale of each of the amino acid of the amino acid sequence divided by the number of the amino acids. For example, the average hydrophobic score of the amino acids 1-9 of the N-terminal end of the amino acid sequence of a signal peptide can be calculated by adding the hydrophobic score or each of the nine amino acids divided by nine.

The polarity is calculated according to Zimmerman Polarity index (Zimmerman J. M., Eliezer N., Simha R.; J. Theor. Biol. 21:170-201(1968)). In some embodiments, average polarity of an amino acid sequence can be calculated by adding the polarity value calculated according to Zimmerman Polarity index of each of the amino acid of the amino acid sequence divided by the number of the amino acids. For example, the average polarity of the amino acids 1-9 of the N-terminal end of the amino acid sequence of a signal peptide can be calculated by adding the average polarity of each of the nine amino acids of the amino acids 1-9 of the N-terminal end, divided by nine. The polarity of amino acids according to Zimmerman Polarity index is as follows:

TABLE B
Amino Acid Polarity

| Amino Acid | One Letter Code | Polarity |
|---|---|---|
| Isoleucine | I | 0.13 |
| Valine | V | 0.13 |
| Leucine | L | 0.13 |
| Phenylalanine | F | 0.35 |
| Cysteine | C | 1.48 |
| Methionine | M | 1.43 |
| Alanine | A | 0 |
| Glycine | G | 0 |
| Threonine | T | 1.66 |
| Serine | S | 1.67 |
| Tryptophan | W | 2.1 |
| Tyrosine | Y | 1.61 |
| Proline | P | 1.58 |
| Histidine | H | 51.6 |
| Glutamic acid | E | 49.9 |
| Glutamine | Q | 3.53 |
| Aspartic acid | D | 49.7 |
| Asparagine | N | 3.38 |
| Lysine | K | 49.5 |
| Arginine | R | 52 |

In some instances, a naturally occurring signal peptide of Insulin-like Growth Factor 1 (IGF-1) may be modified by one or more substitutions, deletions, and/or insertions, wherein the naturally occurring signal peptide of IGF-1 is referred to the amino acids 1-20 of the IGF-1 amino acid sequence in the Uniprot database as P05019 and in the Genbank database as NM_001111285.3. In some instances, the amino acid sequence of IGF-1 signal peptide may be modified by the one or more substitutions, deletions, and/or insertions selected from the group consisting of G2L, K3-, SSL, T9L, Q10L, and C15-. In some embodiments, the wild type (WT) IGF-1 signal peptide amino acid sequence comprises a sequence comprising SEQ ID NO: 46. In some instances, a modified IGF-1 signal peptide has an amino acid sequence comprising a sequence comprising SEQ ID NO: 41 encoded by the DNA sequence as shown in SEQ ID NO: 42. In some instances, a modified IGF-1 signal peptide has an amino acid sequence comprising a sequence comprising SEQ ID NO: 48 encoded by the DNA sequence as shown in SEQ ID NO: 49.

SEQ ID NO: 41
Met-Leu-Ile-Leu-Leu-Leu-Pro-Leu-Leu-Leu-
Phe-Lys-Cys-Phe-Cys-Asp-Phe-Leu-Lys
SEQ ID NO: 42
ATGCTGATTCTGCTGCTGCCCCTGCTGCTGTTCAAGTGC
TCTGCGACTTCCTGAAA
SEQ ID NO: 48
MTILFLTMVISYFGCMKA
SEQ ID NO: 49
ATGACCATCCTGTTTCTGACAATGGTCATCAGCTACTTC
GGCTGCATGAAGGCC

In some instances, the pro-peptide of IGF-1 may be modified. In some embodiments, a naturally occurring amino acid sequence of the pro-peptide of IGF-1, which does not have the function of a signal peptide in nature (Uniprot database as P05019), is modified by deletion of ten amino acid residues (VKMHTMSSSH (SEQ ID NO: 45) flanking 22-31 in the N-terminal end of the pro-peptide and has preferably the amino acid sequence as shown in SEQ ID NO: 43 encoded by the DNA sequence as shown in SEQ ID NO: 44.

SEQ ID NO: 43
Met-Leu-Phe-Tyr-Leu-Ala-Leu-Cys-Leu-Leu-
Thr-Phe-Thr-Ser-Ser-Ala-Thr-Ala
SEQ ID NO: 44
ATGCTGTTCTATCTGGCCCTGTGCCTGCTGACCTTTACC
AGCTCTGCTACCGCC

In some instances, an mRNA comprising a nucleic acid sequence encoding the pro-peptide of IGF-1 and a nucleic acid sequence encoding the mature IGF-1, but not comprising a nucleic acid sequence encoding an E-peptide of IGF-1 may refer to an mRNA which comprises a nucleotide sequence encoding the pro-peptide of human IGF-1 having 27 amino acids and a nucleotide sequence encoding the mature human IGF-1 having 70 amino acids, but does not comprise a nucleotide sequence encoding an E-peptide of human IGF-1 i.e., does not comprise a nucleotide sequence encoding an Ea-, Eb-, or Ec-domain. The nucleotide sequence encoding the pro-peptide of human IGF-1 having 27 amino acids and the nucleotide sequence encoding the mature human IGF-1 having 70 amino acids may be codon-optimized.

In some instances, a naturally occurring signal peptide of Interleukin 4 (IL-4) may be modified by one or more substitutions, deletions, and/or insertions, wherein the naturally occurring signal peptide of IL-4 is referred to the amino acids 1-24 of the IL-4 amino acid sequence in the Uniprot database as P05112 and in the Genbank database as NM_000589.4. In some instances, the amino acid sequence of IL-4 signal peptide may be modified by the one or more substitutions, deletions, and/or insertions of one or more amino acid residues.

In some instances, a naturally occurring signal peptide of interleukin 2 (IL-2) may be modified by one or more substitutions, deletions, and/or insertions, wherein the naturally occurring signal peptide of IL-2 is referred to the amino acids 1-20 of the IL-2 amino acid sequence in the Uniprot database as P60568 or Q0GK43 and in the Genbank database as NM_000586.3. In some instances, the amino acid sequence of IL-2 signal peptide may be modified by the one or more substitutions, deletions, and/or insertions selected from the group consisting of Y2L, R3K, R3-, M4L, Q5L, S8L, S8A, −13A, L14T, L16A, V17-, and V17A. In some instances, the wild type (WT) IL-2 signal peptide is encoded by a DNA sequence comprising SEQ ID NO: 113. In some instances, a modified IL-2 signal peptide has an amino acid sequence comprising a sequence comprising SEQ ID NO: 112. In some instances, a modified IL-2 signal peptide is encoded by a DNA sequence comprising SEQ ID NO: 114 (Y2L/R3-/M4L/Q5L/S8A/-A13/L14T/L16A and V17A).

In some instances, a naturally occurring signal peptide of Interleukin 12 (IL-12) may be modified by one or more substitutions, deletions, and/or insertions, wherein the naturally occurring signal peptide of IL-12 is referred to the amino acids 1-22 of the IL-12 amino acid sequence in the Genbank database as NM_000882.4 or in the Genbank database as NM_002187.2. In some instances, the amino acid sequence of IL-12 signal peptide may be modified by the one or more substitutions, deletions, and/or insertions of one or more amino acid residues.

### Expression Vector and Production of RNA Constructs

Provided herein are compositions comprising recombinant polynucleic acid constructs encoding recombinant RNA constructs described herein. Provided herein are compositions comprising recombinant polynucleic acid constructs encoding recombinant RNA constructs comprising a first RNA sequence, a second RNA sequence, and a linker RNA sequence, wherein the linker RNA sequence connects the first RNA sequence and the second RNA sequence. In some instances, a linker has a structure independently selected from the group consisting of Formula (I): XₘCAACAAXₚ, wherein X is any nucleotide, m is an integer from 1 to 12, and n is an integer from 0 to 4 (SEQ ID NO: 151); and Formula (II): XₚTCCCXᵣ, wherein X is any nucleotide, p is an integer from 0 to 17, and r is an integer from 0 to 13 (SEQ ID NO: 152). In some instances, a linker may comprise a sequence comprising ACAACAA (SEQ ID NO: 23). In some instances, the first RNA sequence or the second RNA sequence may encode a gene of interest. In some embodiments, the first RNA sequence or the second RNA sequence may be an mRNA encoding a gene of interest. In some instances, the first RNA sequence or the second RNA sequence may comprise one or more genetic elements that modulate the expression of a target RNA. In some embodiments, the first RNA sequence or the second RNA sequence may comprise one or more siRNAs each capable of binding to a target RNA. For example, an mRNA encoding a gene of interest can be an mRNA of IL-4, IL-2, IL-12, or IGF-1. For example, a target RNA can be TNF-alpha mRNA, ALK2 mRNA, Turbo GFP mRNA, VEGFA mRNA, c-Myc mRNA, KRAS mRNA, Akt1 mRNA, Akt2 mRNA, or Akt3 mRNA.

In related aspects, recombinant polynucleic acid constructs encoding recombinant RNA constructs may encode 1, 2, 3, 4, 5, or more siRNA species. In related aspects, recombinant polynucleic acid constructs encoding recombinant RNA constructs may encode 1 siRNA species directed to a target mRNA. In related aspects, recombinant polynucleic acid constructs encoding recombinant RNA constructs may encode 3 siRNAs, each directed to a target mRNA. In related aspects, each of the siRNA species may comprise the same sequence, different sequence, or a combination thereof. For example, recombinant polynucleic acid constructs encoding recombinant RNA constructs may encode 3 siRNAs, each directed to the same region or sequence of the target mRNA. For example, recombinant polynucleic acid constructs encoding recombinant RNA constructs may encode 3 siRNAs, each directed to a different region or sequence of the target mRNA. In some aspects, recombinant polynucleic acid constructs encoding recombinant RNA constructs may encode 3 siRNA species, wherein each of the 3 siRNA species is directed to a different target mRNA. In some embodiments, a target mRNA may be TNF-alpha, ALK2, Turbo GFP mRNA, VEGFA mRNA, c-Myc mRNA, KRAS mRNA, Akt1 mRNA, Akt2 mRNA, or Akt3 mRNA. In related aspects, recombinant polynucleic acid constructs may comprise a sequence selected from the group consisting of SEQ ID NOs: 10-18 and 93-100.

The polynucleic acid constructs, described herein, can be obtained by any method known in the art, such as by chemically synthesizing the DNA chain, by PCR, or by the Gibson Assembly method. The advantage of constructing polynucleic acid constructs by chemical synthesis or a combination of PCR method or Gibson Assembly method is that the codons may be optimized to ensure that the fusion protein is expressed at a high level in a host cell. Codon optimization can refer to a process of modifying a nucleic acid sequence for expression in a host cell of interest by replacing at least one codon (e.g., more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database,” and these tables can be adapted in a number of ways. Computer algorithms for codon optimizing a particular sequence for expression in a particular host cell are also available, such as Gene Forge® (Aptagen, PA) and GeneOptimizer® (ThermoFischer, MA). Once obtained polynucleotides can be incorporated into suitable vectors. Vectors as used herein can refer to naturally occurring or synthetically generated constructs for uptake, proliferation, expression or transmission of nucleic acids in vivo or in vitro, e.g., plasmids, minicircles, phagemids, cosmids, artificial chromosomes/mini-chromosomes, bacteriophages, viruses such as baculovirus, retrovirus, adenovirus, adeno-associated virus, herpes simplex virus, bacteriophages. Methods used to construct vectors are well known to a person skilled in the art and described in various publications. In particular techniques for constructing suitable vectors, including a description of the functional and regulatory components such as promoters, enhancers, termination and polyadenylation signals, selection markers, origins of replication, and splicing signals, are known to the person skilled in the art. A variety of vectors are well known in the art and some are commercially available from companies such as Agilent Technologies, Santa Clara, Calif.; Invitrogen, Carlsbad, Calif; Promega, Madison, Wis.; Thermo Fisher Scientific; or Invivogen, San Diego, Calif A non-limiting examples of vectors for in vitro transcription includes pT7CFE1-CHis, pMX (such as pMA-T, pMA-RQ, pMC, pMK, pMS, pMZ), pEVL, pSP73, pSP72, pSP64, and pGEM (such as pGEM®-4Z, pGEM®-5Zf(+), pGEM®-11Zf(+), pGEM®-9Zf(−), pGEM®-3Zf(+/−), pGEM®-7Zf(+/−)). In some instances, recombinant polynucleic acid constructs may be DNA.

The polynucleic acid constructs, as described herein, can be circular or linear. For example, circular polynucleic acid constructs may include vector system such as pMX, pMA-T, pMA-RQ, or pT7CFE1-CHis. For example, linear polynucleic acid constructs may include linear vector such as pEVL or linearized vectors. In some instances, recombinant polynucleic acid constructs may further comprise a promoter. In some instances, the promoter may be present upstream of or 5′ to the sequence encoding for the first RNA sequence and the second RNA sequence. Non-limiting examples of a promoter can include T3, T7, SP6, P60, Syn5, and KP34. In some instances, recombinant polynucleic acid constructs provided herein may comprise a T7 promoter comprising a sequence comprising TAATACGACTCACTATA (SEQ ID NO: 20). In some instances, recombinant polynucleic acid constructs further comprises a sequence encoding a Kozak sequence. A Kozak sequence may refer to a nucleic acid sequence motif that functions as the protein translation initiation site. Kozak sequences are described at length in the literature, e.g., by Kozak, M., Gene 299(1-2):1-34, incorporated herein by reference herein in its entirety. In some embodiments, recombinant polynucleic acid constructs comprises a sequence encoding a Kozak sequence comprising a sequence comprising GCCACC (SEQ ID NO: 19). In some instances, recombinant polynucleic acid constructs described herein may be codon-optimized.

Provided herein are compositions comprising recombinant polynucleic acid constructs encoding RNA constructs described herein comprising one or more nucleic acid sequence encoding an siRNA capable of binding to a target RNA and one or more nucleic acid sequence encoding a gene of interest, wherein the siRNA capable of binding to a target RNA is not a part of an intron sequence encoded by the gene of interest. In some instances, the gene of interest is expressed without RNA splicing. In some instances, the siRNA capable of binding to a target RNA is not encoded by or comprised of an intron sequence of the gene of interest. In some instances, the siRNA capable of binding to a target RNA binds to an exon of a target mRNA. In some instances, the siRNA capable of binding to a target RNA specifically binds to one target RNA. In some instances, recombinant polynucleic acid constructs may comprise a nucleic acid sequence comprising a sequence selected from the group consisting of SEQ ID NOs: 10-18 and 101-108.

Provided herein are methods of producing RNA construct compositions described herein. For example, recombinant RNA constructs may be produced by in vitro transcription from a polynucleic acid construct comprising a promoter for an RNA polymerase, at least one nucleic acid sequence encoding a gene of interest, at least one nucleic acid sequence encoding an siRNA capable of binding to a target mRNA, and a nucleic acid sequence encoding poly(A) tail. In vitro transcription reaction may further comprise an RNA polymerase, a mixture of nucleotide triphosphates (NTPs), and/or a capping enzyme. Details of producing RNAs using in vitro transcription as well as isolating and purifying transcribed RNAs is well known in the art and can be found, for example, in Beckert & Masquida ((2011) Synthesis of RNA by In vitro Transcription. RNA. Methods in Molecular Biology (Methods and Protocols), vol 703. Humana Press). A non-limiting list of in vitro transcript kits includes MEGAscript™ T3 Transcription Kit, MEGAscript T7 kit, MEGAscript™ SP6 Transcription Kit, MAXlscript™ T3 Transcription Kit, MAXIscript™ T7 Transcription Kit, MAXIscript™ SP6 Transcription Kit, MAXIscript™ T7/T3 Transcription Kit, MAXlscript™ SP6/T7 Transcription Kit, mMESSAGE mMACHINE™ T3 Transcription Kit, mMESSAGE mMACHINE™ T7 Transcription Kit, mMESSAGE mMACHINE™ SP6 Transcription Kit, MEGAshortscript™ T7 Transcription Kit, HiScribe™ T7 High Yield RNA Synthesis Kit, HiScribe™ T7 In Vitro Transcription Kit, AmpliScribe™ T7-Flash™ Transcription Kit, AmpliScribe™ T7 High Yield Transcription Kit, AmpliScribe™ T7-Flash™ Biotin-RNA Transcription Kit, T7 Transcription Kit, HighYield T7 RNA Synthesis Kit, DuraScribe® T7 Transcription Kit, etc.

The in vitro transcription reaction can further comprise a transcription buffer system, nucleotide triphosphates (NTPs), and an RNase inhibitor. In some embodiments, the transcription buffer system may comprise dithiothreitol (DTT) and magnesium ions. The NTPs can be naturally occurring or non-naturally occurring (modified) NTPs. Non-limiting examples of non-naturally occurring (modified) NTPs include N¹-methylpseudouridine, pseudouridine, N¹-ethylpseudouridine, N¹-methoxymethylpseudouridine, N¹-propylpseudouridine, 2-thiouridine, 4-thiouridine, 5-methoxyuridine, 5-methylurdine, 5-carboxymethylesteruridine, 5-formyluridine, 5-carboxyuridine, 5-hydroxyuridine, 5-bromouridine, 5-Iodouridine, 5,6-dihydrouridine, 6-azauridine, thienouridine, 3-methyluridine, 1-carboxymethyl-pseudouridine, 4-thio-1-methyl-pseudouridine, 2-thio-1-methyl-pseudouridine, dihydrouridine, dihydropseudouridine, 2-methoxyuridine, 2-methoxy-4-thio-uridine, 4-methoxy-pseudouridine, 4-methoxy-2-thio-pseudouridine, 5-methylcytidine, 5-methoxycytidine, 5-hydroxymethylcytidine, 5-formylcytidine, 5-carboxy cytidine, 5-hydroxycytidine, 5-iodocytidine, 5-bromocytidine, 2-thiocytidine, 5-azacytidine, pseudoisocytidine, 3-methyl-cytidine, N⁴-acetylcytidine, 5-formylcytidine, N⁴-methylcytidine, 5-hydroxymethylcytidine, 1-methyl-pseudoisocytidine, 4-methoxy-pseudoisocytidine, and 4-methoxy-1-methyl-pseudoisocytidine, N¹-methyladenosine, N⁶-methyladenosine, N⁶-methyl-2-aminoadenosine, N⁶-isopentenyladenosine, N⁶,N⁶-dimethyladenosine, 7-methyladenine, 2-methylthio-adenine, and 2-methoxy-adenine. Non-limiting examples of DNA-dependent RNA polymerase include T3, T7, SP6, P60, Syn5, and KP34 RNA polymerases. In some embodiments, the RNA polymerase is selected from the group consisting of T3 RNA polymerase, T7 RNA polymerase, SP6 RNA polymerase, P60 RNA polymerase, Syn5 RNA polymerase, and KP34 RNA polymerase.

Transcribed RNAs, as described herein, may be isolated and purified from the in vitro transcription reaction mixture. For example, transcribed RNAs may be isolated and purified using column purification. Details of isolating and purifying transcribed RNAs from in vitro transcription reaction mixture is well known in the art and any commercially available kits may be used. A non-limiting list of RNA purification kits includes MEGAclear kit, Monarch® RNA Cleanup Kit, EasyPure® RNA Purification Kit, NucleoSpin® RNA Clean-up, etc.

### Therapeutic Applications

Provided herein are compositions useful in the treatment of a disease or condition. In some aspects, compositions are present or administered in an amount sufficient to treat or prevent a disease or condition. In some aspects, provided herein, is a method of treating a disease or condition comprising administering to a subject in need thereof the composition or the pharmaceutical composition described herein. In some aspects, provided herein, is the composition or the pharmaceutical composition described herein for use in a method of treating a disease or a condition in a subject in need thereof. In some aspects, provided herein, is the use of the composition or the pharmaceutical composition described herein for the manufacture of a medicament for treating a disease or a condition in a subject in need thereof. In some embodiments, the disease or condition comprises a skin disease or condition. In some embodiments, the skin disease or condition comprises an inflammatory skin disorder. In some embodiments, an inflammatory skin disorder comprises psoriasis. In some embodiments, the disease or condition comprises a muscular disease or condition. In some embodiments, the muscular disease or condition comprises a skeletal muscle disorder. In some embodiments, the skeletal muscle disorder comprises fibrodysplasia ossificans progressiva (FOP). In some embodiments, the disease or condition comprises cancer. In some embodiments, the cancer comprises glioblastoma, human tongue squamous carcinoma, human lung carcinoma, or human monocyte leukemia. Provided herein are recombinant polynucleic acid or RNA construct compositions comprising a first RNA sequence, a second RNA sequence, and a linker RNA sequence, wherein the linker RNA sequence links the first RNA sequence and the second RNA sequence. In some instances, the first RNA sequence or the second RNA sequence may encode a gene of interest. In some embodiments, the gene of interest may comprise IL-4, IL-2, IL-12, or IGF-1. In some instances, the first RNA sequence or the second RNA sequence may comprise a genetic element that can reduce expression of a gene associated with a disease or condition described herein. In some embodiments, the genetic element that can reduce expression of a gene associated with a disease or condition may comprise siRNA targeting TNF-alpha mRNA or a functional variant. In some embodiments, the genetic element that can reduce expression of a gene associated with a disease or condition may comprise siRNA targeting ALK2 mRNA or a functional variant. In some embodiments, the genetic element that can reduce expression of a gene associated with a disease or condition may comprise siRNA targeting VEGFA mRNA or a functional variant. In some embodiments, the genetic element that can reduce expression of a gene associated with a disease or condition may comprise siRNA targeting c-Myc mRNA or a functional variant. In some embodiments, the genetic element that can reduce expression of a gene associated with a disease or condition may comprise siRNA targeting KRAS mRNA or a functional variant. In some embodiments, the genetic element that can reduce expression of a gene associated with a disease or condition may comprise siRNA targeting Akt1 mRNA or a functional variant. In some embodiments, the genetic element that can reduce expression of a gene associated with a disease or condition may comprise siRNA targeting Akt2 mRNA or a functional variant. In some embodiments, the genetic element that can reduce expression of a gene associated with a disease or condition may comprise siRNA targeting Akt3 mRNA or a functional variant.

Also provided herein are pharmaceutical compositions comprising any recombinant RNA construct composition described herein and a pharmaceutically acceptable excipient. A pharmaceutical composition can denote a mixture or solution comprising a therapeutically effective amount of an active pharmaceutical ingredient together with one or more pharmaceutically acceptable excipients to be administered to a subject in need thereof. The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. The term “Pharmaceutically acceptable” can refer to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. A pharmaceutically acceptable excipient can denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products. Pharmaceutical compositions can facilitate administration of the compound to an organism and can be formulated in a conventional manner using one or more pharmaceutically acceptable inactive ingredients that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In some embodiments, pharmaceutical compositions can be formulated by dissolving active substances (e.g., recombinant polynucleic acid or RNA constructs described herein) in aqueous solution for injection into disease tissues or disease cells. In some embodiments, pharmaceutical compositions can be formulated by dissolving active substances (e.g., recombinant polynucleic acid or RNA constructs described herein) in aqueous solution for direct injection into disease tissues or disease cells.

Also provided herein are methods of treating a disease or condition in a subject in need thereof, comprising administering to the subject a therapeutically effective amount of polynucleic acid construct or recombinant RNA construct compositions or pharmaceutical compositions described herein. The terms “effective amount” or “therapeutically effective amount,” as used herein, refer to a sufficient amount of an agent or a compound being administered which will relieve to some extent one or more of the symptoms of the disease or the condition being treated; for example a reduction and/or alleviation of one or more signs, symptoms, or causes of a disease, or any other desired alteration of a biological system. For example, an “effective amount” for therapeutic uses can be an amount of an agent that provides a clinically significant decrease in one or more disease symptoms. An appropriate “effective” amount may be determined using techniques, such as a dose escalation study, in individual cases.

The terms “treat,” “treating” or “treatment,” as used herein, include alleviating, abating or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. In some embodiments, treating a disease or condition comprises reducing the size of disease tissues or disease cells. In some embodiments, treating a disease or a condition in a subject comprises increasing the survival of a subject. In some embodiments, treating a disease or condition comprises reducing or ameliorating the severity of a disease, delaying onset of a disease, inhibiting the progression of a disease, reducing hospitalization of or hospitalization length for a subject, improving the quality of life of a subject, reducing the number of symptoms associated with a disease, reducing or ameliorating the severity of a symptom associated with a disease, reducing the duration of a symptom associated with a disease, preventing the recurrence of a symptom associated with a disease, inhibiting the development or onset of a symptom of a disease, or inhibiting of the progression of a symptom associated with a disease. In some embodiments, treating a cancer comprises reducing the size of tumor or increasing survival of a patient with a cancer.

In some cases, a subject can encompass mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some cases, the mammal is a human. In some cases, the subject may be an animal. In some cases, an animal may comprise human beings and non-human animals. In one embodiment, a non-human animal may be a mammal, for example a rodent such as rat or a mouse. In another embodiment, a non-human animal may be a mouse. In some instances, the subject is a mammal. In some instances, the subject is a human. In some instances, the subject is an adult, a child, or an infant. In some instances, the subject is a companion animal. In some instances, the subject is a feline, a canine, or a rodent. In some instances, the subject is a dog or a cat.

In some aspects, provided herein, is a method of treating a disease or condition in a subject, comprising administering to the subject recombinant RNA construct compositions or pharmaceutical compositions, described herein, comprising an mRNA encoding a gene of interest and siRNA capable of binding to a target mRNA. In some embodiments, the target mRNA comprises an mRNA of TNF-alpha, ALK2, VEGFA, c-Myc, KRAS, Akt1, Akt2, Akt3, or a functional variant thereof. In some embodiments, the mRNA encoding the gene of interest encodes IGF-1 or a functional variant thereof. In some embodiments, the mRNA encoding the gene of interest encodes a cytokine. In some embodiments, the cytokine is an IL-4 or a functional variant thereof. In some embodiments, the cytokine is an IL-2 or a functional variant thereof. In some embodiments, the mRNA encoding the gene of interest encodes a cytokine. In some embodiments, the cytokine is an IL-12 or a functional variant thereof.

In some aspects, provided herein, is a method of treating a disease or condition in a subject, the method comprising administering to the subject recombinant RNA compositions or pharmaceutical compositions described herein comprising an mRNA encoding IL-4 and siRNA capable of binding to an mRNA of TNF-alpha. In some aspects, provided herein, is a method of treating a disease or condition in a subject, the method comprising administering to the subject recombinant RNA construct compositions or pharmaceutical compositions, described herein, comprising an mRNA encoding IGF-1 and siRNA capable of binding to an mRNA of a ALK2. In some aspects, provided herein, is a method of treating a disease or condition in a subject, the method comprising administering to the subject recombinant RNA construct compositions or pharmaceutical compositions, described herein, comprising an mRNA encoding IL-2 and siRNA capable of binding to an mRNA of a VEGFA. In some aspects, provided herein, is a method of treating a disease or condition in a subject, the method comprising administering to the subject recombinant RNA construct compositions or pharmaceutical compositions, described herein, comprising an mRNA encoding IL-12 and siRNA capable of binding to an mRNA of a c-Myc, KRAS, Akt1, Akt2, and/or Akt2. In some aspects, provided herein, is a method of treating a disease or condition in a subject, the method comprising administering to the subject recombinant RNA construct compositions or pharmaceutical compositions, described herein, comprising an mRNA encoding IL-12 and siRNA capable of binding to an mRNA of a c-Myc, KRAS, Akt1, Akt2, and Akt2. In some embodiments, the disease or condition comprises a skin disease or condition, a muscular disease or condition, or cancer. In some embodiments, the disease or condition comprises a skin disease or condition, or a muscular disease or condition. In some embodiments, the skin disease or condition comprises an inflammatory skin disorder. In some embodiments, an inflammatory skin disorder comprises psoriasis. In some embodiments, the muscular disease or condition comprises a skeletal muscle disorder. In some embodiments, the skeletal muscle disorder comprises fibrodysplasia ossificans progressiva (FOP). In some embodiments, the disease or condition comprises cancer. In some embodiments, the cancer comprises glioblastoma, human tongue squamous carcinoma, human lung carcinoma, or human monocyte leukemia.

In some aspects, compositions or pharmaceutical compositions administered to a subject in need thereof comprise recombinant polynucleic acid constructs or RNA constructs comprising: (i) an IL-4 mRNA; and (ii) at least one siRNA capable of binding to a TNF-alpha mRNA. In related aspects, recombinant polynucleic acid constructs or RNA constructs may encode or comprise at least 1, 2, 3, 4, 5, 6 or more siRNAs. In related aspects, recombinant polynucleic acid constructs or RNA constructs may encode or comprise 1 siRNA directed to a TNF-alpha mRNA. In related aspects, recombinant polynucleic acid constructs or RNA constructs may encode or comprise 3 siRNAs, each directed to a TNF-alpha mRNA. In related aspects, each of the at least 3 siRNAs may be the same, different, or a combination thereof. In related aspects, recombinant RNA constructs may comprise a sequence as set forth in SEQ ID NO: 1, SEQ ID NO: 2, SEQ ID NO: 76, or SEQ ID NO: 77 (Cpd.1 or Cpd.2). In related aspects, recombinant polynucleic acid constructs may comprise a sequence as set forth in SEQ ID NO: 10 or SEQ ID NO: 11 (Cpd.1 or Cpd.2).

In some aspects, compositions or pharmaceutical compositions administered to a subject in need thereof comprise recombinant polynucleic acid constructs or RNA constructs comprising: (i) an IGF-1 mRNA; and (ii) at least one siRNA capable of binding to an ALK2 mRNA. In related aspects, recombinant polynucleic acid constructs or RNA constructs may encode or comprise at least 1, 2, 3, 4, 5, 6, or more siRNAs. In related aspects, recombinant polynucleic acid constructs or RNA constructs may encode or comprise 1 siRNA directed to an ALK2 mRNA. In related aspects, recombinant polynucleic acid constructs or RNA constructs may encode or comprise 3 siRNAs, each directed to an ALK2 mRNA. In related aspects, each of the at least 3 siRNAs may be the same, different, or a combination thereof. In related aspects, recombinant RNA constructs may comprise a sequence as set forth in SEQ ID NO: 3, SEQ ID NO: 4, SEQ ID NO: 78, or SEQ ID NO: 79 (Cpd.3 or Cpd.4). In related aspects, recombinant polynucleic acid constructs may comprise a sequence as set forth in SEQ ID NO: 12 or SEQ ID NO: 13 (Cpd.3 or Cpd.4).

In some aspects, compositions or pharmaceutical compositions administered to a subject in need thereof comprise recombinant polynucleic acid constructs or RNA constructs comprising: (i) an IGF-1 mRNA; and (ii) at least one siRNA capable of binding to a Turbo GFP mRNA. In related aspects, recombinant polynucleic acid constructs or RNA constructs may encode or comprise at least 1, 2, 3, 4, 5, 6, or more siRNAs. In related aspects, recombinant polynucleic acid constructs or RNA constructs may encode or comprise 1 siRNA directed to a Turbo GFP mRNA. In related aspects, recombinant polynucleic acid constructs or RNA constructs may encode or comprise 3 siRNAs, each directed to a Turbo GFP mRNA. In related aspects, each of the at least 3 siRNAs may be the same, different, or a combination thereof. In related aspects, recombinant RNA constructs may comprise a sequence as set forth in SEQ ID NO: 5, SEQ ID NO: 6, SEQ ID NO: 7, SEQ ID NO: 8, SEQ ID NO: 9, SEQ ID NO: 80, SEQ ID NO: 81, SEQ ID NO: 82, SEQ ID NO: 83, or SEQ ID NO: 84 (Cpd.5-Cpd.9). In related aspects, recombinant polynucleic acid constructs may comprise a sequence as set forth in SEQ ID NO: 14, SEQ ID NO: 15, SEQ ID NO: 16, SEQ ID NO: 17, or SEQ ID NO: 18 (Cpd.5-Cpd.9).

In some aspects, compositions or pharmaceutical compositions administered to a subject in need thereof comprise recombinant polynucleic acid constructs or RNA constructs comprising: (i) an IL-2 mRNA; and (ii) at least one siRNA capable of binding to a VEGFA mRNA. In related aspects, recombinant polynucleic acid constructs or RNA constructs may encode or comprise at least 1, 2, 3, 4, 5, 6 or more siRNAs. In related aspects, recombinant polynucleic acid constructs or RNA constructs may encode or comprise 1 siRNA directed to a VEGFA mRNA. In related aspects, recombinant polynucleic acid constructs or RNA constructs may encode or comprise 3 siRNAs, each directed to a VEGFA mRNA. In related aspects, each of the at least 3 siRNAs may be the same, different, or a combination thereof. In related aspects, recombinant RNA constructs may comprise a sequence as set forth in SEQ ID NO: 85, SEQ ID NO: 86, SEQ ID NO: 87, SEQ ID NO: 88, SEQ ID NO: 89, SEQ ID NO: 90, SEQ ID NO: 101, SEQ ID NO: 102, SEQ ID NO: 103, SEQ ID NO: 104, SEQ ID NO: 105, or SEQ ID NO: 106 (Cpd.10, Cpd.11, Cpd. 12, Cpd.13, Cpd.14, or Cpd.15). In related aspects, recombinant polynucleic acid constructs may comprise a sequence as set forth in SEQ ID NO: 93, SEQ ID NO: 94, SEQ ID NO: 95, SEQ ID NO: 96, SEQ ID NO: 97, or SEQ ID NO: 98 (Cpd.10, Cpd.11, Cpd. 12, Cpd.13, Cpd.14, or Cpd.15).

In some aspects, compositions or pharmaceutical compositions administered to a subject in need thereof comprise recombinant polynucleic acid constructs or RNA constructs comprising: (i) an IL-12 mRNA; and (ii) at least one siRNA capable of binding to a c-Myc, KRAS, Akt1, Akt2, and/or Akt3 mRNA. In related aspects, recombinant polynucleic acid constructs or RNA constructs may encode or comprise at least 1, 2, 3, 4, 5, 6 or more siRNAs. In related aspects, recombinant polynucleic acid constructs or RNA constructs may encode or comprise 1 siRNA directed to a c-Myc, KRAS, Akt1, Akt2, and/or Akt3 mRNA. In related aspects, recombinant polynucleic acid constructs or RNA constructs may encode or comprise 3 siRNAs, each directed to a c-Myc, KRAS, Akt1, Akt2, and/or Akt3 mRNA. In related aspects, each of the at least 3 siRNAs may be the same, different, or a combination thereof. In related aspects, recombinant polynucleic acid or RNA constructs may encode or comprise at least 3 siRNAs, each directed to one mRNA selected from c-Myc, KRAS, Akt1, Akt2, and Akt3 mRNAs. In related aspects, recombinant polynucleic acid or RNA constructs may encode or comprise at least 3 siRNAs, each directed to one mRNA selected from c-Myc, KRAS, pan-Akt (i.e., binds to Akt1, Akt2, and Akt3) mRNAs. In related aspects, recombinant RNA constructs may comprise a sequence as set forth in SEQ ID NO: 91, SEQ ID NO: 92, SEQ ID NO: 107, or SEQ ID NO: 108 (Cpd.16 or Cpd.17). In related aspects, recombinant polynucleic acid constructs may comprise a sequence as set forth in SEQ ID NO: 100 or SEQ ID NO: 101 (Cpd.16 or Cpd.17).

Recombinant RNA construct compositions described herein may be administered as a combination therapy. Combination therapies with two or more therapeutic agents or therapies may use agents and therapies that work by different mechanisms of action. Combination therapies using agents or therapies with different mechanisms of action can result in additive or synergetic effects. Combination therapies may allow for a lower dose of each agent than is used in monotherapy, thereby reducing toxic side effects and/or increasing the therapeutic index of the agent(s). Combination therapies can decrease the likelihood that resistant disease cells will develop. In some instances, combination therapies comprise a therapeutic agent or therapy that affects the immune response (e.g., enhances or activates the response) and a therapeutic agent that affects (e.g., inhibits or kills) the disease cells. In some instances, combination therapies may comprise (i) recombinant RNA compositions or pharmaceutical compositions described herein; and (ii) one or more additional therapies known in the art for the diseases described herein. In some embodiments, recombinant RNA compositions or pharmaceutical compositions described herein may be administered to a subject with a disease or condition prior to, concurrently with, and/or subsequently to, administration of one or more additional therapies for combination therapies. In some embodiments, the one or more additional therapies may comprise 1, 2, 3, or more additional therapeutic agents or therapies.

Compositions and pharmaceutical compositions described herein can be administered to a subject using any suitable methods known in the art. Suitable formulations for use in the present invention and methods of delivery are generally well known in the art. For example, compositions described herein can be administered to the subject in a variety of ways, including parenterally, intravenously, intradermally, intramuscularly, colonically, rectally, or intraperitoneally. In some embodiments, compositions described herein is administered by intraperitoneal injection, intramuscular injection, subcutaneous injection, or intravenous injection of the subject. In some embodiments, compositions described herein can be administered parenterally, intravenously, intramuscularly or orally. In some embodiments, compositions described herein can be administered via injection into disease tissues or cells. In some embodiments, compositions described herein can be administered as an aqueous solution for injection into disease tissues or cells.

Any of compositions and pharmaceutical compositions described herein may be provided together with an instruction manual. The instruction manual may comprise guidance for the skilled person or attending physician how to treat (or prevent) a disease or a disorder as described herein (e.g., a cancer) in accordance with the present invention. In some embodiments, the instruction manual may comprise guidance as to the herein described mode of delivery/administration and delivery/administration regimen, respectively (e.g., route of delivery/administration, dosage regimen, time of delivery/administration, frequency of delivery/administration, etc.). In some embodiments, the instruction manual may comprise the instruction that how compositions of the present invention is to be administrated or injected and/or is prepared for administration or injection. In principle, what has been described herein elsewhere with respect to the mode of delivery/administration and delivery/administration regimen, respectively, may be comprised as respective instructions in the instruction manual.

Compositions and pharmaceutical compositions described herein can be used in a gene therapy. In certain embodiments, compositions comprising recombinant polynucleic acids or RNA constructs described herein can be delivered to a cell in gene therapy vectors. Gene therapy vectors and methods of gene delivery are well known in the art. Non-limiting examples of these methods include viral vector delivery systems including DNA and RNA viruses, which have either episomal or integrated genomes after delivery to the cell, non-viral vector delivery systems including DNA plasmids, naked nucleic acid, and nucleic acid complexed with a delivery vehicle, transposon system (for delivery and integration into the host genomes; Moriarity, et al. (2013) Nucleic Acids Res 41(8), e92, Aronovich, et al., (2011) Hum. Mol. Genet. 20 (R1), R14-R20), retrovirus-mediated DNA transfer (e.g., Moloney Mouse Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus; see e.g., Kay et al. (1993) Science 262, 117-119, Anderson (1992) Science 256, 808-813), and DNA virus-mediated DNA transfer including adenovirus, herpes virus, parvovirus and adeno-associated virus (e.g., Ali et al. (1994) Gene Therapy 1, 367-384). Viral vectors also include but are not limited to adeno-associated virus, adenoviral virus, lentivirus, retroviral, and herpes simplex virus vectors. Vectors capable of integration in the host genome include but are not limited to retrovirus or lentivirus.

In some embodiments, compositions comprising recombinant polynucleic acid or RNA constructs described herein can be delivered to a cell via direct DNA transfer (Wolff et al. (1990) Science 247, 1465-1468). Recombinant polynucleic acid or RNA constructs can be delivered to cells following mild mechanical disruption of the cell membrane, temporarily permeabilizing the cells. Such a mild mechanical disruption of the membrane can be accomplished by gently forcing cells through a small aperture (Sharei et al. PLOS ONE (2015) 10(4), e0118803). In another embodiment, compositions comprising recombinant polynucleic acid or RNA constructs described herein can be delivered to a cell via liposome-mediated DNA transfer (e.g., Gao & Huang (1991) Biochem. Ciophys. Res. Comm. 179, 280-285, Crystal (1995) Nature Med. 1, 15-17, Caplen et al. (1995) Nature Med. 3, 39-46). A liposome can encompass a variety of single and multilamellar lipid vehicles formed by the generation of enclosed lipid bilayers or aggregates. Recombinant polynucleic acid or RNA constructs can be encapsulated in the aqueous interior of a liposome, interspersed within the lipid bilayer of a liposome, attached to a liposome via a linking molecule that is associated with both the liposome and the oligonucleotide, entrapped in a liposome, or complexed with a liposome.

### Modulation of Gene Expression

Provided herein are methods of expressing an siRNA and an mRNA from a single RNA transcript in a cell, comprising introducing into the cell compositions comprising any recombinant polynucleic acid or RNA constructs described herein. Further provided herein are methods of modulating expression of two or more genes in a cell, comprising introducing into the cell compositions comprising recombinant polynucleic acid or RNA constructs encoding or comprising a first RNA sequence, a second RNA sequence, and a linker RNA sequence, wherein the linker RNA links the first RNA sequence and the second RNA sequence, wherein the first RNA sequence encodes a gene of interest; wherein the second RNA sequence encodes a small interfering RNA (siRNA) capable of binding to a target messenger RNA (mRNA), and wherein the target mRNA is different from an mRNA encoded by the gene of interest, thereby modulating the expression of the target mRNA and the gene of interest from a single RNA transcript. In some instances, expression of a polynucleic acid, gene, DNA, or RNA, as used herein, can refer to transcription and/or translation of the polynucleic acid, gene, DNA, or RNA. In some instances, modulating, increasing, upregulating, decreasing, or downregulating expression of a polynucleic acid, gene such as a gene of interest, DNA, or RNA such as a target mRNA, as used herein, can refer to modulating, increasing, upregulating, decreasing, downregulating the level of protein encoded by a polynucleic acid, gene such as a gene of interest, DNA, or RNA such as a target mRNA by affecting transcription and/or translation of the polynucleic acid, gene such as a gene of interest, DNA, or RNA such as a target mRNA. In some instances, inhibiting expression of a polynucleic acid, gene such as a gene of interest, DNA, or RNA such as a target mRNA can refer to affecting transcription and/or translation of the polynucleic acid, gene such as a gene of interest, DNA, or RNA such as a target mRNA such that the level of protein encoded by the polynucleic acid, gene such as a gene of interest, DNA, or RNA such as a target mRNA is reduced or abolished.

For example, provided herein, are methods of modulating expression of two or more genes in a cell, comprising introducing into the cell compositions comprising recombinant polynucleic acid or RNA constructs encoding or comprising a first RNA sequence, a second RNA sequence, and a linker RNA sequence, wherein the linker RNA sequence links the first RNA sequence and the second RNA sequence, wherein the first RNA sequence encodes a IL-4, and wherein the second RNA sequence encodes a small interfering RNA (siRNA) capable of binding to a TNF-alpha mRNA, thereby modulating the expression of the TNF-alpha mRNA and IL-4 from a single RNA transcript.

For example, provided herein, are methods of modulating expression of two or more genes in a cell, comprising introducing into the cell compositions comprising recombinant polynucleic acid or RNA constructs encoding or comprising a first RNA sequence, a second RNA sequence, and a linker RNA sequence, wherein the linker RNA sequence links the first RNA sequence and the second RNA sequence, wherein the first RNA sequence encodes a IGF-1, and wherein the second RNA sequence encodes a small interfering RNA (siRNA) capable of binding to an ALK2 mRNA, thereby modulating the expression of the ALK2 mRNA and IGF-1 from a single RNA transcript.

For example, provided herein, are methods of modulating expression of two or more genes in a cell, comprising introducing into the cell compositions comprising recombinant polynucleic acid or RNA constructs encoding or comprising a first RNA sequence, a second RNA sequence, and a linker RNA sequence, wherein the linker RNA sequence links the first RNA sequence and the second RNA sequence, wherein the first RNA sequence encodes a IGF-1, and wherein the second RNA sequence encodes a small interfering RNA (siRNA) capable of binding to a Turbo GFP mRNA, thereby modulating the expression of the Turbo GFP mRNA and IGF-1 from a single RNA transcript.

For example, provided herein, are methods of modulating expression of two or more genes in a cell, comprising introducing into the cell compositions comprising recombinant polynucleic acid or RNA constructs encoding or comprising a first RNA sequence, a second RNA sequence, and a linker RNA sequence, wherein the linker RNA sequence links the first RNA sequence and the second RNA sequence, wherein the first RNA sequence encodes a IL-2, and wherein the second RNA sequence encodes a small interfering RNA (siRNA) capable of binding to a VEGFA mRNA, thereby modulating the expression of the VEGFA mRNA and IL-2 from a single RNA transcript.

For example, provided herein, are methods of modulating expression of two or more genes in a cell, comprising introducing into the cell compositions comprising recombinant polynucleic acid or RNA constructs encoding or comprising a first RNA sequence, a second RNA sequence, and a linker RNA sequence, wherein the linker RNA sequence links the first RNA sequence and the second RNA sequence, wherein the first RNA sequence encodes a IL-12, and wherein the second RNA sequence encodes a small interfering RNA (siRNA) capable of binding to a c-Myc, KRAS, Akt1, Akt2, and/or Akt3 mRNA, thereby modulating the expression of the VEGFA mRNA and IL-12 from a single RNA transcript. In some embodiments, the second RNA sequence may encode one or more small interfering RNAs (siRNAs), each capable of binding to a c-Myc, KRAS, Akt1, Akt2, and/or Akt3 mRNA, thereby modulating the expression of the VEGFA mRNA and IL-12 from a single RNA transcript. In some embodiments, the second RNA sequence may encode one or more small interfering RNAs (siRNAs), wherein each of the one or more siRNAs may bind to one mRNA selected from c-Myc, KRAS, Akt1, Akt2, and Akt3 mRNAs. In some embodiments, the second RNA sequence may encode one or more small interfering RNAs (siRNAs), wherein each of the one or more siRNAs may bind to one mRNA selected from c-Myc, KRAS, pan-Akt (i.e., binds to Akt1, Akt2, and Akt3) mRNAs.

Provided herein are methods of modulating expression of two or more genes in a cell, comprising introducing into the cell compositions comprising recombinant polynucleic acid or RNA constructs comprising a first RNA sequence, a second RNA sequence, and a linker RNA sequence, wherein the linker RNA sequence links the first RNA sequence and the second RNA sequence, wherein the first RNA encodes IL-4, and wherein the second RNA encodes a small interfering RNA (siRNA) capable of binding to a TNF-alpha mRNA; wherein the expression of IL-4 and TNF-alpha is modulated simultaneously, i.e., the expression of IL-4 is upregulated and the expression of TNF-alpha is downregulated simultaneously. In related aspects, recombinant polynucleic acid or RNA constructs may encode or comprise at least 1, 2, 3, 4, 5, 6, or more siRNAs. In related aspects, recombinant polynucleic acid or RNA constructs may encode or comprise 3 siRNAs, each directed to the same region of a TNF-alpha mRNA. In related aspects, recombinant polynucleic acid or RNA constructs may encode or comprise 3 siRNAs, each directed to a different region of a TNF-alpha mRNA. In related aspects, each of the at least 3 siRNAs may be directed to the same, different, or a combination thereof. In related aspects, recombinant RNA constructs may comprise a sequence comprising SEQ ID NO: 1, SEQ ID NO: 2, SEQ ID NO: 76, or SEQ ID NO: 77 (Cpd.1 or Cpd.2). In related aspects, recombinant polynucleic acid constructs may comprise a sequence comprising SEQ ID NO: 10 or SEQ ID NO: 11 (Cpd.1 or Cpd.2).

Also provided herein are methods of modulating expression of two or more genes in a cell, comprising introducing into the cell compositions comprising recombinant polynucleic acid or RNA constructs comprising a first RNA sequence, a second RNA sequence, and a linker RNA sequence, wherein the linker RNA sequence links the first RNA sequence and the second RNA sequence, wherein the first RNA encodes IGF-1, and wherein the second RNA encodes a small interfering RNA (siRNA) capable of binding to an ALK2 mRNA; wherein the expression of IGF-1 and ALK2 is modulated simultaneously, i.e., the expression of IGF-1 is upregulated and the expression of ALK2 is downregulated simultaneously. In related aspects, recombinant polynucleic acid or RNA constructs may encode or comprise at least 1, 2, 3, 4, 5, 6, or more siRNAs. In related aspects, recombinant polynucleic acid or RNA constructs may encode or comprise 3 siRNAs, each directed to the same region of an ALK2 mRNA. In related aspects, recombinant polynucleic acid or RNA constructs may encode or comprise 3 siRNAs, each directed to a different region of an ALK2 mRNA. In related aspects, each of the at least 3 siRNAs may be directed to the same, different, or a combination thereof. In related aspects, recombinant RNA constructs may comprise a sequence comprising SEQ ID NO: 3, SEQ ID NO: 4, SEQ ID NO: 78, or SEQ ID NO: 79 (Cpd.3 or Cpd.4). In related aspects, recombinant polynucleic acid constructs may comprise a sequence comprising SEQ ID NO: 12 or SEQ ID NO: 13 (Cpd.3 or Cpd.4).

Also provided herein are methods of modulating expression of two or more genes in a cell, comprising introducing into the cell compositions comprising recombinant polynucleic acid or RNA constructs comprising a first RNA sequence, a second RNA sequence, and a linker RNA sequence, wherein the linker RNA sequence links the first RNA sequence and the second RNA sequence, wherein the first RNA encodes IGF-1, and wherein the second RNA encodes a small interfering RNA (siRNA) capable of binding to a Turbo GFP mRNA; wherein the expression of IGF-1 and Turbo GFP is modulated simultaneously, i.e., the expression of IGF-1 is unregulated and the expression of Turbo GFP is downregulated simultaneously. In related aspects, recombinant polynucleic acid or RNA constructs may encode or comprise at least 1, 2, 3, 4, 5, 6, or more siRNAs. In related aspects, recombinant polynucleic acid or RNA constructs may encode or comprise 3 siRNAs, each directed to the same region of a Turbo GFP mRNA. In related aspects, recombinant polynucleic acid or RNA constructs may encode or comprise 3 siRNAs, each directed to a different region of a Turbo GFP mRNA. In related aspects, each of the at least 3 siRNAs may be directed to the same, different, or a combination thereof. In related aspects, recombinant RNA constructs may comprise a sequence selected from the group consisting of SEQ ID NOs: 5-9 and 80-84 (Cpd.5-Cpd.9). In related aspects, recombinant polynucleic acid constructs may comprise a sequence selected from the group consisting of SEQ ID NOs: 14-18 (Cpd.5-Cpd.9).

Also provided herein are methods of modulating expression of two or more genes in a cell, comprising introducing into the cell compositions comprising recombinant polynucleic acid or RNA constructs comprising a first RNA sequence, a second RNA sequence, and a linker RNA sequence, wherein the linker RNA sequence links the first RNA sequence and the second RNA sequence, wherein the first RNA encodes IL-2, and wherein the second RNA encodes a small interfering RNA (siRNA) capable of binding to a VEGFA mRNA; wherein the expression of IL-2 and VEGFA is modulated simultaneously, i.e., the expression of IL-2 is upregulated and the expression of VEGFA is downregulated simultaneously. In related aspects, recombinant polynucleic acid or RNA constructs may encode or comprise at least 1, 2, 3, 4, 5, 6, or more siRNAs. In related aspects, recombinant polynucleic acid or RNA constructs may encode or comprise 3 siRNAs, each directed to the same region of a VEGFA mRNA. In related aspects, recombinant polynucleic acid or RNA constructs may encode or comprise 3 siRNAs, each directed to a different region of a VEGFA mRNA. In related aspects, each of the at least 3 siRNAs may be directed to the same, different, or a combination thereof. In related aspects, recombinant RNA constructs may comprise a sequence comprising SEQ ID NO: 85, SEQ ID NO: 86, SEQ ID NO: 87, SEQ ID NO: 88, SEQ ID NO: 89, SEQ ID NO: 90, SEQ ID NO: 101, SEQ ID NO: 102, SEQ ID NO: 103, SEQ ID NO: 104, SEQ ID NO: 105, or SEQ ID NO: 106 (Cpd.10, Cpd.11, Cpd. 12, Cpd.13, Cpd.14, or Cpd.15). In related aspects, recombinant polynucleic acid constructs may comprise a sequence comprising SEQ ID NO: 93, SEQ ID NO: 94, SEQ ID NO: 95, SEQ ID NO: 96, SEQ ID NO: 97, or SEQ ID NO: 98 (Cpd.10, Cpd.11, Cpd. 12, Cpd.13, Cpd.14, or Cpd.15).

Also provided herein are methods of modulating expression of two or more genes in a cell, comprising introducing into the cell compositions comprising recombinant polynucleic acid or RNA constructs comprising a first RNA sequence, a second RNA sequence, and a linker RNA sequence, wherein the linker RNA sequence links the first RNA sequence and the second RNA sequence, wherein the first RNA encodes IL-12, and wherein the second RNA encodes a small interfering RNA (siRNA) capable of binding to a c-Myc, KRAS, Akt1, Akt2, and/or Akt3 mRNA; wherein the expression of IL-12 and c-Myc, KRAS, Akt1, Akt2, and/or Akt3 is modulated simultaneously, i.e., the expression of IL-12 is upregulated and the expression of c-Myc, KRAS, Akt1, Akt2, and/or Akt3 is downregulated simultaneously. In some embodiments, the expression of IL-12 is upregulated and the expression of c-Myc, KRAS, Akt1, Akt2, and Akt3 is downregulated simultaneously. In related aspects, recombinant polynucleic acid or RNA constructs may encode or comprise at least 1, 2, 3, 4, 5, 6, or more siRNAs. In related aspects, recombinant polynucleic acid or RNA constructs may encode or comprise 3 siRNAs, each directed to the same region of a c-Myc, KRAS, Akt1, Akt2, and/or Akt3 mRNA. In related aspects, recombinant polynucleic acid or RNA constructs may encode or comprise 3 siRNAs, each directed to a different region of a c-Myc, KRAS, Akt1, Akt2, and/or Akt3 mRNA. In related aspects, each of the at least 3 siRNAs may be directed to the same, different, or a combination thereof. In related aspects, recombinant polynucleic acid or RNA constructs may encode or comprise at least 3 siRNAs, each directed to one mRNA selected from c-Myc, KRAS, Akt1, Akt2, and Akt3 mRNAs. In related aspects, recombinant polynucleic acid or RNA constructs may encode or comprise at least 3 siRNAs, each directed to one mRNA selected from c-Myc, KRAS, pan-Akt (i.e., binds to Akt1, Akt2, and Akt3) mRNAs. In related aspects, recombinant RNA constructs may comprise a sequence comprising SEQ ID NO: 91, SEQ ID NO: 92, SEQ ID NO: 107, or SEQ ID NO: 108 (Cpd.16 or Cpd.17). In related aspects, recombinant polynucleic acid constructs may comprise a sequence comprising SEQ ID NO: 100 or SEQ ID NO: 101 (Cpd.16 or Cpd.17).

Provided herein are methods of upregulating and downregulating expression of two or more genes in a cell, comprising introducing into the cell compositions comprising recombinant polynucleic acid or RNA constructs encoding or comprising a first RNA sequence, a second RNA sequence, and a linker RNA sequence, wherein the linker RNA sequence links the first RNA sequence and the second RNA sequence, wherein the first RNA encodes a gene of interest (e.g., IL-4, IL-2, IL-12, or IGF-1), and wherein the second RNA encodes a small interfering RNA (siRNA) capable of binding to a target mRNA (e.g., TNF-alpha, ALK2, Turbo GFP, VEGFA, c-Myc, KRAS, Akt1, Akt2, or Akt3); wherein the target mRNA is different from an mRNA encoded by the gene of interest, and wherein the expression of the target mRNA is downregulated and the expression of the gene of interest is upregulated simultaneously. In some embodiments, the expression of the target mRNA is downregulated by the siRNA capable of binding to the target mRNA. In some embodiments, the expression of the gene of interest is upregulated by expressing an mRNA or a protein encoded by the gene of interest.

### Illustrative Embodiments

In some aspects, provided herein, is a composition comprising a recombinant RNA construct comprising a first RNA sequence, a second RNA sequence, and a linker RNA sequence, wherein: (i) the first RNA sequence is a first small interfering RNA (siRNA) sequence; (ii) the second RNA sequence is a second siRNA sequence or a first messenger RNA (mRNA) sequence encoding a gene of interest (GOI); and (iii) the linker RNA sequence links the first RNA sequence and the second RNA sequence, wherein the linker RNA sequence has a structure selected from the group consisting of: Formula (I): XₘCAACAAXₙ, wherein X is any nucleotide, m is an integer from 1 to 12, and n is an integer from 0 to 4 (SEQ ID NO: 151); and Formula (II): XₚTCCCXᵣ, wherein X is any nucleotide, p is an integer from 0 to 17, and r is an integer from 0 to 13 (SEQ ID NO: 152).

In some aspects, provided herein, is a composition comprising a recombinant RNA construct comprising a first RNA sequence, a second RNA sequence, and a linker RNA sequence, wherein: (i) the first RNA sequence is a first small interfering RNA (siRNA) sequence; (ii) the second RNA sequence is a second siRNA sequence or a first messenger RNA (mRNA) sequence encoding a gene of interest (GOI); and (iii) the linker RNA sequence links the first RNA sequence and the second RNA sequence, wherein the linker RNA sequence comprises or consists of ACAACAA (SEQ ID NO: 23).

In some aspects, provided herein, is a composition comprising a recombinant RNA construct comprising a first RNA sequence, a second RNA sequence, and a linker RNA sequence, wherein: (i) the first RNA sequence is a first small interfering (siRNA) sequence; (ii) the second RNA sequence is a second siRNA sequence or a first messenger (mRNA) sequence encoding a gene of interest (GOI); and (iii) the linker RNA sequence links the first RNA sequence and the second RNA sequence, wherein (a) the linker RNA sequence is not TTTATCTTAGAGGCATATCCCTACGTACCAACAA (SEQ ID NO: 22) or ATAGTGAGTCGTATTAACGTACCAACAA (SEQ ID NO: 21); or (b) the linker RNA sequence does not form a secondary structure according to RNAfold WebServer.

In some embodiments, the second RNA sequence is a second siRNA sequence. In some embodiments, the linker RNA sequence comprises or consists of ACAACAA (SEQ ID NO: 23), ATCCCTACGTACCAACAA (SEQ ID NO: 67), ACGTACCAACAA (SEQ ID NO: 68), TCCC (SEQ ID NO: 69), or ACAACAATCCC (SEQ ID NO: 70). In some embodiments, the recombinant RNA construct further comprises a first mRNA sequence encoding a GOI. In some embodiments, the second RNA sequence is a first mRNA sequence encoding a GOI.

In some embodiments, the linker RNA sequence comprises or consists of ACAACAA (SEQ ID NO: 23), ATAGTGAGTCGTATTATCCC (SEQ ID NO: 72), ATAGTGAGTCGTATTAACAACAATCCC (SEQ ID NO: 73), ATAGTGAGTCGTATTAACAACAA (SEQ ID NO: 74), ATAGTGAGTCGTATTAATCCCTACGTACCAACAA (SEQ ID NO: 75), or ATAGTGAGTCGTATTAACGTACCAACAA (SEQ ID NO: 21).

In some embodiments, the recombinant RNA construct further comprises a second mRNA sequence encoding a GOI. In some embodiments, the recombinant RNA construct further comprises a second siRNA sequence. In some embodiments, the recombinant RNA construct comprises a third siRNA sequence. In some embodiments, the recombinant RNA construct further comprises four, five, or more siRNA sequences. In some embodiments, each of the siRNA sequences binds to a target RNA and modulates the expression of the target RNA.

In some embodiments, each of the siRNA sequences is capable of binding to: (a) different target RNAs; (b) different regions of the same target RNA; (c) the same region of the same target RNA; or (d) any combinations thereof. In some embodiments, the siRNA sequences of (c) are the same. In some embodiments, the recombinant RNA construct comprises three, four, five, or more mRNA sequences, each encoding a GOI. In some embodiments, each of the mRNA sequences encodes the same GOI. In some embodiments, each of the mRNA sequences encodes a different GOI.

In some embodiments, the length of the linker RNA sequence between siRNA sequences is from about 4 to about 27 nucleotides. In some embodiments, the length of the linker RNA sequence between siRNA sequences is from about 4 to about 18 nucleotides. In some embodiments, m is 1 and n is 0. In some embodiments, the linker RNA sequence between siRNA sequences is ACAACAATCCC (SEQ ID NO: 70). In some embodiments, the linker RNA sequence is ACAACAA (SEQ ID NO: 23). In some embodiments, the linker RNA sequence comprises a sequence selected from the group consisting of SEQ ID NOs: 23 and 67-75. In some embodiments, the linker RNA sequence comprises or consists of a sequence according to SEQ ID NO: 23. In some embodiments, the linker RNA sequence comprises or consists of a sequence according to SEQ ID NO: 67. In some embodiments, the linker RNA sequence comprises or consists of a sequence according to SEQ ID NO: 68. In some embodiments, the linker RNA sequence comprises or consists of a sequence according to SEQ ID NO: 69. In some embodiments, the linker RNA sequence comprises or consists of a sequence according to SEQ ID NO: 70.

In some embodiments, expression of a target RNA targeted by the siRNA is lower using a recombinant RNA construct comprising the linker RNA sequence according to SEQ ID NO: 67 compared to (i) expression of the target RNA targeted by the siRNA using a recombinant RNA construct comprising the linker RNA sequence according to SEQ ID NO: 68, (ii) expression of the target RNA targeted by the siRNA using a recombinant RNA construct comprising the linker RNA sequence according to SEQ ID NO: 69, (iii) expression of the target RNA targeted by the siRNA using a recombinant RNA construct comprising the linker RNA sequence according to SEQ ID NO: 70, and/or (iv) expression of the target RNA targeted by the siRNA using a recombinant RNA construct comprising the linker RNA sequence according to SEQ ID NO: 23.

In some embodiments, expression of a first mRNA sequence encoding a GOI is higher using a recombinant RNA construct comprising the linker RNA sequence according to SEQ ID NO: 70 compared to (i) expression of the first mRNA sequence encoding the GOI using a recombinant RNA construct comprising the linker RNA sequence according to SEQ ID NO: 67, (ii) expression of the first mRNA sequence encoding the GOI using a recombinant RNA construct comprising the linker RNA sequence according to SEQ ID NO: 68, (iii) expression of the first mRNA sequence encoding the GOI using a recombinant RNA construct comprising the linker RNA sequence according to SEQ ID NO: 69, and/or (iv) expression of the first mRNA sequence encoding the GOI using a recombinant RNA construct comprising the linker RNA sequence according to SEQ ID NO: 23.

In some embodiments, expression of a target RNA targeted by the siRNA is lower using a recombinant RNA construct comprising the linker RNA sequence according to SEQ ID NO: 23 compared to (i) expression of the target RNA targeted by the siRNA using a recombinant RNA construct comprising the linker RNA sequence according to SEQ ID NO: 68, (ii) expression of the target RNA targeted by the siRNA using a recombinant RNA construct comprising the linker RNA sequence according to SEQ ID NO: 69, and/or (iii) expression of the target RNA targeted by the siRNA using a recombinant RNA construct comprising the linker RNA sequence according to SEQ ID NO: 70.

In some embodiments, expression of a target RNA targeted by the siRNA is lower using a recombinant RNA construct comprising the linker RNA sequence according to SEQ ID NO: 23 compared to (i) expression of the target RNA targeted by the siRNA using a recombinant RNA construct comprising the linker RNA sequence according to SEQ ID NO: 69, and/or (ii) expression of the target RNA targeted by the siRNA using a recombinant RNA construct comprising the linker RNA sequence according to SEQ ID NO: 70.

In some embodiments, expression of a first mRNA sequence encoding a GOI is higher using a recombinant RNA construct comprising the linker RNA sequence according to SEQ ID NO: 23 compared to (i) expression of the first mRNA sequence encoding the GOI using a recombinant RNA construct comprising the linker RNA sequence according to SEQ ID NO: 67, (ii) expression of the first mRNA sequence encoding the GOI using a recombinant RNA construct comprising the linker RNA sequence according to SEQ ID NO: 68, and/or (iii) expression of the first mRNA sequence encoding the GOI using a recombinant RNA construct comprising the linker RNA sequence according to SEQ ID NO: 69.

In some embodiments, the linker RNA sequence is selected based on a desired expression level of the first mRNA sequence encoding the GOI and/or a desired expression level of the target RNA targeted by the siRNA or desired expression level of a protein encoded by the target RNA targeted by the siRNA.

In some embodiments, the expression of the GOI is modulated. In some embodiments, the expression of the GOI is upregulated by expressing a protein encoded by the GOI. In some embodiments, the expression of the target RNA is modulated. In some embodiments, the expression of the target RNA is downregulated by the siRNA sequences capable of binding to the target RNA. In some embodiments, the siRNA sequences capable of binding to the target RNA do not inhibit the expression of the GOI.

In some embodiments, the RNA linker sequence between siRNA sequences does not form a secondary structure according to RNAfold WebServer. In some embodiments, an siRNA sequence forms a secondary structure according to RNAfold WebServer. In some embodiments, the siRNA sequence comprises a hairpin structure or a loop structure. In some embodiments, the siRNA sequences comprise one or more short or small hairpin RNAs (shRNAs).

In some embodiments, the recombinant RNA construct is cleaved. In some embodiments, the recombinant RNA construct is cleaved by an intracellular protein. In some embodiments, the recombinant RNA construct is cleaved by an endogenous protein. In some embodiments, the recombinant RNA construct is cleaved by an endogenous DICER.

In some embodiments, the cleavage of the recombinant RNA construct is enhanced compared to the cleavage of an RNA construct that does not comprise a linker having a structure selected from the group consisting of Formula (I) and Formula (II). In some embodiments, the cleavage of the recombinant RNA construct is enhanced compared to the cleavage of an RNA construct that does not comprise a linker comprising a sequence comprising ACAACAA (SEQ ID NO: 23). In some embodiments, the cleavage of the recombinant RNA construct is enhanced compared to the cleavage of an RNA construct comprising a linker that forms a secondary structure.

In some embodiments, the expression of the gene of interest is enhanced compared to the expression of a gene of interest from an RNA construct that does not comprise a linker having a structure selected from the group consisting of Formula (I) and Formula (II). In some embodiments, the expression of the gene of interest is enhanced compared to the expression of a gene of interest from an RNA construct that does not comprise a linker comprising a sequence comprising ACAACAA (SEQ ID NO: 23).

In some embodiments, the GOI comprises Interleukin 4 (IL-4), Interleukin 2 (IL-2), Interleukin 12 (IL-12), or Insulin-like Growth Factor 1 (IGF1). In some embodiments, the target RNA is a noncoding RNA. In some embodiments, the target RNA is a messenger RNA (mRNA). In some embodiments, the target RNA is an mRNA encoding a protein selected from the group consisting of Tumor Necrosis Factor alpha (TNF-α), Activin Receptor-like Kinase 2 (ALK2), Vascular Endothelial Growth Factor A (VEGFA), Cellular Myelocytomatosis (c-Myc), Kirsten Rat Sarcoma (KRAS), Protein kinase B-1 (Akt1), Akt2, and Akt3.

In some embodiments, the siRNA sequences capable of binding to the target RNA bind to an exon of the target RNA. In some embodiments, the siRNA sequences capable of binding to the target RNA specifically bind to one target RNA. In some embodiments, the siRNA sequences capable of binding to the target RNA are not encoded by or comprised of an intron sequence of the gene of interest. In some embodiments, the GOI is expressed without RNA splicing.

In some embodiments, the first RNA sequence is present downstream or 3′ of the second RNA sequence. In some embodiments, the RNA construct comprises an internal ribosome entry site (IRES) downstream or 3′ of the second RNA sequence. In some embodiments, the RNA construct comprises an internal ribosome entry site (IRES) immediately upstream or 5′ of the first RNA sequence. In some embodiments, the first RNA sequence is present upstream or 5′ of the second RNA sequence. In some embodiments, the RNA construct comprises an internal ribosome entry site (IRES) upstream or 5′ of the first RNA sequence.

In some embodiments, the RNA construct further comprises a poly(A) tail, a 5′ cap, or a Kozak sequence. In some embodiments, the first RNA sequence and the second RNA sequence are both recombinant. In some embodiments, the siRNA comprises a sense strand sequence selected from the group consisting of SEQ ID NOs: 50-57 and 127-132.

In some aspects, provided herein, is a composition for use in modulating the expression of two or more genes in a cell. In some aspects, provided herein, is a pharmaceutical composition comprising a therapeutically effective amount of any one of the compositions described herein and a pharmaceutically acceptable excipient. In some aspects, provided herein, is a cell comprising any one of the compositions described herein. In some aspects, provided herein, is a vector comprising a recombinant polynucleic acid construct encoding any one of the compositions described herein.

In some aspects, provided herein, is a method of producing an siRNA and an mRNA from a single RNA transcript in a cell, comprising introducing into the cell any one of the compositions described herein or the vectors described herein. In some aspects, provided herein, is a method of modulating protein expression comprising introducing any one of the compositions described herein or any one of the vectors described herein into a cell, wherein the expression of a protein encoded by the target RNA is decreased. In some aspects, provided herein, is a method of modulating protein expression comprising introducing any one of the compositions described herein or any one of the vectors described herein into a cell, wherein the expression of a protein encoded by a gene of interest (GOI) is increased. In some aspects, provided herein, is a method of modulating protein expression comprising introducing any one of the compositions described herein or any one of the vectors described herein into a cell, wherein the expression of a protein encoded by the target RNA is decreased, and wherein the expression of a protein encoded by a gene of interest (GOI) is increased.

In some aspects, provided herein, is a method of treating a disease or condition comprising administering to a subject in need thereof any one of the compositions described herein or any one of the pharmaceutical compositions described herein.

In some embodiments, the disease or condition comprises a skin disease or condition or a muscular disease or condition. In some embodiments, the skin disease or condition comprises an inflammatory skin disorder. In some embodiments, the inflammatory skin disorder comprises psoriasis. In some embodiments, the muscular disease or condition comprises a skeletal muscle disorder. In some embodiments, the skeletal muscle disorder comprises fibrodysplasia ossificans progressiva (FOP). In some embodiments, the disease or condition comprises cancer. In some embodiments, the cancer comprises glioblastoma, human tongue squamous carcinoma, human lung carcinoma, or human monocyte leukemia. In some embodiments, the subject is a human.

In some aspects, provided herein, is a composition comprising a recombinant RNA construct comprising a first RNA sequence, a first linker RNA sequence, a second RNA sequence, and a second linker RNA sequence, wherein: (i) the first RNA sequence is a messenger RNA (mRNA) encoding Interleukin 4 (IL-4); (ii) the second RNA sequence comprises two or more small interfering RNAs (siRNAs) capable of binding to a Tumor Necrosis Factor alpha (TNF-α) mRNA; (iii) the first linker RNA sequence is present between the first RNA sequence and the second RNA sequence; and (iv) the second linker RNA sequence links each of the two or more siRNAs and comprises a sequence according to SEQ ID NO: 23.

In some aspects, provided herein, is a composition comprising a recombinant RNA construct comprising a first RNA sequence, a first linker RNA sequence, a second RNA sequence, and a second linker RNA sequence, wherein: (i) the first RNA sequence is a messenger RNA (mRNA) encoding Insulin-like Growth Factor 1 (IGF1); (ii) the second RNA sequence comprises two or more small interfering RNAs (siRNAs) capable of binding to a Activin Receptor-like Kinase 2 (ALK2) mRNA; (iii) the first linker RNA sequence is present between the first RNA sequence and the second RNA sequence; and (iv) the second linker RNA sequence links each of the two or more siRNAs and comprises a sequence according to SEQ ID NO: 23.

In some aspects, provided herein, is a composition comprising a recombinant RNA construct comprising a first RNA sequence, a first linker RNA sequence, a second RNA sequence, and a second linker RNA sequence, wherein: (i) the first RNA sequence is a messenger RNA (mRNA) encoding Interleukin 2 (IL-2); (ii) the second RNA sequence comprises two or more small interfering RNAs (siRNAs) capable of binding to a Vascular Endothelial Growth Factor A (VEGFA) mRNA; (iii) the first linker RNA sequence is present between the first RNA sequence and the second RNA sequence; and (iv) the second linker RNA sequence links each of the two or more siRNAs and comprises a sequence selected from the group consisting of SEQ ID NOs: 23 and 67-70.

In some aspects, provided herein, is a composition comprising a recombinant RNA construct comprising a first RNA sequence, a first linker RNA sequence, a second RNA sequence, and a second linker RNA sequence, wherein: (i) the first RNA sequence is a messenger RNA (mRNA) encoding Interleukin 12 (IL-12); (ii) the second RNA sequence comprises two or more small interfering RNAs (siRNAs) capable of binding to an mRNA of Cellular Myelocytomatosis (c-Myc), Kirsten Rat Sarcoma (KRAS), Protein kinase B-1 (Akt1), Akt2, and/or Akt3; (iii) the first linker RNA sequence is present between the first RNA sequence and the second RNA sequence; and (iv) the second linker RNA sequence links each of the two or more siRNAs and comprises a sequence according to SEQ ID NO: 23.

In some aspects, provided herein, is a composition comprising a recombinant polynucleic acid construct comprising a nucleic acid sequence selected from the group consisting of SEQ ID NOs: 1-18 and 76-108.

### EXAMPLES

These examples are provided for illustrative purposes only and not to limit the scope of the claims provided herein.

### Example 1: Construct Design, Sequence, and Synthesis

Construct Design

Constructs were designed to express both siRNAs and genes of interest simultaneously from a single transcript generated by in vitro transcription (Table 1; SEQ ID NOs: 1-9 and 76-84). IL-4 and IGF-1 coding sequences originate from Homo sapiens and no changes in the resulting amino acid sequences were introduced for IL-4 (hIL4: NP_000580.1; SEQ ID NO: 26 and 27). To increase secretion of mRNA-induced IGF-1 (NP_000609.1) out of the transfected cell, the endogenous IGF-1 pre-domain (signal peptide; SEQ ID NO: 28 and 29) was exchanged by BDNF (NP_733931.1; SEQ ID NO: 30 and 31) signal peptide (BDNF-pro-IGF-1) in the mRNA construct. Furthermore, the construct contained the sequence encoding the full coding sequence of mature human IGF-1 with 70 amino acids (SEQ ID NO: 30 and 31). No C-terminal E-domain was added to the construct. The siRNA target sequence for TNF-alpha (NM_000594.3; SEQ ID NO: 32) and ALK2 (NM_001105.4; SEQ ID NO: 33) originate from Homo sapiens and no changes to the sequences introduced. Turbo GFP sequence was derived from marine copepod Pontellina plumate (SEQ ID NO: 34).

A polynucleic acid construct may comprise a Kozak sequence, (5′ GCCACC 3′; SEQ ID NO:19). In addition, a polynucleic acid construct may comprise a T7 promoter sequence (5′ TAATACGACTCACTATA 3′; SEQ ID NO:20) upstream of the gene of interest sequence, for RNA polymerase binding and successful in vitro transcription of both the gene of interest and siRNA in a single transcript. An alternative promoter e.g., SP6, T3, P60, Syn5, and KP34 may be used. A transcription template was generated by PCR to produce mRNA, using primers designed to flank the T7 promoter, gene of interest, and siRNA sequences. The reverse primer includes a stretch of thymidine (T) base (120) (SEQ ID NO: 156) to add the 120 bp length of poly(A) tail (SEQ ID NO: 155) to the mRNA. Some of the polynucleotide or RNA constructs were engineered to include siRNA designs described in Cheng, et al. (2018) J. Mater. Chem. B., 6, 4638-4644, and further comprising one or more gene of interest upstream of the siRNA sequence with linkers to connect different RNA segments (gene of interest mRNA to siRNA (SEQ ID NO:21), or siRNA to siRNA (SEQ ID NO:22)), refereed as A1-linker hereafter. In some constructs, a novel linker sequence was designed to connect different RNA segments (e.g., to connect mRNA encoding a gene of interest and siRNA and to connect siRNA and siRNA), referred as A2-linker hereafter (SEQ ID NO:23). Recombinant constructs may encode or comprise more than one siRNA sequence targeting the same or different target mRNA. Likewise, constructs may comprise nucleic acid sequences of two or more genes of interest.

Construct Synthesis

The constructs as shown in Table 1 (Compound ID numbers Cpd.1-Cpd.17) are synthesized in pMA-RQ plasmid-backbone vector by GeneArt, Germany (Thermo Fisher Scientific) or in pUC-GW-Kan backbone vector by GeneWiz, China containing a T7 RNA polymerase promoter with codon optimization on open reading frame (ORF) using GeneOptimizer algorithm. Table 1 shows, for each compound (Cpd.), protein to be downregulated through siRNA binding to the corresponding mRNA (siRNA target), siRNA position in the compound, the number of siRNAs in the compound, gene of interest and respective indication. The sequences of each construct are shown in Table 2 and Table 6, and annotated as indicated below the table (SEQ ID NO: 1-9, 85-92, 76-84 and 85-92). The plasmid-backbone sequences of each construct are shown in Table 3 and compound sequence are in bold and underlined (SEQ ID NO: 10-18; 93-100).

TABLE 1
Summary of Compounds

| Compound | siRNA | Linker | siRNA | # of | Gene of | |
|---|---|---|---|---|---|---|
| ID | Target | Name | position | siRNAs | Interest | Indication |
| 1 | TNF-alpha | A1 | 3′ | 3× | IL-4 | Psoriasis |
| 2 | TNF-alpha | A2 | 3′ | 3× | IL-4 | Psoriasis |
| 3 | ALK2 | A1 | 3′ | 3× | IGF-1 | FOP |
| 4 | ALK2 | A2 | 3′ | 3× | IGF-1 | FOP |
| 5 | Turbo GFP | A1 | 3′ | 1× | IGF-1 | NA |
| 6 | Turbo GFP | A2 | 3′ | 1× | IGF-1 | NA |
| 7 | Turbo GFP | A1 | 3′ | 2× | IGF-1 | NA |
| 8 | Turbo GFP | A1 | 3′ | 2× | IGF-1 | NA |
| 9 | Turbo GFP | A2 | 3′ | 2× | IGF-1 | NA |
| 10 | VEGFA | A1 | 3′ | 3× | IL-2 | Oncology |
| 11 | VEGFA | A2 | 3′ | 3× | IL-2 | Oncology |
| 12 | VEGFA | B | 3′ | 3× | IL-2 | Oncology |
| 13 | VEGFA | C | 3′ | 3× | IL-2 | Oncology |
| 14 | VEGFA | D | 3′ | 3× | IL-2 | Oncology |
| 15 | VEGFA | E | 3′ | 3× | IL-2 | Oncology |
| 16 | 1× c- | A1 | 3′ | 3× | IL-12 | Oncology |
| | Myc/1× | | | | | |
| | KRAS/1× | | | | | |
| | pan-Akt | | | | | |
| 17 | 1× c- | A2 | 3′ | 3× | IL-12 | Oncology |
| | Myc/1× | | | | | |
| | KRAS/1× | | | | | |
| | Akt | | | | | |

TNF-alpha: Tumor necrosis factor-alpha, IL-4: Interleukin 4, ALK2: Activin receptor-like kinase-2, FOP: Fibrodysplasia ossificans progressiva, IGF-1: Insulin like growth factor-1, Turbo GFP: Turbo green fluorescent protein (derived from copepod Pontellina plumate), NA: Not available. VEGFA: Vascular endothelial growth factor A, c-Myc: Cellular myelocytomatosis, KRAS: Kirsten rat sarcoma, Akt: Protein kinase B, pan-Akt: Akt1, Akt2, and Akt3.

TABLE 2
Sequences of Compounds

| SEQ ID NO | Compound | Sequence (5′ to 3′) |
|---|---|---|
| 1 | Compound 1 | GCCACCATGGGACTGACATCTCAACTGCTGCCTCCACTGTTCTTTCTGC |
| | | TGGCCTGCGCCGGCAATTTTGTGCACGGCCACAAGTGCGACATCACCCT |
| | | GCAAGAGATCATCAAGACCCTGAACAGCCTGACCGAGCAGAAAACCCTG |
| | | TGCACCGAGCTGACCGTGACCGATATCTTTGCCGCCAGCAAGAACACAA |
| | | CCGAGAAAGAGACATTCTGCAGAGCCGCCACCGTGCTGAGACAGTTCTA |
| | | CAGCCACCACGAGAAGGACACCAGATGCCTGGGAGCTACAGCCCAGCAG |
| | | TTCCACAGACACAAGCAGCTGATCCGGTTCCTGAAGCGGCTGGACAGAA |
| | | ATCTGTGGGGACTCGCCGGCCTGAATAGCTGCCCTGTGAAAGAGGCCAA |
| | | CCAGTCTACCCTGGAAAACTTCCTGGAACGGCTGAAAACCATCATGCGC |
| | | GAGAAGTACAGCAAGTGCAGCAGCTGAATAGTGAGTCGTATTAACGTAC |
| | | CAACAAGGCGTGGAGCTGAGAGATAAACTTGTTATCTCTCAGCTCCACG |
| | | CCTTTATCTTAGAGGCATATCCCTACGTACCAACAAGGGCCTGTACCTC |
| | | ATCTACTACTTGAGTAGATGAGGTACAGGCCCTTTATCTTAGAGGCATA |
| | | TCCCTACGTACCAACAAGGTATGAGCCCATCTATCTACTTGAGATAGAT |
| | | GGGCTCATACCTTTATCTTAGAGGCATATCCCTTTTATCTTAGAGGCAT |
| | | ATCCCT |
| 2 | Compound 2 | GCCACCATGGGACTGACATCTCAACTGCTGCCTCCACTGTTCTTTCTGC |
| | | TGGCCTGCGCCGGCAATTTTGTGCACGGCCACAAGTGCGACATCACCCT |
| | | GCAAGAGATCATCAAGACCCTGAACAGCCTGACCGAGCAGAAAACCCTG |
| | | TGCACCGAGCTGACCGTGACCGATATCTTTGCCGCCAGCAAGAACACAA |
| | | CCGAGAAAGAGACATTCTGCAGAGCCGCCACCGTGCTGAGACAGTTCTA |
| | | CAGCCACCACGAGAAGGACACCAGATGCCTGGGAGCTACAGCCCAGCAG |
| | | TTCCACAGACACAAGCAGCTGATCCGGTTCCTGAAGCGGCTGGACAGAA |
| | | ATCTGTGGGGACTCGCCGGCCTGAATAGCTGCCCTGTGAAAGAGGCCAA |
| | | CCAGTCTACCCTGGAAAACTTCCTGGAACGGCTGAAAACCATCATGCGC |
| | | GAGAAGTACAGCAAGTGCAGCAGCTGAACAACAAGGCGTGGAGCTGAGA |
| | | GATAAACTTGTTATCTCTCAGCTCCACGCCACAACAAGGGCCTGTACCT |
| | | CATCTACTACTTGAGTAGATGAGGTACAGGCCCACAACAAGGTATGAGC |
| | | CCATCTATCTACTTGAGATAGATGGGCTCATACCACAACAATTTATCTT |
| | | AGAGGCATATCCCT |
| 3 | Compound 3 | GCCACCATGACCATCCTGTTTCTGACAATGGTCATCAGCTACTTCGGCT |
| | | GCATGAAGGCCGTGAAGATGCACACCATGAGCAGCAGCCACCTGTTCTA |
| | | TCTGGCCCTGTGCCTGCTGACCTTTACCAGCTCTGCTACCGCCGGACCT |
| | | GAGACACTTTGTGGCGCTGAACTGGTGGACGCCCTGCAGTTTGTGTGTG |
| | | GCGACAGAGGCTTCTACTTCAACAAGCCCACAGGCTACGGCAGCAGCTC |
| | | TAGAAGGGCTCCTCAGACCGGAATCGTGGACGAGTGCTGCTTCAGAAGC |
| | | TGCGACCTGCGGCGGCTGGAAATGTATTGTGCCCCTCTGAAGCCTGCCA |
| | | AGAGCGCCTAAATAGTGAGTCGTATTAACGTACCAACAAGGCCTCATTA |
| | | TTCTCTCTACTTGAGAGAGAATAATGAGGCCTTTATCTTAGAGGCATAT |
| | | CCCTACGTACCAACAAGTGTTCGCAGTATGTCTTACTTGAAGACATACT |
| | | GCGAACACTTTATCTTAGAGGCATATCCCTACGTACCAACAAGCCTGCC |
| | | TGCTGGGAGTTACTTGAACTCCCAGCAGGCAGGCTTTATCTTAGAGGCA |
| | | TATCCCTTTTATCTTAGAGGCATATCCCT |
| 4 | Compound 4 | GCCACCATGACCATCCTGTTTCTGACAATGGTCATCAGCTACTTCGGCT |
| | | GCATGAAGGCCGTGAAGATGCACACCATGAGCAGCAGCCACCTGTTCTA |
| | | TCTGGCCCTGTGCCTGCTGACCTTTACCAGCTCTGCTACCGCCGGACCT |
| | | GAGACACTTTGTGGCGCTGAACTGGTGGACGCCCTGCAGTTTGTGTGTG |
| | | GCGACAGAGGCTTCTACTTCAACAAGCCCACAGGCTACGGCAGCAGCTC |
| | | TAGAAGGGCTCCTCAGACCGGAATCGTGGACGAGTGCTGCTTCAGAAGC |
| | | TGCGACCTGCGGCGGCTGGAAATGTATTGTGCCCCTCTGAAGCCTGCCA |
| | | AGAGCGCCTAAACAACAAGGCCTCATTATTCTCTCTACTTGAGAGAGAA |
| | | TAATGAGGCCACAACAAGTGTTCGCAGTATGTCTTACTTGAAGACATAC |
| | | TGCGAACACACAACAAGCCTGCCTGCTGGGAGTTACTTGAACTCCCAGC |
| | | AGGCAGGCACAACAATTTATCTTAGAGGCATATCCCT |
| 5 | Compound 5 | GCCACCATGGGCAAGATTAGCAGCCTGCCTACACAGCTGTTCAAGTGCT |
| | | GCTTCTGCGACTTCCTGAAAGTGAAGATGCACACCATGAGCAGCAGCCA |
| | | CCTGTTCTATCTGGCCCTGTGCCTGCTGACCTTTACCAGCTCTGCTACC |
| | | GCCGGACCTGAGACACTTTGTGGCGCTGAACTGGTGGACGCCCTGCAGT |
| | | TTGTGTGTGGCGACAGAGGCTTCTACTTCAACAAGCCCACAGGCTACGG |
| | | CAGCAGCTCTAGAAGGGCTCCTCAGACCGGAATCGTGGACGAGTGCTGT |
| | | TTCAGAAGCTGCGACCTGCGGCGGCTGGAAATGTATTGTGCCCCTCTGA |
| | | AGCCTGCCAAGAGCGCCTAAATAGTGAGTCGTATTAACGTACCAACAAC |
| | | AACAAGATGAAGAGCACCAAACTTGTTGGTGCTCTTCATCTTGTTGTTT |
| | | ATCTTAGAGGCATATCCCTTTTATCTTAGAGGCATATCCCT |
| 6 | Compound 6 | GCCACCATGGGCAAGATTAGCAGCCTGCCTACACAGCTGTTCAAGTGCT |
| | | GCTTCTGCGACTTCCTGAAAGTGAAGATGCACACCATGAGCAGCAGCCA |
| | | CCTGTTCTATCTGGCCCTGTGCCTGCTGACCTTTACCAGCTCTGCTACC |
| | | GCCGGACCTGAGACACTTTGTGGCGCTGAACTGGTGGACGCCCTGCAGT |
| | | TTGTGTGTGGCGACAGAGGCTTCTACTTCAACAAGCCCACAGGCTACGG |
| | | CAGCAGCTCTAGAAGGGCTCCTCAGACCGGAATCGTGGACGAGTGCTGT |
| | | TTCAGAAGCTGCGACCTGCGGCGGCTGGAAATGTATTGTGCCCCTCTGA |
| | | AGCCTGCCAAGAGCGCCTAAACAACAACAACAAGATGAAGAGCACCAAA |
| | | CTTGTTGGTGCTCTTCATCTTGTTGACAACAATTTATCTTAGAGGCATA |
| | | TCCCT |
| 7 | Compound 7 | GCCACCATGGGCAAGATTAGCAGCCTGCCTACACAGCTGTTCAAGTGCT |
| | | GCTTCTGCGACTTCCTGAAAGTGAAGATGCACACCATGAGCAGCAGCCA |
| | | CCTGTTCTATCTGGCCCTGTGCCTGCTGACCTTTACCAGCTCTGCTACC |
| | | GCCGGACCTGAGACACTTTGTGGCGCTGAACTGGTGGACGCCCTGCAGT |
| | | TTGTGTGTGGCGACAGAGGCTTCTACTTCAACAAGCCCACAGGCTACGG |
| | | CAGCAGCTCTAGAAGGGCTCCTCAGACCGGAATCGTGGACGAGTGCTGT |
| | | TTCAGAAGCTGCGACCTGCGGCGGCTGGAAATGTATTGTGCCCCTCTGA |
| | | AGCCTGCCAAGAGCGCCTAAATAGTGAGTCGTATTAACGTACCAACAAC |
| | | AACAAGATGAAGAGCACCAAACTTGTTGGTGCTCTTCATCTTGTTGTTT |
| | | ATCTTAGAGGCATATCCCTACGTACCAACAACAACAAGATGAAGAGCAC |
| | | CAAACTTGTTGGTGCTCTTCATCTTGTTGTTTATCTTAGAGGCATATCC |
| | | CTTTTATCTTAGAGGCATATCCCT |
| 8 | Compound 8 | GCCACCATGGGCAAGATTAGCAGCCTGCCTACACAGCTGTTCAAGTGCT |
| | | GCTTCTGCGACTTCCTGAAAGTGAAGATGCACACCATGAGCAGCAGCCA |
| | | CCTGTTCTATCTGGCCCTGTGCCTGCTGACCTTTACCAGCTCTGCTACC |
| | | GCCGGACCTGAGACACTTTGTGGCGCTGAACTGGTGGACGCCCTGCAGT |
| | | TTGTGTGTGGCGACAGAGGCTTCTACTTCAACAAGCCCACAGGCTACGG |
| | | CAGCAGCTCTAGAAGGGCTCCTCAGACCGGAATCGTGGACGAGTGCTGT |
| | | TTCAGAAGCTGCGACCTGCGGCGGCTGGAAATGTATTGTGCCCCTCTGA |
| | | AGCCTGCCAAGAGCGCCTAAATAGTGAGTCGTATTAACGTACCAACAAC |
| | | AACAAGATGAAGAGCACCAAACTTGTTGGTGCTCTTCATCTTGTTGTTT |
| | | ATCTTAGAGGCATATCCCTACGTACCAACAAGGACAGCCACATGCACTT |
| | | CAAACTTGTTGAAGTGCATGTGGCTGTCCTTTATCTTAGAGGCATATCC |
| | | CTTTTATCTTAGAGGCATATCCCT |
| 9 | Compound 9 | GCCACCATGGGCAAGATTAGCAGCCTGCCTACACAGCTGTTCAAGTGCT |
| | | GCTTCTGCGACTTCCTGAAAGTGAAGATGCACACCATGAGCAGCAGCCA |
| | | CCTGTTCTATCTGGCCCTGTGCCTGCTGACCTTTACCAGCTCTGCTACC |
| | | GCCGGACCTGAGACACTTTGTGGCGCTGAACTGGTGGACGCCCTGCAGT |
| | | TTGTGTGTGGCGACAGAGGCTTCTACTTCAACAAGCCCACAGGCTACGG |
| | | CAGCAGCTCTAGAAGGGCTCCTCAGACCGGAATCGTGGACGAGTGCTGT |
| | | TTCAGAAGCTGCGACCTGCGGCGGCTGGAAATGTATTGTGCCCCTCTGA |
| | | AGCCTGCCAAGAGCGCCTAAACAACAACAACAAGATGAAGAGCACCAAA |
| | | CTTGTTGGTGCTCTTCATCTTGTTGACAACAACAACAAGATGAAGAGCA |
| | | CCAAACTTGTTGGTGCTCTTCATCTTGTTGACAACAATTTATCTTAGAG |
| | | GCATATCCCT |
| 85 | Compound 10 | GCCACCATGTTGTTGCTGCTGCTCGCCTGTATTGCCCTGGCCTCTACAG |
| | | CCGCCGCTACAAATTCTGCCCCTACCAGCAGCTCCACCAAGAAAACCCA |
| | | GCTGCAACTGGAACATCTGCTGCTGGACCTGCAGATGATCCTGAACGGC |
| | | ATCAACAACTACAAGAACCCCAAGCTGACCCGGATGCTGACCTTCAAGT |
| | | TCTACATGCCCAAGAAGGCCACCGAGCTGAAGCACCTCCAGTGCCTGGA |
| | | AGAGGAACTGAAGCCCCTGGAAGAAGTGCTGAATCTGGCCCAGAGCAAG |
| | | AACTTCCACCTGAGGCCTAGGGACCTGATCAGCAACATCAACGTGATCG |
| | | TGCTGGAACTGAAAGGCAGCGAGACAACCTTCATGTGCGAGTACGCCGA |
| | | CGAGACAGCTACCATCGTGGAATTTCTGAACCGGTGGATCACCTTCTGC |
| | | CAGAGCATCATCAGCACCCTGACCTGAATAGTGAGTCGTATTAACGTAC |
| | | CAACAAGGAGGGCAGAATCATCACGAAGTGGTGAAGTACTTGACTTCAC |
| | | CACTTCGTGATGATTCTGCCCTCCTTTATCTTAGAGGCATATCCCTACG |
| | | TACCAACAAGAGATGAGCTTCCTACAGCACAACAAATGTGACTTGCACA |
| | | TTTGTTGTGCTGTAGGAAGCTCATCTCTTTATCTTAGAGGCATATCCCT |
| | | ACGTACCAACAAGTACAAGATCCGCAGACGTGTAAATGTTCCACTTGGG |
| | | AACATTTACACGTCTGCGGATCTTGTACTTTATCTTAGAGGCATATCCC |
| | | TTTTATCTTAGAGGCATATCCCT |
| 86 | Compound 11 | GCCACCATGTTGTTGCTGCTGCTCGCCTGTATTGCCCTGGCCTCTACAG |
| | | CCGCCGCTACAAATTCTGCCCCTACCAGCAGCTCCACCAAGAAAACCCA |
| | | GCTGCAACTGGAACATCTGCTGCTGGACCTGCAGATGATCCTGAACGGC |
| | | ATCAACAACTACAAGAACCCCAAGCTGACCCGGATGCTGACCTTCAAGT |
| | | TCTACATGCCCAAGAAGGCCACCGAGCTGAAGCACCTCCAGTGCCTGGA |
| | | AGAGGAACTGAAGCCCCTGGAAGAAGTGCTGAATCTGGCCCAGAGCAAG |
| | | AACTTCCACCTGAGGCCTAGGGACCTGATCAGCAACATCAACGTGATCG |
| | | TGCTGGAACTGAAAGGCAGCGAGACAACCTTCATGTGCGAGTACGCCGA |
| | | CGAGACAGCTACCATCGTGGAATTTCTGAACCGGTGGATCACCTTCTGC |
| | | CAGAGCATCATCAGCACCCTGACCTGAACAACAAGGAGGGCAGAATCAT |
| | | CACGAAGTGGTGAAGTACTTGACTTCACCACTTCGTGATGATTCTGCCC |
| | | TCCACAACAAGAGATGAGCTTCCTACAGCACAACAAATGTGACTTGCAC |
| | | ATTTGTTGTGCTGTAGGAAGCTCATCTCACAACAAGTACAAGATCCGCA |
| | | GACGTGTAAATGTTCCACTTGGGAACATTTACACGTCTGCGGATCTTGT |
| | | ACACAACAATTTATCTTAGAGGCATATCCCTCTGGGCCTCATGGGCCTT |
| | | CCGCTCACTGCCCGCTTTCCAGTCGGGAAACCTGTCGTGCCAGCTGCAT |
| | | TAACATGGTCATAGCTG |
| 87 | Compound 12 | GCCACCATGTTGTTGCTGCTGCTCGCCTGTATTGCCCTGGCCTCTACAG |
| | | CCGCCGCTACAAATTCTGCCCCTACCAGCAGCTCCACCAAGAAAACCCA |
| | | GCTGCAACTGGAACATCTGCTGCTGGACCTGCAGATGATCCTGAACGGC |
| | | ATCAACAACTACAAGAACCCCAAGCTGACCCGGATGCTGACCTTCAAGT |
| | | TCTACATGCCCAAGAAGGCCACCGAGCTGAAGCACCTCCAGTGCCTGGA |
| | | AGAGGAACTGAAGCCCCTGGAAGAAGTGCTGAATCTGGCCCAGAGCAAG |
| | | AACTTCCACCTGAGGCCTAGGGACCTGATCAGCAACATCAACGTGATCG |
| | | TGCTGGAACTGAAAGGCAGCGAGACAACCTTCATGTGCGAGTACGCCGA |
| | | CGAGACAGCTACCATCGTGGAATTTCTGAACCGGTGGATCACCTTCTGC |
| | | CAGAGCATCATCAGCACCCTGACCTGAATAGTGAGTCGTATTAGGAGGG |
| | | CAGAATCATCACGAAGTGGTGAAGTACTTGACTTCACCACTTCGTGATG |
| | | ATTCTGCCCTCCATCCCTACGTACCAACAAGAGATGAGCTTCCTACAGC |
| | | ACAACAAATGTGACTTGCACATTTGTTGTGCTGTAGGAAGCTCATCTCA |
| | | TCCCTACGTACCAACAAGTACAAGATCCGCAGACGTGTAAATGTTCCAC |
| | | TTGGGAACATTTACACGTCTGCGGATCTTGTACTTTATCTTAGAGGCAT |
| 88 | Compound 13 | GCCACCATGTTGTTGCTGCTGCTCGCCTGTATTGCCCTGGCCTCTACAG |
| | | CCGCCGCTACAAATTCTGCCCCTACCAGCAGCTCCACCAAGAAAACCCA |
| | | GCTGCAACTGGAACATCTGCTGCTGGACCTGCAGATGATCCTGAACGGC |
| | | ATCAACAACTACAAGAACCCCAAGCTGACCCGGATGCTGACCTTCAAGT |
| | | TCTACATGCCCAAGAAGGCCACCGAGCTGAAGCACCTCCAGTGCCTGGA |
| | | AGAGGAACTGAAGCCCCTGGAAGAAGTGCTGAATCTGGCCCAGAGCAAG |
| | | AACTTCCACCTGAGGCCTAGGGACCTGATCAGCAACATCAACGTGATCG |
| | | TGCTGGAACTGAAAGGCAGCGAGACAACCTTCATGTGCGAGTACGCCGA |
| | | CGAGACAGCTACCATCGTGGAATTTCTGAACCGGTGGATCACCTTCTGC |
| | | CAGAGCATCATCAGCACCCTGACCTGAATAGTGAGTCGTATTAGGAGGG |
| | | CAGAATCATCACGAAGTGGTGAAGTACTTGACTTCACCACTTCGTGATG |
| | | ATTCTGCCCTCCACGTACCAACAAGAGATGAGCTTCCTACAGCACAACA |
| | | AATGTGACTTGCACATTTGTTGTGCTGTAGGAAGCTCATCTCACGTACC |
| | | AACAAGTACAAGATCCGCAGACGTGTAAATGTTCCACTTGGGAACATTT |
| | | ACACGTCTGCGGATCTTGTACTTTATCTTAGAGGCAT |
| 89 | Compound 14 | GCCACCATGTTGTTGCTGCTGCTCGCCTGTATTGCCCTGGCCTCTACAG |
| | | CCGCCGCTACAAATTCTGCCCCTACCAGCAGCTCCACCAAGAAAACCCA |
| | | GCTGCAACTGGAACATCTGCTGCTGGACCTGCAGATGATCCTGAACGGC |
| | | ATCAACAACTACAAGAACCCCAAGCTGACCCGGATGCTGACCTTCAAGT |
| | | TCTACATGCCCAAGAAGGCCACCGAGCTGAAGCACCTCCAGTGCCTGGA |
| | | AGAGGAACTGAAGCCCCTGGAAGAAGTGCTGAATCTGGCCCAGAGCAAG |
| | | AACTTCCACCTGAGGCCTAGGGACCTGATCAGCAACATCAACGTGATCG |
| | | TGCTGGAACTGAAAGGCAGCGAGACAACCTTCATGTGCGAGTACGCCGA |
| | | CGAGACAGCTACCATCGTGGAATTTCTGAACCGGTGGATCACCTTCTGC |
| | | CAGAGCATCATCAGCACCCTGACCTGAATAGTGAGTCGTATTATCCCGG |
| | | AGGGCAGAATCATCACGAAGTGGTGAAGTACTTGACTTCACCACTTCGT |
| | | GATGATTCTGCCCTCCTCCCGAGATGAGCTTCCTACAGCACAACAAATG |
| | | TGACTTGCACATTTGTTGTGCTGTAGGAAGCTCATCTCTCCCGTACAAG |
| | | ATCCGCAGACGTGTAAATGTTCCACTTGGGAACATTTACACGTCTGCGG |
| | | ATCTTGTACTTTATCTTAGAGGCAT |
| 90 | Compound 15 | GCCACCATGTTGTTGCTGCTGCTCGCCTGTATTGCCCTGGCCTCTACAG |
| | | CCGCCGCTACAAATTCTGCCCCTACCAGCAGCTCCACCAAGAAAACCCA |
| | | GCTGCAACTGGAACATCTGCTGCTGGACCTGCAGATGATCCTGAACGGC |
| | | ATCAACAACTACAAGAACCCCAAGCTGACCCGGATGCTGACCTTCAAGT |
| | | TCTACATGCCCAAGAAGGCCACCGAGCTGAAGCACCTCCAGTGCCTGGA |
| | | AGAGGAACTGAAGCCCCTGGAAGAAGTGCTGAATCTGGCCCAGAGCAAG |
| | | AACTTCCACCTGAGGCCTAGGGACCTGATCAGCAACATCAACGTGATCG |
| | | TGCTGGAACTGAAAGGCAGCGAGACAACCTTCATGTGCGAGTACGCCGA |
| | | CGAGACAGCTACCATCGTGGAATTTCTGAACCGGTGGATCACCTTCTGC |
| | | CAGAGCATCATCAGCACCCTGACCTGAATAGTGAGTCGTATTAACAACA |
| | | ATCCCGGAGGGCAGAATCATCACGAAGTGGTGAAGTACTTGACTTCACC |
| | | ACTTCGTGATGATTCTGCCCTCCACAACAATCCCGAGATGAGCTTCCTA |
| | | CAGCACAACAAATGTGACTTGCACATTTGTTGTGCTGTAGGAAGCTCAT |
| | | CTCACAACAATCCCGTACAAGATCCGCAGACGTGTAAATGTTCCACTTG |
| | | GGAACATTTACACGTCTGCGGATCTTGTACTTTATCTTAGAGGCAT |
| 91 | Compound 16 | GCCACCATGTGTCACCAGCAGCTGGTCATCAGCTGGTTCAGCCTGGTGT |
| | | TCCTGGCCTCTCCTCTGGTGGCCATCTGGGAGCTGAAGAAAGACGTGTA |
| | | CGTGGTGGAACTGGACTGGTATCCCGATGCTCCTGGCGAGATGGTGGTG |
| | | CTGACCTGCGATACCCCTGAAGAGGACGGCATCACCTGGACACTGGATC |
| | | AGTCTAGCGAGGTGCTCGGCAGCGGCAAGACCCTGACCATCCAAGTGAA |
| | | AGAGTTTGGCGACGCCGGCCAGTACACCTGTCACAAAGGCGGAGAAGTG |
| | | CTGAGCCACAGCCTGCTGCTGCTCCACAAGAAAGAGGATGGCATTTGGA |
| | | GCACCGACATCCTGAAGGACCAGAAAGAGCCCAAGAACAAGACCTTCCT |
| | | GAGATGCGAGGCCAAGAACTACAGCGGCCGGTTCACATGTTGGTGGCTG |
| | | ACCACCATCAGCACCGACCTGACCTTCAGCGTGAAGTCCAGCAGAGGCA |
| | | GCAGTGATCCTCAGGGCGTTACATGTGGCGCCGCTACACTGTCTGCCGA |
| | | AAGAGTGCGGGGCGACAACAAAGAATACGAGTACAGCGTGGAATGCCAA |
| | | GAGGACAGCGCCTGTCCAGCCGCCGAAGAGTCTCTGCCTATCGAAGTGA |
| | | TGGTGGACGCCGTGCACAAGCTGAAGTACGAGAACTACACCTCCAGCTT |
| | | TTTCATCCGGGACATCATCAAGCCCGATCCTCCAAAGAACCTGCAGCTG |
| | | AAGCCTCTGAAGAACAGCAGACAGGTGGAAGTGTCCTGGGAGTACCCCG |
| | | ACACCTGGTCTACACCCCACAGCTACTTCAGCCTGACCTTTTGCGTGCA |
| | | AGTGCAGGGCAAGTCCAAGCGCGAGAAAAAGGACCGGGTGTTCACCGAC |
| | | AAGACCAGCGCCACCGTGATCTGCAGAAAGAACGCCAGCATCAGCGTCA |
| | | GAGCCCAGGACCGGTACTACAGCAGCTCTTGGAGCGAATGGGCCAGCGT |
| | | GCCATGTTCTGGTGGCGGAGGATCTGGCGGAGGTGGAAGCGGCGGAGGC |
| | | GGATCTAGAAATCTGCCTGTGGCCACTCCTGATCCTGGCATGTTCCCTT |
| | | GTCTGCACCACAGCCAGAACCTGCTGAGAGCCGTGTCCAACATGCTGCA |
| | | GAAGGCCAGACAGACCCTGGAATTCTACCCCTGCACCAGCGAGGAAATC |
| | | GACCACGAGGACATCACCAAGGATAAGACCAGCACCGTGGAAGCCTGCC |
| | | TGCCTCTGGAACTGACCAAGAACGAGAGCTGCCTGAACAGCCGGGAAAC |
| | | CAGCTTCATCACCAACGGCTCTTGCCTGGCCAGCAGAAAGACCTCCTTC |
| | | ATGATGGCCCTGTGCCTGAGCAGCATCTACGAGGACCTGAAGATGTACC |
| | | AGGTGGAATTCAAGACCATGAACGCCAAGCTGCTGATGGACCCCAAGCG |
| | | GCAGATCTTCCTGGACCAGAATATGCTGGCCGTGATCGACGAGCTGATG |
| | | CAGGCCCTGAACTTCAACAGCGAGACAGTGCCCCAGAAGTCTAGCCTGG |
| | | AAGAACCCGACTTCTACAAGACCAAGATCAAGCTGTGCATCCTGCTGCA |
| | | CGCCTTCCGGATCAGAGCCGTGACCATCGACAGAGTGATGAGCTACCTG |
| | | AACGCCTCCTGAATAGTGAGTCGTATTAACGTACCAACAAGGACGACGA |
| | | GACCTTCATCAAACTTGTTGATGAAGGTCTCGTCGTCCTTTATCTTAGA |
| | | GGCATATCCCTACGTACCAACAAGTGCAATGAGGGACCAGTACAACTTG |
| | | TGTACTGGTCCCTCATTGCACTTTATCTTAGAGGCATATCCCTACGTAC |
| | | CAACAATTCTACAACCAGGACCATGAGACTTGCTCATGGTCCTGGTTGT |
| | | AGAATTTATCTTAGAGGCATATCCCTTTTATCTTAGAGGCATATCCCT |
| 92 | Compound 17 | GCCACCATGTGTCACCAGCAGCTGGTCATCAGCTGGTTCAGCCTGGTGT |
| | | TCCTGGCCTCTCCTCTGGTGGCCATCTGGGAGCTGAAGAAAGACGTGTA |
| | | CGTGGTGGAACTGGACTGGTATCCCGATGCTCCTGGCGAGATGGTGGTG |
| | | CTGACCTGCGATACCCCTGAAGAGGACGGCATCACCTGGACACTGGATC |
| | | AGTCTAGCGAGGTGCTCGGCAGCGGCAAGACCCTGACCATCCAAGTGAA |
| | | AGAGTTTGGCGACGCCGGCCAGTACACCTGTCACAAAGGCGGAGAAGTG |
| | | CTGAGCCACAGCCTGCTGCTGCTCCACAAGAAAGAGGATGGCATTTGGA |
| | | GCACCGACATCCTGAAGGACCAGAAAGAGCCCAAGAACAAGACCTTCCT |
| | | GAGATGCGAGGCCAAGAACTACAGCGGCCGGTTCACATGTTGGTGGCTG |
| | | ACCACCATCAGCACCGACCTGACCTTCAGCGTGAAGTCCAGCAGAGGCA |
| | | GCAGTGATCCTCAGGGCGTTACATGTGGCGCCGCTACACTGTCTGCCGA |
| | | AAGAGTGCGGGGCGACAACAAAGAATACGAGTACAGCGTGGAATGCCAA |
| | | GAGGACAGCGCCTGTCCAGCCGCCGAAGAGTCTCTGCCTATCGAAGTGA |
| | | TGGTGGACGCCGTGCACAAGCTGAAGTACGAGAACTACACCTCCAGCTT |
| | | TTTCATCCGGGACATCATCAAGCCCGATCCTCCAAAGAACCTGCAGCTG |
| | | AAGCCTCTGAAGAACAGCAGACAGGTGGAAGTGTCCTGGGAGTACCCCG |
| | | ACACCTGGTCTACACCCCACAGCTACTTCAGCCTGACCTTTTGCGTGCA |
| | | AGTGCAGGGCAAGTCCAAGCGCGAGAAAAAGGACCGGGTGTTCACCGAC |
| | | AAGACCAGCGCCACCGTGATCTGCAGAAAGAACGCCAGCATCAGCGTCA |
| | | GAGCCCAGGACCGGTACTACAGCAGCTCTTGGAGCGAATGGGCCAGCGT |
| | | GCCATGTTCTGGTGGCGGAGGATCTGGCGGAGGTGGAAGCGGCGGAGGC |
| | | GGATCTAGAAATCTGCCTGTGGCCACTCCTGATCCTGGCATGTTCCCTT |
| | | GTCTGCACCACAGCCAGAACCTGCTGAGAGCCGTGTCCAACATGCTGCA |
| | | GAAGGCCAGACAGACCCTGGAATTCTACCCCTGCACCAGCGAGGAAATC |
| | | GACCACGAGGACATCACCAAGGATAAGACCAGCACCGTGGAAGCCTGCC |
| | | TGCCTCTGGAACTGACCAAGAACGAGAGCTGCCTGAACAGCCGGGAAAC |
| | | CAGCTTCATCACCAACGGCTCTTGCCTGGCCAGCAGAAAGACCTCCTTC |
| | | ATGATGGCCCTGTGCCTGAGCAGCATCTACGAGGACCTGAAGATGTACC |
| | | AGGTGGAATTCAAGACCATGAACGCCAAGCTGCTGATGGACCCCAAGCG |
| | | GCAGATCTTCCTGGACCAGAATATGCTGGCCGTGATCGACGAGCTGATG |
| | | CAGGCCCTGAACTTCAACAGCGAGACAGTGCCCCAGAAGTCTAGCCTGG |
| | | AAGAACCCGACTTCTACAAGACCAAGATCAAGCTGTGCATCCTGCTGCA |
| | | CGCCTTCCGGATCAGAGCCGTGACCATCGACAGAGTGATGAGCTACCTG |
| | | AACGCCTCCTGAACAACAAGGACGACGAGACCTTCATCAAACTTGTTGA |
| | | TGAAGGTCTCGTCGTCCACAACAAGTGCAATGAGGGACCAGTACAACTT |
| | | GTGTACTGGTCCCTCATTGCACACAACAATTCTACAACCAGGACCATGA |
| | | GACTTGCTCATGGTCCTGGTTGTAGAAACAACAATTTATCTTAGAGGCA |
| | | TATCCCT |

Bold = Sense siRNA strand
Bold and Italics = anti-Sense siRNA strand
Underline = Signal peptide
Italics = Kozak sequence

TABLE 3
Plasmid Vector Sequences for Compounds

| SEQ ID NO | Compound | Sequence (5′ to 3′) |
|---|---|---|
| 10 | Compound 1 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGACTGACATCTCAACTGCTGCCTCCACTGTTCTTTCT |
| | | GCTGGCCTGCGCCGGCAATTTTGTGCACGGCCACAAGTGCGACATCACCCTG |
| | | CAAGAGATCATCAAGACCCTGAACAGCCTGACCGAGCAGAAAACCCTGTGCA |
| | | CCGAGCTGACCGTGACCGATATCTTTGCCGCCAGCAAGAACACAACCGAGAA |
| | | AGAGACATTCTGCAGAGCCGCCACCGTGCTGAGACAGTTCTACAGCCACCAC |
| | | GAGAAGGACACCAGATGCCTGGGAGCTACAGCCCAGCAGTTCCACAGACACA |
| | | AGCAGCTGATCCGGTTCCTGAAGCGGCTGGACAGAAATCTGTGGGGACTCGC |
| | | CGGCCTGAATAGCTGCCCTGTGAAAGAGGCCAACCAGTCTACCCTGGAAAAC |
| | | TTCCTGGAACGGCTGAAAACCATCATGCGCGAGAAGTACAGCAAGTGCAGCA |
| | | GCTGAATAGTGAGTCGTATTAACGTACCAACAAGGCGTGGAGCTGAGAGATA |
| | | AACTTGTTATCTCTCAGCTCCACGCCTTTATCTTAGAGGCATATCCCTACGT |
| | | ACCAACAAGGGCCTGTACCTCATCTACTACTTGAGTAGATGAGGTACAGGCC |
| | | CTTTATCTTAGAGGCATATCCCTACGTACCAACAAGGTATGAGCCCATCTAT |
| | | CTACTTGAGATAGATGGGCTCATACCTTTATCTTAGAGGCATATCCCTTTTA |
| | | TCTTAGAGGCATATCCCTCTGGGCCTCATGGGCCTTCCGCTCACTGCCCGCT |
| | | TTCCAGTCGGGAAACCTGTCGTGCCAGCTGCATTAACATGGTCATAGCTGTT |
| | | TCCTTGCGTATTGGGCGCTCTCCGCTTCCTCGCTCACTGACTCGCTGCGCTC |
| | | GGTCGTTCGGGTAAAGCCTGGGGTGCCTAATGAGCAAAAGGCCAGCAAAAGG |
| | | CCAGGAACCGTAAAAAGGCCGCGTTGCTGGCGTTTTTCCATAGGCTCCGCCC |
| | | CCCTGACGAGCATCACAAAAATCGACGCTCAAGTCAGAGGTGGCGAAACCCG |
| | | ACAGGACTATAAAGATACCAGGCGTTTCCCCCTGGAAGCTCCCTCGTGCGCT |
| | | CTCCTGTTCCGACCCTGCCGCTTACCGGATACCTGTCCGCCTTTCTCCCTTC |
| | | GGGAAGCGTGGCGCTTTCTCATAGCTCACGCTGTAGGTATCTCAGTTCGGTG |
| | | TAGGTCGTTCGCTCCAAGCTGGGCTGTGTGCACGAACCCCCCGTTCAGCCCG |
| | | ACCGCTGCGCCTTATCCGGTAACTATCGTCTTGAGTCCAACCCGGTAAGACA |
| | | CGACTTATCGCCACTGGCAGCAGCCACTGGTAACAGGATTAGCAGAGCGAGG |
| | | TATGTAGGCGGTGCTACAGAGTTCTTGAAGTGGTGGCCTAACTACGGCTACA |
| | | CTAGAAGAACAGTATTTGGTATCTGCGCTCTGCTGAAGCCAGTTACCTTCGG |
| | | AAAAAGAGTTGGTAGCTCTTGATCCGGCAAACAAACCACCGCTGGTAGCGGT |
| | | GGTTTTTTTGTTTGCAAGCAGCAGATTACGCGCAGAAAAAAAGGATCTCAAG |
| | | AAGATCCTTTGATCTTTTCTACGGGGTCTGACGCTCAGTGGAACGAAAACTC |
| | | ACGTTAAGGGATTTTGGTCATGAGATTATCAAAAAGGATCTTCACCTAGATC |
| | | CTTTTAAATTAAAAATGAAGTTTTAAATCAATCTAAAGTATATATGAGTAAA |
| | | CTTGGTCTGACAGTTACCAATGCTTAATCAGTGAGGCACCTATCTCAGCGAT |
| | | CTGTCTATTTCGTTCATCCATAGTTGCCTGACTCCCCGTCGTGTAGATAACT |
| | | ACGATACGGGAGGGCTTACCATCTGGCCCCAGTGCTGCAATGATACCGCGAG |
| | | AACCACGCTCACCGGCTCCAGATTTATCAGCAATAAACCAGCCAGCCGGAAG |
| | | GGCCGAGCGCAGAAGTGGTCCTGCAACTTTATCCGCCTCCATCCAGTCTATT |
| | | AATTGTTGCCGGGAAGCTAGAGTAAGTAGTTCGCCAGTTAATAGTTTGCGCA |
| | | ACGTTGTTGCCATTGCTACAGGCATCGTGGTGTCACGCTCGTCGTTTGGTAT |
| | | GGCTTCATTCAGCTCCGGTTCCCAACGATCAAGGCGAGTTACATGATCCCCC |
| | | ATGTTGTGCAAAAAAGCGGTTAGCTCCTTCGGTCCTCCGATCGTTGTCAGAA |
| | | GTAAGTTGGCCGCAGTGTTATCACTCATGGTTATGGCAGCACTGCATAATTC |
| | | TCTTACTGTCATGCCATCCGTAAGATGCTTTTCTGTGACTGGTGAGTACTCA |
| | | ACCAAGTCATTCTGAGAATAGTGTATGCGGCGACCGAGTTGCTCTTGCCCGG |
| | | CGTCAATACGGGATAATACCGCGCCACATAGCAGAACTTTAAAAGTGCTCAT |
| | | CATTGGAAAACGTTCTTCGGGGCGAAAACTCTCAAGGATCTTACCGCTGTTG |
| | | AGATCCAGTTCGATGTAACCCACTCGTGCACCCAACTGATCTTCAGCATCTT |
| | | TTACTTTCACCAGCGTTTCTGGGTGAGCAAAAACAGGAAGGCAAAATGCCGC |
| | | AAAAAAGGGAATAAGGGCGACACGGAAATGTTGAATACTCATACTCTTCCTT |
| | | TTTCAATATTATTGAAGCATTTATCAGGGTTATTGTCTCATGAGCGGATACA |
| | | TATTTGAATGTATTTAGAAAAATAAACAAATAGGGGTTCCGCGCACATTTCC |
| | | CCGAAAAGTGCCAC |
| 11 | Compound 2 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGACTGACATCTCAACTGCTGCCTCCACTGTTCTTTCT |
| | | GCTGGCCTGCGCCGGCAATTTTGTGCACGGCCACAAGTGCGACATCACCCTG |
| | | CAAGAGATCATCAAGACCCTGAACAGCCTGACCGAGCAGAAAACCCTGTGCA |
| | | CCGAGCTGACCGTGACCGATATCTTTGCCGCCAGCAAGAACACAACCGAGAA |
| | | AGAGACATTCTGCAGAGCCGCCACCGTGCTGAGACAGTTCTACAGCCACCAC |
| | | GAGAAGGACACCAGATGCCTGGGAGCTACAGCCCAGCAGTTCCACAGACACA |
| | | AGCAGCTGATCCGGTTCCTGAAGCGGCTGGACAGAAATCTGTGGGGACTCGC |
| | | CGGCCTGAATAGCTGCCCTGTGAAAGAGGCCAACCAGTCTACCCTGGAAAAC |
| | | TTCCTGGAACGGCTGAAAACCATCATGCGCGAGAAGTACAGCAAGTGCAGCA |
| | | GCTGAACAACAAGGCGTGGAGCTGAGAGATAAACTTGTTATCTCTCAGCTCC |
| | | ACGCCACAACAAGGGCCTGTACCTCATCTACTACTTGAGTAGATGAGGTACA |
| | | GGCCCACAACAAGGTATGAGCCCATCTATCTACTTGAGATAGATGGGCTCAT |
| | | ACCACAACAATTTATCTTAGAGGCATATCCCTCTGGGCCTCATGGGCCTTCC |
| | | GCTCACTGCCCGCTTTCCAGTCGGGAAACCTGTCGTGCCAGCTGCATTAACA |
| | | TGGTCATAGCTGTTTCCTTGCGTATTGGGCGCTCTCCGCTTCCTCGCTCACT |
| | | GACTCGCTGCGCTCGGTCGTTCGGGTAAAGCCTGGGGTGCCTAATGAGCAAA |
| | | AGGCCAGCAAAAGGCCAGGAACCGTAAAAAGGCCGCGTTGCTGGCGTTTTTC |
| | | CATAGGCTCCGCCCCCCTGACGAGCATCACAAAAATCGACGCTCAAGTCAGA |
| | | GGTGGCGAAACCCGACAGGACTATAAAGATACCAGGCGTTTCCCCCTGGAAG |
| | | CTCCCTCGTGCGCTCTCCTGTTCCGACCCTGCCGCTTACCGGATACCTGTCC |
| | | GCCTTTCTCCCTTCGGGAAGCGTGGCGCTTTCTCATAGCTCACGCTGTAGGT |
| | | ATCTCAGTTCGGTGTAGGTCGTTCGCTCCAAGCTGGGCTGTGTGCACGAACC |
| | | CCCCGTTCAGCCCGACCGCTGCGCCTTATCCGGTAACTATCGTCTTGAGTCC |
| | | AACCCGGTAAGACACGACTTATCGCCACTGGCAGCAGCCACTGGTAACAGGA |
| | | TTAGCAGAGCGAGGTATGTAGGCGGTGCTACAGAGTTCTTGAAGTGGTGGCC |
| | | TAACTACGGCTACACTAGAAGAACAGTATTTGGTATCTGCGCTCTGCTGAAG |
| | | CCAGTTACCTTCGGAAAAAGAGTTGGTAGCTCTTGATCCGGCAAACAAACCA |
| | | CCGCTGGTAGCGGTGGTTTTTTTGTTTGCAAGCAGCAGATTACGCGCAGAAA |
| | | AAAAGGATCTCAAGAAGATCCTTTGATCTTTTCTACGGGGTCTGACGCTCAG |
| | | TGGAACGAAAACTCACGTTAAGGGATTTTGGTCATGAGATTATCAAAAAGGA |
| | | TCTTCACCTAGATCCTTTTAAATTAAAAATGAAGTTTTAAATCAATCTAAAG |
| | | TATATATGAGTAAACTTGGTCTGACAGTTACCAATGCTTAATCAGTGAGGCA |
| | | CCTATCTCAGCGATCTGTCTATTTCGTTCATCCATAGTTGCCTGACTCCCCG |
| | | TCGTGTAGATAACTACGATACGGGAGGGCTTACCATCTGGCCCCAGTGCTGC |
| | | AATGATACCGCGAGAACCACGCTCACCGGCTCCAGATTTATCAGCAATAAAC |
| | | CAGCCAGCCGGAAGGGCCGAGCGCAGAAGTGGTCCTGCAACTTTATCCGCCT |
| | | CCATCCAGTCTATTAATTGTTGCCGGGAAGCTAGAGTAAGTAGTTCGCCAGT |
| | | TAATAGTTTGCGCAACGTTGTTGCCATTGCTACAGGCATCGTGGTGTCACGC |
| | | TCGTCGTTTGGTATGGCTTCATTCAGCTCCGGTTCCCAACGATCAAGGCGAG |
| | | TTACATGATCCCCCATGTTGTGCAAAAAAGCGGTTAGCTCCTTCGGTCCTCC |
| | | GATCGTTGTCAGAAGTAAGTTGGCCGCAGTGTTATCACTCATGGTTATGGCA |
| | | GCACTGCATAATTCTCTTACTGTCATGCCATCCGTAAGATGCTTTTCTGTGA |
| | | CTGGTGAGTACTCAACCAAGTCATTCTGAGAATAGTGTATGCGGCGACCGAG |
| | | TTGCTCTTGCCCGGCGTCAATACGGGATAATACCGCGCCACATAGCAGAACT |
| | | TTAAAAGTGCTCATCATTGGAAAACGTTCTTCGGGGCGAAAACTCTCAAGGA |
| | | TCTTACCGCTGTTGAGATCCAGTTCGATGTAACCCACTCGTGCACCCAACTG |
| | | ATCTTCAGCATCTTTTACTTTCACCAGCGTTTCTGGGTGAGCAAAAACAGGA |
| | | AGGCAAAATGCCGCAAAAAAGGGAATAAGGGCGACACGGAAATGTTGAATAC |
| | | TCATACTCTTCCTTTTTCAATATTATTGAAGCATTTATCAGGGTTATTGTCT |
| | | CATGAGCGGATACATATTTGAATGTATTTAGAAAAATAAACAAATAGGGGTT |
| | | CCGCGCACATTTCCCCGAAAAGTGCCAC |
| 12 | Compound 3 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGACCATCCTGTTTCTGACAATGGTCATCAGCTACTTCGG |
| | | CTGCATGAAGGCCGTGAAGATGCACACCATGAGCAGCAGCCACCTGTTCTAT |
| | | CTGGCCCTGTGCCTGCTGACCTTTACCAGCTCTGCTACCGCCGGACCTGAGA |
| | | CACTTTGTGGCGCTGAACTGGTGGACGCCCTGCAGTTTGTGTGTGGCGACAG |
| | | AGGCTTCTACTTCAACAAGCCCACAGGCTACGGCAGCAGCTCTAGAAGGGCT |
| | | CCTCAGACCGGAATCGTGGACGAGTGCTGCTTCAGAAGCTGCGACCTGCGGC |
| | | GGCTGGAAATGTATTGTGCCCCTCTGAAGCCTGCCAAGAGCGCCTAAATAGT |
| | | GAGTCGTATTAACGTACCAACAAGGCCTCATTATTCTCTCTACTTGAGAGAG |
| | | AATAATGAGGCCTTTATCTTAGAGGCATATCCCTACGTACCAACAAGTGTTC |
| | | GCAGTATGTCTTACTTGAAGACATACTGCGAACACTTTATCTTAGAGGCATA |
| | | TCCCTACGTACCAACAAGCCTGCCTGCTGGGAGTTACTTGAACTCCCAGCAG |
| | | GCAGGCTTTATCTTAGAGGCATATCCCTTTTATCTTAGAGGCATATCCCTCT |
| | | GGGCCTCATGGGCCTTCCGCTCACTGCCCGCTTTCCAGTCGGGAAACCTGTC |
| | | GTGCCAGCTGCATTAACATGGTCATAGCTGTTTCCTTGCGTATTGGGCGCTC |
| | | TCCGCTTCCTCGCTCACTGACTCGCTGCGCTCGGTCGTTCGGGTAAAGCCTG |
| | | GGGTGCCTAATGAGCAAAAGGCCAGCAAAAGGCCAGGAACCGTAAAAAGGCC |
| | | GCGTTGCTGGCGTTTTTCCATAGGCTCCGCCCCCCTGACGAGCATCACAAAA |
| | | ATCGACGCTCAAGTCAGAGGTGGCGAAACCCGACAGGACTATAAAGATACCA |
| | | GGCGTTTCCCCCTGGAAGCTCCCTCGTGCGCTCTCCTGTTCCGACCCTGCCG |
| | | CTTACCGGATACCTGTCCGCCTTTCTCCCTTCGGGAAGCGTGGCGCTTTCTC |
| | | ATAGCTCACGCTGTAGGTATCTCAGTTCGGTGTAGGTCGTTCGCTCCAAGCT |
| | | GGGCTGTGTGCACGAACCCCCCGTTCAGCCCGACCGCTGCGCCTTATCCGGT |
| | | AACTATCGTCTTGAGTCCAACCCGGTAAGACACGACTTATCGCCACTGGCAG |
| | | CAGCCACTGGTAACAGGATTAGCAGAGCGAGGTATGTAGGCGGTGCTACAGA |
| | | GTTCTTGAAGTGGTGGCCTAACTACGGCTACACTAGAAGAACAGTATTTGGT |
| | | ATCTGCGCTCTGCTGAAGCCAGTTACCTTCGGAAAAAGAGTTGGTAGCTCTT |
| | | GATCCGGCAAACAAACCACCGCTGGTAGCGGTGGTTTTTTTGTTTGCAAGCA |
| | | GCAGATTACGCGCAGAAAAAAAGGATCTCAAGAAGATCCTTTGATCTTTTCT |
| | | ACGGGGTCTGACGCTCAGTGGAACGAAAACTCACGTTAAGGGATTTTGGTCA |
| | | TGAGATTATCAAAAAGGATCTTCACCTAGATCCTTTTAAATTAAAAATGAAG |
| | | TTTTAAATCAATCTAAAGTATATATGAGTAAACTTGGTCTGACAGTTACCAA |
| | | TGCTTAATCAGTGAGGCACCTATCTCAGCGATCTGTCTATTTCGTTCATCCA |
| | | TAGTTGCCTGACTCCCCGTCGTGTAGATAACTACGATACGGGAGGGCTTACC |
| | | ATCTGGCCCCAGTGCTGCAATGATACCGCGAGAACCACGCTCACCGGCTCCA |
| | | GATTTATCAGCAATAAACCAGCCAGCCGGAAGGGCCGAGCGCAGAAGTGGTC |
| | | CTGCAACTTTATCCGCCTCCATCCAGTCTATTAATTGTTGCCGGGAAGCTAG |
| | | AGTAAGTAGTTCGCCAGTTAATAGTTTGCGCAACGTTGTTGCCATTGCTACA |
| | | GGCATCGTGGTGTCACGCTCGTCGTTTGGTATGGCTTCATTCAGCTCCGGTT |
| | | CCCAACGATCAAGGCGAGTTACATGATCCCCCATGTTGTGCAAAAAAGCGGT |
| | | TAGCTCCTTCGGTCCTCCGATCGTTGTCAGAAGTAAGTTGGCCGCAGTGTTA |
| | | TCACTCATGGTTATGGCAGCACTGCATAATTCTCTTACTGTCATGCCATCCG |
| | | TAAGATGCTTTTCTGTGACTGGTGAGTACTCAACCAAGTCATTCTGAGAATA |
| | | GTGTATGCGGCGACCGAGTTGCTCTTGCCCGGCGTCAATACGGGATAATACC |
| | | GCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACC |
| | | CACTCGTGCACCCAACTGATCTTCAGCATCTTTTACTTTCACCAGCGTTTCT |
| | | GGGTGAGCAAAAACAGGAAGGCAAAATGCCGCAAAAAAGGGAATAAGGGCGA |
| | | CACGGAAATGTTGAATACTCATACTCTTCCTTTTTCAATATTATTGAAGCAT |
| | | TTATCAGGGTTATTGTCTCATGAGCGGATACATATTTGAATGTATTTAGAAA |
| | | AATAAACAAATAGGGGTTCCGCGCACATTTCCCCGAAAAGTGCCAC |
| 13 | Compound 4 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGACCATCCTGTTTCTGACAATGGTCATCAGCTACTTCGG |
| | | CTGCATGAAGGCCGTGAAGATGCACACCATGAGCAGCAGCCACCTGTTCTAT |
| | | CTGGCCCTGTGCCTGCTGACCTTTACCAGCTCTGCTACCGCCGGACCTGAGA |
| | | CACTTTGTGGCGCTGAACTGGTGGACGCCCTGCAGTTTGTGTGTGGCGACAG |
| | | AGGCTTCTACTTCAACAAGCCCACAGGCTACGGCAGCAGCTCTAGAAGGGCT |
| | | CCTCAGACCGGAATCGTGGACGAGTGCTGCTTCAGAAGCTGCGACCTGCGGC |
| | | GGCTGGAAATGTATTGTGCCCCTCTGAAGCCTGCCAAGAGCGCCTAAACAAC |
| | | AAGGCCTCATTATTCTCTCTACTTGAGAGAGAATAATGAGGCCACAACAAGT |
| | | GTTCGCAGTATGTCTTACTTGAAGACATACTGCGAACACACAACAAGCCTGC |
| | | CTGCTGGGAGTTACTTGAACTCCCAGCAGGCAGGCACAACAATTTATCTTAG |
| | | AGGCATATCCCTCTGGGCCTCATGGGCCTTCCGCTCACTGCCCGCTTTCCAG |
| | | TCGGGAAACCTGTCGTGCCAGCTGCATTAACATGGTCATAGCTGTTTCCTTG |
| | | CGTATTGGGCGCTCTCCGCTTCCTCGCTCACTGACTCGCTGCGCTCGGTCGT |
| | | TCGGGTAAAGCCTGGGGTGCCTAATGAGCAAAAGGCCAGCAAAAGGCCAGGA |
| | | ACCGTAAAAAGGCCGCGTTGCTGGCGTTTTTCCATAGGCTCCGCCCCCCTGA |
| | | CGAGCATCACAAAAATCGACGCTCAAGTCAGAGGTGGCGAAACCCGACAGGA |
| | | CTATAAAGATACCAGGCGTTTCCCCCTGGAAGCTCCCTCGTGCGCTCTCCTG |
| | | TTCCGACCCTGCCGCTTACCGGATACCTGTCCGCCTTTCTCCCTTCGGGAAG |
| | | CGTGGCGCTTTCTCATAGCTCACGCTGTAGGTATCTCAGTTCGGTGTAGGTC |
| | | GTTCGCTCCAAGCTGGGCTGTGTGCACGAACCCCCCGTTCAGCCCGACCGCT |
| | | GCGCCTTATCCGGTAACTATCGTCTTGAGTCCAACCCGGTAAGACACGACTT |
| | | ATCGCCACTGGCAGCAGCCACTGGTAACAGGATTAGCAGAGCGAGGTATGTA |
| | | GGCGGTGCTACAGAGTTCTTGAAGTGGTGGCCTAACTACGGCTACACTAGAA |
| | | GAACAGTATTTGGTATCTGCGCTCTGCTGAAGCCAGTTACCTTCGGAAAAAG |
| | | AGTTGGTAGCTCTTGATCCGGCAAACAAACCACCGCTGGTAGCGGTGGTTTT |
| | | TTTGTTTGCAAGCAGCAGATTACGCGCAGAAAAAAAGGATCTCAAGAAGATC |
| | | CTTTGATCTTTTCTACGGGGTCTGACGCTCAGTGGAACGAAAACTCACGTTA |
| | | AGGGATTTTGGTCATGAGATTATCAAAAAGGATCTTCACCTAGATCCTTTTA |
| | | AATTAAAAATGAAGTTTTAAATCAATCTAAAGTATATATGAGTAAACTTGGT |
| | | CTGACAGTTACCAATGCTTAATCAGTGAGGCACCTATCTCAGCGATCTGTCT |
| | | ATTTCGTTCATCCATAGTTGCCTGACTCCCCGTCGTGTAGATAACTACGATA |
| | | CGGGAGGGCTTACCATCTGGCCCCAGTGCTGCAATGATACCGCGAGAACCAC |
| | | GCTCACCGGCTCCAGATTTATCAGCAATAAACCAGCCAGCCGGAAGGGCCGA |
| | | GCGCAGAAGTGGTCCTGCAACTTTATCCGCCTCCATCCAGTCTATTAATTGT |
| | | TGCCGGGAAGCTAGAGTAAGTAGTTCGCCAGTTAATAGTTTGCGCAACGTTG |
| | | TTGCCATTGCTACAGGCATCGTGGTGTCACGCTCGTCGTTTGGTATGGCTTC |
| | | ATTCAGCTCCGGTTCCCAACGATCAAGGCGAGTTACATGATCCCCCATGTTG |
| | | TGCAAAAAAGCGGTTAGCTCCTTCGGTCCTCCGATCGTTGTCAGAAGTAAGT |
| | | TGGCCGCAGTGTTATCACTCATGGTTATGGCAGCACTGCATAATTCTCTTAC |
| | | TGTCATGCCATCCGTAAGATGCTTTTCTGTGACTGGTGAGTACTCAACCAAG |
| | | TCATTCTGAGAATAGTGTATGCGGCGACCGAGTTGCTCTTGCCCGGCGTCAA |
| | | TACGGGATAATACCGCGCCACATAGCAGAACTTTAAAAGTGCTCATCATTGG |
| | | AAAACGTTCTTCGGGGCGAAAACTCTCAAGGATCTTACCGCTGTTGAGATCC |
| | | AGTTCGATGTAACCCACTCGTGCACCCAACTGATCTTCAGCATCTTTTACTT |
| | | TCACCAGCGTTTCTGGGTGAGCAAAAACAGGAAGGCAAAATGCCGCAAAAAA |
| | | GGGAATAAGGGCGACACGGAAATGTTGAATACTCATACTCTTCCTTTTTCAA |
| | | TATTATTGAAGCATTTATCAGGGTTATTGTCTCATGAGCGGATACATATTTG |
| | | AATGTATTTAGAAAAATAAACAAATAGGGGTTCCGCGCACATTTCCCCGAAA |
| | | AGTGCCAC |
| 14 | Compound 5 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGCAAGATTAGCAGCCTGCCTACACAGCTGTTCAAGTG |
| | | CTGCTTCTGCGACTTCCTGAAAGTGAAGATGCACACCATGAGCAGCAGCCAC |
| | | CTGTTCTATCTGGCCCTGTGCCTGCTGACCTTTACCAGCTCTGCTACCGCCG |
| | | GACCTGAGACACTTTGTGGCGCTGAACTGGTGGACGCCCTGCAGTTTGTGTG |
| | | TGGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGCG |
| | | ACCTGCGGCGGCTGGAAATGTATTGTGCCCCTCTGAAGCCTGCCAAGAGCGC |
| | | CTAAATAGTGAGTCGTATTAACGTACCAACAACAACAAGATGAAGAGCACCA |
| | | AACTTGTTGGTGCTCTTCATCTTGTTGTTTATCTTAGAGGCATATCCCTTTT |
| | | ATCTTAGAGGCATATCCCTCTGGGCCTCATGGGCCTTCCGCTCACTGCCCGC |
| | | TTTCCAGTCGGGAAACCTGTCGTGCCAGCTGCATTAACATGGTCATAGCTGT |
| | | TTCCTTGCGTATTGGGCGCTCTCCGCTTCCTCGCTCACTGACTCGCTGCGCT |
| | | CGGTCGTTCGGGTAAAGCCTGGGGTGCCTAATGAGCAAAAGGCCAGCAAAAG |
| | | GCCAGGAACCGTAAAAAGGCCGCGTTGCTGGCGTTTTTCCATAGGCTCCGCC |
| | | CCCCTGACGAGCATCACAAAAATCGACGCTCAAGTCAGAGGTGGCGAAACCC |
| | | GACAGGACTATAAAGATACCAGGCGTTTCCCCCTGGAAGCTCCCTCGTGCGC |
| | | TCTCCTGTTCCGACCCTGCCGCTTACCGGATACCTGTCCGCCTTTCTCCCTT |
| | | CGGGAAGCGTGGCGCTTTCTCATAGCTCACGCTGTAGGTATCTCAGTTCGGT |
| | | GTAGGTCGTTCGCTCCAAGCTGGGCTGTGTGCACGAACCCCCCGTTCAGCCC |
| | | GACCGCTGCGCCTTATCCGGTAACTATCGTCTTGAGTCCAACCCGGTAAGAC |
| | | ACGACTTATCGCCACTGGCAGCAGCCACTGGTAACAGGATTAGCAGAGCGAG |
| | | GTATGTAGGCGGTGCTACAGAGTTCTTGAAGTGGTGGCCTAACTACGGCTAC |
| | | ACTAGAAGAACAGTATTTGGTATCTGCGCTCTGCTGAAGCCAGTTACCTTCG |
| | | GAAAAAGAGTTGGTAGCTCTTGATCCGGCAAACAAACCACCGCTGGTAGCGG |
| | | TGGTTTTTTTGTTTGCAAGCAGCAGATTACGCGCAGAAAAAAAGGATCTCAA |
| | | GAAGATCCTTTGATCTTTTCTACGGGGTCTGACGCTCAGTGGAACGAAAACT |
| | | CACGTTAAGGGATTTTGGTCATGAGATTATCAAAAAGGATCTTCACCTAGAT |
| | | CCTTTTAAATTAAAAATGAAGTTTTAAATCAATCTAAAGTATATATGAGTAA |
| | | ACTTGGTCTGACAGTTACCAATGCTTAATCAGTGAGGCACCTATCTCAGCGA |
| | | TCTGTCTATTTCGTTCATCCATAGTTGCCTGACTCCCCGTCGTGTAGATAAC |
| | | TACGATACGGGAGGGCTTACCATCTGGCCCCAGTGCTGCAATGATACCGCGA |
| | | GAACCACGCTCACCGGCTCCAGATTTATCAGCAATAAACCAGCCAGCCGGAA |
| | | GGGCCGAGCGCAGAAGTGGTCCTGCAACTTTATCCGCCTCCATCCAGTCTAT |
| | | TAATTGTTGCCGGGAAGCTAGAGTAAGTAGTTCGCCAGTTAATAGTTTGCGC |
| | | AACGTTGTTGCCATTGCTACAGGCATCGTGGTGTCACGCTCGTCGTTTGGTA |
| | | TGGCTTCATTCAGCTCCGGTTCCCAACGATCAAGGCGAGTTACATGATCCCC |
| | | CATGTTGTGCAAAAAAGCGGTTAGCTCCTTCGGTCCTCCGATCGTTGTCAGA |
| | | AGTAAGTTGGCCGCAGTGTTATCACTCATGGTTATGGCAGCACTGCATAATT |
| | | CTCTTACTGTCATGCCATCCGTAAGATGCTTTTCTGTGACTGGTGAGTACTC |
| | | AACCAAGTCATTCTGAGAATAGTGTATGCGGCGACCGAGTTGCTCTTGCCCG |
| | | GCGTCAATACGGGATAATACCGCGCCACATAGCAGAACTTTAAAAGTGCTCA |
| | | TCATTGGAAAACGTTCTTCGGGGCGAAAACTCTCAAGGATCTTACCGCTGTT |
| | | GAGATCCAGTTCGATGTAACCCACTCGTGCACCCAACTGATCTTCAGCATCT |
| | | TTTACTTTCACCAGCGTTTCTGGGTGAGCAAAAACAGGAAGGCAAAATGCCG |
| | | CAAAAAAGGGAATAAGGGCGACACGGAAATGTTGAATACTCATACTCTTCCT |
| | | TTTTCAATATTATTGAAGCATTTATCAGGGTTATTGTCTCATGAGCGGATAC |
| | | ATATTTGAATGTATTTAGAAAAATAAACAAATAGGGGTTCCGCGCACATTTC |
| | | CCCGAAAAGTGCCAC |
| 15 | Compound 6 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGCAAGATTAGCAGCCTGCCTACACAGCTGTTCAAGTG |
| | | CTGCTTCTGCGACTTCCTGAAAGTGAAGATGCACACCATGAGCAGCAGCCAC |
| | | CTGTTCTATCTGGCCCTGTGCCTGCTGACCTTTACCAGCTCTGCTACCGCCG |
| | | GACCTGAGACACTTTGTGGCGCTGAACTGGTGGACGCCCTGCAGTTTGTGTG |
| | | TGGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGCG |
| | | ACCTGCGGCGGCTGGAAATGTATTGTGCCCCTCTGAAGCCTGCCAAGAGCGC |
| | | CTAAACAACAACAACAAGATGAAGAGCACCAAACTTGTTGGTGCTCTTCATC |
| | | TTGTTGACAACAATTTATCTTAGAGGCATATCCCTCTGGGCCTCATGGGCCT |
| | | TCCGCTCACTGCCCGCTTTCCAGTCGGGAAACCTGTCGTGCCAGCTGCATTA |
| | | ACATGGTCATAGCTGTTTCCTTGCGTATTGGGCGCTCTCCGCTTCCTCGCTC |
| | | ACTGACTCGCTGCGCTCGGTCGTTCGGGTAAAGCCTGGGGTGCCTAATGAGC |
| | | AAAAGGCCAGCAAAAGGCCAGGAACCGTAAAAAGGCCGCGTTGCTGGCGTTT |
| | | TTCCATAGGCTCCGCCCCCCTGACGAGCATCACAAAAATCGACGCTCAAGTC |
| | | AGAGGTGGCGAAACCCGACAGGACTATAAAGATACCAGGCGTTTCCCCCTGG |
| | | AAGCTCCCTCGTGCGCTCTCCTGTTCCGACCCTGCCGCTTACCGGATACCTG |
| | | TCCGCCTTTCTCCCTTCGGGAAGCGTGGCGCTTTCTCATAGCTCACGCTGTA |
| | | GGTATCTCAGTTCGGTGTAGGTCGTTCGCTCCAAGCTGGGCTGTGTGCACGA |
| | | ACCCCCCGTTCAGCCCGACCGCTGCGCCTTATCCGGTAACTATCGTCTTGAG |
| | | TCCAACCCGGTAAGACACGACTTATCGCCACTGGCAGCAGCCACTGGTAACA |
| | | GGATTAGCAGAGCGAGGTATGTAGGCGGTGCTACAGAGTTCTTGAAGTGGTG |
| | | GCCTAACTACGGCTACACTAGAAGAACAGTATTTGGTATCTGCGCTCTGCTG |
| | | AAGCCAGTTACCTTCGGAAAAAGAGTTGGTAGCTCTTGATCCGGCAAACAAA |
| | | CCACCGCTGGTAGCGGTGGTTTTTTTGTTTGCAAGCAGCAGATTACGCGCAG |
| | | AAAAAAAGGATCTCAAGAAGATCCTTTGATCTTTTCTACGGGGTCTGACGCT |
| | | CAGTGGAACGAAAACTCACGTTAAGGGATTTTGGTCATGAGATTATCAAAAA |
| | | GGATCTTCACCTAGATCCTTTTAAATTAAAAATGAAGTTTTAAATCAATCTA |
| | | AAGTATATATGAGTAAACTTGGTCTGACAGTTACCAATGCTTAATCAGTGAG |
| | | GCACCTATCTCAGCGATCTGTCTATTTCGTTCATCCATAGTTGCCTGACTCC |
| | | CCGTCGTGTAGATAACTACGATACGGGAGGGCTTACCATCTGGCCCCAGTGC |
| | | TGCAATGATACCGCGAGAACCACGCTCACCGGCTCCAGATTTATCAGCAATA |
| | | AACCAGCCAGCCGGAAGGGCCGAGCGCAGAAGTGGTCCTGCAACTTTATCCG |
| | | CCTCCATCCAGTCTATTAATTGTTGCCGGGAAGCTAGAGTAAGTAGTTCGCC |
| | | AGTTAATAGTTTGCGCAACGTTGTTGCCATTGCTACAGGCATCGTGGTGTCA |
| | | CGCTCGTCGTTTGGTATGGCTTCATTCAGCTCCGGTTCCCAACGATCAAGGC |
| | | GAGTTACATGATCCCCCATGTTGTGCAAAAAAGCGGTTAGCTCCTTCGGTCC |
| | | TCCGATCGTTGTCAGAAGTAAGTTGGCCGCAGTGTTATCACTCATGGTTATG |
| | | GCAGCACTGCATAATTCTCTTACTGTCATGCCATCCGTAAGATGCTTTTCTG |
| | | TGACTGGTGAGTACTCAACCAAGTCATTCTGAGAATAGTGTATGCGGCGACC |
| | | GAGTTGCTCTTGCCCGGCGTCAATACGGGATAATACCGCGCCACATAGCAGA |
| | | ACTTTAAAAGTGCTCATCATTGGAAAACGTTCTTCGGGGCGAAAACTCTCAA |
| | | GGATCTTACCGCTGTTGAGATCCAGTTCGATGTAACCCACTCGTGCACCCAA |
| | | CTGATCTTCAGCATCTTTTACTTTCACCAGCGTTTCTGGGTGAGCAAAAACA |
| | | GGAAGGCAAAATGCCGCAAAAAAGGGAATAAGGGCGACACGGAAATGTTGAA |
| | | TACTCATACTCTTCCTTTTTCAATATTATTGAAGCATTTATCAGGGTTATTG |
| | | TCTCATGAGCGGATACATATTTGAATGTATTTAGAAAAATAAACAAATAGGG |
| | | GTTCCGCGCACATTTCCCCGAAAAGTGCCAC |
| 16 | Compound 7 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGCAAGATTAGCAGCCTGCCTACACAGCTGTTCAAGTG |
| | | CTGCTTCTGCGACTTCCTGAAAGTGAAGATGCACACCATGAGCAGCAGCCAC |
| | | CTGTTCTATCTGGCCCTGTGCCTGCTGACCTTTACCAGCTCTGCTACCGCCG |
| | | GACCTGAGACACTTTGTGGCGCTGAACTGGTGGACGCCCTGCAGTTTGTGTG |
| | | TGGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGCG |
| | | ACCTGCGGCGGCTGGAAATGTATTGTGCCCCTCTGAAGCCTGCCAAGAGCGC |
| | | CTAAATAGTGAGTCGTATTAACGTACCAACAACAACAAGATGAAGAGCACCA |
| | | AACTTGTTGGTGCTCTTCATCTTGTTGTTTATCTTAGAGGCATATCCCTACG |
| | | TACCAACAACAACAAGATGAAGAGCACCAAACTTGTTGGTGCTCTTCATCTT |
| | | GTTGTTTATCTTAGAGGCATATCCCTTTTATCTTAGAGGCATATCCCTCTGG |
| | | GCCTCATGGGCCTTCCGCTCACTGCCCGCTTTCCAGTCGGGAAACCTGTCGT |
| | | GCCAGCTGCATTAACATGGTCATAGCTGTTTCCTTGCGTATTGGGCGCTCTC |
| | | CGCTTCCTCGCTCACTGACTCGCTGCGCTCGGTCGTTCGGGTAAAGCCTGGG |
| | | GTGCCTAATGAGCAAAAGGCCAGCAAAAGGCCAGGAACCGTAAAAAGGCCGC |
| | | GTTGCTGGCGTTTTTCCATAGGCTCCGCCCCCCTGACGAGCATCACAAAAAT |
| | | CGACGCTCAAGTCAGAGGTGGCGAAACCCGACAGGACTATAAAGATACCAGG |
| | | CGTTTCCCCCTGGAAGCTCCCTCGTGCGCTCTCCTGTTCCGACCCTGCCGCT |
| | | TACCGGATACCTGTCCGCCTTTCTCCCTTCGGGAAGCGTGGCGCTTTCTCAT |
| | | AGCTCACGCTGTAGGTATCTCAGTTCGGTGTAGGTCGTTCGCTCCAAGCTGG |
| | | GCTGTGTGCACGAACCCCCCGTTCAGCCCGACCGCTGCGCCTTATCCGGTAA |
| | | CTATCGTCTTGAGTCCAACCCGGTAAGACACGACTTATCGCCACTGGCAGCA |
| | | GCCACTGGTAACAGGATTAGCAGAGCGAGGTATGTAGGCGGTGCTACAGAGT |
| | | TCTTGAAGTGGTGGCCTAACTACGGCTACACTAGAAGAACAGTATTTGGTAT |
| | | CTGCGCTCTGCTGAAGCCAGTTACCTTCGGAAAAAGAGTTGGTAGCTCTTGA |
| | | TCCGGCAAACAAACCACCGCTGGTAGCGGTGGTTTTTTTGTTTGCAAGCAGC |
| | | AGATTACGCGCAGAAAAAAAGGATCTCAAGAAGATCCTTTGATCTTTTCTAC |
| | | GGGGTCTGACGCTCAGTGGAACGAAAACTCACGTTAAGGGATTTTGGTCATG |
| | | AGATTATCAAAAAGGATCTTCACCTAGATCCTTTTAAATTAAAAATGAAGTT |
| | | TTAAATCAATCTAAAGTATATATGAGTAAACTTGGTCTGACAGTTACCAATG |
| | | CTTAATCAGTGAGGCACCTATCTCAGCGATCTGTCTATTTCGTTCATCCATA |
| | | GTTGCCTGACTCCCCGTCGTGTAGATAACTACGATACGGGAGGGCTTACCAT |
| | | CTGGCCCCAGTGCTGCAATGATACCGCGAGAACCACGCTCACCGGCTCCAGA |
| | | TTTATCAGCAATAAACCAGCCAGCCGGAAGGGCCGAGCGCAGAAGTGGTCCT |
| | | GCAACTTTATCCGCCTCCATCCAGTCTATTAATTGTTGCCGGGAAGCTAGAG |
| | | TAAGTAGTTCGCCAGTTAATAGTTTGCGCAACGTTGTTGCCATTGCTACAGG |
| | | CATCGTGGTGTCACGCTCGTCGTTTGGTATGGCTTCATTCAGCTCCGGTTCC |
| | | CAACGATCAAGGCGAGTTACATGATCCCCCATGTTGTGCAAAAAAGCGGTTA |
| | | GCTCCTTCGGTCCTCCGATCGTTGTCAGAAGTAAGTTGGCCGCAGTGTTATC |
| | | ACTCATGGTTATGGCAGCACTGCATAATTCTCTTACTGTCATGCCATCCGTA |
| | | AGATGCTTTTCTGTGACTGGTGAGTACTCAACCAAGTCATTCTGAGAATAGT |
| | | GTATGCGGCGACCGAGTTGCTCTTGCCCGGCGTCAATACGGGATAATACCGC |
| | | GCCACATAGCAGAACTTTAAAAGTGCTCATCATTGGAAAACGTTCTTCGGGG |
| | | CGAAAACTCTCAAGGATCTTACCGCTGTTGAGATCCAGTTCGATGTAACCCA |
| | | CTCGTGCACCCAACTGATCTTCAGCATCTTTTACTTTCACCAGCGTTTCTGG |
| | | GTGAGCAAAAACAGGAAGGCAAAATGCCGCAAAAAAGGGAATAAGGGCGACA |
| | | CGGAAATGTTGAATACTCATACTCTTCCTTTTTCAATATTATTGAAGCATTT |
| | | ATCAGGGTTATTGTCTCATGAGCGGATACATATTTGAATGTATTTAGAAAAA |
| | | TAAACAAATAGGGGTTCCGCGCACATTTCCCCGAAAAGTGCCAC |
| 17 | Compound 8 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGCAAGATTAGCAGCCTGCCTACACAGCTGTTCAAGTG |
| | | CTGCTTCTGCGACTTCCTGAAAGTGAAGATGCACACCATGAGCAGCAGCCAC |
| | | CTGTTCTATCTGGCCCTGTGCCTGCTGACCTTTACCAGCTCTGCTACCGCCG |
| | | GACCTGAGACACTTTGTGGCGCTGAACTGGTGGACGCCCTGCAGTTTGTGTG |
| | | TGGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGCG |
| | | ACCTGCGGCGGCTGGAAATGTATTGTGCCCCTCTGAAGCCTGCCAAGAGCGC |
| | | CTAAATAGTGAGTCGTATTAACGTACCAACAACAACAAGATGAAGAGCACCA |
| | | AACTTGTTGGTGCTCTTCATCTTGTTGTTTATCTTAGAGGCATATCCCTACG |
| | | TACCAACAAGGACAGCCACATGCACTTCAAACTTGTTGAAGTGCATGTGGCT |
| | | GTCCTTTATCTTAGAGGCATATCCCTTTTATCTTAGAGGCATATCCCTCTGG |
| | | GCCTCATGGGCCTTCCGCTCACTGCCCGCTTTCCAGTCGGGAAACCTGTCGT |
| | | GCCAGCTGCATTAACATGGTCATAGCTGTTTCCTTGCGTATTGGGCGCTCTC |
| | | CGCTTCCTCGCTCACTGACTCGCTGCGCTCGGTCGTTCGGGTAAAGCCTGGG |
| | | GTGCCTAATGAGCAAAAGGCCAGCAAAAGGCCAGGAACCGTAAAAAGGCCGC |
| | | GTTGCTGGCGTTTTTCCATAGGCTCCGCCCCCCTGACGAGCATCACAAAAAT |
| | | CGACGCTCAAGTCAGAGGTGGCGAAACCCGACAGGACTATAAAGATACCAGG |
| | | CGTTTCCCCCTGGAAGCTCCCTCGTGCGCTCTCCTGTTCCGACCCTGCCGCT |
| | | TACCGGATACCTGTCCGCCTTTCTCCCTTCGGGAAGCGTGGCGCTTTCTCAT |
| | | AGCTCACGCTGTAGGTATCTCAGTTCGGTGTAGGTCGTTCGCTCCAAGCTGG |
| | | GCTGTGTGCACGAACCCCCCGTTCAGCCCGACCGCTGCGCCTTATCCGGTAA |
| | | CTATCGTCTTGAGTCCAACCCGGTAAGACACGACTTATCGCCACTGGCAGCA |
| | | GCCACTGGTAACAGGATTAGCAGAGCGAGGTATGTAGGCGGTGCTACAGAGT |
| | | TCTTGAAGTGGTGGCCTAACTACGGCTACACTAGAAGAACAGTATTTGGTAT |
| | | CTGCGCTCTGCTGAAGCCAGTTACCTTCGGAAAAAGAGTTGGTAGCTCTTGA |
| | | TCCGGCAAACAAACCACCGCTGGTAGCGGTGGTTTTTTTGTTTGCAAGCAGC |
| | | AGATTACGCGCAGAAAAAAAGGATCTCAAGAAGATCCTTTGATCTTTTCTAC |
| | | GGGGTCTGACGCTCAGTGGAACGAAAACTCACGTTAAGGGATTTTGGTCATG |
| | | AGATTATCAAAAAGGATCTTCACCTAGATCCTTTTAAATTAAAAATGAAGTT |
| | | TTAAATCAATCTAAAGTATATATGAGTAAACTTGGTCTGACAGTTACCAATG |
| | | CTTAATCAGTGAGGCACCTATCTCAGCGATCTGTCTATTTCGTTCATCCATA |
| | | GTTGCCTGACTCCCCGTCGTGTAGATAACTACGATACGGGAGGGCTTACCAT |
| | | CTGGCCCCAGTGCTGCAATGATACCGCGAGAACCACGCTCACCGGCTCCAGA |
| | | TTTATCAGCAATAAACCAGCCAGCCGGAAGGGCCGAGCGCAGAAGTGGTCCT |
| | | GCAACTTTATCCGCCTCCATCCAGTCTATTAATTGTTGCCGGGAAGCTAGAG |
| | | TAAGTAGTTCGCCAGTTAATAGTTTGCGCAACGTTGTTGCCATTGCTACAGG |
| | | CATCGTGGTGTCACGCTCGTCGTTTGGTATGGCTTCATTCAGCTCCGGTTCC |
| | | CAACGATCAAGGCGAGTTACATGATCCCCCATGTTGTGCAAAAAAGCGGTTA |
| | | GCTCCTTCGGTCCTCCGATCGTTGTCAGAAGTAAGTTGGCCGCAGTGTTATC |
| | | ACTCATGGTTATGGCAGCACTGCATAATTCTCTTACTGTCATGCCATCCGTA |
| | | AGATGCTTTTCTGTGACTGGTGAGTACTCAACCAAGTCATTCTGAGAATAGT |
| | | GTATGCGGCGACCGAGTTGCTCTTGCCCGGCGTCAATACGGGATAATACCGC |
| | | GCCACATAGCAGAACTTTAAAAGTGCTCATCATTGGAAAACGTTCTTCGGGG |
| | | CGAAAACTCTCAAGGATCTTACCGCTGTTGAGATCCAGTTCGATGTAACCCA |
| | | CTCGTGCACCCAACTGATCTTCAGCATCTTTTACTTTCACCAGCGTTTCTGG |
| | | GTGAGCAAAAACAGGAAGGCAAAATGCCGCAAAAAAGGGAATAAGGGCGACA |
| | | CGGAAATGTTGAATACTCATACTCTTCCTTTTTCAATATTATTGAAGCATTT |
| | | ATCAGGGTTATTGTCTCATGAGCGGATACATATTTGAATGTATTTAGAAAAA |
| | | TAAACAAATAGGGGTTCCGCGCACATTTCCCCGAAAAGTGCCAC |
| 18 | Compound 9 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGCAAGATTAGCAGCCTGCCTACACAGCTGTTCAAGTG |
| | | CTGCTTCTGCGACTTCCTGAAAGTGAAGATGCACACCATGAGCAGCAGCCAC |
| | | CTGTTCTATCTGGCCCTGTGCCTGCTGACCTTTACCAGCTCTGCTACCGCCG |
| | | GACCTGAGACACTTTGTGGCGCTGAACTGGTGGACGCCCTGCAGTTTGTGTG |
| | | TGGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGCG |
| | | ACCTGCGGCGGCTGGAAATGTATTGTGCCCCTCTGAAGCCTGCCAAGAGCGC |
| | | CTAAACAACAACAACAAGATGAAGAGCACCAAACTTGTTGGTGCTCTTCATC |
| | | TTGTTGACAACAACAACAAGATGAAGAGCACCAAACTTGTTGGTGCTCTTCA |
| | | TCTTGTTGACAACAATTTATCTTAGAGGCATATCCCTCTGGGCCTCATGGGC |
| | | CTTCCGCTCACTGCCCGCTTTCCAGTCGGGAAACCTGTCGTGCCAGCTGCAT |
| | | TAACATGGTCATAGCTGTTTCCTTGCGTATTGGGCGCTCTCCGCTTCCTCGC |
| | | TCACTGACTCGCTGCGCTCGGTCGTTCGGGTAAAGCCTGGGGTGCCTAATGA |
| | | GCAAAAGGCCAGCAAAAGGCCAGGAACCGTAAAAAGGCCGCGTTGCTGGCGT |
| | | TTTTCCATAGGCTCCGCCCCCCTGACGAGCATCACAAAAATCGACGCTCAAG |
| | | TCAGAGGTGGCGAAACCCGACAGGACTATAAAGATACCAGGCGTTTCCCCCT |
| | | GGAAGCTCCCTCGTGCGCTCTCCTGTTCCGACCCTGCCGCTTACCGGATACC |
| | | TGTCCGCCTTTCTCCCTTCGGGAAGCGTGGCGCTTTCTCATAGCTCACGCTG |
| | | TAGGTATCTCAGTTCGGTGTAGGTCGTTCGCTCCAAGCTGGGCTGTGTGCAC |
| | | GAACCCCCCGTTCAGCCCGACCGCTGCGCCTTATCCGGTAACTATCGTCTTG |
| | | AGTCCAACCCGGTAAGACACGACTTATCGCCACTGGCAGCAGCCACTGGTAA |
| | | CAGGATTAGCAGAGCGAGGTATGTAGGCGGTGCTACAGAGTTCTTGAAGTGG |
| | | TGGCCTAACTACGGCTACACTAGAAGAACAGTATTTGGTATCTGCGCTCTGC |
| | | TGAAGCCAGTTACCTTCGGAAAAAGAGTTGGTAGCTCTTGATCCGGCAAACA |
| | | AACCACCGCTGGTAGCGGTGGTTTTTTTGTTTGCAAGCAGCAGATTACGCGC |
| | | AGAAAAAAAGGATCTCAAGAAGATCCTTTGATCTTTTCTACGGGGTCTGACG |
| | | CTCAGTGGAACGAAAACTCACGTTAAGGGATTTTGGTCATGAGATTATCAAA |
| | | AAGGATCTTCACCTAGATCCTTTTAAATTAAAAATGAAGTTTTAAATCAATC |
| | | TAAAGTATATATGAGTAAACTTGGTCTGACAGTTACCAATGCTTAATCAGTG |
| | | AGGCACCTATCTCAGCGATCTGTCTATTTCGTTCATCCATAGTTGCCTGACT |
| | | CCCCGTCGTGTAGATAACTACGATACGGGAGGGCTTACCATCTGGCCCCAGT |
| | | GCTGCAATGATACCGCGAGAACCACGCTCACCGGCTCCAGATTTATCAGCAA |
| | | TAAACCAGCCAGCCGGAAGGGCCGAGCGCAGAAGTGGTCCTGCAACTTTATC |
| | | CGCCTCCATCCAGTCTATTAATTGTTGCCGGGAAGCTAGAGTAAGTAGTTCG |
| | | CCAGTTAATAGTTTGCGCAACGTTGTTGCCATTGCTACAGGCATCGTGGTGT |
| | | CACGCTCGTCGTTTGGTATGGCTTCATTCAGCTCCGGTTCCCAACGATCAAG |
| | | GCGAGTTACATGATCCCCCATGTTGTGCAAAAAAGCGGTTAGCTCCTTCGGT |
| | | CCTCCGATCGTTGTCAGAAGTAAGTTGGCCGCAGTGTTATCACTCATGGTTA |
| | | TGGCAGCACTGCATAATTCTCTTACTGTCATGCCATCCGTAAGATGCTTTTC |
| | | TGTGACTGGTGAGTACTCAACCAAGTCATTCTGAGAATAGTGTATGCGGCGA |
| | | CCGAGTTGCTCTTGCCCGGCGTCAATACGGGATAATACCGCGCCACATAGCA |
| | | GAACTTTAAAAGTGCTCATCATTGGAAAACGTTCTTCGGGGCGAAAACTCTC |
| | | AAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAA |
| | | CAGGAAGGCAAAATGCCGCAAAAAAGGGAATAAGGGCGACACGGAAATGTTG |
| | | AATACTCATACTCTTCCTTTTTCAATATTATTGAAGCATTTATCAGGGTTAT |
| | | TGTCTCATGAGCGGATACATATTTGAATGTATTTAGAAAAATAAACAAATAG |
| | | GGGTTCCGCGCACATTTCCCCGAAAAGTGCCAC |
| 93 | Compound 10 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGAAGGAAGGCCGTCAAGGC |
| | | CGCATGCCACCATGTTGTTGCTGCTGCTCGCCTGTATTGCCCTGGCCTCTAC |
| | | AGCCGCCGCTACAAATTCTGCCCCTACCAGCAGCTCCACCAAGAAAACCCAG |
| | | CTGCAACTGGAACATCTGCTGCTGGACCTGCAGATGATCCTGAACGGCATCA |
| | | ACAACTACAAGAACCCCAAGCTGACCCGGATGCTGACCTTCAAGTTCTACAT |
| | | GCCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTGA |
| | | GGCCTAGGGACCTGATCAGCAACATCAACGTGATCGTGCTGGAACTGAAAGG |
| | | CAGCGAGACAACCTTCATGTGCGAGTACGCCGACGAGACAGCTACCATCGTG |
| | | GAATTTCTGAACCGGTGGATCACCTTCTGCCAGAGCATCATCAGCACCCTGA |
| | | CCTGAATAGTGAGTCGTATTAACGTACCAACAAGGAGGGCAGAATCATCACG |
| | | AAGTGGTGAAGTACTTGACTTCACCACTTCGTGATGATTCTGCCCTCCTTTA |
| | | TCTTAGAGGCATATCCCTACGTACCAACAAGAGATGAGCTTCCTACAGCACA |
| | | ACAAATGTGACTTGCACATTTGTTGTGCTGTAGGAAGCTCATCTCTTTATCT |
| | | TAGAGGCATATCCCTACGTACCAACAAGTACAAGATCCGCAGACGTGTAAAT |
| | | GTTCCACTTGGGAACATTTACACGTCTGCGGATCTTGTACTTTATCTTAGAG |
| | | GCATATCCCTTTTATCTTAGAGGCATATCCCTCTGGGCCTCATGGGCCTTCC |
| | | TTTCACTGCCCGCTTTCCAGTCGGGAAACCTGTCGTGCCAGCTGCATTAACA |
| | | TGGTCATAGCTGTTTCCTTGCGTATTGGGCGCTCTCCGCTTCCTCGCTCACT |
| | | GACTCGCTGCGCTCGGTCGTTCGGGTAAAGCCTGGGGTGCCTAATGAGCAAA |
| | | AGGCCAGCAAAAGGCCAGGAACCGTAAAAAGGCCGCGTTGCTGGCGTTTTTC |
| | | CATAGGCTCCGCCCCCCTGACGAGCATCACAAAAATCGACGCTCAAGTCAGA |
| | | GGTGGCGAAACCCGACAGGACTATAAAGATACCAGGCGTTTCCCCCTGGAAG |
| | | CTCCCTCGTGCGCTCTCCTGTTCCGACCCTGCCGCTTACCGGATACCTGTCC |
| | | GCCTTTCTCCCTTCGGGAAGCGTGGCGCTTTCTCATAGCTCACGCTGTAGGT |
| | | ATCTCAGTTCGGTGTAGGTCGTTCGCTCCAAGCTGGGCTGTGTGCACGAACC |
| | | CCCCGTTCAGCCCGACCGCTGCGCCTTATCCGGTAACTATCGTCTTGAGTCC |
| | | AACCCGGTAAGACACGACTTATCGCCACTGGCAGCAGCCACTGGTAACAGGA |
| | | TTAGCAGAGCGAGGTATGTAGGCGGTGCTACAGAGTTCTTGAAGTGGTGGCC |
| | | TAACTACGGCTACACTAGAAGAACAGTATTTGGTATCTGCGCTCTGCTGAAG |
| | | CCAGTTACCTTCGGAAAAAGAGTTGGTAGCTCTTGATCCGGCAAACAAACCA |
| | | CCGCTGGTAGCGGTGGTTTTTTTGTTTGCAAGCAGCAGATTACGCGCAGAAA |
| | | AAAAGGATCTCAAGAAGATCCTTTGATCTTTTCTACGGGGTCTGACGCTCAG |
| | | TGGAACGAAAACTCACGTTAAGGGATTTTGGTCATGAGATTATCAAAAAGGA |
| | | TCTTCACCTAGATCCTTTTAAATTAAAAATGAAGTTTTAAATCAATCTAAAG |
| | | TATATATGAGTAAACTTGGTCTGACAGTTATTAGAAAAATTCATCCAGCAGA |
| | | CGATAAAACGCAATACGCTGGCTATCCGGTGCCGCAATGCCATACAGCACCA |
| | | GAAAACGATCCGCCCATTCGCCGCCCAGTTCTTCCGCAATATCACGGGTGGC |
| | | CAGCGCAATATCCTGATAACGATCCGCCACGCCCAGACGGCCGCAATCAATA |
| | | AAGCCGCTAAAACGGCCATTTTCCACCATAATGTTCGGCAGGCACGCATCAC |
| | | CATGGGTCACCACCAGATCTTCGCCATCCGGCATGCTCGCTTTCAGACGCGC |
| | | AAACAGCTCTGCCGGTGCCAGGCCCTGATGTTCTTCATCCAGATCATCCTGA |
| | | TCCACCAGGCCCGCTTCCATACGGGTACGCGCACGTTCAATACGATGTTTCG |
| | | CCTGATGATCAAACGGACAGGTCGCCGGGTCCAGGGTATGCAGACGACGCAT |
| | | GGCATCCGCCATAATGCTCACTTTTTCTGCCGGCGCCAGATGGCTAGACAGC |
| | | AGATCCTGACCCGGCACTTCGCCCAGCAGCAGCCAATCACGGCCCGCTTCGG |
| | | TCACCACATCCAGCACCGCCGCACACGGAACACCGGTGGTGGCCAGCCAGCT |
| | | CAGACGCGCCGCTTCATCCTGCAGCTCGTTCAGCGCACCGCTCAGATCGGTT |
| | | TTCACAAACAGCACCGGACGACCCTGCGCGCTCAGACGAAACACCGCCGCAT |
| | | CAGAGCAGCCAATGGTCTGCTGCGCCCAATCATAGCCAAACAGACGTTCCAC |
| | | CCACGCTGCCGGGCTACCCGCATGCAGGCCATCCTGTTCAATCATACTCTTC |
| | | CTTTTTCAATATTATTGAAGCATTTATCAGGGTTATTGTCTCATGAGCGGAT |
| | | ACATATTTGAATGTATTTAGAAAAATAAACAAATAGGGGTTCCGCGCACATT |
| | | TCCCCGAAAAGTGCCAC |
| 94 | Compound 11 | TCGCGCGTTTCGGTGATGACGGTGAAAACCTCTGACACATGCAGCTCCCGGA |
| | (pUC-GW- | GACTGTCACAGCTTGTCTGTAAGCGGATGCCGGGAGCAGACAAGCCCGTCAG |
| | Kan) | GGCGCGTCAGCGGGTGTTGGCGGGTGTCGGGGCTGGCTTAACTATGCGGCAT |
| | | CAGAGCAGATTGTACTGAGAGTGCACCATATGCGGTGTGAAATACCGCACAG |
| | | ATGCGTAAGGAGAAAATACCGCATCAGGCGCCATTCGCCATTCAGGCTGCGC |
| | | AACTGTTGGGAAGGGCGATCGGTGCGGGCCTCTTCGCTATTACGCCAGCTGG |
| | | CGAAAGGGGGATGTGCTGCAAGGCGATTAAGTTGGGTAACGCCAGGGTTTTC |
| | | CCAGTCACGACGTTGTAAAACGACGGCCAGTGAATTGACGCGTATTGGGATT |
| | | AATACGACTCACTATAGGGCGAATTGGCGGAAGGCCGTCAAGGCCGCATGCC |
| | | ACCATGTTGTTGCTGCTGCTCGCCTGTATTGCCCTGGCCTCTACAGCCGCCG |
| | | CTACAAATTCTGCCCCTACCAGCAGCTCCACCAAGAAAACCCAGCTGCAACT |
| | | GGAACATCTGCTGCTGGACCTGCAGATGATCCTGAACGGCATCAACAACTAC |
| | | AAGAACCCCAAGCTGACCCGGATGCTGACCTTCAAGTTCTACATGCCCAAGA |
| | | AGGCCACCGAGCTGAAGCACCTCCAGTGCCTGGAAGAGGAACTGAAGCCCCT |
| | | GGAAGAAGTGCTGAATCTGGCCCAGAGCAAGAACTTCCACCTGAGGCCTAGG |
| | | GACCTGATCAGCAACATCAACGTGATCGTGCTGGAACTGAAAGGCAGCGAGA |
| | | CAACCTTCATGTGCGAGTACGCCGACGAGACAGCTACCATCGTGGAATTTCT |
| | | GAACCGGTGGATCACCTTCTGCCAGAGCATCATCAGCACCCTGACCTGAACA |
| | | ACAAGGAGGGCAGAATCATCACGAAGTGGTGAAGTACTTGACTTCACCACTT |
| | | CGTGATGATTCTGCCCTCCACAACAAGAGATGAGCTTCCTACAGCACAACAA |
| | | ATGTGACTTGCACATTTGTTGTGCTGTAGGAAGCTCATCTCACAACAAGTAC |
| | | AAGATCCGCAGACGTGTAAATGTTCCACTTGGGAACATTTACACGTCTGCGG |
| | | ATCTTGTACACAACAATTTATCTTAGAGGCATATCCCTCTGGGCCTCATGGG |
| | | CCTTCCGCTCACTGCCCGCTTTCCAGTCGGGAAACCTGTCGTGCCAGCTGCA |
| | | TTAACATGGTCATAGCTGATCCCAATGGCGCGCCGAGCTTGGCTCGAGCATG |
| | | GTCATAGCTGTTTCCTGTGTGAAATTGTTATCCGCTCACAATTCCACACAAC |
| | | ATACGAGCCGGAAGCATAAAGTGTAAAGCCTGGGGTGCCTAATGAGTGAGCT |
| | | AACTCACATTAATTGCGTTGCGCTCACTGCCCGCTTTCCAGTCGGGAAACCT |
| | | GTCGTGCCAGCTGCATTAATGAATCGGCCAACGCGCGGGGAGAGGCGGTTTG |
| | | CGTATTGGGCGCTGTTCCGCTTCCTCGCTCACTGACTCGCTGCGCTCGGTCG |
| | | TTCGGCTGCGGCGAGCGGTATCAGCTCACTCAAAGGCGGTAATACGGTTATC |
| | | CACAGAATCAGGGGATAACGCAGGAAAGAACATGTGAGCAAAAGGCCAGCAA |
| | | AAGGCCAGGAACCGTAAAAAGGCCGCGTTGCTGGCGTTTTTCCATAGGCTCC |
| | | GCCCCCCTGACGAGCATCACAAAAATCGACGCTCAAGTCAGAGGTGGCGAAA |
| | | CCCGACAGGACTATAAAGATACCAGGCGTTTCCCCCTGGAAGCTCCCTCGTG |
| | | CGCTCTCCTGTTCCGACCCTGCCGCTTACCGGATACCTGTCCGCCTTTCTCC |
| | | CTTCGGGAAGCGTGGCGCTTTCTCATAGCTCACGCTGTAGGTATCTCAGTTC |
| | | GGTGTAGGTCGTTCGCTCCAAGCTGGGCTGTGTGCACGAACCCCCCGTTCAG |
| | | CCCGACCGCTGCGCCTTATCCGGTAACTATCGTCTTGAGTCCAACCCGGTAA |
| | | GACACGACTTATCGCCACTGGCAGCAGCCACTGGTAACAGGATTAGCAGAGC |
| | | GAGGTATGTAGGCGGTGCTACAGAGTTCTTGAAGTGGTGGCCTAACTACGGC |
| | | TACACTAGAAGAACAGTATTTGGTATCTGCGCTCTGCTGAAGCCAGTTACCT |
| | | TCGGAAAAAGAGTTGGTAGCTCTTGATCCGGCAAACAAACCACCGCTGGTAG |
| | | CGGTGGTTTTTTTGTTTGCAAGCAGCAGATTACGCGCAGAAAAAAAGGATCT |
| | | CAAGAAGATCCTTTGATCTTTTCTACGGGGTCTGACGCTCAGTGGAACGAAA |
| | | ACTCACGTTAAGGGATTTTGGTCATGAGATTATCAAAAAGGATCTTCACCTA |
| | | GATCCTTTTAAATTAAAAATGAAGTTTTAAATCAATCTAAAGTATATATGAG |
| | | TAAACTTGGTCTGACAGTTAGAAAAACTCATCGAGCATCAAATGAAACTGCA |
| | | ATTTATTCATATCAGGATTATCAATACCATATTTTTGAAAAAGCCGTTTCTG |
| | | TAATGAAGGAGAAAACTCACCGAGGCAGTTCCATAGGATGGCAAGATCCTGG |
| | | TATCGGTCTGCGATTCCGACTCGTCCAACATCAATACAACCTATTAATTTCC |
| | | CCTCGTCAAAAATAAGGTTATCAAGTGAGAAATCACCATGAGTGACGACTGA |
| | | ATCCGGTGAGAATGGCAAAAGTTTATGCATTTCTTTCCAGACTTGTTCAACA |
| | | GGCCAGCCATTACGCTCGTCATCAAAATCACTCGCATCAACCAAACCGTTAT |
| | | TCATTCGTGATTGCGCCTGAGCGAAACGAAATACGCGATCGCTGTTAAAAGG |
| | | ACAATTACAAACAGGAATCGAATGCAACCGGCGCAGGAACACTGCCAGCGCA |
| | | TCAACAATATTTTCACCTGAATCAGGATATTCTTCTAATACCTGGAATGCTG |
| | | TTTTCCCAGGGATCGCAGTGGTGAGTAACCATGCATCATCAGGAGTACGGAT |
| | | AAAATGCTTGATGGTCGGAAGAGGCATAAATTCCGTCAGCCAGTTTAGTCTG |
| | | ACCATCTCATCTGTAACATCATTGGCAACGCTACCTTTGCCATGTTTCAGAA |
| | | ACAACTCTGGCGCATCGGGCTTCCCATACAATCGATAGATTGTCGCACCTGA |
| | | TTGCCCGACATTATCGCGAGCCCATTTATACCCATATAAATCAGCATCCATG |
| | | TTGGAATTTAATCGCGGCCTAGAGCAAGACGTTTCCCGTTGAATATGGCTCA |
| | | TACTCTTCCTTTTTCAATATTATTGAAGCATTTATCAGGGTTATTGTCTCAT |
| | | GAGCGGATACATATTTGAATGTATTTAGAAAAATAAACAAATAGGGGTTCCG |
| | | CGCACATTTCCCCGAAAAGTGCCACCTGACGTCTAAGAAACCATTATTATCA |
| | | TGACATTAACCTATAAAAATAGGCGTATCACGAGGCCCTTTTGTC |
| 95 | Compound 12 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGTTGTTGCTGCTGCTCGCCTGTATTGCCCTGGCCTCTAC |
| | | AGCCGCCGCTACAAATTCTGCCCCTACCAGCAGCTCCACCAAGAAAACCCAG |
| | | CTGCAACTGGAACATCTGCTGCTGGACCTGCAGATGATCCTGAACGGCATCA |
| | | ACAACTACAAGAACCCCAAGCTGACCCGGATGCTGACCTTCAAGTTCTACAT |
| | | GCCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTGA |
| | | GGCCTAGGGACCTGATCAGCAACATCAACGTGATCGTGCTGGAACTGAAAGG |
| | | CAGCGAGACAACCTTCATGTGCGAGTACGCCGACGAGACAGCTACCATCGTG |
| | | GAATTTCTGAACCGGTGGATCACCTTCTGCCAGAGCATCATCAGCACCCTGA |
| | | CCTGAATAGTGAGTCGTATTAGGAGGGCAGAATCATCACGAAGTGGTGAAGT |
| | | ACTTGACTTCACCACTTCGTGATGATTCTGCCCTCCATCCCTACGTACCAAC |
| | | AAGAGATGAGCTTCCTACAGCACAACAAATGTGACTTGCACATTTGTTGTGC |
| | | TGTAGGAAGCTCATCTCATCCCTACGTACCAACAAGTACAAGATCCGCAGAC |
| | | GTGTAAATGTTCCACTTGGGAACATTTACACGTCTGCGGATCTTGTACTTTA |
| | | TCTTAGAGGCATCTGGGCCTCATGGGCCTTCCGCTCACTGCCCGCTTTCCAG |
| | | TCGGGAAACCTGTCGTGCCAGCTGCATTAACATGGTCATAGCTGTTTCCTTG |
| | | CGTATTGGGCGCTCTCCGCTTCCTCGCTCACTGACTCGCTGCGCTCGGTCGT |
| | | TCGGGTAAAGCCTGGGGTGCCTAATGAGCAAAAGGCCAGCAAAAGGCCAGGA |
| | | ACCGTAAAAAGGCCGCGTTGCTGGCGTTTTTCCATAGGCTCCGCCCCCCTGA |
| | | CGAGCATCACAAAAATCGACGCTCAAGTCAGAGGTGGCGAAACCCGACAGGA |
| | | CTATAAAGATACCAGGCGTTTCCCCCTGGAAGCTCCCTCGTGCGCTCTCCTG |
| | | TTCCGACCCTGCCGCTTACCGGATACCTGTCCGCCTTTCTCCCTTCGGGAAG |
| | | CGTGGCGCTTTCTCATAGCTCACGCTGTAGGTATCTCAGTTCGGTGTAGGTC |
| | | GTTCGCTCCAAGCTGGGCTGTGTGCACGAACCCCCCGTTCAGCCCGACCGCT |
| | | GCGCCTTATCCGGTAACTATCGTCTTGAGTCCAACCCGGTAAGACACGACTT |
| | | ATCGCCACTGGCAGCAGCCACTGGTAACAGGATTAGCAGAGCGAGGTATGTA |
| | | GGCGGTGCTACAGAGTTCTTGAAGTGGTGGCCTAACTACGGCTACACTAGAA |
| | | GAACAGTATTTGGTATCTGCGCTCTGCTGAAGCCAGTTACCTTCGGAAAAAG |
| | | AGTTGGTAGCTCTTGATCCGGCAAACAAACCACCGCTGGTAGCGGTGGTTTT |
| | | TTTGTTTGCAAGCAGCAGATTACGCGCAGAAAAAAAGGATCTCAAGAAGATC |
| | | CTTTGATCTTTTCTACGGGGTCTGACGCTCAGTGGAACGAAAACTCACGTTA |
| | | AGGGATTTTGGTCATGAGATTATCAAAAAGGATCTTCACCTAGATCCTTTTA |
| | | AATTAAAAATGAAGTTTTAAATCAATCTAAAGTATATATGAGTAAACTTGGT |
| | | CTGACAGTTACCAATGCTTAATCAGTGAGGCACCTATCTCAGCGATCTGTCT |
| | | ATTTCGTTCATCCATAGTTGCCTGACTCCCCGTCGTGTAGATAACTACGATA |
| | | CGGGAGGGCTTACCATCTGGCCCCAGTGCTGCAATGATACCGCGAGAACCAC |
| | | GCTCACCGGCTCCAGATTTATCAGCAATAAACCAGCCAGCCGGAAGGGCCGA |
| | | GCGCAGAAGTGGTCCTGCAACTTTATCCGCCTCCATCCAGTCTATTAATTGT |
| | | TGCCGGGAAGCTAGAGTAAGTAGTTCGCCAGTTAATAGTTTGCGCAACGTTG |
| | | TTGCCATTGCTACAGGCATCGTGGTGTCACGCTCGTCGTTTGGTATGGCTTC |
| | | ATTCAGCTCCGGTTCCCAACGATCAAGGCGAGTTACATGATCCCCCATGTTG |
| | | TGCAAAAAAGCGGTTAGCTCCTTCGGTCCTCCGATCGTTGTCAGAAGTAAGT |
| | | TGGCCGCAGTGTTATCACTCATGGTTATGGCAGCACTGCATAATTCTCTTAC |
| | | TGTCATGCCATCCGTAAGATGCTTTTCTGTGACTGGTGAGTACTCAACCAAG |
| | | TCATTCTGAGAATAGTGTATGCGGCGACCGAGTTGCTCTTGCCCGGCGTCAA |
| | | TACGGGATAATACCGCGCCACATAGCAGAACTTTAAAAGTGCTCATCATTGG |
| | | AAAACGTTCTTCGGGGCGAAAACTCTCAAGGATCTTACCGCTGTTGAGATCC |
| | | AGTTCGATGTAACCCACTCGTGCACCCAACTGATCTTCAGCATCTTTTACTT |
| | | TCACCAGCGTTTCTGGGTGAGCAAAAACAGGAAGGCAAAATGCCGCAAAAAA |
| | | GGGAATAAGGGCGACACGGAAATGTTGAATACTCATACTCTTCCTTTTTCAA |
| | | TATTATTGAAGCATTTATCAGGGTTATTGTCTCATGAGCGGATACATATTTG |
| | | AATGTATTTAGAAAAATAAACAAATAGGGGTTCCGCGCACATTTCCCCGAAA |
| | | AGTGCCAC |
| 96 | Compound 13 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGTTGTTGCTGCTGCTCGCCTGTATTGCCCTGGCCTCTAC |
| | | AGCCGCCGCTACAAATTCTGCCCCTACCAGCAGCTCCACCAAGAAAACCCAG |
| | | CTGCAACTGGAACATCTGCTGCTGGACCTGCAGATGATCCTGAACGGCATCA |
| | | ACAACTACAAGAACCCCAAGCTGACCCGGATGCTGACCTTCAAGTTCTACAT |
| | | GCCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTGA |
| | | GGCCTAGGGACCTGATCAGCAACATCAACGTGATCGTGCTGGAACTGAAAGG |
| | | CAGCGAGACAACCTTCATGTGCGAGTACGCCGACGAGACAGCTACCATCGTG |
| | | GAATTTCTGAACCGGTGGATCACCTTCTGCCAGAGCATCATCAGCACCCTGA |
| | | CCTGAATAGTGAGTCGTATTAGGAGGGCAGAATCATCACGAAGTGGTGAAGT |
| | | ACTTGACTTCACCACTTCGTGATGATTCTGCCCTCCACGTACCAACAAGAGA |
| | | TGAGCTTCCTACAGCACAACAAATGTGACTTGCACATTTGTTGTGCTGTAGG |
| | | AAGCTCATCTCACGTACCAACAAGTACAAGATCCGCAGACGTGTAAATGTTC |
| | | CACTTGGGAACATTTACACGTCTGCGGATCTTGTACTTTATCTTAGAGGCAT |
| | | CTGGGCCTCATGGGCCTTCCGCTCACTGCCCGCTTTCCAGTCGGGAAACCTG |
| | | TCGTGCCAGCTGCATTAACATGGTCATAGCTGTTTCCTTGCGTATTGGGCGC |
| | | TCTCCGCTTCCTCGCTCACTGACTCGCTGCGCTCGGTCGTTCGGGTAAAGCC |
| | | TGGGGTGCCTAATGAGCAAAAGGCCAGCAAAAGGCCAGGAACCGTAAAAAGG |
| | | CCGCGTTGCTGGCGTTTTTCCATAGGCTCCGCCCCCCTGACGAGCATCACAA |
| | | AAATCGACGCTCAAGTCAGAGGTGGCGAAACCCGACAGGACTATAAAGATAC |
| | | CAGGCGTTTCCCCCTGGAAGCTCCCTCGTGCGCTCTCCTGTTCCGACCCTGC |
| | | CGCTTACCGGATACCTGTCCGCCTTTCTCCCTTCGGGAAGCGTGGCGCTTTC |
| | | TCATAGCTCACGCTGTAGGTATCTCAGTTCGGTGTAGGTCGTTCGCTCCAAG |
| | | CTGGGCTGTGTGCACGAACCCCCCGTTCAGCCCGACCGCTGCGCCTTATCCG |
| | | GTAACTATCGTCTTGAGTCCAACCCGGTAAGACACGACTTATCGCCACTGGC |
| | | AGCAGCCACTGGTAACAGGATTAGCAGAGCGAGGTATGTAGGCGGTGCTACA |
| | | GAGTTCTTGAAGTGGTGGCCTAACTACGGCTACACTAGAAGAACAGTATTTG |
| | | GTATCTGCGCTCTGCTGAAGCCAGTTACCTTCGGAAAAAGAGTTGGTAGCTC |
| | | TTGATCCGGCAAACAAACCACCGCTGGTAGCGGTGGTTTTTTTGTTTGCAAG |
| | | CAGCAGATTACGCGCAGAAAAAAAGGATCTCAAGAAGATCCTTTGATCTTTT |
| | | CTACGGGGTCTGACGCTCAGTGGAACGAAAACTCACGTTAAGGGATTTTGGT |
| | | CATGAGATTATCAAAAAGGATCTTCACCTAGATCCTTTTAAATTAAAAATGA |
| | | AGTTTTAAATCAATCTAAAGTATATATGAGTAAACTTGGTCTGACAGTTACC |
| | | AATGCTTAATCAGTGAGGCACCTATCTCAGCGATCTGTCTATTTCGTTCATC |
| | | CATAGTTGCCTGACTCCCCGTCGTGTAGATAACTACGATACGGGAGGGCTTA |
| | | CCATCTGGCCCCAGTGCTGCAATGATACCGCGAGAACCACGCTCACCGGCTC |
| | | CAGATTTATCAGCAATAAACCAGCCAGCCGGAAGGGCCGAGCGCAGAAGTGG |
| | | TCCTGCAACTTTATCCGCCTCCATCCAGTCTATTAATTGTTGCCGGGAAGCT |
| | | AGAGTAAGTAGTTCGCCAGTTAATAGTTTGCGCAACGTTGTTGCCATTGCTA |
| | | CAGGCATCGTGGTGTCACGCTCGTCGTTTGGTATGGCTTCATTCAGCTCCGG |
| | | TTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGT |
| | | TATCACTCATGGTTATGGCAGCACTGCATAATTCTCTTACTGTCATGCCATC |
| | | CGTAAGATGCTTTTCTGTGACTGGTGAGTACTCAACCAAGTCATTCTGAGAA |
| | | TAGTGTATGCGGCGACCGAGTTGCTCTTGCCCGGCGTCAATACGGGATAATA |
| | | CCGCGCCACATAGCAGAACTTTAAAAGTGCTCATCATTGGAAAACGTTCTTC |
| | | GGGGCGAAAACTCTCAAGGATCTTACCGCTGTTGAGATCCAGTTCGATGTAA |
| | | CCCACTCGTGCACCCAACTGATCTTCAGCATCTTTTACTTTCACCAGCGTTT |
| | | CTGGGTGAGCAAAAACAGGAAGGCAAAATGCCGCAAAAAAGGGAATAAGGGC |
| | | GACACGGAAATGTTGAATACTCATACTCTTCCTTTTTCAATATTATTGAAGC |
| | | ATTTATCAGGGTTATTGTCTCATGAGCGGATACATATTTGAATGTATTTAGA |
| | | AAAATAAACAAATAGGGGTTCCGCGCACATTTCCCCGAAAAGTGCCAC |
| 97 | Compound 14 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGTTGTTGCTGCTGCTCGCCTGTATTGCCCTGGCCTCTAC |
| | | AGCCGCCGCTACAAATTCTGCCCCTACCAGCAGCTCCACCAAGAAAACCCAG |
| | | CTGCAACTGGAACATCTGCTGCTGGACCTGCAGATGATCCTGAACGGCATCA |
| | | ACAACTACAAGAACCCCAAGCTGACCCGGATGCTGACCTTCAAGTTCTACAT |
| | | GCCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTGA |
| | | GGCCTAGGGACCTGATCAGCAACATCAACGTGATCGTGCTGGAACTGAAAGG |
| | | CAGCGAGACAACCTTCATGTGCGAGTACGCCGACGAGACAGCTACCATCGTG |
| | | GAATTTCTGAACCGGTGGATCACCTTCTGCCAGAGCATCATCAGCACCCTGA |
| | | CCTGAATAGTGAGTCGTATTATCCCGGAGGGCAGAATCATCACGAAGTGGTG |
| | | AAGTACTTGACTTCACCACTTCGTGATGATTCTGCCCTCCTCCCGAGATGAG |
| | | CTTCCTACAGCACAACAAATGTGACTTGCACATTTGTTGTGCTGTAGGAAGC |
| | | TCATCTCTCCCGTACAAGATCCGCAGACGTGTAAATGTTCCACTTGGGAACA |
| | | TTTACACGTCTGCGGATCTTGTACTTTATCTTAGAGGCATCTGGGCCTCATG |
| | | GGCCTTCCGCTCACTGCCCGCTTTCCAGTCGGGAAACCTGTCGTGCCAGCTG |
| | | CATTAACATGGTCATAGCTGTTTCCTTGCGTATTGGGCGCTCTCCGCTTCCT |
| | | CGCTCACTGACTCGCTGCGCTCGGTCGTTCGGGTAAAGCCTGGGGTGCCTAA |
| | | TGAGCAAAAGGCCAGCAAAAGGCCAGGAACCGTAAAAAGGCCGCGTTGCTGG |
| | | CGTTTTTCCATAGGCTCCGCCCCCCTGACGAGCATCACAAAAATCGACGCTC |
| | | AAGTCAGAGGTGGCGAAACCCGACAGGACTATAAAGATACCAGGCGTTTCCC |
| | | CCTGGAAGCTCCCTCGTGCGCTCTCCTGTTCCGACCCTGCCGCTTACCGGAT |
| | | ACCTGTCCGCCTTTCTCCCTTCGGGAAGCGTGGCGCTTTCTCATAGCTCACG |
| | | CTGTAGGTATCTCAGTTCGGTGTAGGTCGTTCGCTCCAAGCTGGGCTGTGTG |
| | | CACGAACCCCCCGTTCAGCCCGACCGCTGCGCCTTATCCGGTAACTATCGTC |
| | | TTGAGTCCAACCCGGTAAGACACGACTTATCGCCACTGGCAGCAGCCACTGG |
| | | TAACAGGATTAGCAGAGCGAGGTATGTAGGCGGTGCTACAGAGTTCTTGAAG |
| | | TGGTGGCCTAACTACGGCTACACTAGAAGAACAGTATTTGGTATCTGCGCTC |
| | | TGCTGAAGCCAGTTACCTTCGGAAAAAGAGTTGGTAGCTCTTGATCCGGCAA |
| | | ACAAACCACCGCTGGTAGCGGTGGTTTTTTTGTTTGCAAGCAGCAGATTACG |
| | | CGCAGAAAAAAAGGATCTCAAGAAGATCCTTTGATCTTTTCTACGGGGTCTG |
| | | ACGCTCAGTGGAACGAAAACTCACGTTAAGGGATTTTGGTCATGAGATTATC |
| | | AAAAAGGATCTTCACCTAGATCCTTTTAAATTAAAAATGAAGTTTTAAATCA |
| | | ATCTAAAGTATATATGAGTAAACTTGGTCTGACAGTTACCAATGCTTAATCA |
| | | GTGAGGCACCTATCTCAGCGATCTGTCTATTTCGTTCATCCATAGTTGCCTG |
| | | ACTCCCCGTCGTGTAGATAACTACGATACGGGAGGGCTTACCATCTGGCCCC |
| | | AGTGCTGCAATGATACCGCGAGAACCACGCTCACCGGCTCCAGATTTATCAG |
| | | CAATAAACCAGCCAGCCGGAAGGGCCGAGCGCAGAAGTGGTCCTGCAACTTT |
| | | ATCCGCCTCCATCCAGTCTATTAATTGTTGCCGGGAAGCTAGAGTAAGTAGT |
| | | TCGCCAGTTAATAGTTTGCGCAACGTTGTTGCCATTGCTACAGGCATCGTGG |
| | | TGTCACGCTCGTCGTTTGGTATGGCTTCATTCAGCTCCGGTTCCCAACGATC |
| | | AAGGCGAGTTACATGATCCCCCATGTTGTGCAAAAAAGCGGTTAGCTCCTTC |
| | | GGTCCTCCGATCGTTGTCAGAAGTAAGTTGGCCGCAGTGTTATCACTCATGG |
| | | TTATGGCAGCACTGCATAATTCTCTTACTGTCATGCCATCCGTAAGATGCTT |
| | | TTCTGTGACTGGTGAGTACTCAACCAAGTCATTCTGAGAATAGTGTATGCGG |
| | | CGACCGAGTTGCTCTTGCCCGGCGTCAATACGGGATAATACCGCGCCACATA |
| | | GCAGAACTTTAAAAGTGCTCATCATTGGAAAACGTTCTTCGGGGCGAAAACT |
| | | CTCAAGGATCTTACCGCTGTTGAGATCCAGTTCGATGTAACCCACTCGTGCA |
| | | CCCAACTGATCTTCAGCATCTTTTACTTTCACCAGCGTTTCTGGGTGAGCAA |
| | | AAACAGGAAGGCAAAATGCCGCAAAAAAGGGAATAAGGGCGACACGGAAATG |
| | | TTGAATACTCATACTCTTCCTTTTTCAATATTATTGAAGCATTTATCAGGGT |
| | | TATTGTCTCATGAGCGGATACATATTTGAATGTATTTAGAAAAATAAACAAA |
| | | TAGGGGTTCCGCGCACATTTCCCCGAAAAGTGCCAC |
| 98 | Compound 15 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGTTGTTGCTGCTGCTCGCCTGTATTGCCCTGGCCTCTAC |
| | | AGCCGCCGCTACAAATTCTGCCCCTACCAGCAGCTCCACCAAGAAAACCCAG |
| | | CTGCAACTGGAACATCTGCTGCTGGACCTGCAGATGATCCTGAACGGCATCA |
| | | ACAACTACAAGAACCCCAAGCTGACCCGGATGCTGACCTTCAAGTTCTACAT |
| | | GCCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTGA |
| | | GGCCTAGGGACCTGATCAGCAACATCAACGTGATCGTGCTGGAACTGAAAGG |
| | | CAGCGAGACAACCTTCATGTGCGAGTACGCCGACGAGACAGCTACCATCGTG |
| | | GAATTTCTGAACCGGTGGATCACCTTCTGCCAGAGCATCATCAGCACCCTGA |
| | | CCTGAATAGTGAGTCGTATTAACAACAATCCCGGAGGGCAGAATCATCACGA |
| | | AGTGGTGAAGTACTTGACTTCACCACTTCGTGATGATTCTGCCCTCCACAAC |
| | | AATCCCGAGATGAGCTTCCTACAGCACAACAAATGTGACTTGCACATTTGTT |
| | | GTGCTGTAGGAAGCTCATCTCACAACAATCCCGTACAAGATCCGCAGACGTG |
| | | TAAATGTTCCACTTGGGAACATTTACACGTCTGCGGATCTTGTACTTTATCT |
| | | TAGAGGCATCTGGGCCTCATGGGCCTTCCGCTCACTGCCCGCTTTCCAGTCG |
| | | GGAAACCTGTCGTGCCAGCTGCATTAACATGGTCATAGCTGTTTCCTTGCGT |
| | | ATTGGGCGCTCTCCGCTTCCTCGCTCACTGACTCGCTGCGCTCGGTCGTTCG |
| | | GGTAAAGCCTGGGGTGCCTAATGAGCAAAAGGCCAGCAAAAGGCCAGGAACC |
| | | GTAAAAAGGCCGCGTTGCTGGCGTTTTTCCATAGGCTCCGCCCCCCTGACGA |
| | | GCATCACAAAAATCGACGCTCAAGTCAGAGGTGGCGAAACCCGACAGGACTA |
| | | TAAAGATACCAGGCGTTTCCCCCTGGAAGCTCCCTCGTGCGCTCTCCTGTTC |
| | | CGACCCTGCCGCTTACCGGATACCTGTCCGCCTTTCTCCCTTCGGGAAGCGT |
| | | GGCGCTTTCTCATAGCTCACGCTGTAGGTATCTCAGTTCGGTGTAGGTCGTT |
| | | CGCTCCAAGCTGGGCTGTGTGCACGAACCCCCCGTTCAGCCCGACCGCTGCG |
| | | CCTTATCCGGTAACTATCGTCTTGAGTCCAACCCGGTAAGACACGACTTATC |
| | | GCCACTGGCAGCAGCCACTGGTAACAGGATTAGCAGAGCGAGGTATGTAGGC |
| | | GGTGCTACAGAGTTCTTGAAGTGGTGGCCTAACTACGGCTACACTAGAAGAA |
| | | CAGTATTTGGTATCTGCGCTCTGCTGAAGCCAGTTACCTTCGGAAAAAGAGT |
| | | TGGTAGCTCTTGATCCGGCAAACAAACCACCGCTGGTAGCGGTGGTTTTTTT |
| | | GTTTGCAAGCAGCAGATTACGCGCAGAAAAAAAGGATCTCAAGAAGATCCTT |
| | | TGATCTTTTCTACGGGGTCTGACGCTCAGTGGAACGAAAACTCACGTTAAGG |
| | | GATTTTGGTCATGAGATTATCAAAAAGGATCTTCACCTAGATCCTTTTAAAT |
| | | TAAAAATGAAGTTTTAAATCAATCTAAAGTATATATGAGTAAACTTGGTCTG |
| | | ACAGTTACCAATGCTTAATCAGTGAGGCACCTATCTCAGCGATCTGTCTATT |
| | | TCGTTCATCCATAGTTGCCTGACTCCCCGTCGTGTAGATAACTACGATACGG |
| | | GAGGGCTTACCATCTGGCCCCAGTGCTGCAATGATACCGCGAGAACCACGCT |
| | | CACCGGCTCCAGATTTATCAGCAATAAACCAGCCAGCCGGAAGGGCCGAGCG |
| | | CAGAAGTGGTCCTGCAACTTTATCCGCCTCCATCCAGTCTATTAATTGTTGC |
| | | CGGGAAGCTAGAGTAAGTAGTTCGCCAGTTAATAGTTTGCGCAACGTTGTTG |
| | | CCATTGCTACAGGCATCGTGGTGTCACGCTCGTCGTTTGGTATGGCTTCATT |
| | | CAGCTCCGGTTCCCAACGATCAAGGCGAGTTACATGATCCCCCATGTTGTGC |
| | | AAAAAAGCGGTTAGCTCCTTCGGTCCTCCGATCGTTGTCAGAAGTAAGTTGG |
| | | CCGCAGTGTTATCACTCATGGTTATGGCAGCACTGCATAATTCTCTTACTGT |
| | | CATGCCATCCGTAAGATGCTTTTCTGTGACTGGTGAGTACTCAACCAAGTCA |
| | | TTCTGAGAATAGTGTATGCGGCGACCGAGTTGCTCTTGCCCGGCGTCAATAC |
| | | GGGATAATACCGCGCCACATAGCAGAACTTTAAAAGTGCTCATCATTGGAAA |
| | | ACGTTCTTCGGGGCGAAAACTCTCAAGGATCTTACCGCTGTTGAGATCCAGT |
| | | TCGATGTAACCCACTCGTGCACCCAACTGATCTTCAGCATCTTTTACTTTCA |
| | | CCAGCGTTTCTGGGTGAGCAAAAACAGGAAGGCAAAATGCCGCAAAAAAGGG |
| | | AATAAGGGCGACACGGAAATGTTGAATACTCATACTCTTCCTTTTTCAATAT |
| | | TATTGAAGCATTTATCAGGGTTATTGTCTCATGAGCGGATACATATTTGAAT |
| | | GTATTTAGAAAAATAAACAAATAGGGGTTCCGCGCACATTTCCCCGAAAAGT |
| | | GCCAC |
| 99 | Compound 16 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGTGTCACCAGCAGCTGGTCATCAGCTGGTTCAGCCTGGT |
| | | GTTCCTGGCCTCTCCTCTGGTGGCCATCTGGGAGCTGAAGAAAGACGTGTAC |
| | | GTGGTGGAACTGGACTGGTATCCCGATGCTCCTGGCGAGATGGTGGTGCTGA |
| | | CCTGCGATACCCCTGAAGAGGACGGCATCACCTGGACACTGGATCAGTCTAG |
| | | CGAGGTGCTCGGCAGCGGCAAGACCCTGACCATCCAAGTGAAAGAGTTTGGC |
| | | GACGCCGGCCAGTACACCTGTCACAAAGGCGGAGAAGTGCTGAGCCACAGCC |
| | | TGCTGCTGCTCCACAAGAAAGAGGATGGCATTTGGAGCACCGACATCCTGAA |
| | | GGACCAGAAAGAGCCCAAGAACAAGACCTTCCTGAGATGCGAGGCCAAGAAC |
| | | TACAGCGGCCGGTTCACATGTTGGTGGCTGACCACCATCAGCACCGACCTGA |
| | | CCTTCAGCGTGAAGTCCAGCAGAGGCAGCAGTGATCCTCAGGGCGTTACATG |
| | | TGGCGCCGCTACACTGTCTGCCGAAAGAGTGCGGGGCGACAACAAAGAATAC |
| | | GAGTACAGCGTGGAATGCCAAGAGGACAGCGCCTGTCCAGCCGCCGAAGAGT |
| | | CTCTGCCTATCGAAGTGATGGTGGACGCCGTGCACAAGCTGAAGTACGAGAA |
| | | CTACACCTCCAGCTTTTTCATCCGGGACATCATCAAGCCCGATCCTCCAAAG |
| | | AACCTGCAGCTGAAGCCTCTGAAGAACAGCAGACAGGTGGAAGTGTCCTGGG |
| | | AGTACCCCGACACCTGGTCTACACCCCACAGCTACTTCAGCCTGACCTTTTG |
| | | CGTGCAAGTGCAGGGCAAGTCCAAGCGCGAGAAAAAGGACCGGGTGTTCACC |
| | | GACAAGACCAGCGCCACCGTGATCTGCAGAAAGAACGCCAGCATCAGCGTCA |
| | | GAGCCCAGGACCGGTACTACAGCAGCTCTTGGAGCGAATGGGCCAGCGTGCC |
| | | ATGTTCTGGTGGCGGAGGATCTGGCGGAGGTGGAAGCGGCGGAGGCGGATCT |
| | | AGAAATCTGCCTGTGGCCACTCCTGATCCTGGCATGTTCCCTTGTCTGCACC |
| | | ACAGCCAGAACCTGCTGAGAGCCGTGTCCAACATGCTGCAGAAGGCCAGACA |
| | | GACCCTGGAATTCTACCCCTGCACCAGCGAGGAAATCGACCACGAGGACATC |
| | | ACCAAGGATAAGACCAGCACCGTGGAAGCCTGCCTGCCTCTGGAACTGACCA |
| | | AGAACGAGAGCTGCCTGAACAGCCGGGAAACCAGCTTCATCACCAACGGCTC |
| | | TTGCCTGGCCAGCAGAAAGACCTCCTTCATGATGGCCCTGTGCCTGAGCAGC |
| | | ATCTACGAGGACCTGAAGATGTACCAGGTGGAATTCAAGACCATGAACGCCA |
| | | AGCTGCTGATGGACCCCAAGCGGCAGATCTTCCTGGACCAGAATATGCTGGC |
| | | CGTGATCGACGAGCTGATGCAGGCCCTGAACTTCAACAGCGAGACAGTGCCC |
| | | CAGAAGTCTAGCCTGGAAGAACCCGACTTCTACAAGACCAAGATCAAGCTGT |
| | | GCATCCTGCTGCACGCCTTCCGGATCAGAGCCGTGACCATCGACAGAGTGAT |
| | | GAGCTACCTGAACGCCTCCTGAATAGTGAGTCGTATTAACGTACCAACAAGG |
| | | ACGACGAGACCTTCATCAAACTTGTTGATGAAGGTCTCGTCGTCCTTTATCT |
| | | TAGAGGCATATCCCTACGTACCAACAAGTGCAATGAGGGACCAGTACAACTT |
| | | GTGTACTGGTCCCTCATTGCACTTTATCTTAGAGGCATATCCCTACGTACCA |
| | | ACAATTCTACAACCAGGACCATGAGACTTGCTCATGGTCCTGGTTGTAGAAT |
| | | TTATCTTAGAGGCATATCCCTTTTATCTTAGAGGCATATCCCTCTGGGCCTC |
| | | ATGGGCCTTCCGCTCACTGCCCGCTTTCCAGTCGGGAAACCTGTCGTGCCAG |
| | | CTGCATTAACATGGTCATAGCTGTTTCCTTGCGTATTGGGCGCTCTCCGCTT |
| | | CCTCGCTCACTGACTCGCTGCGCTCGGTCGTTCGGGTAAAGCCTGGGGTGCC |
| | | TAATGAGCAAAAGGCCAGCAAAAGGCCAGGAACCGTAAAAAGGCCGCGTTGC |
| | | TGGCGTTTTTCCATAGGCTCCGCCCCCCTGACGAGCATCACAAAAATCGACG |
| | | CTCAAGTCAGAGGTGGCGAAACCCGACAGGACTATAAAGATACCAGGCGTTT |
| | | CCCCCTGGAAGCTCCCTCGTGCGCTCTCCTGTTCCGACCCTGCCGCTTACCG |
| | | GATACCTGTCCGCCTTTCTCCCTTCGGGAAGCGTGGCGCTTTCTCATAGCTC |
| | | ACGCTGTAGGTATCTCAGTTCGGTGTAGGTCGTTCGCTCCAAGCTGGGCTGT |
| | | GTGCACGAACCCCCCGTTCAGCCCGACCGCTGCGCCTTATCCGGTAACTATC |
| | | GTCTTGAGTCCAACCCGGTAAGACACGACTTATCGCCACTGGCAGCAGCCAC |
| | | TGGTAACAGGATTAGCAGAGCGAGGTATGTAGGCGGTGCTACAGAGTTCTTG |
| | | AAGTGGTGGCCTAACTACGGCTACACTAGAAGAACAGTATTTGGTATCTGCG |
| | | CTCTGCTGAAGCCAGTTACCTTCGGAAAAAGAGTTGGTAGCTCTTGATCCGG |
| | | CAAACAAACCACCGCTGGTAGCGGTGGTTTTTTTGTTTGCAAGCAGCAGATT |
| | | ACGCGCAGAAAAAAAGGATCTCAAGAAGATCCTTTGATCTTTTCTACGGGGT |
| | | CTGACGCTCAGTGGAACGAAAACTCACGTTAAGGGATTTTGGTCATGAGATT |
| | | ATCAAAAAGGATCTTCACCTAGATCCTTTTAAATTAAAAATGAAGTTTTAAA |
| | | TCAATCTAAAGTATATATGAGTAAACTTGGTCTGACAGTTACCAATGCTTAA |
| | | TCAGTGAGGCACCTATCTCAGCGATCTGTCTATTTCGTTCATCCATAGTTGC |
| | | CTGACTCCCCGTCGTGTAGATAACTACGATACGGGAGGGCTTACCATCTGGC |
| | | CCCAGTGCTGCAATGATACCGCGAGAACCACGCTCACCGGCTCCAGATTTAT |
| | | CAGCAATAAACCAGCCAGCCGGAAGGGCCGAGCGCAGAAGTGGTCCTGCAAC |
| | | TTTATCCGCCTCCATCCAGTCTATTAATTGTTGCCGGGAAGCTAGAGTAAGT |
| | | AGTTCGCCAGTTAATAGTTTGCGCAACGTTGTTGCCATTGCTACAGGCATCG |
| | | TGGTGTCACGCTCGTCGTTTGGTATGGCTTCATTCAGCTCCGGTTCCCAACG |
| | | ATCAAGGCGAGTTACATGATCCCCCATGTTGTGCAAAAAAGCGGTTAGCTCC |
| | | TTCGGTCCTCCGATCGTTGTCAGAAGTAAGTTGGCCGCAGTGTTATCACTCA |
| | | TGGTTATGGCAGCACTGCATAATTCTCTTACTGTCATGCCATCCGTAAGATG |
| | | CTTTTCTGTGACTGGTGAGTACTCAACCAAGTCATTCTGAGAATAGTGTATG |
| | | CGGCGACCGAGTTGCTCTTGCCCGGCGTCAATACGGGATAATACCGCGCCAC |
| | | ATAGCAGAACTTTAAAAGTGCTCATCATTGGAAAACGTTCTTCGGGGCGAAA |
| | | ACTCTCAAGGATCTTACCGCTGTTGAGATCCAGTTCGATGTAACCCACTCGT |
| | | GCACCCAACTGATCTTCAGCATCTTTTACTTTCACCAGCGTTTCTGGGTGAG |
| | | CAAAAACAGGAAGGCAAAATGCCGCAAAAAAGGGAATAAGGGCGACACGGAA |
| | | ATGTTGAATACTCATACTCTTCCTTTTTCAATATTATTGAAGCATTTATCAG |
| | | GGTTATTGTCTCATGAGCGGATACATATTTGAATGTATTTAGAAAAATAAAC |
| | | AAATAGGGGTTCCGCGCACATTTCCCCGAAAAGTGCCAC |
| 100 | Compound 17 | CTAAATTGTAAGCGTTAATATTTTGTTAAAATTCGCGTTAAATTTTTGTTAA |
| | (pMA-RQ) | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGTGTCACCAGCAGCTGGTCATCAGCTGGTTCAGCCTGGT |
| | | GTTCCTGGCCTCTCCTCTGGTGGCCATCTGGGAGCTGAAGAAAGACGTGTAC |
| | | GTGGTGGAACTGGACTGGTATCCCGATGCTCCTGGCGAGATGGTGGTGCTGA |
| | | CCTGCGATACCCCTGAAGAGGACGGCATCACCTGGACACTGGATCAGTCTAG |
| | | CGAGGTGCTCGGCAGCGGCAAGACCCTGACCATCCAAGTGAAAGAGTTTGGC |
| | | GACGCCGGCCAGTACACCTGTCACAAAGGCGGAGAAGTGCTGAGCCACAGCC |
| | | TGCTGCTGCTCCACAAGAAAGAGGATGGCATTTGGAGCACCGACATCCTGAA |
| | | GGACCAGAAAGAGCCCAAGAACAAGACCTTCCTGAGATGCGAGGCCAAGAAC |
| | | TACAGCGGCCGGTTCACATGTTGGTGGCTGACCACCATCAGCACCGACCTGA |
| | | CCTTCAGCGTGAAGTCCAGCAGAGGCAGCAGTGATCCTCAGGGCGTTACATG |
| | | TGGCGCCGCTACACTGTCTGCCGAAAGAGTGCGGGGCGACAACAAAGAATAC |
| | | GAGTACAGCGTGGAATGCCAAGAGGACAGCGCCTGTCCAGCCGCCGAAGAGT |
| | | CTCTGCCTATCGAAGTGATGGTGGACGCCGTGCACAAGCTGAAGTACGAGAA |
| | | CTACACCTCCAGCTTTTTCATCCGGGACATCATCAAGCCCGATCCTCCAAAG |
| | | AACCTGCAGCTGAAGCCTCTGAAGAACAGCAGACAGGTGGAAGTGTCCTGGG |
| | | AGTACCCCGACACCTGGTCTACACCCCACAGCTACTTCAGCCTGACCTTTTG |
| | | CGTGCAAGTGCAGGGCAAGTCCAAGCGCGAGAAAAAGGACCGGGTGTTCACC |
| | | GACAAGACCAGCGCCACCGTGATCTGCAGAAAGAACGCCAGCATCAGCGTCA |
| | | GAGCCCAGGACCGGTACTACAGCAGCTCTTGGAGCGAATGGGCCAGCGTGCC |
| | | ATGTTCTGGTGGCGGAGGATCTGGCGGAGGTGGAAGCGGCGGAGGCGGATCT |
| | | AGAAATCTGCCTGTGGCCACTCCTGATCCTGGCATGTTCCCTTGTCTGCACC |
| | | ACAGCCAGAACCTGCTGAGAGCCGTGTCCAACATGCTGCAGAAGGCCAGACA |
| | | GACCCTGGAATTCTACCCCTGCACCAGCGAGGAAATCGACCACGAGGACATC |
| | | ACCAAGGATAAGACCAGCACCGTGGAAGCCTGCCTGCCTCTGGAACTGACCA |
| | | AGAACGAGAGCTGCCTGAACAGCCGGGAAACCAGCTTCATCACCAACGGCTC |
| | | TTGCCTGGCCAGCAGAAAGACCTCCTTCATGATGGCCCTGTGCCTGAGCAGC |
| | | ATCTACGAGGACCTGAAGATGTACCAGGTGGAATTCAAGACCATGAACGCCA |
| | | AGCTGCTGATGGACCCCAAGCGGCAGATCTTCCTGGACCAGAATATGCTGGC |
| | | CGTGATCGACGAGCTGATGCAGGCCCTGAACTTCAACAGCGAGACAGTGCCC |
| | | CAGAAGTCTAGCCTGGAAGAACCCGACTTCTACAAGACCAAGATCAAGCTGT |
| | | GCATCCTGCTGCACGCCTTCCGGATCAGAGCCGTGACCATCGACAGAGTGAT |
| | | GAGCTACCTGAACGCCTCCTGAACAACAAGGACGACGAGACCTTCATCAAAC |
| | | TTGTTGATGAAGGTCTCGTCGTCCACAACAAGTGCAATGAGGGACCAGTACA |
| | | ACTTGTGTACTGGTCCCTCATTGCACACAACAATTCTACAACCAGGACCATG |
| | | AGACTTGCTCATGGTCCTGGTTGTAGAAACAACAATTTATCTTAGAGGCATA |
| | | TCCCTCTGGGCCTCATGGGCCTTCCGCTCACTGCCCGCTTTCCAGTCGGGAA |
| | | ACCTGTCGTGCCAGCTGCATTAACATGGTCATAGCTGTTTCCTTGCGTATTG |
| | | GGCGCTCTCCGCTTCCTCGCTCACTGACTCGCTGCGCTCGGTCGTTCGGGTA |
| | | AAGCCTGGGGTGCCTAATGAGCAAAAGGCCAGCAAAAGGCCAGGAACCGTAA |
| | | AAAGGCCGCGTTGCTGGCGTTTTTCCATAGGCTCCGCCCCCCTGACGAGCAT |
| | | CACAAAAATCGACGCTCAAGTCAGAGGTGGCGAAACCCGACAGGACTATAAA |
| | | GATACCAGGCGTTTCCCCCTGGAAGCTCCCTCGTGCGCTCTCCTGTTCCGAC |
| | | CCTGCCGCTTACCGGATACCTGTCCGCCTTTCTCCCTTCGGGAAGCGTGGCG |
| | | CTTTCTCATAGCTCACGCTGTAGGTATCTCAGTTCGGTGTAGGTCGTTCGCT |
| | | CCAAGCTGGGCTGTGTGCACGAACCCCCCGTTCAGCCCGACCGCTGCGCCTT |
| | | ATCCGGTAACTATCGTCTTGAGTCCAACCCGGTAAGACACGACTTATCGCCA |
| | | CTGGCAGCAGCCACTGGTAACAGGATTAGCAGAGCGAGGTATGTAGGCGGTG |
| | | CTACAGAGTTCTTGAAGTGGTGGCCTAACTACGGCTACACTAGAAGAACAGT |
| | | ATTTGGTATCTGCGCTCTGCTGAAGCCAGTTACCTTCGGAAAAAGAGTTGGT |
| | | AGCTCTTGATCCGGCAAACAAACCACCGCTGGTAGCGGTGGTTTTTTTGTTT |
| | | GCAAGCAGCAGATTACGCGCAGAAAAAAAGGATCTCAAGAAGATCCTTTGAT |
| | | CTTTTCTACGGGGTCTGACGCTCAGTGGAACGAAAACTCACGTTAAGGGATT |
| | | TTGGTCATGAGATTATCAAAAAGGATCTTCACCTAGATCCTTTTAAATTAAA |
| | | AATGAAGTTTTAAATCAATCTAAAGTATATATGAGTAAACTTGGTCTGACAG |
| | | TTACCAATGCTTAATCAGTGAGGCACCTATCTCAGCGATCTGTCTATTTCGT |
| | | TCATCCATAGTTGCCTGACTCCCCGTCGTGTAGATAACTACGATACGGGAGG |
| | | GCTTACCATCTGGCCCCAGTGCTGCAATGATACCGCGAGAACCACGCTCACC |
| | | GGCTCCAGATTTATCAGCAATAAACCAGCCAGCCGGAAGGGCCGAGCGCAGA |
| | | AGTGGTCCTGCAACTTTATCCGCCTCCATCCAGTCTATTAATTGTTGCCGGG |
| | | AAGCTAGAGTAAGTAGTTCGCCAGTTAATAGTTTGCGCAACGTTGTTGCCAT |
| | | TGCTACAGGCATCGTGGTGTCACGCTCGTCGTTTGGTATGGCTTCATTCAGC |
| | | TCCGGTTCCCAACGATCAAGGCGAGTTACATGATCCCCCATGTTGTGCAAAA |
| | | AAGCGGTTAGCTCCTTCGGTCCTCCGATCGTTGTCAGAAGTAAGTTGGCCGC |
| | | AGTGTTATCACTCATGGTTATGGCAGCACTGCATAATTCTCTTACTGTCATG |
| | | CCATCCGTAAGATGCTTTTCTGTGACTGGTGAGTACTCAACCAAGTCATTCT |
| | | GAGAATAGTGTATGCGGCGACCGAGTTGCTCTTGCCCGGCGTCAATACGGGA |
| | | TAATACCGCGCCACATAGCAGAACTTTAAAAGTGCTCATCATTGGAAAACGT |
| | | TCTTCGGGGCGAAAACTCTCAAGGATCTTACCGCTGTTGAGATCCAGTTCGA |
| | | TGTAACCCACTCGTGCACCCAACTGATCTTCAGCATCTTTTACTTTCACCAG |
| | | CGTTTCTGGGTGAGCAAAAACAGGAAGGCAAAATGCCGCAAAAAAGGGAATA |
| | | AGGGCGACACGGAAATGTTGAATACTCATACTCTTCCTTTTTCAATATTATT |
| | | GAAGCATTTATCAGGGTTATTGTCTCATGAGCGGATACATATTTGAATGTAT |
| | | TTAGAAAAATAAACAAATAGGGGTTCCGCGCACATTTCCCCGAAAAGTGCCA |
| | | C |

Bold and underline = compound sequence

### Example 2: In Vitro Transcription of RNA Constructs and Data Analysis

PCR-based in vitro transcription was carried out using the pMA-RQ/pUC-GW-Kan vectors encoding Cpd.1-Cpd.17 to produce RNA constructs. A transcription template was generated by PCR using the forward and reverse primers in Table 4 (SEQ ID NOs: 24 and 25). The poly(A) tail was encoded in the template resulting in a 120 bp poly(A) tail (SEQ ID NO: 155). Optimizations were made as needed to achieve specific amplification given the repetitive sequence of siRNA flanking regions. Optimizations include: 1) decreasing the amount of plasmid DNA of vector, 2) changing the DNA polymerase (Q5 hot start polymerase, New England Biolabs), 3) reducing denaturation time (30 seconds to 10 seconds) and extension time (45 seconds/kb to 10 seconds/kb) for each cycle of PCR, 4) increasing the annealing (10 seconds to 30 seconds) for each cycle of PCR, and 5) increasing the final extension time (up to 15 minutes) for each cycle of PCR. In addition, to avoid non-specific primer binding, the PCR reaction mixture was prepared on ice including thawing reagents, and the number of PCR cycles was reduced to 25.

For in vitro transcription, T7 RNA polymerase (MEGAscript kit, Thermo Fisher Scientific) was used at 37° C. for 2 hours. Synthesized RNAs were chemically modified with 100% N¹-methylpseudo-UTP and co-transcriptionally capped with an anti-reverse CAP analog (ARCA; [m₂^{7,3′-O}G(5′)ppp(5′)G]) at the 5′ end (Jena Bioscience). To generate unmodified RNA to measure immunogenicity, canonical dUTP was used instead of N¹-methylpseudo-UTP. After in vitro transcription, the RNA constructs were column-purified using MEGAclear kit (Thermo Fisher Scientific) and quantified using Nanophotometer-N⁶⁰ (Implen).

TABLE 4
Primers for Template Generation

| SEQ | | |
|---|---|---|
| ID | Primer | |
| NO | Direction | Sequence (5′ to 3′) |
| 24 | Forward | GCTGCAAGGCGATTAAGTTG |
| 25 | Reverse | U(2′OMe)U(2′OMe)U(2′OMe) |
| | | TTTTTTTTTTTTTTTTTTTTTT |
| | | TTTTTTTTTTTTTTTTTTTTTTT |
| | | TTTTTTTTTTTTTTTTTTTTTTT |
| | | TTTTTTTTTTTTTTTTTTTTTTT |
| | | TTTTTTTTTTTTTTTTTTTTTTT |
| | | TTTCAGCTATGACCATGTTAATG |
| | | CAG |

Using in vitro transcription, Cpd.1-Cpd.17 were generated as RNA constructs (200-500 μg) and tested in various in vitro models specified below for IL-4, IGF1, IL-2 and IL-12 expression and combinatorial effect of IL-4, IGF1, IL-2 and IL-12 overexpression in parallel to TNF-alpha, ALK2, Turbo-GFP, VEGFA, c-Myc, KRAS, Akt1, Akt2, and Akt3 down regulation.

Determination of Molecular weight of constructs was performed as below. The molecular weight of each construct was determined from each sequence by determining the total number of each base (A, C, G, T or N¹-UTP) present in each sequence and multiply the number by respective molecular weight (e.g., A: 347.2 g/mol; C 323.2 g/mol; G 363.2 g/mol; N¹-UTP:338.2 g/mol). The molecular weight was determined by the sum of all weights obtained for each base and ARCA molecular weight of 817.4 g/mol. The molecular weight of each construct was used to calculate the amount of RNA used for transfection in each well to nanomolar (nM) concentration.

Data were analyzed using GraphPad Prism 8 (San Diego, USA). For the estimation of the protein levels using ELISA in the standard or the sample, the mean absorbance value of the blank was subtracted from the mean absorbance of the standards or the samples. A standard curve was generated and plotted using a four parameters nonlinear regression according to manufacturer's protocol. To determine the concentration of proteins in each sample, the concentration of the different protein was interpolated from the standard curve. The final protein concentration of the sample was calculated by multiplication with the dilution factor. Statistical analysis was carried out using by Student's t-test or one-way ANOVA followed by Dunnet's multiple comparing tests.

### Example 3: In Vitro Transfection of THP-1 Cells and Endogenous TNF-Alpha Expression Model in THP-1 Cells

In Vitro Transfection of THP-1 Cells

Human monocyte leukemia cell line THP-1 (Sigma-Aldrich, Cat. #88081201) was maintained in growth medium (RPMI 1640 supplemented with 10% FBS and 2 mM glutamine). The cells were seeded at 30,000 THP-1 cells in a 96 well cell culture plate 72 hours before transfection and activated with 50 nM of phorbol 12-myristate 13-acetate (PMA) (Sigma-Aldrich, Cat. #P8139) diluted in growth medium. The cells were transfected with specific RNA constructs (600 ng/well) and scrambled siRNA (600 ng/well; Sigma, Cat.#SIC002) using Lipofectamine 2000 (Thermo Fisher Scientific). 100 μl of DMEM was removed from each well and replaced with 50 μl of Opti-MEM (Thermo Fisher Scientific) and 50 μl RNA and Lipofectamine 2000 complex in Opti-MEM. After 5 hours, the medium was replaced with fresh growth medium supplemented with 50 nM PMA and the plates were incubated at 37° C. in a humidified atmosphere containing 5% CO₂ for 24 hours.

Endogenous TNF-Alpha Expression Model in THP-1 Cells

For the endogenous secretion of TNF-alpha in THP-1 cells, THP-1 cells were stimulated with E. coli-derived lipopolysaccharide (LPS-L4391; Sigma Aldrich) at 10 μg/mL final concentration with R848 (TLR7/8 agonist; Invivogen) at 1 μg/mL final concentration and incubated for 90 minutes. The induced production of TNF-alpha corresponds to the physiological conditions observed in psoriasis. Post stimulation, 50 μl of media was removed and replaced with the transfection complex containing specific RNA constructs (Cpd.1 and Cpd.2) complexed with Lipofectamine 2000 in Opti-MEM and incubated at 37° C. in a humidified atmosphere containing 5% CO 2 for 24 hours. Post transfection, the cell culture supernatant was collected and quantified for TNF-alpha (target gene to downregulate) and IL-4 (Gene of Interest to overexpress) by ELISA. The TNF-alpha levels in non-transfected, but stimulated samples were used as control and set to 100% and percent of TNF-alpha knock down was calculated.

Results

The effect of Cpd.1 (comprising 3× siRNA targeting TNF-alpha at 3′ and IL-4 coding sequence with A1-linker) and Cpd.2 (comprising 3× siRNA targeting TNF-alpha at 3′ and IL-4 coding sequence with A2-linker) in downregulation of TNF-alpha was evaluated in THP-1 cells stimulated with 10 μg/mL LPS and 1 μg/mL R848 to induce endogenous TNF-alpha secretion. The established THP-1 model mimics the physiological immune condition of psoriasis. As demonstrated in FIG. 2A, Cpd.1 and Cpd.2 downregulated the expression (>80%) of endogenous TNF-alpha expression in THP-1 cells (P<0.001). The same cell culture supernatant was measured for IL-4 expression and it was confirmed that IL-4 expression was not impaired (FIG. 2B). It's noted that the level of IL-4 expression by Cpd.2 is 2.5-fold higher than the level of IL-4 expression by Cpd.1 as shown in FIG. 2B (P<0.01).

### Example 4: In Vitro Transfection of HEK-293 and TNF-Alpha Overexpression Model in HEK-293 Cells

In Vitro Transfection of HEK-293 Cells

Human embryonic kidney cells 293 (HEK-293; ATCC CRL-1573) were maintained in Dulbecco's Modified Eagle's medium (DMEM, Biochrom) supplemented with 10% (v/v) Fetal Bovine Serum (FBS) and Penicillin-Streptomycin-Amphotericin B mixture (882087, Biozym Scientific). Cells were seeded at 20,000 cell/well in a 96 well culture plate and incubated at 37° C. in a humidified atmosphere containing 5% CO₂ for 24 hours prior to transfection. Cells were grown in DMEM growth medium containing 10% of FBS without antibiotics to reach confluency <60% before transfection. Thereafter, HEK-293 cells were transfected with specific RNA constructs (TNF-alpha mRNA, Cpd.1 or Cpd.2) with varying concentrations (600-1500 ng) using Lipofectamine 2000 (Invitrogen) following the manufacturer's instructions with the RNA to Lipofectamine ratio of 1:1 w/v. 100 μl of DMEM was removed and replaced with 50 μl of Opti-MEM and 50 μl RNA and Lipofectamine 2000 complex in Opti-MEM (Thermo Fisher Scientific). After 5 hours, the medium was replaced by fresh medium and the plates were incubated at 37° C. in a humidified atmosphere containing 5% CO₂ for 24 hours.

TNF-Alpha Overexpression Model in HEK-293 Cells

To assess the simultaneous effect of TNF-alpha RNA interference (RNAi) and IL-4 expression of Cpd.1 and Cpd.2 in HEK-293 cells, the TNF-alpha overexpression model was established using TNF-alpha mRNA transfection (600 ng/well). To assess the capability of Cpd.1 and Cpd.2 containing TNF-alpha targeting siRNA in TNF-alpha downregulation and simultaneous IL-4 expression, the cells were co-transfected with Cpd.1 and Cpd.2 (900 ng/well) and TNF-alpha mRNA (600 ng/well). Post transfection, the cells were incubated at 37° C. in a humidified atmosphere containing 5% CO₂ for 24 hours followed by quantification of TNF-alpha (target gene to downregulate) and IL-4 (Gene of Interest to overexpress) by ELISA in the same cell culture supernatant. The TNF-alpha levels in non-transfected samples were used as control and set to 100% and percent of TNF-alpha knock down was calculated.

Results

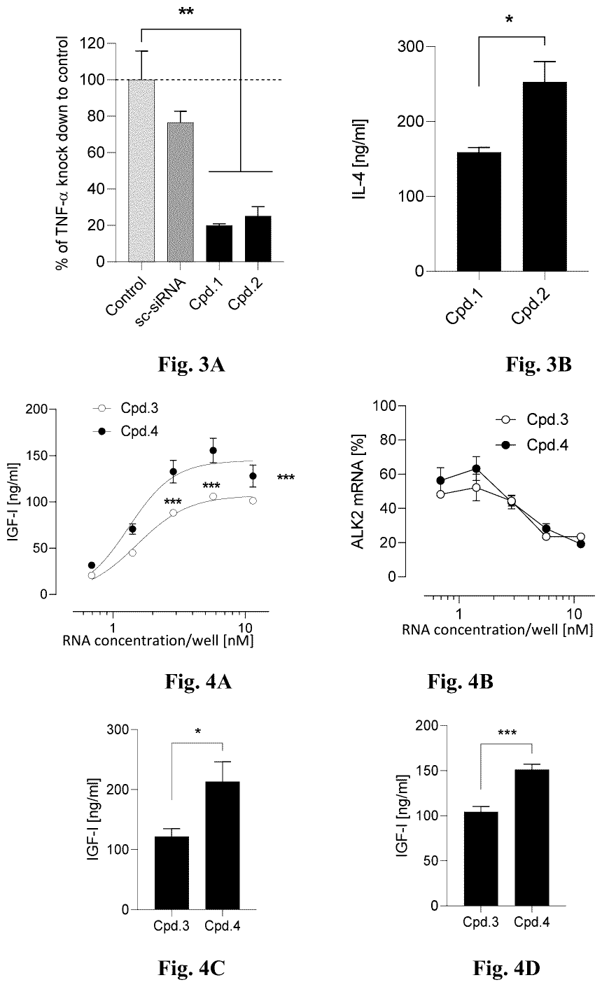

Cpd.1 and Cpd.2 comprising TNF-alpha-targeting siRNA and IL-4 protein coding sequence either with A1-linker or A2-linker, respectively, were tested for TNF-alpha downregulation and IL-4 expression at the same time in HEK-293 cells (900 ng/well) with exogenously delivered TNF-alpha mRNA (600 ng/well). As shown in FIG. 3A, both Cpd.1 and Cpd.2 downregulated the TNF-alpha level compared to untreated control up to 80% (P<0.01). In addition, the level of IL-4 expression by Cpd.2 was 1.6-fold higher than the level of IL-4 expression by Cpd.1, as shown in FIG. 3B (P<0.05).

### Example 5: In Vitro Transfection of A549 Cells and Endogenous ALK2 Expression Model in A549 Cells

In Vitro Transfection of A549 Cells

A549 cells are typical alveolar type II (ATII) cells derived from human lung carcinoma. Since A549 cells express endogenous ALK2 RNA transcripts (a therapeutic target for fibrodysplasia ossificans progressiva, FOP) at a moderate level, A549 cells were used to study the effect of Cpd.3 and Cpd.4 in degrading the ALK2 mRNA in parallel to measuring IGF-1 expression. The A549 cells (Sigma-Aldrich, Buchs Switzerland Cat. #6012804) were maintained on Dulbecco's Modified Eagle's medium-high glucose (DMEM, Sigma-Aldrich, Buchs Switzerland cat #D0822) supplemented with 10% FBS (Thermo Fisher Scientific, Basel, Switzerland; cat. #10500-064). To assess Cpd.3 and Cpd.4 activity, the A549 cells were plated at a density of 10,000 cells/well in a regular growth medium 24 hours prior to transfection. Thereafter, cells were transfected with increasing concentration of Cpd.3 and Cpd.4 (0.65, 1.33, 2.7, 5.4 and 10.8 nM) using Lipofectamine 2000 (www.invitrogen.com) following the manufacturer's instructions. 100 μl of DMEM were removed and 50 μl of Opti-MEM (www.thermofisher.com) was added to each well followed by 50 μl RNA and Lipofectamine 2000 complex in Opti-MEM. After 5 hours of incubation, the medium was replaced by fresh growth medium and the plates were incubated for 24 hours at 37° C. in a humidified atmosphere containing 5% CO₂, followed by IGF-1 quantification by ELISA and ALK2 mRNA by relative quantification using qPCR with primers targeting human ALK2 mRNA (Forward primer: 5′-GACGTGGAGTATGGCACTATCG-3′ and Reverse primer: 5′-CACTCCAACAGTGTAATCTGGCG-3′; SEQ ID NOs: 35 and 36, respectively) using SYBR 1-Step Cells to CT kit (Thermo Fisher Scientific, Basel, Switzerland; cat. #A25599). The human 18S rRNA was used as a reference control (Forward primer: 5′-ACCCGTTGAACCCCATTCGTGA-3′ and Reverse primer: 5′-GCCTCACTAAACCATCCAATCGG-3′; SEQ ID NOs: 37 and 38, respectively).

Results

Dose response results showed that the effect of A2-linker in Cpd.4 towards IGF-1 expression is significantly higher than A1-linker in Cpd.3 in A549 cells (FIG. 4A; P<0.001). In the same cell culture lysate, the RNA interference of Cpd.3 and Cpd.4 against remaining ALK-2 expression was assessed by relative quantification. As demonstrated in FIG. 4B, both Cpd.3 and Cpd.4 equally downregulated the endogenous ALK2 RNA transcripts expression up to 75%.

In an analysis in A549 cells with 12 replicates for 1.33 nM/well, Cpd.4 treated cells showed 1.5-fold higher IGF-1 expression and secretion compared to Cpd.3 (FIG. 4D, P<0.001). To confirm the effect of A2-linker in IGF-1 expression in another cell type, the same experiment (1.33 nM/well, 12 replicates) was conducted in HEK293 cells as described in Example 4. The results show that the level of IGF-1 expression and secretion from cells treated with Cpd.4 is 1.8-fold higher than by Cpd.3 (FIG. 4C, P<0.05).

### Example 6: In Vitro Transfection of SCC-4 Cells and Combinatorial Effect of IGF1 Secretion and Turbo GFP Downregulation in SCC-4 Cells in Turbo GFP Overexpression Model

In Vitro Transfection of SCC-4 Cells

Human tongue squamous carcinoma (SCC-4; Sigma cat #89062002 CRL-1573) cells were maintained in Dulbecco's Modified Eagle's high glucose medium (DMEM, Sigma Aldricht) supplemented with HAM F12 (1:1)+2 mM Glutamine+10% Fetal Bovine Serum (FBS)+0.4 μg/ml hydrocortisone. Cells were seeded at 15,000 cell/well in a 96 well culture plate and incubated at 37° C. in a humidified atmosphere containing 5% CO₂ for 24 hours prior to transfection. Cells were grown in DMEM/HAM F-12 growth medium to reach confluency <70% before transfection. Thereafter, SCC-4 cells were co-transfected with Turbo GFP mRNA (0.3 μg) to establish Turbo GFP overexpression model and specific RNA constructs (Cpd.5 to Cpd.9, 30 nM/well) which comprise IGF-1 protein encoding RNA sequence and 1× or 2× siRNA against Turbo GFP using Lipofectamine 2000 (Invitrogen) following the manufacturer's instructions. Universal scrambled siRNA (Sigma, Cat. #SIC002) was used as control (30 nM/well). The RNA to Lipofectamine ratio was 1:1 w/v. 100 μl of DMEM was removed and replaced with 50 μl of Opti-MEM and 50 μl RNA and Lipofectamine 2000 complex in Opti-MEM (Thermo Fisher Scientific). After 5 hours, the medium was replaced by fresh growth medium without FBS and the plates were incubated at 37° C. in a humidified atmosphere containing 5% CO₂ for 24 hours. The Turbo GFP positive cells in non-transfected samples were used as control and set to 0% and percent of Turbo GFP knock down was calculated.

Results

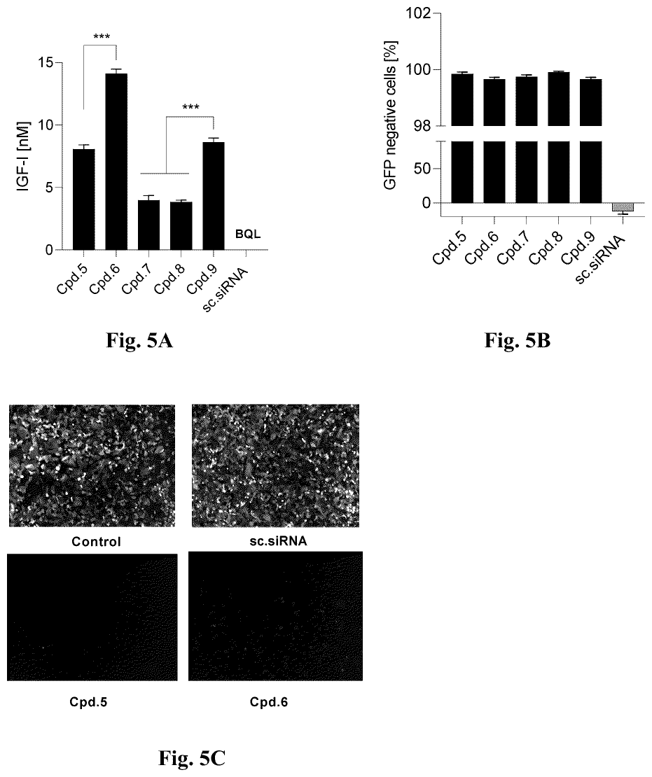

The effect of linkers A1 (Cpd.5, Cpd.7 and Cpd.8) and A2 (Cpd.6 and Cpd.9) on IGF-1 expression were assessed in SCC-4 cells. The data demonstrate that A2-linker containing Cpd.6 and Cpd.9 showed increased IGF1 levels compared to their A1-linker counterparts from 1.8-fold to 2.2-fold higher (FIG. 5A; P<0.001). No difference was shown in number of siRNA (1× or 2× siRNA) and siRNA sequence difference (Cpd.7 has same siRNA sequence repeated twice whereas Cpd. 8 has two different siRNA sequences) with respect to reduced IGF-1 levels of A1 linker compared to A2 linker. All tested constructs resulted in complete Turbo GFP down regulation (98-99%; FIG. 4B). FIG. 4C shows representative microscopical images of control, sc.siRNA, Cpd.5 and Cpd.6 with Turbo GFP expression in greyscale.

### Example 7: Comparative Analysis of Compounds with Different Linkers (Cpd.10 to Cpd.15) for IL-2 Expression and Endogenous VEGFA Downregulation in A549 Cells

In Vitro Transfection of A549 Cells

A549 cells were cultured and transfected as described above. The growth medium did not contain FBS to avoid FBS-derived VEGFA effect in the experiment. A549 cells were used as these cells endogenously express VEGFA up to 50-100 ng/mL in vitro. To evaluate the impact of different linkers (A1, A2, B-E) on simultaneous VEGFA RNA interference (RNAi) and IL-2 expression, A549 cells were transfected with increasing concentrations (1, 3, and 30 nM) of Cpd.10-Cpd.15. Cells were then incubated at 37° C. in a humidified atmosphere containing 5% CO₂ for 24 hours, followed by quantification of VEGFA (ThermoFisher Cat. #KHG0112) and IL-2 (ThermoFisher Cat. #887025) present in the same cell culture supernatant by using specific ELISAs. VEGFA levels from untransfected cells were set to 100% and downregulation of VEGFA level was normalized to untransfected samples (basal level).

Results

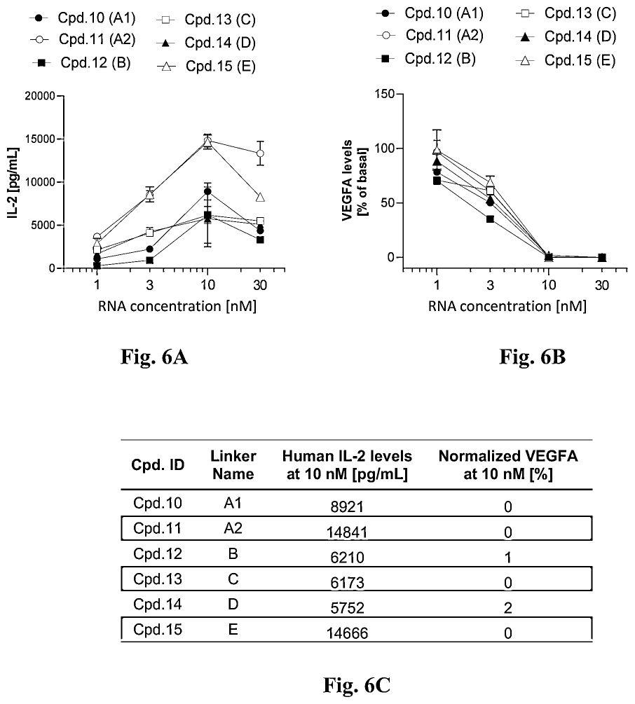

The effect of linkers A1 (Cpd.10), A2 (Cpd.11), B (Cpd.12), C (Cpd.13), D (Cpd.14), and E (Cpd.15) on IL-2 expression were assessed in A549 cells. The data demonstrate that cells transfected with A2-linker containing Cpd.11 and E-linker containing Cpd.15 showed increased IL-2 levels (from 1.5-fold to 2.5-fold higher) compared to cells transfected with other linkers (FIGS. 6A and 6C). To assess the effect of linkers on the inhibition of endogenous VEGFA expression, a dose response study was performed in A549 cells. Cells transfected with compounds with different linkers (10 nM and 30 nM concentration) exhibited complete VEGFA downregulation (98-99%) (FIGS. 6B and 6C). However, Cpd.12 containing B-linker showed higher potency of 65% VEGFA downregulation at 3 nM concentration whereas Cpd.10 (A1; 50%), Cpd.11 (A2; 40%), Cpd.13 (C; 39%), Cpd.14 (D; 46%) and Cpd.15 (E; 31%) revealed reduced potency in VEGFA downregulation. A similar trend of improved VEGFA downregulation (30%) was noted at 1 nM concentration for Cpd.12 (containing B-linker) compared to compounds with other linkers. Nevertheless, IL-2 expression from Cpd.12 was −9 fold lower than IL-2 expression compared to Cpd.11 and Cpd.15 at 3 nM concentration (FIG. 6A).

### Example 8: Comparative Analysis of Compounds with Different Linkers (Cpd.10 to Cpd.15) for IL-2 Expression and Endogenous VEGFA Downregulation in SCC-4 Cells

In Vitro Transfection of SCC-4 Cells

SCC-4 cells were cultured and transfected as described above. The growth medium did not contain FBS to avoid FBS-derived VEGFA in the experiment. SCC-4 cells were used as these cells endogenously express VEGFA up to 800 ng/mL in vitro. To evaluate the impact of different linkers (A1, A2, B-E) on simultaneous VEGFA RNA interference (RNAi) and IL-2 expression, SCC-4 cells were transfected with increasing concentrations (1, 3, 10 and 30 nM) of Cpd.10-Cpd.15. Cells were then incubated at 37° C. in a humidified atmosphere containing 5% CO₂ for 24 hours, followed by quantification of VEGFA (ThermoFisher Cat. #KHG0112) and IL-2 (ThermoFisher Cat. #887025) present in the same cell culture supernatant by using specific ELISAs. VEGFA levels from untransfected cells were set to 100% and downregulation of VEGFA level normalized to untransfected samples (basal level).

Results

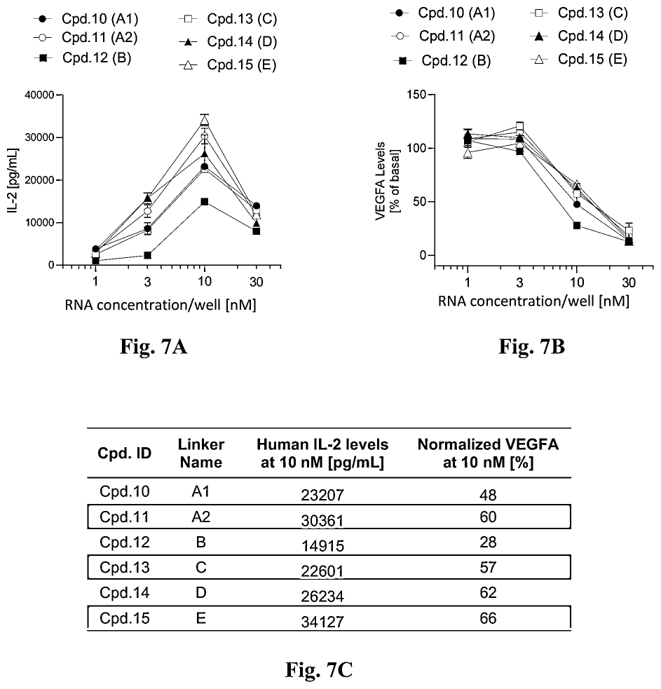

The effect of linkers A1 (Cpd.10), A2 (Cpd.11), B (Cpd.12), C (Cpd.13), D (Cpd.14) and E (Cpd.15) on IL-2 expression were assessed in SCC-4 cells. Similar to A549 cells, cells transfected with Cpd.11 containing A2-linker and Cpd.15 containing E-linker showed increased IL-2 levels (from 1.2-fold to 2.5-fold higher) compared to cells transfected with other linkers (FIGS. 7A and 7C). Cpd.12 containing B-linker resulted in decreased IL-2 levels compared to Cpd.11 containing A2-linker (14915 pg/mL vs. 30361 pg/mL) and Cpd.15 containing E-linker (14915 pg/mL vs. 34127 pg/mL) at 3 nM concentration. Assessment of endogenous VEGFA expression inhibition in SCC-4 cells revealed that Cpd. 12 containing B-linker showed higher potency of 72% VEGFA downregulation at 10 nM concentration whereas Cpd.10 (A1; 52%), Cpd.11 (A2; 40%), Cpd.13 (C; 47%), Cpd.14 (D; 38%) and Cpd.15 (E; 34%) revealed reduced potency in VEGFA downregulation. All compounds with different linkers exhibited equivalent level of VEGFA downregulation (80-90%) at 30 nM concentration (FIG. 7B).

### Example 9: In Vitro Transfection of Primary HSMM Cells and Assessment of IGF-1 Expression from Compounds with Different Linkers

In Vitro Transfection of HSMM Cells

Adult Human Skeletal Muscle Myoblast (HSMM, CC-2580, Lonza, Switzerland) cells are primary skeletal muscle cells isolated from the upper arm or leg muscle of normal healthy donors. HSMM cells are disease-relevant cell type for Fibrodysplasia ossificans progressiva (FOP) disease. The impact of A1 and A2 linkers on protein expression was evaluated in primary cells in addition to curated cell lines. HSMM cells were maintained in Skeletal Growth medium (SkGM) which contains Skeletal Muscle—2 Basal Medium (SkBM, CC-3246, Lonza, Switzerland), supplemented with Skeletal Muscle−2 SingleQuots Kit (SkGM-Kit, CC-3244, Lonza, Switzerland). The addition of differentiation medium (growth medium+2% heat activated Horse Serum; 26050-070, Life Technologies, Switzerland) differentiate the primary myoblasts into multinucleated myotubes which is relevant for FOP. To assess Cpd.3 and Cpd.4 activity, HSMM cells were plated in a 96 well cell culture plate with 10,000 cells/well 24 hours prior to transfection. Cells were grown in SkGM growth medium (GM) or Differentiation Medium (DM). Thereafter, cells were transfected with increasing concentration of Cpd.3 and Cpd.4 (0.1, 0.3, 1, 3, 10 and 30 nM) using Lipofectamine 2000 following the manufacturer's instructions. 100 μl of SkGM were removed and 50 μl of Opti-MEM (www.thermofisher.com) was added to each well followed by 50 μl of RNA and Lipofectamine 2000 complex in Opti-MEM. After 5 hours of incubation, the medium was replaced by fresh growth medium and the plates were incubated for 24 hours at 37° C. in a humidified atmosphere containing 5% CO₂, followed by IGF-1 quantification by ELISA in cell culture supernatants (Mediagnost, Germany; Cat. #E20).

Results

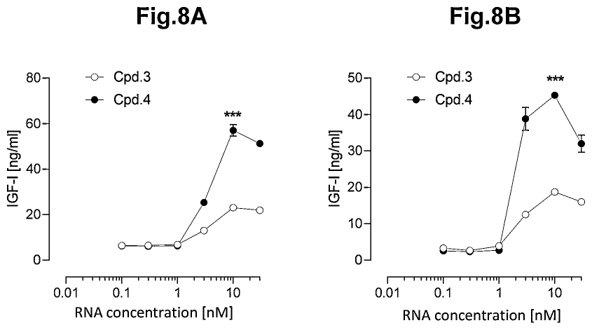

Dose response results showed that the effect of A2-linker in Cpd.4 on IGF-1 expression is significantly higher than the effect of A1-linker in Cpd.3 in HSMM cells cultivated in HSMM growth medium (FIG. 8A; P<0.001; Cmax at 10 nM is 57 ng/mL vs. 23 ng/mL). The effect of A2-linker over A1-linker in IGF-1 expression in differentiated HSMM was conducted in HSMM cells cultivated in differentiation medium and the results show that the level of IGF-1 expression and secretion from cells treated with Cpd.4 containing A2-linker is 2-fold higher than the level of IGF-1 expression and secretion from cells treated with Cpd.3 containing A1-linker at 10 nM concentration (FIG. 8B, P<0.001).

### Example 10: The Time-Course Effect of A1 and A2 Linker on Cytokine Expression and RNA Interference of Multiple siRNA Targets in In Vitro Glioblastoma Cancer Model, A172 Cells

In Vitro Transfection of A172 Cells

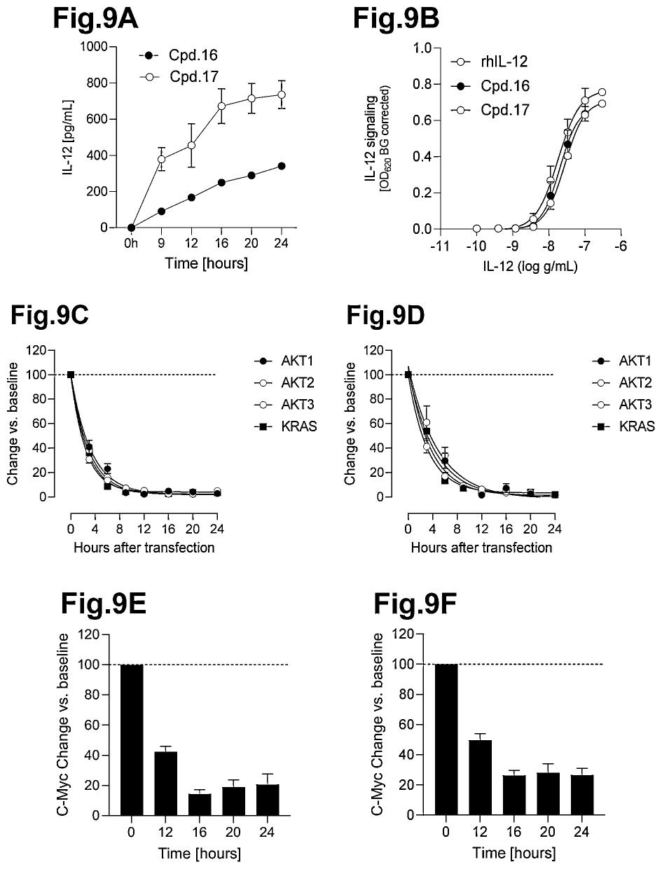

Human glioblastoma cell line (A172; ECACC, Cat. #88062428) was derived from a glioblastoma removed from a 53-year-old male. A172 cells were maintained in RPMI 1640 medium supplemented with 10% FBS and 2 mM glutamine. Cells were seeded at 20,000 cell/well in a 96 well culture plate and incubated at 37° C. in a humidified atmosphere containing 5% CO₂ for 24 hours prior to transfection. Cells were grown in RPMI growth medium to reach confluency <70% before transfection. Thereafter, A172 cells were transfected with Cpd.16 (A1 linker; IL-12 mRNA+1× c-Myc siRNA+1×KRAS siRNA+1× pan-Akt siRNA) and Cpd.17 (A2 linker; IL-12 mRNA+1× c-Myc siRNA+1×KRAS siRNA+1× pan-Akt siRNA) with the concentration of 10 nM using Lipofectamine 2000 (Invitrogen) following the manufacturer's instructions with the RNA to Lipofectamine ratio of 1:1 w/v. 100 μl of RPMI was removed and replaced with 90 μl of Opti-MEM (Thermo Fisher Scientific, Switzerland, Cat #31985-070) and 10 μl RNA and Lipofectamine 2000 complex in Opti-MEM. After 6 hours, the medium was replaced by fresh growth medium and the plates were incubated at 37° C. in a humidified atmosphere containing 5% CO 2 for 24 hours. For time-course assessment, samples were collected from 0-24 hours as specified in FIG. 9A. ELISA was performed to quantify human IL-12p70 (ThermoFisher Cat. #88-7126) levels present in the cell culture supernatant. The respective cell lysates were also processed to measure RNA abundance of siRNA target genes (c-Myc, KRAS, Akt1, Akt2 and Akt3 by relative quantification against untransfected samples by RT-qPCR using Cells-to-CT™ 1-Step Power SYBR Green kit (ThermoFisher Cat. #A25599) and primers (primer sequence details are listed in Table 5). The human 18s rRNA was used as a reference control. One phase decay analysis was performed in GraphPad Prism v.9.2 to calculate the half-life of siRNA of target genes post-transfection.

TABLE 5
Primers used in qPCR assay

| | | | SEQ |
|---|---|---|---|
| Gene | Primer | | ID |
| Name | Direction | Sequence (5′ to 3′) | NO |
| KRAS | Forward | GTACAGTGCAATGAGGGACCA | 139 |
| | Reverse | CACAAAGAAAGCCCTCCCCA | 140 |
| Akt1 | Forward | GAGATGGACTTCCGGTCGG | 141 |
| | Reverse | GTCACGCGGTGCTTGGG | 142 |
| Akt2 | Forward | CGAGGACCCCATGGACTACA | 143 |
| | Reverse | TTTAGCCCGTGCCTTGCTG | 144 |
| Akt3 | Forward | GGCAAGAAGAGGAGAGAATGAAT | 145 |
| | Reverse | TGGGTTGTAGAGGCATCCATC | 146 |
| c-Myc | Forward | ACTGTATGTGGAGCGGCTTC | 147 |
| | Reverse | CAGGTACAAGCTGGAGGTGG | 148 |
| 18s | Forward | ACCCGTTGAACCCCATTCGTGA | 149 |
| | Reverse | GCCTCACTAAACCATCCAATCGG | 150 |

Results

The time-course effect of Cpd.16 (A1-linker) and Cpd.17 (A2-linker) comprising IL-12 mRNA with 1× siRNA targeting c-Myc, 1× siRNA targeting KRAS, and 1× siRNA targeting pan-Akt (Akt1, Akt2, and Akt3) was evaluated for IL-12 expression and simultaneous downregulation of target genes in A172 cells by transfecting A172 cells with 10 nM of Cpd. 16 or Cpd. 17. The data demonstrate that cells transfected with Cpd.17 (A2-linker) expresses higher level of IL-12 protein (1.4 to 4-fold) than cells transfected with Cpd.16 (A1-linker) at all timepoints tested as shown in FIG. 9A. In the same cell lysate, the RNA interference of Cpd.16 and Cpd.17 against c-Myc, KRAS and pan-Akt (Akt1, Akt2, and Akt3) RNA transcripts was also assessed. As demonstrated in FIGS. 9C-9D, both Cpd.16 and Cpd.17 downregulated endogenous Akt-1, Akt-2, Akt-3, and KRAS by >95% at 24 hour timepoint. One phase analysis revealed that Cpd.16 with A1-linker achieves RNA interference of target genes an hour or two faster than A2-linker (t_{1/2}: 1.7-2.3 h vs. 2.2-3.4 h) but expresses reduced level of IL-12 compared to Cpd.17 with A2-linker. RNA interference of c-Myc targeted by Cpd.16 and Cpd.17 was observed after 12 hours and sustainly decreased endogenous c-Myc mRNA by up to 24 hours (27% vs. 21%). The design of pan-Akt siRNA to target all the three Akt genes (Akt1, Akt2, and Akt3) was successful and experimentally validated, demonstrating the feasibility of designing a specific siRNA targeting multiple genes with similar sequences.

### Example 11: Functional Analysis of IL-12 Derived from Compounds with A1- and A2-Linker in HEK-Blue™ hIL-12 Reporter Assay for STAT4 Activation

HEK-Blue™ hIL-12 Reporter Assay

The functional activity of IL-12 derived from Cpd.16 with A1-linker and Cpd.17 with A2-linker was tested in HEK-Blue™ hIL-12 reporter cells (Invivogen, Cat. Code: hkb-i112), which are designed for studying the activation of human IL-12 receptor by monitoring the activation of STAT-4 pathway. These cells were derived from the human embryonic kidney HEK293 cell line and engineered to express human IL-12R131 and IL-2R132 genes, together with the human JAK2 and STAT4 genes to achieve a totally functional IL-12 signaling cascade. In addition, a STAT4-inducible SEAP reporter gene was introduced. Upon IL-12 activation followed by STAT4, produced SEAP can be determined in real-time with HEK-Blue™ Detection cell culture medium in cell culture supernatant. Stimulation of HEK-Blue™ hIL-12 cells were achieved by recombinant human IL-12 (rhIL-12) or IL-12 derived from cell culture supernatant of HEK293 cells which had been transfected with Cpd.16 or Cpd.17 (0.3 μg/well) with below details.

HEK-Blue™ hIL-2 cells were maintained in Dulbecco's Modified Eagle's medium (DMEM, Sigma Aldrich) supplemented with 10% (v/v) Fetal Bovine Serum (FBS). The antibiotics HEK-Blue™ selection (1:250 dilution with media) were added to the media to select cells containing IL-12R131, IL-2R132, STAT4 and SEAP transgene plasmids. Cells were seeded at 40,000 cell/well in a 96 well culture plate and incubated at 37° C. in a humidified atmosphere containing 5% CO₂ for 24 hours prior to stimulation. Cells were grown in DMEM growth medium containing 10% of FBS to reach confluency <80% before stimulation. Defined concentration (0.001-300 ng) of IL-12 derived from HEK293 cell culture supernatant were collected, diluted in 20 μl of media, and added to culture media of HEK-Blue™ hIL-12 cells to measure IL-12 receptor recruitment followed by STAT4 pathway activation. The rhIL-12 or IL-12 derived from Cpd.16 and Cpd.17 (0.001-300 ng) were tested in parallel. After 2 hours of incubation, SEAP activity was assessed using QUANTI-Blue™ (20 μl cell culture supernatant+180 μl QUANTI-Blue™ solution) and reading the optical density (O.D.) at 620 nm in SpectraMax i3 multi-mode plate reader (Molecular Device). Untransfected samples were used as background control and subtracted from obtained O.D. values in tested samples.

Results

Stimulation of HEK-Blue™ hIL-12 cells with rhIL-12 or IL-12 derived from cell culture supernatant of HEK293 cells that had been transfected with Cpd.16 (A1-linker) or Cpd.17 (A2-linker) was functional as all three tested compounds induced SEAP production in a similar dose-dependent fashion. There was no linker-dependent impact observed on IL-12 functionality (FIG. 9B). In summary, IL-12 derived from Cpd.16 and Cpd.17 are functional and can induce IL-12 signaling cascade similarly to rhIL-2.

The examples and embodiments described herein are for illustrative purposes only and various modifications or changes suggested to persons skilled in the art are to be included within the spirit and purview of this application and scope of the appended claims.

TABLE 6
Table of Sequences Listed

| Protein or | | SEQ ID |
|---|---|---|
| Nucleic Acid | Sequence | NO: |
| Compound 1-17 | See Table 2 and Table 3 | 1-18 |
| nucleic acid | | and 85-100 |
| sequences | | |
| Kozak sequence | GCCACC | 19 |
| T7 promoter | TAATACGACTCACTATA | 20 |
| A1-linker: | ATAGTGAGTCGTATTAACGTACCAACAA | 21 |
| mRNA to | | |
| siRNA linker | | |
| A1-linker: | TTTATCTTAGAGGCATATCCCTACGTACCAACAA | 22 |
| siRNA to | | |
| siRNA linker | | |
| A2-linker | ACAACAA | 23 |
| Forward Primer | GCTGCAAGGCGATTAAGTTG | 24 |
| Reverse Primer | U(2′OMe)U(2′OMe)U(2′OMe)TTTTTTTTTTTTTTTTTTTTTTTTT | 25 |
| | TTTTTTTTTTTTTTTTTTTTTTTTTTTTTTTTTTTTTTTTTTTTTTTTT | |
| | TTTTTTTTTTTTTTTTTTTTTTTTTTTTTTTTTTTTTTCAGCTA | |
| | TGACCATGTTAATGCAG | |
| IL-4 | ATCGTTAGCTTCTCCTGATAAACTAATTGCCTCACATTGTCACTGCAAA | 26 |
| Human IL-4 | TCGACACCTATTAATGGGTCTCACCTCCCAACTGCTTCCCCCTCTGTTC | |
| Nucleotide | TTCCTGCTAGCATGTGCCGGCAACTTTGTCCACGGACACAAGTGCGATA | |
| (Genbank | TCACCTTACAGGAGATCATCAAAACTTTGAACAGCCTCACAGAGCAGAA | |
| NM_000589.4) | GACTCTGTGCACCGAGTTGACCGTAACAGACATCTTTGCTGCCTCCAAG | |
| | AACACAACTGAGAAGGAAACCTTCTGCAGGGCTGCGACTGTGCTCCGGC | |
| | AGTTCTACAGCCACCATGAGAAGGACACTCGCTGCCTGGGTGCGACTGC | |
| | ACAGCAGTTCCACAGGCACAAGCAGCTGATCCGATTCCTGAAACGGCTC | |
| | GACAGGAACCTCTGGGGCCTGGCGGGCTTGAATTCCTGTCCTGTGAAGG | |
| | AAGCCAACCAGAGTACGTTGGAAAACTTCTTGGAAAGGCTAAAGACGAT | |
| | CATGAGAGAGAAATATTCAAAGTGTTCGAGCTGAATATTTTAATTTATG | |
| | AGTTTTTGATAGCTTTATTTTTTAAGTATTTATATATTTATAACTCATC | |
| | ATAAAATAAAGTATATATAGAATCTAA | |
| IL-4 | MGLTSQLLPPLFFLLACAGNFVHGHKCDITLQEIIKTLNSLTEQKTLCT | 27 |
| Human IL-4 | ELTVTDIFAASKNTTEKETFCRAATVLRQFYSHHEKDTRCLGATAQQFH | |
| amino acid | RHKQLIRFLKRLDRNLWGLAGLNSCPVKEANOSTLENFLERLKTIMREK | |
| (Genbank | YSKCSS | |
| NP_000580.1) | | |
| Underlined: | | |
| signal sequence | | |
| IGF-1 | ATGGGAAAAATCAGCAGTCTTCCAACCCAATTATTTAAGTGCTGCTTTT | 28 |
| Human IGF-1 | GTGATTTCTTGAAGGTGAAGATGCACACCATGTCCTCCTCGCATCTCTT | |
| Nucleotide | CTACCTGGCGCTGTGCCTGCTCACCTTCACCAGCTCTGCCACGGCTGGA | |
| (Genbank | CCGGAGACGCTCTGCGGGGCTGAGCTGGTGGATGCTCTTCAGTTCGTGT | |
| NM_000618.4) | GTGGAGACAGGGGCTTTTATTTCAACAAGCCCACAGGGTATGGCTCCAG | |
| | CAGTCGGAGGGCGCCTCAGACAGGCATCGTGGATGAGTGCTGCTTCCGG | |
| | AGCTGTGATCTAAGGAGGCTGGAGATGTATTGCGCACCCCTCAAGCCTG | |
| | CCAAGTCAGCTCGCTCTGTCCGTGCCCAGCGCCACACCGACATGCCCAA | |
| | GACCCAGAAGGAAGTACATTTGAAGAACGCAAGTAGAGGGAGTGCAGGA | |
| | AACAAGAACTACAGGATGTAG | |
| IGF-1 | MGKISSLPTQLFKCCFCDFLKVKMHTMSSSHLFYLALCLLTFTSSATAG | 29 |
| Human IGF-1 | PETLCGAELVDALQFVCGDRGFYFNKPTGYGSSSRRAPQTGIVDECCFR | |
| amino acid | SCDLRRLEMYCAPLKPAKSARSVRAQRHTDMPKTQKEVHLKNASRGSAG | |
| (Genbank | NKNYRM | |
| NP_000609.1) | | |
| Underlined: | | |
| signal sequence | | |
| IGF-1 | ATGACCATCCTGTTTCTGACAATGGTCATCAGCTACTTCGGCTGCATGA | 30 |
| Human IGF-1 | AGGCCGTGAAGATGCACACCATGAGCAGCAGCCACCTGTTCTATCTGGC | |
| Nucleotide | CCTGTGCCTGCTGACCTTTACCAGCTCTGCTACCGCCGGACCTGAGACA | |
| (Optimized | CTTTGTGGCGCTGAACTGGTGGACGCCCTGCAGTTTGTGTGTGGCGACA | |
| IGF1 with | GAGGCTTCTACTTCAACAAGCCCACAGGCTACGGCAGCAGCTCTAGAAG | |
| BDNF SP and | GGCTCCTCAGACCGGAATCGTGGACGAGTGCTGCTTCAGAAGCTGCGAC | |
| without E- | CTGCGGCGGCTGGAAATGTATTGTGCCCCTCTGAAGCCTGCCAAGAGCG | |
| peptide) | CCTAA | |
| IGF-1 | MTILFLTMVISYFGCMKAVKMHTMSSSHLFYLALCLLTFTSSATAGPET | 31 |
| Human IGF-1 | LCGAELVDALQFVCGDRGFYFNKPTGYGSSSRRAPQTGIVDECCFRSCD | |
| amino acid | LRRLEMYCAPLKPAKSA | |
| (Optimized | | |
| IGF1 with | | |
| BDNF SP and | | |
| without E- | | |
| peptide) | | |
| Underlined: | | |
| signal sequence | | |
| Human TNF-a | ATGAGCACTGAAAGCATGATCCGGGACGTGGAGCTGGCCGAGGAGGCGC | 32 |
| Nucleotide | TCCCCAAGAAGACAGGGGGGCCCCAGGGCTCCAGGCGGTGCTTGTTCCT | |
| (Genbank | CAGCCTCTTCTCCTTCCTGATCGTGGCAGGCGCCACCACGCTCTTCTGC | |
| NM_000594.3) | CTGCTGCACTTTGGAGTGATCGGCCCCCAGAGGGAAGAGTTCCCCAGGG | |
| Bold and | ACCTCTCTCTAATCAGCCCTCTGGCCCAGGCAGTCAGATCATCTTCTCG | |
| italicized: | AACCCCGAGTGACAAGCCTGTAGCCCATGTTGTAGCAAACCCTCAAGCT | |
| siRNA binding | GAGGGGCAGCTCCAGTGGCTGAACCGCCGGGCCAATGCCCTCCTGGCCA | |
| regions | ATGGCGTGGAGCTGAGAGATAACCAGCTGGTGGTGCCATCAGAGGGCCT | |
| | GTACCTCATCTACTCCCAGGTCCTCTTCAAGGGCCAAGGCTGCCCCTCC | |
| | ACCCATGTGCTCCTCACCCACACCATCAGCCGCATCGCCGTCTCCTACC | |
| | AGACCAAGGTCAACCTCCTCTCTGCCATCAAGAGCCCCTGCCAGAGGGA | |
| | GACCCCAGAGGGGGCTGAGGCCAAGCCCTGGTATGAGCCCATCTATCTG | |
| | GGAGGGGTCTTCCAGCTGGAGAAGGGTGACCGACTCAGCGCTGAGATCA | |
| | ATCGGCCCGACTATCTCGACTTTGCCGAGTCTGGGCAGGTCTACTTTGG | |
| | GATCATTGCCCTGTGA | |
| Human ALK2 | ATGGTAGATGGAGTGATGATTCTTCCTGTGCTTATCATGATTGCTCTCC | 33 |
| Nucleotide | CCTCCCCTAGTATGGAAGATGAGAAGCCCAAGGTCAACCCCAAACTCTA | |
| (Genbank | CATGTGTGTGTGTGAAGGTCTCTCCTGCGGTAATGAGGACCACTGTGAA | |
| NM_001105.4) | GGCCAGCAGTGCTTTTCCTCACTGAGCATCAACGATGGCTTCCACGTCT | |
| Bold and | ACCAGAAAGGCTGCTTCCAGGTTTATGAGCAGGGAAAGATGACCTGTAA | |
| italicized: | GACCCCGCCGTCCCCTGGCCAAGCCGTGGAGTGCTGCCAAGGGGACTGG | |
| siRNA binding | TGTAACAGGAACATCACGGCCCAGCTGCCCACTAAAGGAAAATCCTTCC | |
| regions | CTGGAACACAGAATTTCCACTTGGAGGTTGGCCTCATTATTCTCTCTGT | |
| | AGTGTTCGCAGTATGTCTTTTAGCCTGCCTGCTGGGAGTTGCTCTCCGA | |
| | AAATTTAAAAGGCGCAACCAAGAACGCCTCAATCCCCGAGACGTGGAGT | |
| | ATGGCACTATCGAAGGGCTCATCACCACCAATGTTGGAGACAGCACTTT | |
| | AGCAGATTTATTGGATCATTCGTGTACATCAGGAAGTGGCTCTGGTCTT | |
| | CCTTTTCTGGTACAAAGAACAGTGGCTCGCCAGATTACACTGTTGGAGT | |
| | GTGTCGGGAAAGGCAGGTATGGTGAGGTGTGGAGGGGCAGCTGGCAAGG | |
| | GGAGAATGTTGCCGTGAAGATCTTCTCCTCCCGTGATGAGAAGTCATGG | |
| | TTCAGGGAAACGGAATTGTACAACACTGTGATGCTGAGGCATGAAAATA | |
| | TCTTAGGTTTCATTGCTTCAGACATGACATCAAGACACTCCAGTACCCA | |
| | GCTGTGGTTAATTACACATTATCATGAAATGGGATCGTTGTACGACTAT | |
| | CTTCAGCTTACTACTCTGGATACAGTTAGCTGCCTTCGAATAGTGCTGT | |
| | CCATAGCTAGTGGTCTTGCACATTTGCACATAGAGATATTTGGGACCCA | |
| | AGGGAAACCAGCCATTGCCCATCGAGATTTAAAGAGCAAAAATATTCTG | |
| | GTTAAGAAGAATGGACAGTGTTGCATAGCAGATTTGGGCCTGGCAGTCA | |
| | TGCATTCCCAGAGCACCAATCAGCTTGATGTGGGGAACAATCCCCGTGT | |
| | GGGCACCAAGCGCTACATGGCCCCCGAAGTTCTAGATGAAACCATCCAG | |
| | GTGGATTGTTTCGATTCTTATAAAAGGGTCGATATTTGGGCCTTTGGAC | |
| | TTGTTTTGTGGGAAGTGGCCAGGCGGATGGTGAGCAATGGTATAGTGGA | |
| | GGATTACAAGCCACCGTTCTACGATGTGGTTCCCAATGACCCAAGTTTT | |
| | GAAGATATGAGGAAGGTAGTCTGTGTGGATCAACAAAGGCCAAACATAC | |
| | CCAACAGATGGTTCTCAGACCCGACATTAACCTCTCTGGCCAAGCTAAT | |
| | GAAAGAATGCTGGTATCAAAATCCATCCGCAAGACTCACAGCACTGCGT | |
| | ATCAAAAAGACTTTGACCAAAATTGATAATTCCCTCGACAAATTGAAAA | |
| | CTGACTGTTGA | |
| Turbo GFP | ATGGAGAGCGACGAGAGCGGCCTGCCCGCCATGGAGATCGAGTGCCGCA | 34 |
| Bold and | TCACCGGCACCCTGAACGGCGTGGAGTTCGAGCTGGTGGGCGGCGGAGA | |
| italicized: | GGGCACCCCCGAGCAGGGCCGCATGACCAACAAGATGAAGAGCACCAAA | |
| siRNA binding | GGCGCCCTGACCTTCAGCCCCTACCTGCTGAGCCACGTGATGGGCTACG | |
| regions | GCTTCTACCACTTCGGCACCTACCCCAGCGGCTACGAGAACCCCTTCCT | |
| | GCACGCCATCAACAACGGCGGCTACACCAACACCCGCATCGAGAAGTAC | |
| | GAGGACGGCGGCGTGCTGCACGTGAGCTTCAGCTACCGCTACGAGGCCG | |
| | GCCGCGTGATCGGCGACTTCAAGGTGATGGGCACCGGCTTCCCCGAGGA | |
| | CAGCGTGATCTTCACCGACAAGATCATCCGCAGCAACGCCACCGTGGAG | |
| | CACCTGCACCCCATGGGCGATAACGATCTGGATGGCAGCTTCACCCGCA | |
| | CCTTCAGCCTGCGCGACGGCGGCTACTACAGCTCCGTGGTGGACAGCCA | |
| | CATGCACTTCAAGAGCGCCATCCACCCCAGCATCCTGCAGAACGGGGGC | |
| | CCCATGTTCGCCTTCCGCCGCGTGGAGGAGGATCACAGCAACACCGAGC | |
| | TGGGCATCGTGGAGTACCAGCACGCCTTCAAGACCCCGGATGCAGATGC | |
| | CGGTGAAGAATAA | |
| ALK2 mRNA | GACGTGGAGTATGGCACTATCG | 35 |
| Forward primer | | |
| ALK2 mRNA | CACTCCAACAGTGTAATCTGGCG | 36 |
| Reverse primer | | |
| human 18S | ACCCGTTGAACCCCATTCGTGA | 37 |
| rRNA Forward | | |
| primer | | |
| human 18S | GCCTCACTAAACCATCCAATCGG | 38 |
| rRNA Reverse | | |
| primer | | |
| tRNA linker | AACAAAGCACCAGTGGTCTAGTGGTAGAATAGTACCCTGCCACGGTACA | 39 |
| | GACCCGGGTTCGATTCCCGGCTGGTGCA | |
| A mature human | GGACCTGAGACACTTTGTGGCGCTGAACTGGTGGACGCCCTGCAGTTTG | 40 |
| IGF-1 coding | TGTGTGGCGACAGAGGCTTCTACTTCAACAAGCCCACAGGCTACGGCAG | |
| sequence | CAGCTCTAGAAGGGCTCCTCAGACCGGAATCGTGGACGAGTGCTGTTTC | |
| | AGAAGCTGCGACCTGCGGCGGCTGGAAATGTATTGTGCCCCTCTGAAGC | |
| | CTGCCAAGAGCGCC | |
| A modified | MLILLLPLLLFKCFCDFLK | 41 |
| signal peptide of | | |
| IGF-1 | | |
| A modified | ATGCTGATTCTGCTGCTGCCCCTGCTGCTGTTCAAGTGCTTCTGCGACT | 42 |
| signal peptide of | TCCTGAAA | |
| IGF-1-coding | | |
| sequence | | |
| A modified | MLFYLALCLLTFTSSATA | 43 |
| IGF-1 pro | | |
| domain | | |
| A modified | ATGCTGTTCTATCTGGCCCTGTGCCTGCTGACCTTTACCAGCTCTGCTAC | 44 |
| IGF-1 pro | CGCC | |
| domain-coding | | |
| sequence | | |
| IGF-1 pro | VKMHTMSSSH | 45 |
| domain | | |
| sequence that is | | |
| deleted | | |
| WT IGF-1 | MGKISSLPTQLFKCCFCDFLK | 46 |
| signal peptide | | |
| WT IGF-1 | ATGGGAAAAATCAGCAGTCTTCCAACCCAATTATTTAAGTGCTGCTTTT | 47 |
| signal peptide | GTGATTTCTTGAAG | |
| coding sequence | | |
| A modified | MTILFLTMVISYFGCMKA | 48 |
| signal peptide of | | |
| IGF-1 | | |
| A modified | ATGACCATCCTGTTTCTGACAATGGTCATCAGCTACTTCGGCTGCATGA | 49 |
| signal peptide of | AGGCC | |
| IGF-1-coding | | |
| sequence | | |
| TNF-alpha | GGCGTGGAGCTGAGAGATAA | 50 |
| siRNA sense | | |
| strand (Cpd. 1-1 | | |
| siRNA and | | |
| Cpd.2-1 siRNA) | | |
| TNF-alpha | GGGCCTGTACCTCATCTACT | 51 |
| siRNA sense | | |
| strand (Cpd.1-2 | | |
| siRNA and | | |
| Cpd.2-2 siRNA) | | |
| TNF-alpha | GGTATGAGCCCATCTATCT | 52 |
| siRNA sense | | |
| strand (Cpd. 1-3 | | |
| siRNA and | | |
| Cpd.2-3 siRNA) | | |
| ALK-2 siRNA | GGCCTCATTATTCTCTCT | 53 |
| sense strand | | |
| (Cpd.3-1 siRNA | | |
| and Cpd.4-1 | | |
| siRNA) | | |
| ALK-2 siRNA | GTGTTCGCAGTATGTCTT | 54 |
| sense strand | | |
| (Cpd.3-2 siRNA | | |
| and Cpd.4-2 | | |
| siRNA) | | |
| ALK-2 siRNA | GCCTGCCTGCTGGGAGTT | 55 |
| sense strand | | |
| (Cpd.3-3 siRNA | | |
| and Cpd.4-3 | | |
| siRNA) | | |
| Turbo GFP | CAACAAGATGAAGAGCACCAA | 56 |
| siRNA sense | | |
| strand (Cpd.5-1 | | |
| siRNA, Cpd.6-1 | | |
| siRNA, Cpd.7-1 | | |
| siRNA, Cpd.8-1 | | |
| siRNA and | | |
| Cpd.9-1 siRNA) | | |
| Turbo GFP | GGACAGCCACATGCACTTCAA | 57 |
| siRNA sense | | |
| strand (Cpd.8-2 | | |
| siRNA) | | |
| TNF-alpha | TTATCTCTCAGCTCCACGCC | 58 |
| siRNA anti- | | |
| sense strand | | |
| (Cpd.1-1 siRNA | | |
| and Cpd.2-1 | | |
| siRNA) | | |
| TNF-alpha | AGTAGATGAGGTACAGGCCC | 59 |
| siRNA anti- | | |
| sense strand | | |
| (Cpd.1-2 siRNA | | |
| and Cpd.2-2 | | |
| siRNA) | | |
| TNF-alpha | AGATAGATGGGCTCATACC | 60 |
| siRNA anti- | | |
| sense strand | | |
| (Cpd.1-3 siRNA | | |
| and Cpd.2-3 | | |
| siRNA) | | |
| ALK-2 siRNA | AGAGAGAATAATGAGGCC | 61 |
| anti-sense strand | | |
| (Cpd.3-1 siRNA | | |
| and Cpd.4-1 | | |
| siRNA) | | |
| ALK-2 siRNA | AAGACATACTGCGAACAC | 62 |
| anti-sense strand | | |
| (Cpd.3-2 siRNA | | |
| and Cpd.4-2 | | |
| siRNA) | | |
| ALK-2 siRNA | AACTCCCAGCAGGCAGGC | 63 |
| anti-sense strand | | |
| (Cpd.3-3 siRNA | | |
| and Cpd.4-3 | | |
| siRNA) | | |
| Turbo GFP | TTGGTGCTCTTCATCTTGTTG | 64 |
| siRNA anti- | | |
| sense strand | | |
| (Cpd.5-1 | | |
| siRNA, Cpd.6-1 | | |
| siRNA, Cpd.7-1 | | |
| siRNA, Cpd.8-1 | | |
| siRNA and | | |
| Cpd.9-1 siRNA) | | |
| Turbo GFP | TTGAAGTGCATGTGGCTGTCC | 65 |
| siRNA anti- | | |
| sense strand | | |
| (Cpd.8-2 | | |
| siRNA) | | |
| Linker | ATAGTGAGTCGTATTA | 66 |
| Linker (B) | ATCCCTACGTACCAACAA | 67 |
| Linker (C) | ACGTACCAACAA | 68 |
| Linker (D) | TCCC | 69 |
| Linker (E) | ACAACAATCCC | 70 |
| Linker | CAACAA | 71 |
| Linker | ATAGTGAGTCGTATTATCCC | 72 |
| Linker | ATAGTGAGTCGTATTAACAACAATCCC | 73 |
| Linker | ATAGTGAGTCGTATTAACAACAA | 74 |
| Linker | ATAGTGAGTCGTATTAATCCCTACGTACCAACAA | 75 |
| Compound 1 | GCCACCAUGGGACUGACAUCUCAACUGCUGCCUCCACUGUUCUUUCUGCUGGCC | 76 |
| RNA sequence | UGCGCCGGCAAUUUUGUGCACGGCCACAAGUGCGACAUCACCCUGCAAGAGAUC | |
| Bold = Sense | AUCAAGACCCUGAACAGCCUGACCGAGCAGAAAACCCUGUGCACCGAGCUGACC | |
| siRNA strand | GUGACCGAUAUCUUUGCCGCCAGCAAGAACACAACCGAGAAAGAGACAUUCUGC | |
| Bold and Italics = | AGAGCCGCCACCGUGCUGAGACAGUUCUACAGCCACCACGAGAAGGACACCAGA | |
| anti-Sense siRNA | UGCCUGGGAGCUACAGCCCAGCAGUUCCACAGACACAAGCAGCUGAUCCGGUUC | |
| strand | CUGAAGCGGCUGGACAGAAAUCUGUGGGGACUCGCCGGCCUGAAUAGCUGCCCU | |
| Underline = | GUGAAAGAGGCCAACCAGUCUACCCUGGAAAACUUCCUGGAACGGCUGAAAACC | |
| Signal peptide | AUCAUGCGCGAGAAGUACAGCAAGUGCAGCAGCUGAAUAGUGAGUCGUAUUAAC | |
| Italics = Kozak | GUACCAACAAGGCGUGGAGCUGAGAGAUAAACUUGUUAUCUCUCAGCUCCACGC | |
| sequence | CUUUAUCUUAGAGGCAUAUCCCUACGUACCAACAAGGGCCUGUACCUCAUCUAC | |
| | UACUUGAGUAGAUGAGGUACAGGCCCUUUAUCUUAGAGGCAUAUCCCUACGUAC | |
| | CAACAAGGUAUGAGCCCAUCUAUCUACUUGAGAUAGAUGGGCUCAUACCUUUAU | |
| | CUUAGAGGCAUAUCCCUUUUAUCUUAGAGGCAUAUCCCU | |
| | (all Us are modified; N¹-methylpseudouridine) | |
| Compound 2 | GCCACCAUGGGACUGACAUCUCAACUGCUGCCUCCACUGUUCUUUCUGCUGGCC | 77 |
| RNA sequence | UGCGCCGGCAAUUUUGUGCACGGCCACAAGUGCGACAUCACCCUGCAAGAGAUC | |
| Bold = Sense | AUCAAGACCCUGAACAGCCUGACCGAGCAGAAAACCCUGUGCACCGAGCUGACC | |
| siRNA strand | GUGACCGAUAUCUUUGCCGCCAGCAAGAACACAACCGAGAAAGAGACAUUCUGC | |
| Bold and Italics = | AGAGCCGCCACCGUGCUGAGACAGUUCUACAGCCACCACGAGAAGGACACCAGA | |
| anti-Sense siRNA | UGCCUGGGAGCUACAGCCCAGCAGUUCCACAGACACAAGCAGCUGAUCCGGUUC | |
| strand | CUGAAGCGGCUGGACAGAAAUCUGUGGGGACUCGCCGGCCUGAAUAGCUGCCCU | |
| Underline = | GUGAAAGAGGCCAACCAGUCUACCCUGGAAAACUUCCUGGAACGGCUGAAAACC | |
| Signal peptide | AUCAUGCGCGAGAAGUACAGCAAGUGCAGCAGCUGAACAACAAGGCGUGGAGCU | |
| Italics = Kozak | GAGAGAUAAACUUGUUAUCUCUCAGCUCCACGCCACAACAAGGGCCUGUACCUC | |
| sequence | AUCUACUACUUGAGUAGAUGAGGUACAGGCCCACAACAAGGUAUGAGCCCAUCU | |
| | AUCUACUUGAGAUAGAUGGGCUCAUACCACAACAAUUUAUCUUAGAGGCAUAUC | |
| | CCU | |
| | (all Us are modified; N¹-methylpseudouridine) | |
| Compound 3 | GCCACCAUGACCAUCCUGUUUCUGACAAUGGUCAUCAGCUACUUCGGCUGCAUG | 78 |
| RNA sequence | AAGGCCGUGAAGAUGCACACCAUGAGCAGCAGCCACCUGUUCUAUCUGGCCCUG | |
| Bold = Sense | UGCCUGCUGACCUUUACCAGCUCUGCUACCGCCGGACCUGAGACACUUUGUGGC | |
| siRNA strand | GCUGAACUGGUGGACGCCCUGCAGUUUGUGUGUGGCGACAGAGGCUUCUACUUC | |
| Bold and Italics = | AACAAGCCCACAGGCUACGGCAGCAGCUCUAGAAGGGCUCCUCAGACCGGAAUC | |
| anti-Sense siRNA | GUGGACGAGUGCUGCUUCAGAAGCUGCGACCUGCGGCGGCUGGAAAUGUAUUGU | |
| strand | GCCCCUCUGAAGCCUGCCAAGAGCGCCUAAAUAGUGAGUCGUAUUAACGUACCA | |
| Underline = | ACAAGGCCUCAUUAUUCUCUCUACUUGAGAGAGAAUAAUGAGGCCUUUAUCUUA | |
| Signal peptide | GAGGCAUAUCCCUACGUACCAACAAGUGUUCGCAGUAUGUCUUACUUGAAGACA | |
| Italics = Kozak | UACUGCGAACACUUUAUCUUAGAGGCAUAUCCCUACGUACCAACAAGCCUGCCU | |
| sequence | GCUGGGAGUUACUUGAACUCCCAGCAGGCAGGCUUUAUCUUAGAGGCAUAUCCC | |
| | UUUUAUCUUAGAGGCAUAUCCCU | |
| | (all Us are modified; N¹-methylpseudouridine) | |
| Compound 4 | GCCACCAUGACCAUCCUGUUUCUGACAAUGGUCAUCAGCUACUUCGGCUGCAUG | 79 |
| RNA sequence | AAGGCCGUGAAGAUGCACACCAUGAGCAGCAGCCACCUGUUCUAUCUGGCCCUG | |
| Bold = Sense | UGCCUGCUGACCUUUACCAGCUCUGCUACCGCCGGACCUGAGACACUUUGUGGC | |
| siRNA strand | GCUGAACUGGUGGACGCCCUGCAGUUUGUGUGUGGCGACAGAGGCUUCUACUUC | |
| Bold and Italics = | AACAAGCCCACAGGCUACGGCAGCAGCUCUAGAAGGGCUCCUCAGACCGGAAUC | |
| anti-Sense siRNA | GUGGACGAGUGCUGCUUCAGAAGCUGCGACCUGCGGCGGCUGGAAAUGUAUUGU | |
| strand | GCCCCUCUGAAGCCUGCCAAGAGCGCCUAAACAACAAGGCCUCAUUAUUCUCUC | |
| Underline = | UACUUGAGAGAGAAUAAUGAGGCCACAACAAGUGUUCGCAGUAUGUCUUACUUG | |
| Signal peptide | AAGACAUACUGCGAACACACAACAAGCCUGCCUGCUGGGAGUUACUUGAACUCC | |
| Italics = Kozak | CAGCAGGCAGGCACAACAAUUUAUCUUAGAGGCAUAUCCCU | |
| sequence | (all Us are modified; N¹-methylpseudouridine) | |
| Compound 5 | GCCACCAUGGGCAAGAUUAGCAGCCUGCCUACACAGCUGUUCAAGUGCUGCUUC | 80 |
| RNA sequence | UGCGACUUCCUGAAAGUGAAGAUGCACACCAUGAGCAGCAGCCACCUGUUCUAU | |
| Bold = Sense | CUGGCCCUGUGCCUGCUGACCUUUACCAGCUCUGCUACCGCCGGACCUGAGACA | |
| siRNA strand | CUUUGUGGCGCUGAACUGGUGGACGCCCUGCAGUUUGUGUGUGGCGACAGAGGC | |
| Bold and Italics = | UUCUACUUCAACAAGCCCACAGGCUACGGCAGCAGCUCUAGAAGGGCUCCUCAG | |
| anti-Sense siRNA | ACCGGAAUCGUGGACGAGUGCUGUUUCAGAAGCUGCGACCUGCGGCGGCUGGAA | |
| strand | AUGUAUUGUGCCCCUCUGAAGCCUGCCAAGAGCGCCUAAAUAGUGAGUCGUAUU | |
| Underline = | AACGUACCAACAACAACAAGAUGAAGAGCACCAAACUUGUUGGUGCUCUUCAUC | |
| Signal peptide | UUGUUGUUUAUCUUAGAGGCAUAUCCCUUUUAUCUUAGAGGCAUAUCCCU | |
| Italics = Kozak | (all Us are modified; N¹-methylpseudouridine) | |
| sequence | | |
| Compound 6 | GCCACCAUGGGCAAGAUUAGCAGCCUGCCUACACAGCUGUUCAAGUGCUGCUUC | 81 |
| RNA sequence | UGCGACUUCCUGAAAGUGAAGAUGCACACCAUGAGCAGCAGCCACCUGUUCUAU | |
| Bold = Sense | CUGGCCCUGUGCCUGCUGACCUUUACCAGCUCUGCUACCGCCGGACCUGAGACA | |
| siRNA strand | CUUUGUGGCGCUGAACUGGUGGACGCCCUGCAGUUUGUGUGUGGCGACAGAGGC | |
| Bold and Italics = | UUCUACUUCAACAAGCCCACAGGCUACGGCAGCAGCUCUAGAAGGGCUCCUCAG | |
| anti-Sense siRNA | ACCGGAAUCGUGGACGAGUGCUGUUUCAGAAGCUGCGACCUGCGGCGGCUGGAA | |
| strand | AUGUAUUGUGCCCCUCUGAAGCCUGCCAAGAGCGCCUAAACAACAACAACAAGA | |
| Underline = | UGAAGAGCACCAAACUUGUUGGUGCUCUUCAUCUUGUUGACAACAAUUUAUCUU | |
| Signal peptide | AGAGGCAUAUCCCU | |
| Italics = Kozak | (all Us are modified; N¹-methylpseudouridine) | |
| sequence | | |
| Compound 7 | GCCACCAUGGGCAAGAUUAGCAGCCUGCCUACACAGCUGUUCAAGUGCUGCUUC | 82 |
| RNA sequence | UGCGACUUCCUGAAAGUGAAGAUGCACACCAUGAGCAGCAGCCACCUGUUCUAU | |
| Bold = Sense | CUGGCCCUGUGCCUGCUGACCUUUACCAGCUCUGCUACCGCCGGACCUGAGACA | |
| siRNA strand | CUUUGUGGCGCUGAACUGGUGGACGCCCUGCAGUUUGUGUGUGGCGACAGAGGC | |
| Bold and Italics = | UUCUACUUCAACAAGCCCACAGGCUACGGCAGCAGCUCUAGAAGGGCUCCUCAG | |
| anti-Sense siRNA | ACCGGAAUCGUGGACGAGUGCUGUUUCAGAAGCUGCGACCUGCGGCGGCUGGAA | |
| strand | AUGUAUUGUGCCCCUCUGAAGCCUGCCAAGAGCGCCUAAAUAGUGAGUCGUAUU | |
| Underline = | AACGUACCAACAACAACAAGAUGAAGAGCACCAAACUUGUUGGUGCUCUUCAUC | |
| Signal peptide | UUGUUGUUUAUCUUAGAGGCAUAUCCCUACGUACCAACAACAACAAGAUGAAGA | |
| Italics = Kozak | GCACCAAACUUGUUGGUGCUCUUCAUCUUGUUGUUUAUCUUAGAGGCAUAUCCC | |
| sequence | UUUUAUCUUAGAGGCAUAUCCCU | |
| | (all Us are modified; N¹-methylpseudouridine) | |
| Compound 8 | GCCACCAUGGGCAAGAUUAGCAGCCUGCCUACACAGCUGUUCAAGUGCUGCUUC | 83 |
| RNA sequence | UGCGACUUCCUGAAAGUGAAGAUGCACACCAUGAGCAGCAGCCACCUGUUCUAU | |
| Bold = Sense | CUGGCCCUGUGCCUGCUGACCUUUACCAGCUCUGCUACCGCCGGACCUGAGACA | |
| siRNA strand | CUUUGUGGCGCUGAACUGGUGGACGCCCUGCAGUUUGUGUGUGGCGACAGAGGC | |
| Bold and Italics = | UUCUACUUCAACAAGCCCACAGGCUACGGCAGCAGCUCUAGAAGGGCUCCUCAG | |
| anti-Sense siRNA | ACCGGAAUCGUGGACGAGUGCUGUUUCAGAAGCUGCGACCUGCGGCGGCUGGAA | |
| strand | AUGUAUUGUGCCCCUCUGAAGCCUGCCAAGAGCGCCUAAAUAGUGAGUCGUAUU | |
| Underline = | AACGUACCAACAACAACAAGAUGAAGAGCACCAAACUUGUUGGUGCUCUUCAUC | |
| Signal peptide | UUGUUGUUUAUCUUAGAGGCAUAUCCCUACGUACCAACAAGGACAGCCACAUGC | |
| Italics = Kozak | ACUUCAAACUUGUUGAAGUGCAUGUGGCUGUCCUUUAUCUUAGAGGCAUAUCCC | |
| sequence | UUUUAUCUUAGAGGCAUAUCCCU | |
| | (all Us are modified; N¹-methylpseudouridine) | |
| Compound 9 | GCCACCAUGGGCAAGAUUAGCAGCCUGCCUACACAGCUGUUCAAGUGCUGCUUC | 84 |
| RNA sequence | UGCGACUUCCUGAAAGUGAAGAUGCACACCAUGAGCAGCAGCCACCUGUUCUAU | |
| Bold = Sense | CUGGCCCUGUGCCUGCUGACCUUUACCAGCUCUGCUACCGCCGGACCUGAGACA | |
| siRNA strand | CUUUGUGGCGCUGAACUGGUGGACGCCCUGCAGUUUGUGUGUGGCGACAGAGGC | |
| Bold and Italics = | UUCUACUUCAACAAGCCCACAGGCUACGGCAGCAGCUCUAGAAGGGCUCCUCAG | |
| anti-Sense siRNA | ACCGGAAUCGUGGACGAGUGCUGUUUCAGAAGCUGCGACCUGCGGCGGCUGGAA | |
| strand | AUGUAUUGUGCCCCUCUGAAGCCUGCCAAGAGCGCCUAAACAACAACAACAAGA | |
| Underline = | UGAAGAGCACCAAACUUGUUGGUGCUCUUCAUCUUGUUGACAACAACAACAAGA | |
| Signal peptide | UGAAGAGCACCAAACUUGUUGGUGCUCUUCAUCUUGUUGACAACAAUUUAUCUU | |
| Italics = Kozak | AGAGGCAUAUCCCU | |
| sequence | (all Us are modified; N¹-methylpseudouridine) | |
| Compound 10 | GCCACCAUGUUGUUGCUGCUGCUCGCCUGUAUUGCCCUGGCCUCUACAGCCGCC | 101 |
| RNA sequence | GCUACAAAUUCUGCCCCUACCAGCAGCUCCACCAAGAAAACCCAGCUGCAACUG | |
| Bold = Sense | GAACAUCUGCUGCUGGACCUGCAGAUGAUCCUGAACGGCAUCAACAACUACAAG | |
| siRNA strand | AACCCCAAGCUGACCCGGAUGCUGACCUUCAAGUUCUACAUGCCCAAGAAGGCC | |
| Bold and Italics = | ACCGAGCUGAAGCACCUCCAGUGCCUGGAAGAGGAACUGAAGCCCCUGGAAGAA | |
| anti-Sense siRNA | GUGCUGAAUCUGGCCCAGAGCAAGAACUUCCACCUGAGGCCUAGGGACCUGAUC | |
| strand | AGCAACAUCAACGUGAUCGUGCUGGAACUGAAAGGCAGCGAGACAACCUUCAUG | |
| Underline = | UGCGAGUACGCCGACGAGACAGCUACCAUCGUGGAAUUUCUGAACCGGUGGAUC | |
| Signal peptide | ACCUUCUGCCAGAGCAUCAUCAGCACCCUGACCUGAAUAGUGAGUCGUAUUAAC | |
| Italics = Kozak | GUACCAACAAGGAGGGCAGAAUCAUCACGAAGUGGUGAAGUACUUGACUUCACC | |
| sequence | ACUUCGUGAUGAUUCUGCCCUCCUUUAUCUUAGAGGCAUAUCCCUACGUACCAA | |
| | CAAGAGAUGAGCUUCCUACAGCACAACAAAUGUGACUUGCACAUUUGUUGUGCU | |
| | GUAGGAAGCUCAUCUCUUUAUCUUAGAGGCAUAUCCCUACGUACCAACAAGUAC | |
| | AAGAUCCGCAGACGUGUAAAUGUUCCACUUGGGAACAUUUACACGUCUGCGGAU | |
| | CUUGUACUUUAUCUUAGAGGCAUAUCCCUUUUAUCUUAGAGGCAUAUCCCU | |
| | (all Us are modified; N¹-methylpseudouridine) | |
| Compound 11 | GCCACCAUGUUGUUGCUGCUGCUCGCCUGUAUUGCCCUGGCCUCUACAGCCGCC | 102 |
| RNA sequence | GCUACAAAUUCUGCCCCUACCAGCAGCUCCACCAAGAAAACCCAGCUGCAACUG | |
| Bold = Sense | GAACAUCUGCUGCUGGACCUGCAGAUGAUCCUGAACGGCAUCAACAACUACAAG | |
| siRNA strand | AACCCCAAGCUGACCCGGAUGCUGACCUUCAAGUUCUACAUGCCCAAGAAGGCC | |
| Bold and Italics = | ACCGAGCUGAAGCACCUCCAGUGCCUGGAAGAGGAACUGAAGCCCCUGGAAGAA | |
| anti-Sense siRNA | GUGCUGAAUCUGGCCCAGAGCAAGAACUUCCACCUGAGGCCUAGGGACCUGAUC | |
| strand | AGCAACAUCAACGUGAUCGUGCUGGAACUGAAAGGCAGCGAGACAACCUUCAUG | |
| Underline | UGCGAGUACGCCGACGAGACAGCUACCAUCGUGGAAUUUCUGAACCGGUGGAUC | |
| Signal peptide | ACCUUCUGCCAGAGCAUCAUCAGCACCCUGACCUGAACAACAAGGAGGGCAGAA | |
| Italics = Kozak | UCAUCACGAAGUGGUGAAGUACUUGACUUCACCACUUCGUGAUGAUUCUGCCCU | |
| sequence | CCACAACAAGAGAUGAGCUUCCUACAGCACAACAAAUGUGACUUGCACAUUUGU | |
| | UGUGCUGUAGGAAGCUCAUCUCACAACAAGUACAAGAUCCGCAGACGUGUAAAU | |
| | GUUCCACUUGGGAACAUUUACACGUCUGCGGAUCUUGUACACAACAAUUUAUCU | |
| | UAGAGGCAUAUCCCUCUGGGCCUCAUGGGCCUUCCGCUCACUGCCCGCUUUCCA | |
| | GUCGGGAAACCUGUCGUGCCAGCUGCAUUAACAUGGUCAUAGCUG | |
| | (all Us are modified; N¹-methylpseudouridine) | |
| Compound 12 | GCCACCAUGUUGUUGCUGCUGCUCGCCUGUAUUGCCCUGGCCUCUACAGCCGCC | 103 |
| RNA sequence | GCUACAAAUUCUGCCCCUACCAGCAGCUCCACCAAGAAAACCCAGCUGCAACUG | |
| Bold = Sense | GAACAUCUGCUGCUGGACCUGCAGAUGAUCCUGAACGGCAUCAACAACUACAAG | |
| siRNA strand | AACCCCAAGCUGACCCGGAUGCUGACCUUCAAGUUCUACAUGCCCAAGAAGGCC | |
| Bold and Italics = | ACCGAGCUGAAGCACCUCCAGUGCCUGGAAGAGGAACUGAAGCCCCUGGAAGAA | |
| anti-Sense siRNA | GUGCUGAAUCUGGCCCAGAGCAAGAACUUCCACCUGAGGCCUAGGGACCUGAUC | |
| strand | AGCAACAUCAACGUGAUCGUGCUGGAACUGAAAGGCAGCGAGACAACCUUCAUG | |
| Underline = | UGCGAGUACGCCGACGAGACAGCUACCAUCGUGGAAUUUCUGAACCGGUGGAUC | |
| Signal peptide | ACCUUCUGCCAGAGCAUCAUCAGCACCCUGACCUGAAUAGUGAGUCGUAUUAGG | |
| Italics = Kozak | AGGGCAGAAUCAUCACGAAGUGGUGAAGUACUUGACUUCACCACUUCGUGAUGA | |
| sequence | UUCUGCCCUCCAUCCCUACGUACCAACAAGAGAUGAGCUUCCUACAGCACAACA | |
| | AAUGUGACUUGCACAUUUGUUGUGCUGUAGGAAGCUCAUCUCAUCCCUACGUAC | |
| | CAACAAGUACAAGAUCCGCAGACGUGUAAAUGUUCCACUUGGGAACAUUUACAC | |
| | GUCUGCGGAUCUUGUACUUUAUCUUAGAGGCAU | |
| | (all Us are modified; N¹-methylpseudouridine) | |
| Compound 13 | GCCACCAUGUUGUUGCUGCUGCUCGCCUGUAUUGCCCUGGCCUCUACAGCCGCC | 104 |
| RNA sequence | GCUACAAAUUCUGCCCCUACCAGCAGCUCCACCAAGAAAACCCAGCUGCAACUG | |
| Bold = Sense | GAACAUCUGCUGCUGGACCUGCAGAUGAUCCUGAACGGCAUCAACAACUACAAG | |
| siRNA strand | AACCCCAAGCUGACCCGGAUGCUGACCUUCAAGUUCUACAUGCCCAAGAAGGCC | |
| Bold and Italics = | ACCGAGCUGAAGCACCUCCAGUGCCUGGAAGAGGAACUGAAGCCCCUGGAAGAA | |
| anti-Sense siRNA | GUGCUGAAUCUGGCCCAGAGCAAGAACUUCCACCUGAGGCCUAGGGACCUGAUC | |
| strand | AGCAACAUCAACGUGAUCGUGCUGGAACUGAAAGGCAGCGAGACAACCUUCAUG | |
| Underline = | UGCGAGUACGCCGACGAGACAGCUACCAUCGUGGAAUUUCUGAACCGGUGGAUC | |
| Signal peptide | ACCUUCUGCCAGAGCAUCAUCAGCACCCUGACCUGAAUAGUGAGUCGUAUUAGG | |
| Italics = Kozak | AGGGCAGAAUCAUCACGAAGUGGUGAAGUACUUGACUUCACCACUUCGUGAUGA | |
| sequence | UUCUGCCCUCCACGUACCAACAAGAGAUGAGCUUCCUACAGCACAACAAAUGUG | |
| | ACUUGCACAUUUGUUGUGCUGUAGGAAGCUCAUCUCACGUACCAACAAGUACAA | |
| | GAUCCGCAGACGUGUAAAUGUUCCACUUGGGAACAUUUACACGUCUGCGGAUCU | |
| | UGUACUUUAUCUUAGAGGCAU | |
| | (all Us are modified; N¹-methylpseudouridine) | |
| Compound 14 | GCCACCAUGUUGUUGCUGCUGCUCGCCUGUAUUGCCCUGGCCUCUACAGCCGCC | 105 |
| RNA sequence | GCUACAAAUUCUGCCCCUACCAGCAGCUCCACCAAGAAAACCCAGCUGCAACUG | |
| Bold = Sense | GAACAUCUGCUGCUGGACCUGCAGAUGAUCCUGAACGGCAUCAACAACUACAAG | |
| siRNA strand | AACCCCAAGCUGACCCGGAUGCUGACCUUCAAGUUCUACAUGCCCAAGAAGGCC | |
| Bold and Italics = | ACCGAGCUGAAGCACCUCCAGUGCCUGGAAGAGGAACUGAAGCCCCUGGAAGAA | |
| anti-Sense siRNA | GUGCUGAAUCUGGCCCAGAGCAAGAACUUCCACCUGAGGCCUAGGGACCUGAUC | |
| strand | AGCAACAUCAACGUGAUCGUGCUGGAACUGAAAGGCAGCGAGACAACCUUCAUG | |
| Underline = | UGCGAGUACGCCGACGAGACAGCUACCAUCGUGGAAUUUCUGAACCGGUGGAUC | |
| Signal peptide | ACCUUCUGCCAGAGCAUCAUCAGCACCCUGACCUGAAUAGUGAGUCGUAUUAUC | |
| Italics = Kozak | CCGGAGGGCAGAAUCAUCACGAAGUGGUGAAGUACUUGACUUCACCACUUCGUG | |
| sequence | AUGAUUCUGCCCUCCUCCCGAGAUGAGCUUCCUACAGCACAACAAAUGUGACUU | |
| | GCACAUUUGUUGUGCUGUAGGAAGCUCAUCUCUCCCGUACAAGAUCCGCAGACG | |
| | UGUAAAUGUUCCACUUGGGAACAUUUACACGUCUGCGGAUCUUGUACUUUAUCU | |
| | UAGAGGCAU | |
| | (all Us are modified; N¹-methylpseudouridine) | |
| Compound 15 | GCCACCAUGUUGUUGCUGCUGCUCGCCUGUAUUGCCCUGGCCUCUACAGCCGCC | 106 |
| RNA sequence | GCUACAAAUUCUGCCCCUACCAGCAGCUCCACCAAGAAAACCCAGCUGCAACUG | |
| Bold = Sense | GAACAUCUGCUGCUGGACCUGCAGAUGAUCCUGAACGGCAUCAACAACUACAAG | |
| siRNA strand | AACCCCAAGCUGACCCGGAUGCUGACCUUCAAGUUCUACAUGCCCAAGAAGGCC | |
| Bold and Italics = | ACCGAGCUGAAGCACCUCCAGUGCCUGGAAGAGGAACUGAAGCCCCUGGAAGAA | |
| anti-Sense siRNA | GUGCUGAAUCUGGCCCAGAGCAAGAACUUCCACCUGAGGCCUAGGGACCUGAUC | |
| strand | AGCAACAUCAACGUGAUCGUGCUGGAACUGAAAGGCAGCGAGACAACCUUCAUG | |
| Underline = | UGCGAGUACGCCGACGAGACAGCUACCAUCGUGGAAUUUCUGAACCGGUGGAUC | |
| Signal peptide | ACCUUCUGCCAGAGCAUCAUCAGCACCCUGACCUGAAUAGUGAGUCGUAUUAAC | |
| Italics = Kozak | AACAAUCCCGGAGGGCAGAAUCAUCACGAAGUGGUGAAGUACUUGACUUCACCA | |
| sequence | CUUCGUGAUGAUUCUGCCCUCCACAACAAUCCCGAGAUGAGCUUCCUACAGCAC | |
| | AACAAAUGUGACUUGCACAUUUGUUGUGCUGUAGGAAGCUCAUCUCACAACAAU | |
| | CCCGUACAAGAUCCGCAGACGUGUAAAUGUUCCACUUGGGAACAUUUACACGUC | |
| | UGCGGAUCUUGUACUUUAUCUUAGAGGCAU | |
| | (all Us are modified; N¹-methylpseudouridine) | |
| Compound 16 | GCCACCAUGUGUCACCAGCAGCUGGUCAUCAGCUGGUUCAGCCUGGUGUUCCUG | 107 |
| RNA sequence | GCCUCUCCUCUGGUGGCCAUCUGGGAGCUGAAGAAAGACGUGUACGUGGUGGAA | |
| Bold = Sense | CUGGACUGGUAUCCCGAUGCUCCUGGCGAGAUGGUGGUGCUGACCUGCGAUACC | |
| siRNA strand | CCUGAAGAGGACGGCAUCACCUGGACACUGGAUCAGUCUAGCGAGGUGCUCGGC | |
| Bold and Italics = | AGCGGCAAGACCCUGACCAUCCAAGUGAAAGAGUUUGGCGACGCCGGCCAGUAC | |
| anti-Sense siRNA | ACCUGUCACAAAGGCGGAGAAGUGCUGAGCCACAGCCUGCUGCUGCUCCACAAG | |
| strand | AAAGAGGAUGGCAUUUGGAGCACCGACAUCCUGAAGGACCAGAAAGAGCCCAAG | |
| Underline = | AACAAGACCUUCCUGAGAUGCGAGGCCAAGAACUACAGCGGCCGGUUCACAUGU | |
| Signal peptide | UGGUGGCUGACCACCAUCAGCACCGACCUGACCUUCAGCGUGAAGUCCAGCAGA | |
| Italics = Kozak | GGCAGCAGUGAUCCUCAGGGCGUUACAUGUGGCGCCGCUACACUGUCUGCCGAA | |
| sequence | AGAGUGCGGGGCGACAACAAAGAAUACGAGUACAGCGUGGAAUGCCAAGAGGAC | |
| | AGCGCCUGUCCAGCCGCCGAAGAGUCUCUGCCUAUCGAAGUGAUGGUGGACGCC | |
| | GUGCACAAGCUGAAGUACGAGAACUACACCUCCAGCUUUUUCAUCCGGGACAUC | |
| | AUCAAGCCCGAUCCUCCAAAGAACCUGCAGCUGAAGCCUCUGAAGAACAGCAGA | |
| | CAGGUGGAAGUGUCCUGGGAGUACCCCGACACCUGGUCUACACCCCACAGCUAC | |
| | UUCAGCCUGACCUUUUGCGUGCAAGUGCAGGGCAAGUCCAAGCGCGAGAAAAAG | |
| | GACCGGGUGUUCACCGACAAGACCAGCGCCACCGUGAUCUGCAGAAAGAACGCC | |
| | AGCAUCAGCGUCAGAGCCCAGGACCGGUACUACAGCAGCUCUUGGAGCGAAUGG | |
| | GCCAGCGUGCCAUGUUCUGGUGGCGGAGGAUCUGGCGGAGGUGGAAGCGGCGGA | |
| | GGCGGAUCUAGAAAUCUGCCUGUGGCCACUCCUGAUCCUGGCAUGUUCCCUUGU | |
| | CUGCACCACAGCCAGAACCUGCUGAGAGCCGUGUCCAACAUGCUGCAGAAGGCC | |
| | AGACAGACCCUGGAAUUCUACCCCUGCACCAGCGAGGAAAUCGACCACGAGGAC | |
| | AUCACCAAGGAUAAGACCAGCACCGUGGAAGCCUGCCUGCCUCUGGAACUGACC | |
| | AAGAACGAGAGCUGCCUGAACAGCCGGGAAACCAGCUUCAUCACCAACGGCUCU | |
| | UGCCUGGCCAGCAGAAAGACCUCCUUCAUGAUGGCCCUGUGCCUGAGCAGCAUC | |
| | UACGAGGACCUGAAGAUGUACCAGGUGGAAUUCAAGACCAUGAACGCCAAGCUG | |
| | CUGAUGGACCCCAAGCGGCAGAUCUUCCUGGACCAGAAUAUGCUGGCCGUGAUC | |
| | GACGAGCUGAUGCAGGCCCUGAACUUCAACAGCGAGACAGUGCCCCAGAAGUCU | |
| | AGCCUGGAAGAACCCGACUUCUACAAGACCAAGAUCAAGCUGUGCAUCCUGCUG | |
| | CACGCCUUCCGGAUCAGAGCCGUGACCAUCGACAGAGUGAUGAGCUACCUGAAC | |
| | GCCUCCUGAAUAGUGAGUCGUAUUAACGUACCAACAAGGACGACGAGACCUUCA | |
| | UCAAACUUGUUGAUGAAGGUCUCGUCGUCCUUUAUCUUAGAGGCAUAUCCCUAC | |
| | GUACCAACAAGUGCAAUGAGGGACCAGUACAACUUGUGUACUGGUCCCUCAUUG | |
| | CACUUUAUCUUAGAGGCAUAUCCCUACGUACCAACAAUUCUACAACCAGGACCA | |
| | UGAGACUUGCUCAUGGUCCUGGUUGUAGAAUUUAUCUUAGAGGCAUAUCCCUUU | |
| | UAUCUUAGAGGCAUAUCCCU | |
| | (all Us are modified; N¹-methylpseudouridine) | |
| Compound 17 | GCCACCAUGUGUCACCAGCAGCUGGUCAUCAGCUGGUUCAGCCUGGUGUUCCUG | 108 |
| RNA sequence | GCCUCUCCUCUGGUGGCCAUCUGGGAGCUGAAGAAAGACGUGUACGUGGUGGAA | |
| Bold = Sense | CUGGACUGGUAUCCCGAUGCUCCUGGCGAGAUGGUGGUGCUGACCUGCGAUACC | |
| siRNA strand | CCUGAAGAGGACGGCAUCACCUGGACACUGGAUCAGUCUAGCGAGGUGCUCGGC | |
| Bold and Italics = | AGCGGCAAGACCCUGACCAUCCAAGUGAAAGAGUUUGGCGACGCCGGCCAGUAC | |
| anti-Sense siRNA | ACCUGUCACAAAGGCGGAGAAGUGCUGAGCCACAGCCUGCUGCUGCUCCACAAG | |
| strand | AAAGAGGAUGGCAUUUGGAGCACCGACAUCCUGAAGGACCAGAAAGAGCCCAAG | |
| Underline = | AACAAGACCUUCCUGAGAUGCGAGGCCAAGAACUACAGCGGCCGGUUCACAUGU | |
| Signal peptide | UGGUGGCUGACCACCAUCAGCACCGACCUGACCUUCAGCGUGAAGUCCAGCAGA | |
| Italics = Kozak | GGCAGCAGUGAUCCUCAGGGCGUUACAUGUGGCGCCGCUACACUGUCUGCCGAA | |
| sequence | AGAGUGCGGGGCGACAACAAAGAAUACGAGUACAGCGUGGAAUGCCAAGAGGAC | |
| | AGCGCCUGUCCAGCCGCCGAAGAGUCUCUGCCUAUCGAAGUGAUGGUGGACGCC | |
| | GUGCACAAGCUGAAGUACGAGAACUACACCUCCAGCUUUUUCAUCCGGGACAUC | |
| | AUCAAGCCCGAUCCUCCAAAGAACCUGCAGCUGAAGCCUCUGAAGAACAGCAGA | |
| | CAGGUGGAAGUGUCCUGGGAGUACCCCGACACCUGGUCUACACCCCACAGCUAC | |
| | UUCAGCCUGACCUUUUGCGUGCAAGUGCAGGGCAAGUCCAAGCGCGAGAAAAAG | |
| | GACCGGGUGUUCACCGACAAGACCAGCGCCACCGUGAUCUGCAGAAAGAACGCC | |
| | AGCAUCAGCGUCAGAGCCCAGGACCGGUACUACAGCAGCUCUUGGAGCGAAUGG | |
| | GCCAGCGUGCCAUGUUCUGGUGGCGGAGGAUCUGGCGGAGGUGGAAGCGGCGGA | |
| | GGCGGAUCUAGAAAUCUGCCUGUGGCCACUCCUGAUCCUGGCAUGUUCCCUUGU | |
| | CUGCACCACAGCCAGAACCUGCUGAGAGCCGUGUCCAACAUGCUGCAGAAGGCC | |
| | AGACAGACCCUGGAAUUCUACCCCUGCACCAGCGAGGAAAUCGACCACGAGGAC | |
| | AUCACCAAGGAUAAGACCAGCACCGUGGAAGCCUGCCUGCCUCUGGAACUGACC | |
| | AAGAACGAGAGCUGCCUGAACAGCCGGGAAACCAGCUUCAUCACCAACGGCUCU | |
| | UGCCUGGCCAGCAGAAAGACCUCCUUCAUGAUGGCCCUGUGCCUGAGCAGCAUC | |
| | UACGAGGACCUGAAGAUGUACCAGGUGGAAUUCAAGACCAUGAACGCCAAGCUG | |
| | CUGAUGGACCCCAAGCGGCAGAUCUUCCUGGACCAGAAUAUGCUGGCCGUGAUC | |
| | GACGAGCUGAUGCAGGCCCUGAACUUCAACAGCGAGACAGUGCCCCAGAAGUCU | |
| | AGCCUGGAAGAACCCGACUUCUACAAGACCAAGAUCAAGCUGUGCAUCCUGCUG | |
| | CACGCCUUCCGGAUCAGAGCCGUGACCAUCGACAGAGUGAUGAGCUACCUGAAC | |
| | GCCUCCUGAACAACAAGGACGACGAGACCUUCAUCAAACUUGUUGAUGAAGGUC | |
| | UCGUCGUCCACAACAAGUGCAAUGAGGGACCAGUACAACUUGUGUACUGGUCCC | |
| | UCAUUGCACACAACAAUUCUACAACCAGGACCAUGAGACUUGCUCAUGGUCCUG | |
| | GUUGUAGAAACAACAAUUUAUCUUAGAGGCAUAUCCCU | |
| | (all Us are modified; N¹-methylpseudouridine) | |
| Human IL-2 | MYRMQLLSCIALSLALVTNSAPTSSSTKKTQLQLEHLLLDLQMILNGINNYKNP | 109 |
| amino acid | KLTRMLTFKFYMPKKATELKHLQCLEEELKPLEEVLNLAQSKNFHLRPRDLISN | |
| (Genbank | INVIVLELKGSETTEMCEYADETATIVEFLNRWITFCQSIISTLT | |
| NM_000586.3) | | |
| Underlined: signal | | |
| sequence | | |
| Mature Human | APTSSSTKKTQLQLEHLLLDLQMILNGINNYKNPKLTRMLTFKFYMPKKATELK | 110 |
| IL-2 amino acid | HLQCLEEELKPLEEVLNLAQSKNFHLRPRDLISNINVIVLELKGSETTEMCEYA | |
| (Genbank | DETATIVEFLNRWITFCQSIISTLT | |
| NM_000586.3) | | |
| Underlined: signal | | |
| sequence | | |
| Human IL-2 | AGTTCCCTATCACTCTCTTTAATCACTACTCACAGTAACCTCAACTCCTGCCAC | 111 |
| nucleic acid | AATGTACAGGATGCAACTCCTGTCTTGCATTGCACTAAGTCTTGCACTTGTCAC | |
| (Genbank | AAACAGTGCACCTACTTCAAGTTCTACAAAGAAAACACAGCTACAACTGGAGCA | |
| NM_000586.3) | TTTACTGCTGGATTTACAGATGATTTTGAATGGAATTAATAATTACAAGAATCC | |
| Underlined: | CAAACTCACCAGGATGCTCACATTTAAGTTTTACATGCCCAAGAAGGCCACAGA | |
| coding sequence | ACTGAAACATCTTCAGTGTCTAGAAGAAGAACTCAAACCTCTGGAGGAAGTGCT | |
| Bold: signal | AAATTTAGCTCAAAGCAAAAACTTTCACTTAAGACCCAGGGACTTAATCAGCAA | |
| sequence | TATCAACGTAATAGTTCTGGAACTAAAGGGATCTGAAACAACATTCATGTGTGA | |
| | ATATGCTGATGAGACAGCAACCATTGTAGAATTTCTGAACAGATGGATTACCTT | |
| | TTGTCAAAGCATCATCTCAACACTGACTTGATAATTAAGTGCTTCCCACTTAAA | |
| | ACATATCAGGCCTTCTATTTATTTAAATATTTAAATTTTATATTTATTGTTGAA | |
| | TGTATGGTTTGCTACCTATTGTAACTATTATTCTTAATCTTAAAACTATAAATA | |
| | TGGATCTTTTATGATTCTTTTTGTAAGCCCTAGGGGCTCTAAAATGGTTTCACT | |
| | TATTTATCCCAAAATATTTATTATTATGTTGAATGTTAAATATAGTATCTATGT | |
| | AGATTGGTTAGTAAAACTATTTAATAAATTTGATAAATATAAAAAAAAAAAAAA | |
| | AAAAAAAAAAAA | |
| Modified IL-2 | MLLLLLACIALASTAAATNS | 112 |
| signal peptide | | |
| (Cpd.3) amino | | |
| acid (Y2L/R3- | | |
| /M4L/Q5L/S8A/- | | |
| A13/L14T/L16A | | |
| and V17A) | | |
| Endogenous IL-2 | ATGTACAGAATGCAGCTGCTGAGCTGTATCGCCCTGTCTCTGGCCCTGGTCACA | 113 |
| signal peptide | AATAGC | |
| nucleic acid | | |
| Modified IL-2 | ATGTTGTTGCTGCTGCTCGCCTGTATTGCCCTGGCCTCTACAGCCGCCGCTACA | 114 |
| signal peptide | AATTCT | |
| nucleic acid | | |
| VEGFA encoding | ATGAACTTTCTGCTGTCTTGGGTGCATTGGAGCCTTGCCTTGCTGCTCTACCTC | 115 |
| DNA sequence | CACCATGCCAAGTGGTCCCAGGCTGCACCCATGGCAGAAGGAGGAGGGCAGAAT | |
| (from Genbank | CATCACGAAGTGGTGAAGTTCATGGATGTCTATCAGCGCAGCTACTGCCATCCA | |
| NM_001171623.1 | ATCGAGACCCTGGTGGACATCTTCCAGGAGTACCCTGATGAGATCGAGTACATC | |
| Bold: signal | TTCAAGCCATCCTGTGTGCCCCTGATGCGATGCGGGGGCTGCTGCAATGACGAG | |
| peptide sequence | GGCCTGGAGTGTGTGCCCACTGAGGAGTCCAACATCACCATGCAGATTATGCGG | |
| Bold and | ATCAAACCTCACCAAGGCCAGCACATAGGAGAGATGAGCTTCCTACAGCACAAC | |
| italicized: siRNA | AAATGTGAATGCAGACCAAAGAAAGATAGAGCAAGACAAGAAAAAAAATCAGTT | |
| binding regions | CGAGGAAAGGGAAAGGGGCAAAAACGAAAGCGCAAGAAATCCCGGTATAAGTCC | |
| | TGGAGCGTGTACGTTGGTGCCCGCTGCTGTCTAATGCCCTGGAGCCTCCCTGGC | |
| | CCCCATCCCTGTGGGCCTTGCTCAGAGCGGAGAAAGCATTTGTTTGTACAAGAT | |
| | CCGCAGACGTGTAAATGTTCCTGCAAAAACACAGACTCGCGTTGCAAGGCGAGG | |
| | CAGCTTGAGTTAAACGAACGTACTTGCAGATGTGACAAGCCGAGGCGGTGA | |
| Human IL-12 | MCPARSLLLVATLVLLDHLSLARNLPVATPDPGMFPCLHHSQNLLRAVSNMLQK | 116 |
| alpha amino acid | ARQTLEFYPCTSEEIDHEDITKDKTSTVEACLPLELTKNESCLNSRETSFITNG | |
| (Genbank | SCLASRKTSFMMALCLSSIYEDLKMYQVEFKTMNAKLLMDPKRQIFLDQNMLAV | |
| NM_000882.4) | IDELMQALNENSETVPQKSSLEEPDFYKTKIKLCILLHAFRIRAVTIDRVMSYL | |
| Underlined: | NAS | |
| signal | | |
| sequence | | |
| Mature Human | RNLPVATPDPGMFPCLHHSQNLLRAVSNMLQKARQTLEFYPCTSEEIDHEDITK | 117 |
| IL-12 alpha | DETSTVEACLPLELTKNESCLNSRETSFITNGSCLASRKTSEMMALCLSSIYED | |
| amino acid | LKMYQVEFKTMNAKLLMDPKRQIFLDQNMLAVIDELMQALNENSETVPQKSSLE | |
| (Genbank | EPDFYKTKIKLCILLHAFRIRAVTIDRVMSYLNAS | |
| NM_000882.4) | | |
| Human IL-12 | ATTTCGCTTTCATTTTGGGCCGAGCTGGAGGCGGCGGGGCCGTCCCGGAACGGC | 118 |
| alpha | TGCGGCCGGGCACCCCGGGAGTTAATCCGAAAGCGCCGCAAGCCCCGCGGGCCG | |
| nucleic acid | GCCGCACCGCACGTGTCACCGAGAAGCTGATGTAGAGAGAGACACAGAAGGAGA | |
| (Genbank | CAGAAAGCAAGAGACCAGAGTCCCGGGAAAGTCCTGCCGCGCCTCGGGACAATT | |
| NM_000882.4) | ATAAAAATGTGGCCCCCTGGGTCAGCCTCCCAGCCACCGCCCTCACCTGCCGCG | |
| Underlined: | GCCACAGGTCTGCATCCAGCGGCTCGCCCTGTGTCCCTGCAGTGCCGGCTCAGC | |
| coding sequence | ATGTGTCCAGCGCGCAGCCTCCTCCTTGTGGCTACCCTGGTCCTCCTGGACCAC | |
| Bold: signal | CTCAGTTTGGCCAGAAACCTCCCCGTGGCCACTCCAGACCCAGGAATGTTCCCA | |
| sequence | TGCCTTCACCACTCCCAAAACCTGCTGAGGGCCGTCAGCAACATGCTCCAGAAG | |
| | GCCAGACAAACTCTAGAATTTTACCCTTGCACTTCTGAAGAGATTGATCATGAA | |
| | GATATCACAAAAGATAAAACCAGCACAGTGGAGGCCTGTTTACCATTGGAATTA | |
| | ACCAAGAATGAGAGTTGCCTAAATTCCAGAGAGACCTCTTTCATAACTAATGGG | |
| | AGTTGCCTGGCCTCCAGAAAGACCTCTTTTATGATGGCCCTGTGCCTTAGTAGT | |
| | ATTTATGAAGACTTGAAGATGTACCAGGTGGAGTTCAAGACCATGAATGCAAAG | |
| | CTTCTGATGGATCCTAAGAGGCAGATCTTTCTAGATCAAAACATGCTGGCAGTT | |
| | ATTGATGAGCTGATGCAGGCCCTGAATTTCAACAGTGAGACTGTGCCACAAAAA | |
| | TCCTCCCTTGAAGAACCGGATTTTTATAAAACTAAAATCAAGCTCTGCATACTT | |
| | CTTCATGCTTTCAGAATTCGGGCAGTGACTATTGATAGAGTGATGAGCTATCTG | |
| | AATGCTTCCTAAAAAGCGAGGTCCCTCCAAACCGTTGTCATTTTTATAAAACTT | |
| | TGAAATGAGGAAACTTTGATAGGATGTGGATTAAGAACTAGGGAGGGGGAAAGA | |
| | AGGATGGGACTATTACATCCACATGATACCTCTGATCAAGTATTTTTGACATTT | |
| | ACTGTGGATAAATTGTTTTTAAGTTTTCATGAATGAATTGCTAAGAAGGGAAAA | |
| | TATCCATCCTGAAGGTGTTTTTCATTCACTTTAATAGAAGGG | |
| Human IL-12 beta | MCHQQLVISWFSLVFLASPLVAIWELKKDVYVVELDWYPDAPGEMVVLTCDTPE | 119 |
| amino acid | EDGITWTLDQSSEVLGSGKTLTIQVKEFGDAGQYTCHKGGEVLSHSLLLLHKKE | |
| (Genbank | DGIWSTDILKDQKEPKNKTFLRCEAKNYSGRFTCWWLTTISTDLTFSVKSSRGS | |
| NM_002187.2) | SDPQGVTCGAATLSAERVRGDNKEYEYSVECQEDSACPAAEESLPIEVMVDAVH | |
| Underlined: signal | KLKYENYTSSFFIRDIIKPDPPKNLQLKPLKNSRQVEVSWEYPDTWSTPHSYES | |
| sequence | LTFCVQVQGKSKREKKDRVFTDKTSATVICRKNASISVRAQDRYYSSSWSEWAS | |
| | VPCS | |
| Mature Human | IWELKKDVYVVELDWYPDAPGEMVVLTCDTPEEDGITWTLDQSSEVLGSGKTLT | 120 |
| IL-12 beta amino | IQVKEFGDAGQYTCHKGGEVLSHSLLLLHKKEDGIWSTDILKDQKEPKNKTELR | |
| acid (Genbank | CEAKNYSGRFTCWWLTTISTDLTFSVKSSRGSSDPQGVTCGAATLSAERVRGDN | |
| NM_002187.2) | KEYEYSVECQEDSACPAAEESLPIEVMVDAVHKLKYENYTSSFFIRDIIKPDPP | |
| | KNLQLKPLKNSRQVEVSWEYPDTWSTPHSYFSLTFCVQVQGKSKREKKDRVFTD | |
| | KTSATVICRKNASISVRAQDRYYSSSWSEWASVPCS | |
| Human IL-12 beta | CTGTTTCAGGGCCATTGGACTCTCCGTCCTGCCCAGAGCAAGATGTGTCACCAG | 121 |
| nucleic acid | CAGTTGGTCATCTCTTGGTTTTCCCTGGTTTTTCTGGCATCTCCCCTCGTGGCC | |
| (Genbank | ATATGGGAACTGAAGAAAGATGTTTATGTCGTAGAATTGGATTGGTATCCGGAT | |
| NM_002187.2) | GCCCCTGGAGAAATGGTGGTCCTCACCTGTGACACCCCTGAAGAAGATGGTATC | |
| Underlined: | ACCTGGACCTTGGACCAGAGCAGTGAGGTCTTAGGCTCTGGCAAAACCCTGACC | |
| coding sequence | ATCCAAGTCAAAGAGTTTGGAGATGCTGGCCAGTACACCTGTCACAAAGGAGGC | |
| Bold: signal | GAGGTTCTAAGCCATTCGCTCCTGCTGCTTCACAAAAAGGAAGATGGAATTTGG | |
| sequence | TCCACTGATATTTTAAAGGACCAGAAAGAACCCAAAAATAAGACCTTTCTAAGA | |
| | TGCGAGGCCAAGAATTATTCTGGACGTTTCACCTGCTGGTGGCTGACGACAATC | |
| | AGTACTGATTTGACATTCAGTGTCAAAAGCAGCAGAGGCTCTTCTGACCCCCAA | |
| | GGGGTGACGTGCGGAGCTGCTACACTCTCTGCAGAGAGAGTCAGAGGGGACAAC | |
| | AAGGAGTATGAGTACTCAGTGGAGTGCCAGGAGGACAGTGCCTGCCCAGCTGCT | |
| | GAGGAGAGTCTGCCCATTGAGGTCATGGTGGATGCCGTTCACAAGCTCAAGTAT | |
| | GAAAACTACACCAGCAGCTTCTTCATCAGGGACATCATCAAACCTGACCCACCC | |
| | AAGAACTTGCAGCTGAAGCCATTAAAGAATTCTCGGCAGGTGGAGGTCAGCTGG | |
| | GAGTACCCTGACACCTGGAGTACTCCACATTCCTACTTCTCCCTGACATTCTGC | |
| | GTTCAGGTCCAGGGCAAGAGCAAGAGAGAAAAGAAAGATAGAGTCTTCACGGAC | |
| | AAGACCTCAGCCACGGTCATCTGCCGCAAAAATGCCAGCATTAGCGTGCGGGCC | |
| | CAGGACCGCTACTATAGCTCATCTTGGAGCGAATGGGCATCTGTGCCCTGCAGT | |
| | TAGGTTCTGATCCAGGATGAAAATTTGGAGGAAAAGTGGAAGATATTAAGCAAA | |
| | ATGTTTAAAGACACAACGGAATAGACCCAAAAAGATAATTTCTATCTGATTTGC | |
| | TTTAAAACGTTTTTTTAGGATCACAATGATATCTTTGCTGTATTTGTATAGTTA | |
| | GATGCTAAATGCTCATTGAAACAATCAGCTAATTTATGTATAGATTTTCCAGCT | |
| | CTCAAGTTGCCATGGGCCTTCATGCTATTTAAATATTTAAGTAATTTATGTATT | |
| | TATTAGTATATTACTGTTATTTAACGTTTGTCTGCCAGGATGTATGGAATGTTT | |
| | CATACTCTTATGACCTGATCCATCAGGATCAGTCCCTATTATGCAAAATGTGAA | |
| | TTTAAT | |
| c-Myc encoding | ATGGATTTTTTTCGGGTAGTGGAAAACCAGCAGCCTCCCGCGACGATGCCCCTC | 122 |
| DNA sequence | AACGTTAGCTTCACCAACAGGAACTATGACCTCGACTACGACTCGGTGCAGCCG | |
| (from Genbank | TATTTCTACTGCGACGAGGAGGAGAACTTCTACCAGCAGCAGCAGCAGAGCGAG | |
| NM_002467.4) | CTGCAGCCCCCGGCGCCCAGCGAGGATATCTGGAAGAAATTCGAGCTGCTGCCC | |
| Bold and | ACCCCGCCCCTGTCCCCTAGCCGCCGCTCCGGGCTCTGCTCGCCCTCCTACGTT | |
| italicized: siRNA | GCGGTCACACCCTTCTCCCTTCGGGGAGACAACGACGGCGGTGGCGGGAGCTTC | |
| binding regions | TCCACGGCCGACCAGCTGGAGATGGTGACCGAGCTGCTGGGAGGAGACATGGTG | |
| | AACCAGAGTTTCATCTGCGACCCGGACGACGAGACCTTCATCAAAAACATCATC | |
| | ATCCAGGACTGTATGTGGAGCGGCTTCTCGGCCGCCGCCAAGCTCGTCTCAGAG | |
| | AAGCTGGCCTCCTACCAGGCTGCGCGCAAAGACAGCGGCAGCCCGAACCCCGCC | |
| | CGCGGCCACAGCGTCTGCTCCACCTCCAGCTTGTACCTGCAGGATCTGAGCGCC | |
| | GCCGCCTCAGAGTGCATCGACCCCTCGGTGGTCTTCCCCTACCCTCTCAACGAC | |
| | AGCAGCTCGCCCAAGTCCTGCGCCTCGCAAGACTCCAGCGCCTTCTCTCCGTCC | |
| | TCGGATTCTCTGCTCTCCTCGACGGAGTCCTCCCCGCAGGGCAGCCCCGAGCCC | |
| | CTGGTGCTCCATGAGGAGACACCGCCCACCACCAGCAGCGACTCTGAGGAGGAA | |
| | CAAGAAGATGAGGAAGAAATCGATGTTGTTTCTGTGGAAAAGAGGCAGGCTCCT | |
| | GGCAAAAGGTCAGAGTCTGGATCACCTTCTGCTGGAGGCCACAGCAAACCTCCT | |
| | CACAGCCCACTGGTCCTCAAGAGGTGCCACGTCTCCACACATCAGCACAACTAC | |
| | GCAGCGCCTCCCTCCACTCGGAAGGACTATCCTGCTGCCAAGAGGGTCAAGTTG | |
| | GACAGTGTCAGAGTCCTGAGACAGATCAGCAACAACCGAAAATGCACCAGCCCC | |
| | AGGTCCTCGGACACCGAGGAGAATGTCAAGAGGCGAACACACAACGTCTTGGAG | |
| | CGCCAGAGGAGGAACGAGCTAAAACGGAGCTTTTTTGCCCTGCGTGACCAGATC | |
| | CCGGAGTTGGAAAACAATGAAAAGGCCCCCAAGGTAGTTATCCTTAAAAAAGCC | |
| | ACAGCATACATCCTGTCCGTCCAAGCAGAGGAGCAAAAGCTCATTTCTGAAGAG | |
| | GACTTGTTGCGGAAACGACGAGAACAGTTGAAACACAAACTTGAACAGCTACGG | |
| | AACTCTTGTGCGTAA | |
| KRAS encoding | ATGACTGAATATAAACTTGTGGTAGTTGGAGCTGGTGGCGTAGGCAAGAGTGCC | 123 |
| DNA sequence | TTGACGATACAGCTAATTCAGAATCATTTTGTGGACGAATATGATCCAACAATA | |
| (from Genbank | GAGGATTCCTACAGGAAGCAAGTAGTAATTGATGGAGAAACCTGTCTCTTGGAT | |
| NM_004985.4) | ATTCTCGACACAGCAGGTCAAGAGGAGTACAGTGCAATGAGGGACCAGTACATG | |
| Bold and | AGGACTGGGGAGGGCTTTCTTTGTGTATTTGCCATAAATAATACTAAATCATTT | |
| italicized: siRNA | GAAGATATTCACCATTATAGAGAACAAATTAAAAGAGTTAAGGACTCTGAAGAT | |
| binding regions | GTACCTATGGTCCTAGTAGGAAATAAATGTGATTTGCCTTCTAGAACAGTAGAC | |
| | ACAAAACAGGCTCAGGACTTAGCAAGAAGTTATGGAATTCCTTTTATTGAAACA | |
| | TCAGCAAAGACAAGACAGGGTGTTGATGATGCCTTCTATACATTAGTTCGAGAA | |
| | ATTCGAAAACATAAAGAAAAGATGAGCAAAGATGGTAAAAAGAAGAAAAAGAAG | |
| | TCAAAGACAAAGTGTGTAATTATGTAA | |
| Akt1 encoding | ATGAGCGACGTGGCTATTGTGAAGGAGGGTTGGCTGCACAAACGAGGGGAGTAC | 124 |
| DNA sequence | ATCAAGACCTGGCGGCCACGCTACTTCCTCCTCAAGAATGATGGCACCTTCATT | |
| (from Genbank | GGCTACAAGGAGCGGCCGCAGGATGTGGACCAACGTGAGGCTCCCCTCAACAAC | |
| NM_005163.2) | TTCTCTGTGGCGCAGTGCCAGCTGATGAAGACGGAGCGGCCCCGGCCCAACACC | |
| Bold and | TTCATCATCCGCTGCCTGCAGTGGACCACTGTCATCGAACGCACCTTCCATGTG | |
| italicized: siRNA | GAGACTCCTGAGGAGCGGGAGGAGTGGACAACCGCCATCCAGACTGTGGCTGAC | |
| binding regions | GGCCTCAAGAAGCAGGAGGAGGAGGAGATGGACTTCCGGTCGGGCTCACCCAGT | |
| | GACAACTCAGGGGCTGAAGAGATGGAGGTGTCCCTGGCCAAGCCCAAGCACCGC | |
| | GTGACCATGAACGAGTTTGAGTACCTGAAGCTGCTGGGCAAGGGCACTTTCGGC | |
| | AAGGTGATCCTGGTGAAGGAGAAGGCCACAGGCCGCTACTACGCCATGAAGATC | |
| | CTCAAGAAGGAAGTCATCGTGGCCAAGGACGAGGTGGCCCACACACTCACCGAG | |
| | AACCGCGTCCTGCAGAACTCCAGGCACCCCTTCCTCACAGCCCTGAAGTACTCT | |
| | TTCCAGACCCACGACCGCCTCTGCTTTGTCATGGAGTACGCCAACGGGGGCGAG | |
| | CTGTTCTTCCACCTGTCCCGGGAGCGTGTGTTCTCCGAGGACCGGGCCCGCTTC | |
| | TATGGCGCTGAGATTGTGTCAGCCCTGGACTACCTGCACTCGGAGAAGAACGTG | |
| | GTGTACCGGGACCTCAAGCTGGAGAACCTCATGCTGGACAAGGACGGGCACATT | |
| | AAGATCACAGACTTCGGGCTGTGCAAGGAGGGGATCAAGGACGGTGCCACCATG | |
| | AAGACCTTTTGCGGCACACCTGAGTACCTGGCCCCCGAGGTGCTGGAGGACAAT | |
| | GACTACGGCCGTGCAGTGGACTGGTGGGGGCTGGGCGTGGTCATGTACGAGATG | |
| | ATGTGCGGTCGCCTGCCCTTCTACAACCAGGACCATGAGAAGCTTTTTGAGCTC | |
| | ATCCTCATGGAGGAGATCCGCTTCCCGCGCACGCTTGGTCCCGAGGCCAAGTCC | |
| | TTGCTTTCAGGGCTGCTCAAGAAGGACCCCAAGCAGAGGCTTGGCGGGGGCTCC | |
| | GAGGACGCCAAGGAGATCATGCAGCATCGCTTCTTTGCCGGTATCGTGTGGCAG | |
| | CACGTGTACGAGAAGAAGCTCAGCCCACCCTTCAAGCCCCAGGTCACGTCGGAG | |
| | ACTGACACCAGGTATTTTGATGAGGAGTTCACGGCCCAGATGATCACCATCACA | |
| | CCACCTGACCAAGATGACAGCATGGAGTGTGTGGACAGCGAGCGCAGGCCCCAC | |
| | TTCCCCCAGTTCTCCTACTCGGCCAGCGGCACGGCCTGA | |
| Akt2 encoding | ATGAATGAGGTGTCTGTCATCAAAGAAGGCTGGCTCCACAAGCGTGGTGAATAC | 125 |
| DNA sequence | ATCAAGACCTGGAGGCCACGGTACTTCCTGCTGAAGAGCGACGGCTCCTTCATT | |
| (from Genbank | GGGTACAAGGAGAGGCCCGAGGCCCCTGATCAGACTCTACCCCCCTTAAACAAC | |
| NM_001626.6) | TTCTCCGTAGCAGAATGCCAGCTGATGAAGACCGAGAGGCCGCGACCCAACACC | |
| Bold and | TTTGTCATACGCTGCCTGCAGTGGACCACAGTCATCGAGAGGACCTTCCACGTG | |
| italicized: siRNA | GATTCTCCAGACGAGAGGGAGGAGTGGATGCGGGCCATCCAGATGGTCGCCAAC | |
| binding regions | AGCCTCAAGCAGCGGGCCCCAGGCGAGGACCCCATGGACTACAAGTGTGGCTCC | |
| Bold, italicized | CCCAGTGACTCCTCCACGACTGAGGAGATGGAAGTGGCGGTCAGCAAGGCACGG | |
| and underlined: | GCTAAAGTGACCATGAATGACTTCGACTATCTCAAACTCCTTGGCAAGGGAACC | |
| Mismatch | TTTGGCAAAGTCATCCTGGTGCGGGAGAAGGCCACTGGCCGCTACTACGCCATG | |
| sequence to the | AAGATCCTGCGGAAGGAAGTCATCATTGCCAAGGATGAAGTCGCTCACACAGTC | |
| target siRNA | ACCGAGAGCCGGGTCCTCCAGAACACCAGGCACCCGTTCCTCACTGCGCTGAAG | |
| | TATGCCTTCCAGACCCACGACCGCCTGTGCTTTGTGATGGAGTATGCCAACGGG | |
| | GGTGAGCTGTTCTTCCACCTGTCCCGGGAGCGTGTCTTCACAGAGGAGCGGGCC | |
| | CGGTTTTATGGTGCAGAGATTGTCTCGGCTCTTGAGTACTTGCACTCGCGGGAC | |
| | GTGGTATACCGCGACATCAAGCTGGAAAACCTCATGCTGGACAAAGATGGCCAC | |
| | ATCAAGATCACTGACTTTGGCCTCTGCAAAGAGGGCATCAGTGACGGGGCCACC | |
| | ATGAAAACCTTCTGTGGGACCCCGGAGTACCTGGCGCCTGAGGTGCTGGAGGAC | |
| | AATGACTATGGCCGGGCCGTGGACTGGTGGGGGCTGGGTGTGGTCATGTACGAG | |
| | ATGATGTGCGGCCGCCTGCCCTTCTACAACCAGGACCACGAGCGCCTCTTCGAG | |
| | CTCATCCTCATGGAAGAGATCCGCTTCCCGCGCACGCTCAGCCCCGAGGCCAAG | |
| | TCCCTGCTTGCTGGGCTGCTTAAGAAGGACCCCAAGCAGAGGCTTGGTGGGGGG | |
| | CCCAGCGATGCCAAGGAGGTCATGGAGCACAGGTTCTTCCTCAGCATCAACTGG | |
| | CAGGACGTGGTCCAGAAGAAGCTCCTGCCACCCTTCAAACCTCAGGTCACGTCC | |
| | GAGGTCGACACAAGGTACTTCGATGATGAATTTACCGCCCAGTCCATCACAATC | |
| | ACACCCCCTGACCGCTATGACAGCCTGGGCTTACTGGAGCTGGACCAGCGGACC | |
| | CACTTCCCCCAGTTCTCCTACTCGGCCAGCATCCGCGAGTGA | |
| Akt3 encoding | ATGAGCGATGTTACCATTGTGAAAGAAGGTTGGGTTCAGAAGAGGGGAGAATAT | 126 |
| DNA sequence | ATAAAAAACTGGAGGCCAAGATACTTCCTTTTGAAGACAGATGGCTCATTCATA | |
| (from Genbank | GGATATAAAGAGAAACCTCAAGATGTGGATTTACCTTATCCCCTCAACAACTTT | |
| NM_005465.7) | TCAGTGGCAAAATGCCAGTTAATGAAAACAGAACGACCAAAGCCAAACACATTT | |
| Bold and | ATAATCAGATGTCTCCAGTGGACTACTGTTATAGAGAGAACATTTCATGTAGAT | |
| italicized: siRNA | ACTCCAGAGGAAAGGGAAGAATGGACAGAAGCTATCCAGGCTGTAGCAGACAGA | |
| binding regions | CTGCAGAGGCAAGAAGAGGAGAGAATGAATTGTAGTCCAACTTCACAAATTGAT | |
| | AATATAGGAGAGGAAGAGATGGATGCCTCTACAACCCATCATAAAAGAAAGACA | |
| | ATGAATGATTTTGACTATTTGAAACTACTAGGTAAAGGCACTTTTGGGAAAGTT | |
| | ATTTTGGTTCGAGAGAAGGCAAGTGGAAAATACTATGCTATGAAGATTCTGAAG | |
| | AAAGAAGTCATTATTGCAAAGGATGAAGTGGCACACACTCTAACTGAAAGCAGA | |
| | GTATTAAAGAACACTAGACATCCCTTTTTAACATCCTTGAAATATTCCTTCCAG | |
| | ACAAAAGACCGTTTGTGTTTTGTGATGGAATATGTTAATGGGGGCGAGCTGTTT | |
| | TTCCATTTGTCGAGAGAGCGGGTGTTCTCTGAGGACCGCACACGTTTCTATGGT | |
| | GCAGAAATTGTCTCTGCCTTGGACTATCTACATTCCGGAAAGATTGTGTACCGT | |
| | GATCTCAAGTTGGAGAATCTAATGCTGGACAAAGATGGCCACATAAAAATTACA | |
| | GATTTTGGACTTTGCAAAGAAGGGATCACAGATGCAGCCACCATGAAGACATTC | |
| | TGTGGCACTCCAGAATATCTGGCACCAGAGGTGTTAGAAGATAATGACTATGGC | |
| | CGAGCAGTAGACTGGTGGGGCCTAGGGGTTGTCATGTATGAAATGATGTGTGGG | |
| | AGGTTACCTTTCTACAACCAGGACCATGAGAAACTTTTTGAATTAATATTAATG | |
| | GAAGACATTAAATTTCCTCGAACACTCTCTTCAGATGCAAAATCATTGCTTTCA | |
| | GGGCTCTTGATAAAGGATCCAAATAAACGCCTTGGTGGAGGACCAGATGATGCA | |
| | AAAGAAATTATGAGACACAGTTTCTTCTCTGGAGTAAACTGGCAAGATGTATAT | |
| | GATAAAAAGCTTGTACCTCCTTTTAAACCTCAAGTAACATCTGAGACAGATACT | |
| | AGATATTTTGATGAAGAATTTACAGCTCAGACTATTACAATAACACCACCTGAA | |
| | AAATATGATGAGGATGGTATGGACTGCATGGACAATGAGAGGCGGCCGCATTTC | |
| | CCTCAATTTTCCTACTCTGCAAGTGGACGAGAATAA | |
| VEGFA-siRNA | GGAGGGCAGAATCATCACGAAGTGGTGAAGT | 127 |
| sense strand | | |
| (Cpd.10-Cpd.15 | | |
| siRNA #1) | | |
| VEGFA-siRNA | GAGATGAGCTTCCTACAGCACAACAAATGTG | 128 |
| sense strand | | |
| (Cpd.10-Cpd.15 | | |
| siRNA #2) | | |
| VEGFA-siRNA | GTACAAGATCCGCAGACGTGTAAATGTTCC | 129 |
| sense strand | | |
| (Cpd.10-Cpd.15 | | |
| siRNA #3) | | |
| c-Myc-siRNA | ACGACGAGACCTTCATCAA | 130 |
| sense strand | | |
| (Cpd.16-Cpd.17 | | |
| siRNA #1) | | |
| KRAS-siRNA | GTGCAATGAGGGACCAGTACA | 131 |
| sense strand | | |
| (Cpd.16-Cpd.17 | | |
| siRNA #2) | | |
| Akt(pan)-siRNA | TTCTACAACCAGGACCATGAG | 132 |
| sense strand | | |
| (Cpd.16-Cpd.17 | | |
| siRNA #3) | | |
| VEGFA-siRNA | ACTTCACCACTTCGTGATGATTCTGCCCTCC | 133 |
| anti-sense strand | | |
| (Cpd.10-Cpd.15 | | |
| siRNA #1) | | |
| VEGFA-siRNA | CACATTTGTTGTGCTGTAGGAAGCTCATCTC | 134 |
| anti-sense strand | | |
| (Cpd.10-Cpd.15 | | |
| siRNA #2) | | |
| VEGFA-siRNA | GGAACATTTACACGTCTGCGGATCTTGTAC | 135 |
| anti-sense strand | | |
| (Cpd.10-Cpd.15 | | |
| siRNA #3) | | |
| c-Myc-siRNA | TTGATGAAGGTCTCGTCGTCC | 136 |
| anti-sense strand | | |
| (Cpd.16-Cpd.17 | | |
| siRNA #1) | | |
| KRAS-siRNA | TGTACTGGTCCCTCATTGCAC | 137 |
| anti-sense strand | | |
| (Cpd.16-Cpd.17 | | |
| siRNA #2) | | |
| Akt(pan)-siRNA | CTCATGGTCCTGGTTGTAGAA | 138 |
| anti-sense strand | | |
| (Cpd.16-Cpd.17 | | |
| siRNA #3) | | |

What is claimed is:

1. A composition comprising a recombinant RNA construct comprising a first RNA sequence, a second RNA sequence, and a linker RNA sequence, wherein:
(i) the first RNA sequence is a first small interfering RNA (siRNA) sequence;
(ii) the second RNA sequence is a second siRNA sequence or a first messenger RNA (mRNA) sequence encoding a gene of interest (GOI); and
(iii) the linker RNA sequence links the first RNA sequence and the second RNA sequence, wherein the linker RNA sequence has a structure selected from the group consisting of:
XₘCAACAAXₙ, Formula (I):
wherein X is any nucleotide, m is an integer from 1 to 12, and n is an integer from 0 to 4 (SEQ ID NO: 151); and
XₚTCCCXᵣ, Formula (II):
wherein X is any nucleotide, p is an integer from 0 to 17, and r is an integer from 0 to 13 (SEQ ID NO: 152).

2. A composition comprising a recombinant RNA construct comprising a first RNA sequence, a second RNA sequence, and a linker RNA sequence, wherein:
(i) the first RNA sequence is a first small interfering RNA (siRNA) sequence;
(ii) the second RNA sequence is a second siRNA sequence or a first messenger RNA (mRNA) sequence encoding a gene of interest (GOI); and
(iii) the linker RNA sequence links the first RNA sequence and the second RNA sequence, wherein the linker RNA sequence comprises or consists of ACAACAA (SEQ ID NO: 23).

3. A composition comprising a recombinant RNA construct comprising a first RNA sequence, a second RNA sequence, and a linker RNA sequence, wherein:
(i) the first RNA sequence is a first small interfering (siRNA) sequence;
(ii) the second RNA sequence is a second siRNA sequence or a first messenger (mRNA) sequence encoding a gene of interest (GOI); and
(iii) the linker RNA sequence links the first RNA sequence and the second RNA sequence, wherein
(a) the linker RNA sequence is not
(SEQ ID NO: 22)
TTTATCTTAGAGGCATATCCCTACGTACCAACAA
or
(SEQ ID NO: 21)
ATAGTGAGTCGTATTAACGTACCAACAA;
or
(b) the linker RNA sequence does not form a secondary structure according to RNAfold WebServer.

4. The composition of any one of claims 1-3, wherein the second RNA sequence is a second siRNA sequence.

5. The composition of claim 4, wherein the linker RNA sequence comprises or consists of
(SEQ ID NO: 23)
ACAACAA,
(SEQ ID NO: 67)
ATCCCTACGTACCAACAA,
(SEQ ID NO: 68)
ACGTACCAACAA,
(SEQ ID NO: 69)
TCCC,
or
(SEQ ID NO: 70)
ACAACAATCCC.

6. The composition of claim 4, wherein the recombinant RNA construct further comprises a first mRNA sequence encoding a GOI.

7. The composition of any one of claims 1-3, wherein the second RNA sequence is a first mRNA sequence encoding a GOI.

8. The composition of claim 7, wherein the linker RNA sequence comprises or consists of
(SEQ ID NO: 23)
ACAACAA,
(SEQ ID NO: 72)
ATAGTGAGTCGTATTATCCC,
(SEQ ID NO: 73)
ATAGTGAGTCGTATTAACAACAATCCC,
(SEQ ID NO: 74)
ATAGTGAGTCGTATTAACAACAA,
(SEQ ID NO: 75)
ATAGTGAGTCGTATTAATCCCTACGTACCAACAA,
or
(SEQ ID NO: 21)
ATAGTGAGTCGTATTAACGTACCAACAA.

9. The composition of any one of claims 6-8, wherein the recombinant RNA construct further comprises a second mRNA sequence encoding a GOI.

10. The composition of any one of claims 7-9, wherein the recombinant RNA construct further comprises a second siRNA sequence.

11. The composition of any one of claim 4-6 or 10, wherein the recombinant RNA construct comprises a third siRNA sequence.

12. The composition of claim 11, wherein the recombinant RNA construct further comprises four, five, or more siRNA sequences.

13. The composition of claim 12, wherein each of the siRNA sequences binds to a target RNA and modulates the expression of the target RNA.

14. The composition of claim 13, wherein each of the siRNA sequences is capable of binding to:
(a) different target RNAs;
(b) different regions of the same target RNA;
(c) the same region of the same target RNA; or
(d) any combinations thereof.

15. The composition of claim 14, wherein the siRNA sequences of (c) are the same.

16. The composition of any one of claims 9-15, wherein the recombinant RNA construct comprises three, four, five, or more mRNA sequences, each encoding a GOI.

17. The composition of claim 16, wherein each of the mRNA sequences encodes the same GOI.

18. The composition of claim 16, wherein each of the mRNA sequences encodes a different GOI.

19. The composition of any one of the preceding claims, wherein the length of the linker RNA sequence between siRNA sequences is from about 4 to about 27 nucleotides.

20. The composition of any one of the preceding claims, wherein the length of the linker RNA sequence between siRNA sequences is from about 4 to about 18 nucleotides.

21. The composition of any one of the preceding claims, wherein m is 1 and n is 0.

22. The composition of claim 2, wherein the linker RNA sequence between siRNA sequences is ACAACAATCCC (SEQ ID NO: 70).

23. The composition of claim 2, wherein the linker RNA sequence is ACAACAA (SEQ ID NO: 23).

24. The composition of any one of the preceding claims, wherein the linker RNA sequence comprises a sequence selected from the group consisting of SEQ ID NOs: 23 and 67-75.

25. The composition of any one of the preceding claims, wherein the linker RNA sequence comprises or consists of a sequence according to SEQ ID NO: 23.

26. The composition of any one of the preceding claims, wherein the linker RNA sequence comprises or consists of a sequence according to SEQ ID NO: 67.

27. The composition of any one of the preceding claims, wherein the linker RNA sequence comprises or consists of a sequence according to SEQ ID NO: 68.

28. The composition of any one of the preceding claims, wherein the linker RNA sequence comprises or consists of a sequence according to SEQ ID NO: 69.

29. The composition of any one of the preceding claims, wherein the linker RNA sequence comprises or consists of a sequence according to SEQ ID NO: 70.

30. The composition of any one of claims 24-29, wherein expression of a target RNA targeted by the siRNA is lower using a recombinant RNA construct comprising the linker RNA sequence according to SEQ ID NO: 67 compared to (i) expression of the target RNA targeted by the siRNA using a recombinant RNA construct comprising the linker RNA sequence according to SEQ ID NO: 68, (ii) expression of the target RNA targeted by the siRNA using a recombinant RNA construct comprising the linker RNA sequence according to SEQ ID NO: 69, (iii) expression of the target RNA targeted by the siRNA using a recombinant RNA construct comprising the linker RNA sequence according to SEQ ID NO: 70, and/or (iv) expression of the target RNA targeted by the siRNA using a recombinant RNA construct comprising the linker RNA sequence according to SEQ ID NO: 23.

31. The composition of any one of claims 24-30, wherein expression of a first mRNA sequence encoding a GOI is higher using a recombinant RNA construct comprising the linker RNA sequence according to SEQ ID NO: 70 compared to (i) expression of the first mRNA sequence encoding the GOI using a recombinant RNA construct comprising the linker RNA sequence according to SEQ ID NO: 67, (ii) expression of the first mRNA sequence encoding the GOI using a recombinant RNA construct comprising the linker RNA sequence according to SEQ ID NO: 68, (iii) expression of the first mRNA sequence encoding the GOI using a recombinant RNA construct comprising the linker RNA sequence according to SEQ ID NO: 69, and/or (iv) expression of the first mRNA sequence encoding the GOI using a recombinant RNA construct comprising the linker RNA sequence according to SEQ ID NO: 23.

32. The composition of any one of claims 24-31, wherein expression of a target RNA targeted by the siRNA is lower using a recombinant RNA construct comprising the linker RNA sequence according to SEQ ID NO: 23 compared to (i) expression of the target RNA targeted by the siRNA using a recombinant RNA construct comprising the linker RNA sequence according to SEQ ID NO: 69, and/or (ii) expression of the target RNA targeted by the siRNA using a recombinant RNA construct comprising the linker RNA sequence according to SEQ ID NO: 70.

33. The composition of any one of claims 24-32, wherein expression of a first mRNA sequence encoding a GOI is higher using a recombinant RNA construct comprising the linker RNA sequence according to SEQ ID NO: 23 compared to (i) expression of the first mRNA sequence encoding the GOI using a recombinant RNA construct comprising the linker RNA sequence according to SEQ ID NO: 67, (ii) expression of the first mRNA sequence encoding the GOI using a recombinant RNA construct comprising the linker RNA sequence according to SEQ ID NO: 68, and/or (iii) expression of the first mRNA sequence encoding the GOI using a recombinant RNA construct comprising the linker RNA sequence according to SEQ ID NO: 69.

34. The composition of any one of the preceding claims, wherein the linker RNA sequence is selected based on a desired expression level of the first mRNA sequence encoding the GOI and/or a desired expression level of the target RNA targeted by the siRNA or desired expression level of a protein encoded by the target RNA targeted by the siRNA.

35. The composition of any one of the preceding claims, wherein the expression of the GOI is modulated.

36. The composition of claim 35, wherein the expression of the GOI is upregulated by expressing a protein encoded by the GOI.

37. The composition of any one of the preceding claims, wherein the expression of the target RNA is modulated.

38. The composition of claim 37, wherein the expression of the target RNA is downregulated by the siRNA sequences capable of binding to the target RNA.

39. The composition of any one of the preceding claims, wherein the siRNA sequences capable of binding to the target RNA do not inhibit the expression of the GOI.

40. The composition of any one of the preceding claims, wherein the RNA linker sequence between siRNA sequences does not form a secondary structure according to RNAfold WebServer.

41. The composition of any one of the preceding claims, wherein an siRNA sequence forms a secondary structure according to RNAfold WebServer.

42. The composition of claim 41, wherein the siRNA sequence comprises a hairpin structure or a loop structure.

43. The composition of any one of the preceding claims, wherein the siRNA sequences comprise one or more short or small hairpin RNAs (shRNAs).

44. The composition of any one of the preceding claims, wherein the recombinant RNA construct is cleaved.

45. The composition of claim 44, wherein the recombinant RNA construct is cleaved by an intracellular protein.

46. The composition of claim 44 or 45, wherein the recombinant RNA construct is cleaved by an endogenous protein.

47. The composition of any one of claims 44-46, wherein the recombinant RNA construct is cleaved by an endogenous DICER.

48. The composition of any one of claims 44-47, wherein the cleavage of the recombinant RNA construct is enhanced compared to the cleavage of an RNA construct that does not comprise a linker having a structure selected from the group consisting of Formula (I) and Formula (II).

49. The composition of any one of claims 44-47, wherein the cleavage of the recombinant RNA construct is enhanced compared to the cleavage of an RNA construct that does not comprise a linker comprising a sequence comprising ACAACAA (SEQ ID NO: 23).

50. The composition of any one of claims 44-47, wherein the cleavage of the recombinant RNA construct is enhanced compared to the cleavage of an RNA construct comprising a linker that forms a secondary structure.

51. The composition of any one of the preceding claims, wherein the expression of the gene of interest is enhanced compared to the expression of a gene of interest from an RNA construct that does not comprise a linker having a structure selected from the group consisting of Formula (I) and Formula (II).

52. The composition of any one of the preceding claims, wherein the expression of the gene of interest is enhanced compared to the expression of a gene of interest from an RNA construct that does not comprise a linker comprising a sequence comprising ACAACAA (SEQ ID NO: 23).

53. The composition of any one of the preceding claims, wherein the GOI comprises Interleukin 4 (IL-4), Interleukin 2 (IL-2), Interleukin 12 (IL-12), or Insulin-like Growth Factor 1 (IGF1).

54. The composition of any one of the preceding claims, wherein the target RNA is a noncoding RNA.

55. The composition of any one of claims 1-53, wherein the target RNA is a messenger RNA (mRNA).

56. The composition of any one of claims 1-53, wherein the target RNA is an mRNA encoding a protein selected from the group consisting of Tumor Necrosis Factor alpha (TNF-α), Activin Receptor-like Kinase 2 (ALK2), Vascular Endothelial Growth Factor A (VEGFA), Cellular Myelocytomatosis (c-Myc), Kirsten Rat Sarcoma (KRAS), Protein kinase B-1 (Akt1), Akt2, and Akt3.

57. The composition of any one of the preceding claims, wherein the siRNA sequences capable of binding to the target RNA bind to an exon of the target RNA.

58. The composition of any one of the preceding claims, wherein the siRNA sequences capable of binding to the target RNA specifically bind to one target RNA.

59. The composition of any one of the preceding claims, wherein the siRNA sequences capable of binding to the target RNA are not encoded by or comprised of an intron sequence of the gene of interest.

60. The composition of any one of the preceding claims, wherein the GOI is expressed without RNA splicing.

61. The composition of any one of the preceding claims, wherein the first RNA sequence is present downstream or 3′ of the second RNA sequence.

62. The composition of claim 61, wherein the RNA construct comprises an internal ribosome entry site (IRES) downstream or 3′ of the second RNA sequence.

63. The composition of claim 61 or 62, wherein the RNA construct comprises an internal ribosome entry site (IRES) immediately upstream or 5′ of the first RNA sequence.

64. The composition of any one of claims 1-60, wherein the first RNA sequence is present upstream or 5′ of the second RNA sequence.

65. The composition of claim 64, wherein the RNA construct comprises an internal ribosome entry site (IRES) upstream or 5′ of the first RNA sequence.

66. The composition of any one of the preceding claims, wherein the RNA construct further comprises a poly(A) tail, a 5′ cap, or a Kozak sequence.

67. The composition of any one of the preceding claims, wherein the first RNA sequence and the second RNA sequence are both recombinant.

68. The composition of any one of the preceding claims, wherein the siRNA comprises a sense strand sequence selected from the group consisting of SEQ ID NOs: 50-57 and 127-132.

69. The composition of any one of the preceding claims for use in modulating the expression of two or more genes in a cell.

70. A pharmaceutical composition comprising a therapeutically effective amount of the composition of any one of claims 1-68 and a pharmaceutically acceptable excipient.

71. A cell comprising the composition of any one of claims 1-68.

72. A vector comprising a recombinant polynucleic acid construct encoding the composition of any one of claims 1-68.

73. A method of producing an siRNA and an mRNA from a single RNA transcript in a cell, comprising introducing into the cell the composition of any one of claims 1-68, or the vector of claim 72.

74. A method of modulating protein expression comprising introducing the composition of any one of claims 1-68 or the vector of claim 72 into a cell, wherein the expression of a protein encoded by the target RNA is decreased.

75. A method of modulating protein expression comprising introducing the composition of any one of claims 1-68 or the vector of claim 72 into a cell, wherein the expression of a protein encoded by a gene of interest (GOI) is increased.

76. A method of modulating protein expression comprising introducing the composition of any one of claims 1-68 or the vector of claim 72 into a cell, wherein the expression of a protein encoded by the target RNA is decreased, and wherein the expression of a protein encoded by a gene of interest (GOI) is increased.

77. A method of treating a disease or condition comprising administering to a subject in need thereof the composition of any one of claims 1-68 or the pharmaceutical composition of claim 70.

78. The method of claim 77, wherein the disease or condition comprises a skin disease or condition or a muscular disease or condition.

79. The method of claim 78, wherein the skin disease or condition comprises an inflammatory skin disorder.

80. The method of claim 79, wherein the inflammatory skin disorder comprises psoriasis.

81. The method of claim 78, wherein the muscular disease or condition comprises a skeletal muscle disorder.

82. The method of claim 81, wherein the skeletal muscle disorder comprises fibrodysplasia ossificans progressiva (FOP).

83. The method of claim 77, wherein the disease or condition comprises cancer.

84. The method of claim 83, wherein the cancer comprises glioblastoma, human tongue squamous carcinoma, human lung carcinoma, or human monocyte leukemia.

85. The method of any one of claims 77-84, wherein the subject is a human.

86. A composition comprising a recombinant RNA construct comprising a first RNA sequence, a first linker RNA sequence, a second RNA sequence, and a second linker RNA sequence, wherein:
(i) the first RNA sequence is a messenger RNA (mRNA) encoding Interleukin 4 (IL-4);
(ii) the second RNA sequence comprises two or more small interfering RNAs (siRNAs) capable of binding to a Tumor Necrosis Factor alpha (TNF-α) mRNA;
(iii) the first linker RNA sequence is present between the first RNA sequence and the second RNA sequence; and
(iv) the second linker RNA sequence links each of the two or more siRNAs and comprises a sequence according to SEQ ID NO: 23.

87. A composition comprising a recombinant RNA construct comprising a first RNA sequence, a first linker RNA sequence, a second RNA sequence, and a second linker RNA sequence, wherein:
(i) the first RNA sequence is a messenger RNA (mRNA) encoding Insulin-like Growth Factor 1 (IGF1);
(ii) the second RNA sequence comprises two or more small interfering RNAs (siRNAs) capable of binding to a Activin Receptor-like Kinase 2 (ALK2) mRNA;
(iii) the first linker RNA sequence is present between the first RNA sequence and the second RNA sequence; and
(iv) the second linker RNA sequence links each of the two or more siRNAs and comprises a sequence according to SEQ ID NO: 23.

88. A composition comprising a recombinant RNA construct comprising a first RNA sequence, a first linker RNA sequence, a second RNA sequence, and a second linker RNA sequence, wherein:
(i) the first RNA sequence is a messenger RNA (mRNA) encoding Interleukin 2 (IL-2);
(ii) the second RNA sequence comprises two or more small interfering RNAs (siRNAs) capable of binding to a Vascular Endothelial Growth Factor A (VEGFA) mRNA;
(iii) the first linker RNA sequence is present between the first RNA sequence and the second RNA sequence; and
(iv) the second linker RNA sequence links each of the two or more siRNAs and comprises a sequence selected from the group consisting of SEQ ID NOs: 23 and 67-70.

89. A composition comprising a recombinant RNA construct comprising a first RNA sequence, a first linker RNA sequence, a second RNA sequence, and a second linker RNA sequence, wherein:
(i) the first RNA sequence is a messenger RNA (mRNA) encoding Interleukin 12 (IL-12);
(ii) the second RNA sequence comprises two or more small interfering RNAs (siRNAs) capable of binding to an mRNA of Cellular Myelocytomatosis (c-Myc), Kirsten Rat Sarcoma (KRAS), Protein kinase B-1 (Akt1), Akt2, and/or Akt3;
(iii) the first linker RNA sequence is present between the first RNA sequence and the second RNA sequence; and
(iv) the second linker RNA sequence links each of the two or more siRNAs and comprises a sequence according to SEQ ID NO: 23.

90. A composition comprising a recombinant polynucleic acid construct comprising a nucleic acid sequence selected from the group consisting of SEQ ID NOs: 1-18 and 76-108.


SEQUENCE LISTING (156 sequences)

SEQ ID NO: 1 - DNA, 741 bp, synthetic construct
  misc_feature 1..741: note = Description of Artificial Sequence: Synthetic polynucleotide
  source 1..741: mol_type = other DNA
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatcccttttatcttagaggcatatccct

SEQ ID NO: 2 - DNA, 651 bp, synthetic construct
  misc_feature 1..651: note = Description of Artificial Sequence: Synthetic polynucleotide
  source 1..651: mol_type = other DNA
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaacaacaaggcgtggagctgagagataaacttgttatctctcagctccacgccacaacaagggcctgtacctcatctactacttgagtagatgaggtacaggcccacaacaaggtatgagcccatctatctacttgagatagatgggctcataccacaacaatttatcttagaggcatatccct

SEQ ID NO: 3 - DNA, 617 bp, synthetic construct
  misc_feature 1..617: note = Description of Artificial Sequence: Synthetic polynucleotide
  source 1..617: mol_type = other DNA
gccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaatagtgagtcgtattaacgtaccaacaaggcctcattattctctctacttgagagagaataatgaggcctttatcttagaggcatatccctacgtaccaacaagtgttcgcagtatgtcttacttgaagacatactgcgaacactttatcttagaggcatatccctacgtaccaacaagcctgcctgctgggagttacttgaactcccagcaggcaggctttatcttagaggcatatcccttttatcttagaggcatatccct

SEQ ID NO: 4 - DNA, 527 bp, synthetic construct
  misc_feature 1..527: note = Description of Artificial Sequence: Synthetic polynucleotide
  source 1..527: mol_type = other DNA
gccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaacaacaaggcctcattattctctctacttgagagagaataatgaggccacaacaagtgttcgcagtatgtcttacttgaagacatactgcgaacacacaacaagcctgcctgctgggagttacttgaactcccagcaggcaggcacaacaatttatcttagaggcatatccct

SEQ ID NO: 5 - DNA, 482 bp, synthetic construct
  misc_feature 1..482: note = Description of Artificial Sequence: Synthetic polynucleotide
  source 1..482: mol_type = other DNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatcccttttatcttagaggcatatccct

SEQ ID NO: 6 - DNA, 446 bp, synthetic construct
  misc_feature 1..446: note = Description of Artificial Sequence: Synthetic polynucleotide
  source 1..446: mol_type = other DNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaacaacaacaacaagatgaagagcaccaaacttgttggtgctcttcatcttgttgacaacaatttatcttagaggcatatccct

SEQ ID NO: 7 - DNA, 563 bp, synthetic construct
  misc_feature 1..563: note = Description of Artificial Sequence: Synthetic polynucleotide
  source 1..563: mol_type = other DNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatcccttttatcttagaggcatatccct

SEQ ID NO: 8 - DNA, 563 bp, synthetic construct
  misc_feature 1..563: note = Description of Artificial Sequence: Synthetic polynucleotide
  source 1..563: mol_type = other DNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaaggacagccacatgcacttcaaacttgttgaagtgcatgtggctgtcctttatcttagaggcatatcccttttatcttagaggcatatccct

SEQ ID NO: 9 - DNA, 500 bp, synthetic construct
  misc_feature 1..500: note = Description of Artificial Sequence: Synthetic polynucleotide
  source 1..500: mol_type = other DNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaacaacaacaacaagatgaagagcaccaaacttgttggtgctcttcatcttgttgacaacaacaacaagatgaagagcaccaaacttgttggtgctcttcatcttgttgacaacaatttatcttagaggcatatccct

SEQ ID NO: 10 - DNA, 3082 bp, synthetic construct
  misc_feature 1..3082: note = Description of Artificial Sequence: Synthetic polynucleotide
  source 1..3082: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 11 - DNA, 2992 bp, synthetic construct
  misc_feature 1..2992: note = Description of Artificial Sequence: Synthetic polynucleotide
  source 1..2992: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaacaacaaggcgtggagctgagagataaacttgttatctctcagctccacgccacaacaagggcctgtacctcatctactacttgagtagatgaggtacaggcccacaacaaggtatgagcccatctatctacttgagatagatgggctcataccacaacaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 12 - DNA, 2958 bp, synthetic construct
  misc_feature 1..2958: note = Description of Artificial Sequence: Synthetic polynucleotide
  source 1..2958: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaatagtgagtcgtattaacgtaccaacaaggcctcattattctctctacttgagagagaataatgaggcctttatcttagaggcatatccctacgtaccaacaagtgttcgcagtatgtcttacttgaagacatactgcgaacactttatcttagaggcatatccctacgtaccaacaagcctgcctgctgggagttacttgaactcccagcaggcaggctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 13 - DNA, 2868 bp, synthetic construct
  misc_feature 1..2868: note = Description of Artificial Sequence: Synthetic polynucleotide
  source 1..2868: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaacaacaaggcctcattattctctctacttgagagagaataatgaggccacaacaagtgttcgcagtatgtcttacttgaagacatactgcgaacacacaacaagcctgcctgctgggagttacttgaactcccagcaggcaggcacaacaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 14 - DNA, 2823 bp, synthetic construct
  misc_feature 1..2823: note = Description of Artificial Sequence: Synthetic polynucleotide
  source 1..2823: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 15 - DNA, 2787 bp, synthetic construct
  misc_feature 1..2787: note = Description of Artificial Sequence: Synthetic polynucleotide
  source 1..2787: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaacaacaacaacaagatgaagagcaccaaacttgttggtgctcttcatcttgttgacaacaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 16 - DNA, 2904 bp, synthetic construct
  misc_feature 1..2904: note = Description of Artificial Sequence: Synthetic polynucleotide
  source 1..2904: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 17 - DNA, 2904 bp, synthetic construct
  misc_feature 1..2904: note = Description of Artificial Sequence: Synthetic polynucleotide
  source 1..2904: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaaggacagccacatgcacttcaaacttgttgaagtgcatgtggctgtcctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 18 - DNA, 2841 bp, synthetic construct
  misc_feature 1..2841: note = Description of Artificial Sequence: Synthetic polynucleotide
  source 1..2841: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaacaacaacaacaagatgaagagcaccaaacttgttggtgctcttcatcttgttgacaacaacaacaagatgaagagcaccaaacttgttggtgctcttcatcttgttgacaacaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 19 - skipped

SEQ ID NO: 20 - DNA, 17 bp, synthetic construct
  misc_feature 1..17: note = Description of Artificial Sequence: Synthetic oligonucleotide
  source 1..17: mol_type = other DNA
taatacgactcactata

SEQ ID NO: 21 - DNA, 28 bp, synthetic construct
  misc_feature 1..28: note = Description of Artificial Sequence: Synthetic oligonucleotide
  source 1..28: mol_type = other DNA
atagtgagtcgtattaacgtaccaacaa

SEQ ID NO: 22 - DNA, 34 bp, synthetic construct
  misc_feature 1..34: note = Description of Artificial Sequence: Synthetic oligonucleotide
  source 1..34: mol_type = other DNA
tttatcttagaggcatatccctacgtaccaacaa

SEQ ID NO: 23 - skipped

SEQ ID NO: 24 - DNA, 20 bp, synthetic construct
  misc_feature 1..20: note = Description of Artificial Sequence: Synthetic primer
  source 1..20: mol_type = other DNA
gctgcaaggcgattaagttg

SEQ ID NO: 25 - DNA, 143 bp, synthetic construct
  misc_feature 1..143: note = Description of Artificial Sequence: Synthetic primer
  source 1..143: mol_type = other DNA
  misc_feature 1..143: note = Combined DNA/RNA Molecule: Synthetic primer
  misc_feature 1..3: note = RNA fragment
  modified_base 1..3: mod_base = um
  misc_feature 4..143: note = DNA fragment
ttttttttttttttttttttttttttttttttttttttttttttttttttttttttttttttttttttttttttttttttttttttttttttttttttttttttttttttttttttttttcagctatgaccatgttaatgcag

SEQ ID NO: 26 - DNA, 615 bp, Homo sapiens
  source 1..615: mol_type = genomic DNA
atcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatctaa

SEQ ID NO: 27 - protein, 153 aa, Homo sapiens [sequence omitted]

SEQ ID NO: 28 - DNA, 462 bp, Homo sapiens
  source 1..462: mol_type = genomic DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtag

SEQ ID NO: 29 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 30 - DNA, 348 bp, synthetic construct
  misc_feature 1..348: note = Description of Artificial Sequence: Synthetic polynucleotide
  source 1..348: mol_type = other DNA
atgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 31 - protein, 115 aa, synthetic construct
  REGION 1..115: note = Description of Artificial Sequence: Synthetic polypeptide
MTILFLTMVISYFGCMKAVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSA

SEQ ID NO: 32 - DNA, 702 bp, Homo sapiens
  source 1..702: mol_type = genomic DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 33 - DNA, 1530 bp, Homo sapiens
  source 1..1530: mol_type = genomic DNA
atggtagatggagtgatgattcttcctgtgcttatcatgattgctctcccctcccctagtatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagccgtggagtgctgccaaggggactggtgtaacaggaacatcacggcccagctgcccactaaaggaaaatccttccctggaacacagaatttccacttggaggttggcctcattattctctctgtagtgttcgcagtatgtcttttagcctgcctgctgggagttgctctccgaaaatttaaaaggcgcaaccaagaacgcctcaatccccgagacgtggagtatggcactatcgaagggctcatcaccaccaatgttggagacagcactttagcagatttattggatcattcgtgtacatcaggaagtggctctggtcttccttttctggtacaaagaacagtggctcgccagattacactgttggagtgtgtcgggaaaggcaggtatggtgaggtgtggaggggcagctggcaaggggagaatgttgccgtgaagatcttctcctcccgtgatgagaagtcatggttcagggaaacggaattgtacaacactgtgatgctgaggcatgaaaatatcttaggtttcattgcttcagacatgacatcaagacactccagtacccagctgtggttaattacacattatcatgaaatgggatcgttgtacgactatcttcagcttactactctggatacagttagctgccttcgaatagtgctgtccatagctagtggtcttgcacatttgcacatagagatatttgggacccaagggaaaccagccattgcccatcgagatttaaagagcaaaaatattctggttaagaagaatggacagtgttgcatagcagatttgggcctggcagtcatgcattcccagagcaccaatcagcttgatgtggggaacaatccccgtgtgggcaccaagcgctacatggcccccgaagttctagatgaaaccatccaggtggattgtttcgattcttataaaagggtcgatatttgggcctttggacttgttttgtgggaagtggccaggcggatggtgagcaatggtatagtggaggattacaagccaccgttctacgatgtggttcccaatgacccaagttttgaagatatgaggaaggtagtctgtgtggatcaacaaaggccaaacatacccaacagatggttctcagacccgacattaacctctctggccaagctaatgaaagaatgctggtatcaaaatccatccgcaagactcacagcactgcgtatcaaaaagactttgaccaaaattgataattccctcgacaaattgaaaactgactgttga

SEQ ID NO: 34 - DNA, 699 bp, Pontellina plumata
  source 1..699: mol_type = other DNA
atggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataa

SEQ ID NO: 35 - DNA, 22 bp, synthetic construct
  misc_feature 1..22: note = Description of Artificial Sequence: Synthetic primer
  source 1..22: mol_type = other DNA
gacgtggagtatggcactatcg

SEQ ID NO: 36 - DNA, 23 bp, synthetic construct
  misc_feature 1..23: note = Description of Artificial Sequence: Synthetic primer
  source 1..23: mol_type = other DNA
cactccaacagtgtaatctggcg

SEQ ID NO: 37 - DNA, 22 bp, synthetic construct
  misc_feature 1..22: note = Description of Artificial Sequence: Synthetic primer
  source 1..22: mol_type = other DNA
acccgttgaaccccattcgtga

SEQ ID NO: 38 - DNA, 23 bp, synthetic construct
  misc_feature 1..23: note = Description of Artificial Sequence: Synthetic primer
  source 1..23: mol_type = other DNA
gcctcactaaaccatccaatcgg

SEQ ID NO: 39 - DNA, 77 bp, synthetic construct
  misc_feature 1..77: note = Description of Artificial Sequence: Synthetic oligonucleotide
  source 1..77: mol_type = other DNA
aacaaagcaccagtggtctagtggtagaatagtaccctgccacggtacagacccgggttcgattcccggctggtgca

SEQ ID NO: 40 - DNA, 210 bp, Homo sapiens
  source 1..210: mol_type = other DNA
ggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcc

SEQ ID NO: 41 - protein, 19 aa, synthetic construct
  REGION 1..19: note = Description of Artificial Sequence: Synthetic peptide
MLILLLPLLLFKCFCDFLK

SEQ ID NO: 42 - DNA, 57 bp, synthetic construct
  misc_feature 1..57: note = Description of Artificial Sequence: Synthetic oligonucleotide
  source 1..57: mol_type = other DNA
atgctgattctgctgctgcccctgctgctgttcaagtgcttctgcgacttcctgaaa

SEQ ID NO: 43 - protein, 18 aa, synthetic construct
  REGION 1..18: note = Description of Artificial Sequence: Synthetic peptide
MLFYLALCLLTFTSSATA

SEQ ID NO: 44 - DNA, 54 bp, synthetic construct
  misc_feature 1..54: note = Description of Artificial Sequence: Synthetic oligonucleotide
  source 1..54: mol_type = other DNA
atgctgttctatctggccctgtgcctgctgacctttaccagctctgctaccgcc

SEQ ID NO: 45 - protein, 10 aa, synthetic construct
  REGION 1..10: note = Description of Artificial Sequence: Synthetic peptide
VKMHTMSSSH

SEQ ID NO: 46 - protein, 21 aa, Homo sapiens
MGKISSLPTQLFKCCFCDFLK

SEQ ID NO: 47 - DNA, 63 bp, Homo sapiens
  source 1..63: mol_type = other DNA
atgggaaaaatcagcagtcttccaacccaattatttaagtgctgcttttgtgatttcttgaag

SEQ ID NO: 48 - protein, 18 aa, synthetic construct
  REGION 1..18: note = Description of Artificial Sequence: Synthetic peptide
MTILFLTMVISYFGCMKA

SEQ ID NO: 49 - DNA, 54 bp, synthetic construct
  misc_feature 1..54: note = Description of Artificial Sequence: Synthetic oligonucleotide
  source 1..54: mol_type = other DNA
atgaccatcctgtttctgacaatggtcatcagctacttcggctgcatgaaggcc

SEQ ID NO: 50 - DNA, 20 bp, synthetic construct
  misc_feature 1..20: note = Description of Artificial Sequence: Synthetic oligonucleotide
  source 1..20: mol_type = other DNA
ggcgtggagctgagagataa

SEQ ID NO: 51 - DNA, 20 bp, synthetic construct
  misc_feature 1..20: note = Description of Artificial Sequence: Synthetic oligonucleotide
  source 1..20: mol_type = other DNA
gggcctgtacctcatctact

SEQ ID NO: 52 - DNA, 19 bp, synthetic construct
  misc_feature 1..19: note = Description of Artificial Sequence: Synthetic oligonucleotide
  source 1..19: mol_type = other DNA
ggtatgagcccatctatct

SEQ ID NO: 53 - DNA, 18 bp, synthetic construct
  misc_feature 1..18: note = Description of Artificial Sequence: Synthetic oligonucleotide
  source 1..18: mol_type = other DNA
ggcctcattattctctct

SEQ ID NO: 54 - DNA, 18 bp, synthetic construct
  misc_feature 1..18: note = Description of Artificial Sequence: Synthetic oligonucleotide
  source 1..18: mol_type = other DNA
gtgttcgcagtatgtctt

SEQ ID NO: 55 - DNA, 18 bp, synthetic construct
  misc_feature 1..18: note = Description of Artificial Sequence: Synthetic oligonucleotide
  source 1..18: mol_type = other DNA
gcctgcctgctgggagtt

SEQ ID NO: 56 - DNA, 21 bp, synthetic construct
  misc_feature 1..21: note = Description of Artificial Sequence: Synthetic oligonucleotide
  source 1..21: mol_type = other DNA
caacaagatgaagagcaccaa

SEQ ID NO: 57 - DNA, 21 bp, synthetic construct
  misc_feature 1..21: note = Description of Artificial Sequence: Synthetic oligonucleotide
  source 1..21: mol_type = other DNA
ggacagccacatgcacttcaa

SEQ ID NO: 58 - DNA, 20 bp, synthetic construct
  misc_feature 1..20: note = Description of Artificial Sequence: Synthetic oligonucleotide
  source 1..20: mol_type = other DNA
ttatctctcagctccacgcc

SEQ ID NO: 59 - DNA, 20 bp, synthetic construct
  misc_feature 1..20: note = Description of Artificial Sequence: Synthetic oligonucleotide
  source 1..20: mol_type = other DNA
agtagatgaggtacaggccc

SEQ ID NO: 60 - DNA, 19 bp, synthetic construct
  misc_feature 1..19: note = Description of Artificial Sequence: Synthetic oligonucleotide
  source 1..19: mol_type = other DNA
agatagatgggctcatacc

SEQ ID NO: 61 - DNA, 18 bp, synthetic construct
  misc_feature 1..18: note = Description of Artificial Sequence: Synthetic oligonucleotide
  source 1..18: mol_type = other DNA
agagagaataatgaggcc

SEQ ID NO: 62 - DNA, 18 bp, synthetic construct
  misc_feature 1..18: note = Description of Artificial Sequence: Synthetic oligonucleotide
  source 1..18: mol_type = other DNA
aagacatactgcgaacac

SEQ ID NO: 63 - DNA, 18 bp, synthetic construct
  misc_feature 1..18: note = Description of Artificial Sequence: Synthetic oligonucleotide
  source 1..18: mol_type = other DNA
aactcccagcaggcaggc

SEQ ID NO: 64 - DNA, 21 bp, synthetic construct
  misc_feature 1..21: note = Description of Artificial Sequence: Synthetic oligonucleotide
  source 1..21: mol_type = other DNA
ttggtgctcttcatcttgttg

SEQ ID NO: 65 - DNA, 21 bp, synthetic construct
  misc_feature 1..21: note = Description of Artificial Sequence: Synthetic oligonucleotide
  source 1..21: mol_type = other DNA
ttgaagtgcatgtggctgtcc

SEQ ID NO: 66 - DNA, 16 bp, synthetic construct
  misc_feature 1..16: note = Description of Artificial Sequence: Synthetic oligonucleotide
  source 1..16: mol_type = other DNA
atagtgagtcgtatta

SEQ ID NO: 67 - DNA, 18 bp, synthetic construct
  misc_feature 1..18: note = Description of Artificial Sequence: Synthetic oligonucleotide
  source 1..18: mol_type = other DNA
atccctacgtaccaacaa

SEQ ID NO: 68 - DNA, 12 bp, synthetic construct
  misc_feature 1..12: note = Description of Artificial Sequence: Synthetic oligonucleotide
  source 1..12: mol_type = other DNA
acgtaccaacaa

SEQ ID NO: 69 - skipped

SEQ ID NO: 70 - DNA, 11 bp, synthetic construct
  misc_feature 1..11: note = Description of Artificial Sequence: Synthetic oligonucleotide
  source 1..11: mol_type = other DNA
acaacaatccc

SEQ ID NO: 71 - skipped

SEQ ID NO: 72 - DNA, 20 bp, synthetic construct
  misc_feature 1..20: note = Description of Artificial Sequence: Synthetic oligonucleotide
  source 1..20: mol_type = other DNA
atagtgagtcgtattatccc

SEQ ID NO: 73 - DNA, 27 bp, synthetic construct
  misc_feature 1..27: note = Description of Artificial Sequence: Synthetic oligonucleotide
  source 1..27: mol_type = other DNA
atagtgagtcgtattaacaacaatccc

SEQ ID NO: 74 - DNA, 23 bp, synthetic construct
  misc_feature 1..23: note = Description of Artificial Sequence: Synthetic oligonucleotide
  source 1..23: mol_type = other DNA
atagtgagtcgtattaacaacaa

SEQ ID NO: 75 - DNA, 34 bp, synthetic construct
  misc_feature 1..34: note = Description of Artificial Sequence: Synthetic oligonucleotide
  source 1..34: mol_type = other DNA
atagtgagtcgtattaatccctacgtaccaacaa

SEQ ID NO: 76 - RNA, 741 nt, synthetic construct
  misc_feature 1..741: note = Description of Artificial Sequence: Synthetic polynucleotide
  source 1..741: mol_type = other RNA
  modified_base 1..741: mod_base = m1f
  misc_difference 1..741: note = T in any of positions 1-741 are replaced with 1-methylpseudouridine.
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatcccttttatcttagaggcatatccct

SEQ ID NO: 77 - RNA, 651 nt, synthetic construct
  misc_feature 1..651: note = Description of Artificial Sequence: Synthetic polynucleotide
  source 1..651: mol_type = other RNA
  misc_difference 1..651: note = T in any of positions 1-651 are replaced with 1-methylpseudouridine.
  modified_base 1..651: mod_base = m1f
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaacaacaaggcgtggagctgagagataaacttgttatctctcagctccacgccacaacaagggcctgtacctcatctactacttgagtagatgaggtacaggcccacaacaaggtatgagcccatctatctacttgagatagatgggctcataccacaacaatttatcttagaggcatatccct

SEQ ID NO: 78 - RNA, 617 nt, synthetic construct
  misc_feature 1..617: note = Description of Artificial Sequence: Synthetic polynucleotide
  source 1..617: mol_type = other RNA
  misc_difference 1..617: note = T in any of positions 1-617 are replaced with 1-methylpseudouridine.
  modified_base 1..617: mod_base = m1f
gccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaatagtgagtcgtattaacgtaccaacaaggcctcattattctctctacttgagagagaataatgaggcctttatcttagaggcatatccctacgtaccaacaagtgttcgcagtatgtcttacttgaagacatactgcgaacactttatcttagaggcatatccctacgtaccaacaagcctgcctgctgggagttacttgaactcccagcaggcaggctttatcttagaggcatatcccttttatcttagaggcatatccct

SEQ ID NO: 79 - RNA, 527 nt, synthetic construct
  misc_feature 1..527: note = Description of Artificial Sequence: Synthetic polynucleotide
  source 1..527: mol_type = other RNA
  misc_difference 1..527: note = T in any of positions 1-527 are replaced with 1-methylpseudouridine.
  modified_base 1..527: mod_base = m1f
gccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaacaacaaggcctcattattctctctacttgagagagaataatgaggccacaacaagtgttcgcagtatgtcttacttgaagacatactgcgaacacacaacaagcctgcctgctgggagttacttgaactcccagcaggcaggcacaacaatttatcttagaggcatatccct

SEQ ID NO: 80 - RNA, 482 nt, synthetic construct
  misc_feature 1..482: note = Description of Artificial Sequence: Synthetic polynucleotide
  source 1..482: mol_type = other RNA
  misc_difference 1..482: note = T in any of positions 1-482 are replaced with 1-methylpseudouridine.
  modified_base 1..482: mod_base = m1f
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatcccttttatcttagaggcatatccct

SEQ ID NO: 81 - RNA, 446 nt, synthetic construct
  misc_feature 1..446: note = Description of Artificial Sequence: Synthetic polynucleotide
  source 1..446: mol_type = other RNA
  misc_difference 1..446: note = T in any of positions 1-446 are replaced with 1-methylpseudouridine.
  modified_base 1..446: mod_base = m1f
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaacaacaacaacaagatgaagagcaccaaacttgttggtgctcttcatcttgttgacaacaatttatcttagaggcatatccct

SEQ ID NO: 82 - RNA, 563 nt, synthetic construct
  misc_feature 1..563: note = Description of Artificial Sequence: Synthetic polynucleotide
  source 1..563: mol_type = other RNA
  misc_difference 1..563: note = T in any of positions 1-563 are replaced with 1-methylpseudouridine.
  modified_base 1..563: mod_base = m1f
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatcccttttatcttagaggcatatccct

SEQ ID NO: 83 - RNA, 563 nt, synthetic construct
  misc_feature 1..563: note = Description of Artificial Sequence: Synthetic polynucleotide
  source 1..563: mol_type = other RNA
  misc_difference 1..563: note = T in any of positions 1-563 are replaced with 1-methylpseudouridine.
  modified_base 1..563: mod_base = m1f
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaaggacagccacatgcacttcaaacttgttgaagtgcatgtggctgtcctttatcttagaggcatatcccttttatcttagaggcatatccct

SEQ ID NO: 84 - RNA, 500 nt, synthetic construct
  misc_feature 1..500: note = Description of Artificial Sequence: Synthetic polynucleotide
  source 1..500: mol_type = other RNA
  misc_difference 1..500: note = T in any of positions 1-500 are replaced with 1-methylpseudouridine.
  modified_base 1..500: mod_base = m1f
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaacaacaacaacaagatgaagagcaccaaacttgttggtgctcttcatcttgttgacaacaacaacaagatgaagagcaccaaacttgttggtgctcttcatcttgttgacaacaatttatcttagaggcatatccct

SEQ ID NO: 85 - DNA, 807 bp, synthetic construct
  misc_feature 1..807: note = Description of Artificial Sequence: Synthetic polynucleotide
  source 1..807: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagggcagaatcatcacgaagtggtgaagtacttgacttcaccacttcgtgatgattctgccctcctttatcttagaggcatatccctacgtaccaacaagagatgagcttcctacagcacaacaaatgtgacttgcacatttgttgtgctgtaggaagctcatctctttatcttagaggcatatccctacgtaccaacaagtacaagatccgcagacgtgtaaatgttccacttgggaacatttacacgtctgcggatcttgtactttatcttagaggcatatcccttttatcttagaggcatatccct

SEQ ID NO: 86 - DNA, 801 bp, synthetic construct
  misc_feature 1..801: note = Description of Artificial Sequence: Synthetic polynucleotide
  source 1..801: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaacaacaaggagggcagaatcatcacgaagtggtgaagtacttgacttcaccacttcgtgatgattctgccctccacaacaagagatgagcttcctacagcacaacaaatgtgacttgcacatttgttgtgctgtaggaagctcatctcacaacaagtacaagatccgcagacgtgtaaatgttccacttgggaacatttacacgtctgcggatcttgtacacaacaatttatcttagaggcatatccctctgggcctcatgggccttccgctcactgcccgctttccagtcgggaaacctgtcgtgccagctgcattaacatggtcatagctg

SEQ ID NO: 87 - DNA, 735 bp, synthetic construct
  misc_feature 1..735: note = Description of Artificial Sequence: Synthetic polynucleotide
  source 1..735: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaggagggcagaatcatcacgaagtggtgaagtacttgacttcaccacttcgtgatgattctgccctccatccctacgtaccaacaagagatgagcttcctacagcacaacaaatgtgacttgcacatttgttgtgctgtaggaagctcatctcatccctacgtaccaacaagtacaagatccgcagacgtgtaaatgttccacttgggaacatttacacgtctgcggatcttgtactttatcttagaggcat

SEQ ID NO: 88 - DNA, 723 bp, synthetic construct
  misc_feature 1..723: note = Description of Artificial Sequence: Synthetic polynucleotide
  source 1..723: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaggagggcagaatcatcacgaagtggtgaagtacttgacttcaccacttcgtgatgattctgccctccacgtaccaacaagagatgagcttcctacagcacaacaaatgtgacttgcacatttgttgtgctgtaggaagctcatctcacgtaccaacaagtacaagatccgcagacgtgtaaatgttccacttgggaacatttacacgtctgcggatcttgtactttatcttagaggcat

SEQ ID NO: 89 - DNA, 711 bp, synthetic construct
  misc_feature 1..711: note = Description of Artificial Sequence: Synthetic polynucleotide
  source 1..711: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattatcccggagggcagaatcatcacgaagtggtgaagtacttgacttcaccacttcgtgatgattctgccctcctcccgagatgagcttcctacagcacaacaaatgtgacttgcacatttgttgtgctgtaggaagctcatctctcccgtacaagatccgcagacgtgtaaatgttccacttgggaacatttacacgtctgcggatcttgtactttatcttagaggcat

SEQ ID NO: 90 - DNA, 732 bp, synthetic construct
  misc_feature 1..732: note = Description of Artificial Sequence: Synthetic polynucleotide
  source 1..732: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacaacaatcccggagggcagaatcatcacgaagtggtgaagtacttgacttcaccacttcgtgatgattctgccctccacaacaatcccgagatgagcttcctacagcacaacaaatgtgacttgcacatttgttgtgctgtaggaagctcatctcacaacaatcccgtacaagatccgcagacgtgtaaatgttccacttgggaacatttacacgtctgcggatcttgtactttatcttagaggcat

SEQ ID NO: 91 - DNA, 1910 bp, synthetic construct
  misc_feature 1..1910: note = Description of Artificial Sequence: Synthetic polynucleotide
  source 1..1910: mol_type = other DNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaaggacgacgagaccttcatcaaacttgttgatgaaggtctcgtcgtcctttatcttagaggcatatccctacgtaccaacaagtgcaatgagggaccagtacaacttgtgtactggtccctcattgcactttatcttagaggcatatccctacgtaccaacaattctacaaccaggaccatgagacttgctcatggtcctggttgtagaatttatcttagaggcatatcccttttatcttagaggcatatccct

SEQ ID NO: 92 - DNA, 1820 bp, synthetic construct
  misc_feature 1..1820: note = Description of Artificial Sequence: Synthetic polynucleotide
  source 1..1820: mol_type = other DNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaacaacaaggacgacgagaccttcatcaaacttgttgatgaaggtctcgtcgtccacaacaagtgcaatgagggaccagtacaacttgtgtactggtccctcattgcacacaacaattctacaaccaggaccatgagacttgctcatggtcctggttgtagaaacaacaatttatcttagaggcatatccct

SEQ ID NO: 93 - DNA, 3085 bp, synthetic construct
  misc_feature 1..3085: note = Description of Artificial Sequence: Synthetic polynucleotide
  source 1..3085: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagggcagaatcatcacgaagtggtgaagtacttgacttcaccacttcgtgatgattctgccctcctttatcttagaggcatatccctacgtaccaacaagagatgagcttcctacagcacaacaaatgtgacttgcacatttgttgtgctgtaggaagctcatctctttatcttagaggcatatccctacgtaccaacaagtacaagatccgcagacgtgtaaatgttccacttgggaacatttacacgtctgcggatcttgtactttatcttagaggcatatcccttttatcttagaggcatatccctctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 94 - DNA, 3477 bp, synthetic construct
  misc_feature 1..3477: note = Description of Artificial Sequence: Synthetic polynucleotide
  source 1..3477: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggattaatacgactcactatagggcgaattggcggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaacaacaaggagggcagaatcatcacgaagtggtgaagtacttgacttcaccacttcgtgatgattctgccctccacaacaagagatgagcttcctacagcacaacaaatgtgacttgcacatttgttgtgctgtaggaagctcatctcacaacaagtacaagatccgcagacgtgtaaatgttccacttgggaacatttacacgtctgcggatcttgtacacaacaatttatcttagaggcatatccctctgggcctcatgggccttccgctcactgcccgctttccagtcgggaaacctgtcgtgccagctgcattaacatggtcatagctgatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 95 - DNA, 3076 bp, synthetic construct
  misc_feature 1..3076: note = Description of Artificial Sequence: Synthetic polynucleotide
  source 1..3076: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaggagggcagaatcatcacgaagtggtgaagtacttgacttcaccacttcgtgatgattctgccctccatccctacgtaccaacaagagatgagcttcctacagcacaacaaatgtgacttgcacatttgttgtgctgtaggaagctcatctcatccctacgtaccaacaagtacaagatccgcagacgtgtaaatgttccacttgggaacatttacacgtctgcggatcttgtactttatcttagaggcatctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 96 - DNA, 3064 bp, synthetic construct
  misc_feature 1..3064: note = Description of Artificial Sequence: Synthetic polynucleotide
  source 1..3064: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaggagggcagaatcatcacgaagtggtgaagtacttgacttcaccacttcgtgatgattctgccctccacgtaccaacaagagatgagcttcctacagcacaacaaatgtgacttgcacatttgttgtgctgtaggaagctcatctcacgtaccaacaagtacaagatccgcagacgtgtaaatgttccacttgggaacatttacacgtctgcggatcttgtactttatcttagaggcatctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 97 - DNA, 3052 bp, synthetic construct
  misc_feature 1..3052: note = Description of Artificial Sequence: Synthetic polynucleotide
  source 1..3052: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattatcccggagggcagaatcatcacgaagtggtgaagtacttgacttcaccacttcgtgatgattctgccctcctcccgagatgagcttcctacagcacaacaaatgtgacttgcacatttgttgtgctgtaggaagctcatctctcccgtacaagatccgcagacgtgtaaatgttccacttgggaacatttacacgtctgcggatcttgtactttatcttagaggcatctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 98 - DNA, 3073 bp, synthetic construct
  misc_feature 1..3073: note = Description of Artificial Sequence: Synthetic polynucleotide
  source 1..3073: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacaacaatcccggagggcagaatcatcacgaagtggtgaagtacttgacttcaccacttcgtgatgattctgccctccacaacaatcccgagatgagcttcctacagcacaacaaatgtgacttgcacatttgttgtgctgtaggaagctcatctcacaacaatcccgtacaagatccgcagacgtgtaaatgttccacttgggaacatttacacgtctgcggatcttgtactttatcttagaggcatctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 99 - DNA, 4251 bp, synthetic construct
  misc_feature 1..4251: note = Description of Artificial Sequence: Synthetic polynucleotide
  source 1..4251: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaaggacgacgagaccttcatcaaacttgttgatgaaggtctcgtcgtcctttatcttagaggcatatccctacgtaccaacaagtgcaatgagggaccagtacaacttgtgtactggtccctcattgcactttatcttagaggcatatccctacgtaccaacaattctacaaccaggaccatgagacttgctcatggtcctggttgtagaatttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 100 - DNA, 4161 bp, synthetic construct
  misc_feature 1..4161: note = Description of Artificial Sequence: Synthetic polynucleotide
  source 1..4161: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaacaacaaggacgacgagaccttcatcaaacttgttgatgaaggtctcgtcgtccacaacaagtgcaatgagggaccagtacaacttgtgtactggtccctcattgcacacaacaattctacaaccaggaccatgagacttgctcatggtcctggttgtagaaacaacaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 101 - RNA, 807 nt, synthetic construct
  misc_feature 1..807: note = Description of Artificial Sequence: Synthetic polynucleotide
  source 1..807: mol_type = other RNA
  misc_difference 1..807: note = T in any of positions 1-807 are replaced with 1-methylpseudouridine.
  modified_base 1..807: mod_base = m1f
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagggcagaatcatcacgaagtggtgaagtacttgacttcaccacttcgtgatgattctgccctcctttatcttagaggcatatccctacgtaccaacaagagatgagcttcctacagcacaacaaatgtgacttgcacatttgttgtgctgtaggaagctcatctctttatcttagaggcatatccctacgtaccaacaagtacaagatccgcagacgtgtaaatgttccacttgggaacatttacacgtctgcggatcttgtactttatcttagaggcatatcccttttatcttagaggcatatccct

SEQ ID NO: 102 - RNA, 801 nt, synthetic construct
  misc_feature 1..801: note = Description of Artificial Sequence: Synthetic polynucleotide
  source 1..801: mol_type = other RNA
  misc_difference 1..801: note = T in any of positions 1-801 are replaced with 1-methylpseudouridine.
  modified_base 1..801: mod_base = m1f
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaacaacaaggagggcagaatcatcacgaagtggtgaagtacttgacttcaccacttcgtgatgattctgccctccacaacaagagatgagcttcctacagcacaacaaatgtgacttgcacatttgttgtgctgtaggaagctcatctcacaacaagtacaagatccgcagacgtgtaaatgttccacttgggaacatttacacgtctgcggatcttgtacacaacaatttatcttagaggcatatccctctgggcctcatgggccttccgctcactgcccgctttccagtcgggaaacctgtcgtgccagctgcattaacatggtcatagctg

SEQ ID NO: 103 - RNA, 735 nt, synthetic construct
  misc_feature 1..735: note = Description of Artificial Sequence: Synthetic polynucleotide
  source 1..735: mol_type = other RNA
  misc_difference 1..735: note = T in any of positions 1-735 are replaced with 1-methylpseudouridine.
  modified_base 1..735: mod_base = m1f
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaggagggcagaatcatcacgaagtggtgaagtacttgacttcaccacttcgtgatgattctgccctccatccctacgtaccaacaagagatgagcttcctacagcacaacaaatgtgacttgcacatttgttgtgctgtaggaagctcatctcatccctacgtaccaacaagtacaagatccgcagacgtgtaaatgttccacttgggaacatttacacgtctgcggatcttgtactttatcttagaggcat

SEQ ID NO: 104 - RNA, 723 nt, synthetic construct
  misc_feature 1..723: note = Description of Artificial Sequence: Synthetic polynucleotide
  source 1..723: mol_type = other RNA
  misc_difference 1..723: note = T in any of positions 1-723 are replaced with 1-methylpseudouridine.
  modified_base 1..723: mod_base = m1f
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaggagggcagaatcatcacgaagtggtgaagtacttgacttcaccacttcgtgatgattctgccctccacgtaccaacaagagatgagcttcctacagcacaacaaatgtgacttgcacatttgttgtgctgtaggaagctcatctcacgtaccaacaagtacaagatccgcagacgtgtaaatgttccacttgggaacatttacacgtctgcggatcttgtactttatcttagaggcat

SEQ ID NO: 105 - RNA, 711 nt, synthetic construct
  misc_feature 1..711: note = Description of Artificial Sequence: Synthetic polynucleotide
  source 1..711: mol_type = other RNA
  misc_difference 1..711: note = T in any of positions 1-711 are replaced with 1-methylpseudouridine.
  modified_base 1..711: mod_base = m1f
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattatcccggagggcagaatcatcacgaagtggtgaagtacttgacttcaccacttcgtgatgattctgccctcctcccgagatgagcttcctacagcacaacaaatgtgacttgcacatttgttgtgctgtaggaagctcatctctcccgtacaagatccgcagacgtgtaaatgttccacttgggaacatttacacgtctgcggatcttgtactttatcttagaggcat

SEQ ID NO: 106 - RNA, 732 nt, synthetic construct
  misc_feature 1..732: note = Description of Artificial Sequence: Synthetic polynucleotide
  source 1..732: mol_type = other RNA
  misc_difference 1..732: note = T in any of positions 1-732 are replaced with 1-methylpseudouridine.
  modified_base 1..732: mod_base = m1f
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacaacaatcccggagggcagaatcatcacgaagtggtgaagtacttgacttcaccacttcgtgatgattctgccctccacaacaatcccgagatgagcttcctacagcacaacaaatgtgacttgcacatttgttgtgctgtaggaagctcatctcacaacaatcccgtacaagatccgcagacgtgtaaatgttccacttgggaacatttacacgtctgcggatcttgtactttatcttagaggcat

SEQ ID NO: 107 - RNA, 1910 nt, synthetic construct
  misc_feature 1..1910: note = Description of Artificial Sequence: Synthetic polynucleotide
  source 1..1910: mol_type = other RNA
  misc_difference 1..1910: note = T in any of positions 1-1910 are replaced with 1-methylpseudouridine.
  modified_base 1..1910: mod_base = m1f
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaaggacgacgagaccttcatcaaacttgttgatgaaggtctcgtcgtcctttatcttagaggcatatccctacgtaccaacaagtgcaatgagggaccagtacaacttgtgtactggtccctcattgcactttatcttagaggcatatccctacgtaccaacaattctacaaccaggaccatgagacttgctcatggtcctggttgtagaatttatcttagaggcatatcccttttatcttagaggcatatccct

SEQ ID NO: 108 - RNA, 1820 nt, synthetic construct
  misc_feature 1..1820: note = Description of Artificial Sequence: Synthetic polynucleotide
  source 1..1820: mol_type = other RNA
  misc_difference 1..1820: note = T in any of positions 1-1820 are replaced with 1-methylpseudouridine.
  modified_base 1..1820: mod_base = m1f
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaacaacaaggacgacgagaccttcatcaaacttgttgatgaaggtctcgtcgtccacaacaagtgcaatgagggaccagtacaacttgtgtactggtccctcattgcacacaacaattctacaaccaggaccatgagacttgctcatggtcctggttgtagaaacaacaatttatcttagaggcatatccct

SEQ ID NO: 109 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 110 - protein, 133 aa, Homo sapiens [sequence omitted]

SEQ ID NO: 111 - DNA, 822 bp, Homo sapiens
  source 1..822: mol_type = genomic DNA
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 112 - protein, 20 aa, synthetic construct
  REGION 1..20: note = Description of Artificial Sequence: Synthetic peptide
MLLLLLACIALASTAAATNS

SEQ ID NO: 113 - DNA, 60 bp, Homo sapiens
  source 1..60: mol_type = other DNA
atgtacagaatgcagctgctgagctgtatcgccctgtctctggccctggtcacaaatagc

SEQ ID NO: 114 - DNA, 60 bp, synthetic construct
  misc_feature 1..60: note = Description of Artificial Sequence: Synthetic oligonucleotide
  source 1..60: mol_type = other DNA
atgttgttgctgctgctcgcctgtattgccctggcctctacagccgccgctacaaattct

SEQ ID NO: 115 - DNA, 699 bp, Homo sapiens
  source 1..699: mol_type = genomic DNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 116 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 117 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 118 - DNA, 1230 bp, Homo sapiens
  source 1..1230: mol_type = genomic DNA
atttcgctttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaaggg

SEQ ID NO: 119 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 120 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 121 - DNA, 1410 bp, Homo sapiens
  source 1..1410: mol_type = genomic DNA
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaat

SEQ ID NO: 122 - DNA, 1365 bp, Homo sapiens
  source 1..1365: mol_type = genomic DNA
atggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 123 - DNA, 567 bp, Homo sapiens
  source 1..567: mol_type = genomic DNA
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 124 - DNA, 1443 bp, Homo sapiens
  source 1..1443: mol_type = genomic DNA
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctga

SEQ ID NO: 125 - DNA, 1446 bp, Homo sapiens
  source 1..1446: mol_type = genomic DNA
atgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcggaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgagtga

SEQ ID NO: 126 - DNA, 1440 bp, Homo sapiens
  source 1..1440: mol_type = genomic DNA
atgagcgatgttaccattgtgaaagaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgctggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatatgatgaggatggtatggactgcatggacaatgagaggcggccgcatttccctcaattttcctactctgcaagtggacgagaataa

SEQ ID NO: 127 - DNA, 31 bp, synthetic construct
  misc_feature 1..31: note = Description of Artificial Sequence: Synthetic oligonucleotide
  source 1..31: mol_type = other DNA
ggagggcagaatcatcacgaagtggtgaagt

SEQ ID NO: 128 - DNA, 31 bp, synthetic construct
  misc_feature 1..31: note = Description of Artificial Sequence: Synthetic oligonucleotide
  source 1..31: mol_type = other DNA
gagatgagcttcctacagcacaacaaatgtg

SEQ ID NO: 129 - DNA, 30 bp, synthetic construct
  misc_feature 1..30: note = Description of Artificial Sequence: Synthetic oligonucleotide
  source 1..30: mol_type = other DNA
gtacaagatccgcagacgtgtaaatgttcc

SEQ ID NO: 130 - DNA, 19 bp, synthetic construct
  misc_feature 1..19: note = Description of Artificial Sequence: Synthetic oligonucleotide
  source 1..19: mol_type = other DNA
acgacgagaccttcatcaa

SEQ ID NO: 131 - DNA, 21 bp, synthetic construct
  misc_feature 1..21: note = Description of Artificial Sequence: Synthetic oligonucleotide
  source 1..21: mol_type = other DNA
gtgcaatgagggaccagtaca

SEQ ID NO: 132 - DNA, 21 bp, synthetic construct
  misc_feature 1..21: note = Description of Artificial Sequence: Synthetic oligonucleotide
  source 1..21: mol_type = other DNA
ttctacaaccaggaccatgag

SEQ ID NO: 133 - DNA, 31 bp, synthetic construct
  misc_feature 1..31: note = Description of Artificial Sequence: Synthetic oligonucleotide
  source 1..31: mol_type = other DNA
acttcaccacttcgtgatgattctgccctcc

SEQ ID NO: 134 - DNA, 31 bp, synthetic construct
  misc_feature 1..31: note = Description of Artificial Sequence: Synthetic oligonucleotide
  source 1..31: mol_type = other DNA
cacatttgttgtgctgtaggaagctcatctc

SEQ ID NO: 135 - DNA, 30 bp, synthetic construct
  misc_feature 1..30: note = Description of Artificial Sequence: Synthetic oligonucleotide
  source 1..30: mol_type = other DNA
ggaacatttacacgtctgcggatcttgtac

SEQ ID NO: 136 - DNA, 21 bp, synthetic construct
  misc_feature 1..21: note = Description of Artificial Sequence: Synthetic oligonucleotide
  source 1..21: mol_type = other DNA
ttgatgaaggtctcgtcgtcc

SEQ ID NO: 137 - DNA, 21 bp, synthetic construct
  misc_feature 1..21: note = Description of Artificial Sequence: Synthetic oligonucleotide
  source 1..21: mol_type = other DNA
tgtactggtccctcattgcac

SEQ ID NO: 138 - DNA, 21 bp, synthetic construct
  misc_feature 1..21: note = Description of Artificial Sequence: Synthetic oligonucleotide
  source 1..21: mol_type = other DNA
ctcatggtcctggttgtagaa

SEQ ID NO: 139 - DNA, 21 bp, synthetic construct
  misc_feature 1..21: note = Description of Artificial Sequence: Synthetic primer
  source 1..21: mol_type = other DNA
gtacagtgcaatgagggacca

SEQ ID NO: 140 - DNA, 20 bp, synthetic construct
  misc_feature 1..20: note = Description of Artificial Sequence: Synthetic primer
  source 1..20: mol_type = other DNA
cacaaagaaagccctcccca

SEQ ID NO: 141 - DNA, 19 bp, synthetic construct
  misc_feature 1..19: note = Description of Artificial Sequence: Synthetic primer
  source 1..19: mol_type = other DNA
gagatggacttccggtcgg

SEQ ID NO: 142 - DNA, 17 bp, synthetic construct
  misc_feature 1..17: note = Description of Artificial Sequence: Synthetic primer
  source 1..17: mol_type = other DNA
gtcacgcggtgcttggg

SEQ ID NO: 143 - DNA, 20 bp, synthetic construct
  misc_feature 1..20: note = Description of Artificial Sequence: Synthetic primer
  source 1..20: mol_type = other DNA
cgaggaccccatggactaca

SEQ ID NO: 144 - DNA, 19 bp, synthetic construct
  misc_feature 1..19: note = Description of Artificial Sequence: Synthetic primer
  source 1..19: mol_type = other DNA
tttagcccgtgccttgctg

SEQ ID NO: 145 - DNA, 23 bp, synthetic construct
  misc_feature 1..23: note = Description of Artificial Sequence: Synthetic primer
  source 1..23: mol_type = other DNA
ggcaagaagaggagagaatgaat

SEQ ID NO: 146 - DNA, 21 bp, synthetic construct
  misc_feature 1..21: note = Description of Artificial Sequence: Synthetic primer
  source 1..21: mol_type = other DNA
tgggttgtagaggcatccatc

SEQ ID NO: 147 - DNA, 20 bp, synthetic construct
  misc_feature 1..20: note = Description of Artificial Sequence: Synthetic primer
  source 1..20: mol_type = other DNA
actgtatgtggagcggcttc

SEQ ID NO: 148 - DNA, 20 bp, synthetic construct
  misc_feature 1..20: note = Description of Artificial Sequence: Synthetic primer
  source 1..20: mol_type = other DNA
caggtacaagctggaggtgg

SEQ ID NO: 149 - DNA, 22 bp, synthetic construct
  misc_feature 1..22: note = Description of Artificial Sequence: Synthetic primer
  source 1..22: mol_type = other DNA
acccgttgaaccccattcgtga

SEQ ID NO: 150 - DNA, 23 bp, synthetic construct
  misc_feature 1..23: note = Description of Artificial Sequence: Synthetic primer
  source 1..23: mol_type = other DNA
gcctcactaaaccatccaatcgg

SEQ ID NO: 151-152 - skipped (2 entries)

SEQ ID NO: 153 - RNA, 220 nt, synthetic construct
  misc_feature 1..220: note = Description of Artificial Sequence: Synthetic polynucleotide
  misc_feature 1..220: note = This sequence may encompass 1-220 nucleotides
  misc_feature 1..220: note = See specification as filed for detailed description of substitutions and preferred embodiments
  source 1..220: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 154 - RNA, 200 nt, synthetic construct
  misc_feature 1..200: note = Description of Artificial Sequence: Synthetic polynucleotide
  misc_feature 1..200: note = This sequence may encompass 1-200 nucleotides
  misc_feature 1..200: note = See specification as filed for detailed description of substitutions and preferred embodiments
  source 1..200: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 155 - RNA, 120 nt, synthetic construct
  misc_feature 1..120: note = Description of Artificial Sequence: Synthetic polynucleotide
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 156 - DNA, 120 bp, synthetic construct
  misc_feature 1..120: note = Description of Artificial Sequence: Synthetic polynucleotide
  source 1..120: mol_type = other DNA
tttttttttttttttttttttttttttttttttttttttttttttttttttttttttttttttttttttttttttttttttttttttttttttttttttttttttttttttttttttttt